(19) United States
(12) Patent Application Publication
(10) Pub. No.: US 2022/0064267 A1
(43) Pub. Date: Mar. 3, 2022

(54) ANTIBODIES AND METHODS FOR TREATMENT OF LYSSAVIRUS INFECTION

(71) Applicants: HUMABS BIOMED SA, Bellinzona (CN); INSTITUT PASTEUR, Paris (FR)

(72) Inventors: Davide CORTI, Bellinzona (CH); Hervé BOURHY, Chaville (FR); Florian SONTHONAX, Cachan (FR); Gabriel LEPOUSEZ, Chaville (FR); Guilherme DIAS DE MELO, Paris (FR)

(21) Appl. No.: 17/286,406

(22) PCT Filed: Oct. 18, 2019
(86) PCT No.: PCT/EP2019/078439

§ 371 Date: Apr. 16, 2021

(30) Foreign Application Priority Data
Oct. 19, 2018 (EP) PCT/EP2018/078751

(51) Int. Cl. C07K 16/10; A61K 45/06; A61P 31/14

(52) U.S. Cl. CPC C07K 16/10; A61P 31/14; A61K 45/06

(57) ABSTRACT

The invention provides antibodies, and antigen-binding fragments thereof, that potently neutralize lyssavirus infection and the use of such antibodies. In particular, the invention provides methods of treatment of lyssavirus infection, such as rabies.

The invention relates to antibodies, and antigen-binding fragments thereof, that potently neutralize lyssavirus infection and to the use of such antibodies. In particular, the invention relates to the treatment of lyssavirus infection, such as rabies.

Rabies is a viral infection, that causes acute inflammation of the brain. Accordingly, rabies is a neurotropic viral disease. Rabies is distributed nearly worldwide and is commonly transmitted to humans from the bite of wild or domestic infected animals, resulting in a devastating disease, which is nearly 100% invariably fatal in individuals who do not receive post-exposure prophylaxis (PEP). Although rabies is preventable with PEP, no proven cure exists after the onset of symptoms (Manning S E, Rupprecht C E, Fishbein D, Recomm C H M, 2008. Human rabies prevention-United states, 2008; Dacheux L, Delmas O, Bourhy H. Human rabies encephalitis prevention and treatment: progress since Pasteur's discovery. Infect Disord Drug Targets. 2011 June; 11(3):251-99). Even with advanced supportive care, the case-fatality rate approaches 100% (Brett W. Petersen and Charles E. Rupprecht, 2011. Human Rabies Epidemiology and Diagnosis, Non-Flavivirus Encephalitis, Dr. Sergey Tkachev (Ed.), InTech, DOI: 10.5772/21708). Consequently, management approaches generally focus on palliation (Jackson A C, Warrell M J, Rupprecht C E, Ertl H C J, Dietzschold B, O'Reilly M, Leach R P, Fu Z F, Wunner W H, Bleck T P, Wilde H. 2003. Management of rabies in humans. Clinical infectious diseases: an official publication of the Infectious Diseases Society of America 36:60-63; Tarantola A, Crabol Y, Mahendra B J, In S, Barennes H, Bourhy H, Peng Y, Ly S, Buchy P. Caring for patients with rabies in developing countries—the neglected importance of palliative care. Trop Med Int Health. 2016 April; 21(4):564-7).

Early symptoms of rabies can include fever, anorexia, nausea, local pain at the site of bite, agitation and depression. These symptoms are followed by one or more of the following symptoms: violent movements, uncontrolled excitement, fear of water, aerophobia, confusion, hyperactivity, an inability to move parts of the body, paralysis, and loss of consciousness. After symptoms appear, rabies almost always results in death. The time between contracting the disease and the start of symptoms can vary from less than one week to more than one year. The time frame typically depends on the distance the virus must travel to reach the central nervous system (Ugolini G, Hemachudha T. Rabies: changing prophylaxis and new insights in pathophysiology. Curr Opin Infect Dis. 2018 February; 31(1):93-101).

Rabies is widespread across the globe and approximately 15 to 29 million people a year are treated after exposure to rabies, usually following a bite from infected animals (dogs, bats, foxes, cats, monkeys raccoons, skunks, cattle, wolves, coyotes and others domestic and wild animals). Some 40,000 to 70,000 people are estimated to die of the disease each year, mainly in Africa, China and India, and 50% cases of rabies worldwide occur in children. These data highlight the significant unmet medical need for a safe, effective and affordable rabies treatment (Hampson K, Coudeville L, Lembo T, Sambo M, Kieffer A, Attlan M, Barrat J, Blanton J D, Briggs D J, Cleaveland S, Costa P, Freuling C M, Hiby E, Knopf L, Leanes F, Meslin F X, Metlin A, Miranda M E, Müller T, Nel L H, Recuenco S, Rupprecht C E, Schumacher C, Taylor L, Vigilato M A, Zinsstag J, Dushoff J; Global Alliance for Rabies Control Partners for Rabies Prevention. Estimating the global burden of endemic canine rabies. PLoS Negl Trop Dis. 2015 Apr. 16; 9(4):e0003709).

Rabies prevention is achieved either by pre- or post-exposure vaccination, mostly using modern, tissue culture-based vaccines. Immunizing before exposure (Pre-exposure prophylaxis (PrEP)) is recommended for those who are at high risk and is achieved by administration of a rabies vaccine (active immunization). The high-risk group includes people who work with bats or who spend prolonged periods in areas of the world where rabies is common. Furthermore, the anti-rabies vaccine is recommended for people travelling to countries in Africa and Asia, where rabies is endemic.

Currently available rabies vaccines for humans mainly comprise range inactivated vaccines, while live attenuated vaccine are still in use for the vaccination of wildlife. Human vaccines mainly comprise human diploid cell vaccine (HDCV), chicken embryo cell vaccine (PCEC) and Vero cell vaccines. Novel vaccines are in development and comprises recombinant vaccines, DNA vaccines and RNA (Alberer M, Gnad-Vogt U, Hong H S, Mehr K T, Backert L, Finak G, Gottardo R, Bica M A, Garofano A, Koch S D, Fotin-Mleczek M, Hoerr I, Clemens R, Sonnenburg von F, 2017. Safety and immunogenicity of a mRNA rabies vaccine in healthy adults: an open-label, non-randomised, prospective, first-in-human phase 1 clinical trial. Lancet 390:1511-1520).

After exposure to the virus, a post-exposure prophylaxis (PEP) with the rabies vaccine and a rabies immunoglobulin (RIG) are the standard treatment preventing the disease, if the person receives the treatment as early as possible after infection, i.e. during the first days after the infection. If left untreated until the start of the symptoms, rabies is nearly 100% fatal. Thus, currently, there is no therapeutic treatment for rabies.

Accordingly, when someone is assumed to be infected by a lyssavirus, post-exposure prophylaxis (PEP) is administered as “standard treatment”, which combines rabies immunoglobulin (RIG), in particular human or equine rabies immunoglobulins (HRIG and ERIG, respectively), with a rabies vaccine. In particular, patients receive one dose of RIG (passive immunization) and several doses of rabies vaccine (active immunization) according to the information of the rabies vaccine manufacturer. In a widely used standard therapy, for example, five doses of the vaccine are administered over a twenty-eight day period, i.e. the first dose of rabies vaccine is given as soon as possible after exposure, preferably day 0, with additional doses on days 3, 7, 14, and 28 after the first (cf. http://www.rki.de/DE/Content/Infekt/EpidBull/Merkblaetter/Ratgeber_Tollwut.html, retrieved at Nov. 12, 2014). Very recently, these recommendations have been revised and shorter course of vaccine administration are now being promoted (Rabies vaccines: WHO position paper—April 2018. Editors: Dr B. Abela Ridder/Neglected Zoonotic Diseases. Number of pages: 18 p. Publication date: 20 Apr. 2018. WHO reference number: No 16, 2018, 93, 201-220). In contrast, rabies immunoglobulin (RIG) for passive immunization is administered only once, preferably at, or as soon as possible after, the initiation of post-exposure vaccination. The dose of human rabies immunoglobulin (HRIG) proposed by the WHO is 20 IU/kg body weight; for equine immunoglobulin (ERIG) and F(ab′)2 products it is 40 IU/kg body weight (cf. http://www.who.int/rabies/human/WHO_strategy_prepost_exposure/en/index1.html #, retrieved at Nov. 12, 2014). Higher doses can reduce vaccine efficacy. All of the rabies immunoglobulins, or as much as anatomically possible to avoid possible compartment syndrome, should be administered into or around the wound site or sites. The remaining immunoglobulin, if any, should be injected intramuscularly at a site distant from the site of vaccine administration. Rabies immunoglobulin may be diluted to a volume sufficient for all wounds to be effectively and safely infiltrated (cf. http://www.who.int/rabies/human/WHO_strategy_prepost_exposure/en/index1.html#, retrieved at Nov. 12, 2014). This is usually successful if administered up to 24-48 hours following exposure. HRIG is widely used, especially in developed countries, and is considered safer than ERIG. The high cost of HRIG and its limited availability prohibit its wide use in developing countries. Moreover, the vaccine and HRIG or ERIG do not effectively protect against infection with different lyssavirus species (protection is inversely related to the genetic distance with the vaccine strain).

Several attempts have tried to treat symptomatic rabies. In 2004 a young patient from Wisconsin survived rabies and her therapy has been dubbed the “Milwaukee protocol” (Willoughby R E, Tieves K S, Hoffman G M, Ghanayem N S, Amlie-Lefond C M, Schwabe M J, Chusid M J, Rupprecht C E. 2005. Survival after treatment of rabies with induction of coma. N Engl J Med 352:2508-2514). The Milwaukee protocol is a treatment regimen for rabies focused on therapeutic coma and the use of N-methyl D-aspartate (NMDA) receptor antagonist therapy. Since the case report was published in 2005, four changes have been made in the protocol to arrive at its current version that includes therapeutic coma, ketamine infusion, amantadine, and the screening/prophylaxis/management of cerebral vasospasm. Critics of the Milwaukee protocol raise concerns of the regimen's lack of efficacy in human rabies, with at least 31 documented failures reported in the literature to date (Zeiler F A, Jackson A C. 2016. Critical Appraisal of the Milwaukee Protocol for Rabies: This Failed Approach Should Be Abandoned. Can J Neurol Sci 43:44-51). Despite initial hope and enthusiasm for the Milwaukee protocol in the treatment of rabies, subsequent trials of this regimen have failed.

In a recent animal study, Yamada and co-authors have investigated the efficacy of Favipiravir (T-507) against RABV (Yamada K, Noguchi K, Komeno T, Furuta Y, Nishizono A. 2016. Efficacy of Favipiravir (T-705) in Rabies Postexposure Prophylaxis. J Infect Dis 213:1253-1261). Favipiravir is predicted to act on viral RNA-dependent RNA polymerase as a chain terminator or mutagen. In this study, Favipiravir was demonstrated to be a potential alternative to rabies immunoglobulin in rabies PEP, but even at a high dose of 300 mg/kg/day this antiviral drug was shown to have little to no effect against RABV infection when administration started 1 or 2 days after viral inoculation.

WO 2016/078761 and De Benedictis et al. (De Benedictis P, Minola A, Rota Nodari E, Aiello R, Zecchin B, Salomoni A, Foglierini M, Agatic G, Vanzetta F, Lavenir R, Lepelletier A, Bentley E, Weiss R, Cattoli G, Capua I, Sallusto F, Wright E, Lanzavecchia A, Bourhy H, Corti D. 2016. Development of broad-spectrum human monoclonal antibodies for rabies post-exposure prophylaxis. EMBO Mol Med 8:407-421) reported on the selection of two highly potent human monoclonal antibodies (RVC58 and RVC20) with the ability to broadly neutralize a large variety of lyssaviruses within a narrow and similar range of antibody concentrations, in contrast to other monoclonal antibodies described in literature (such as CR57, CR4098 and RAB1).

Since there is currently no therapy for lyssavirus infection, there is a need for effective treatment of lyssavirus infection, even if the exposure to the virus was more than 5 days before the first treatment. The development of such a treatment would be of benefit in particular for at least two classes of patients: those with known exposure to a lyssavirus but who have failed to receive prompt post-exposure prophylaxis due to circumstances and who are at increased risk of developing lyssavirus infection, and those who did not recognize contact with the virus and/or present signs (of different severity) of the disease (e.g. individuals infected by unnoticed contacts with infected bats; for example, where dog rabies is controlled RABV of bat origin has become the leading cause of human rabies). In these individuals the lyssavirus might have already reached the CNS tissues and early or late signs of the disease might have also appeared.

In view of the above, it is the object of the invention to provide a new treatment for lyssavirus infection, even if the subject already shows symptoms or if the exposure to the virus was more than 5 days before the first administration. Moreover, it is also an object of the invention to expand the post-exposure treatment window for lyssavirus infection, that is currently limited to the first days after infection. In addition, it is also an object of the invention to provide potent antibodies broadly neutralizing a large variety of lyssaviruses at low doses, for treatment of lyssavirus infection in post-exposure prophylaxis and/or even if the initial administration is more than five days after virus exposure.

This object is achieved by means of the subject-matter set out below and in the appended claims.

### Items of the Invention

The present invention provides in particular the following items:
1. An anti-lyssavirus antibody, or an antigen-binding fragment thereof, for use in the treatment of lyssavirus infection, wherein the antibody, or the antigen-binding fragment thereof, is administered
   (i) into the central nervous system (CNS); and
   (ii) peripherally.
2. The anti-lyssavirus antibody, or the antigen-binding fragment thereof, for use according to item 1, wherein the antibody, or the antigen-binding fragment thereof, is administered for the first time at least five days after exposure to a lyssavirus.
3. The anti-lyssavirus antibody, or the antigen-binding fragment thereof, for use according to item 1, wherein the antibody, or the antigen-binding fragment thereof, is administered for the first time at least six days after exposure to a lyssavirus.
4. The anti-lyssavirus antibody, or the antigen-binding fragment thereof, for use according to item 1, wherein the antibody, or the antigen-binding fragment thereof, is administered for the first time at least seven days after exposure to a lyssavirus.
5. The anti-lyssavirus antibody, or the antigen-binding fragment thereof, for use according to item 1, wherein the antibody, or the antigen-binding fragment thereof, is administered for the first time at least eight days after exposure to a lyssavirus.
6. The anti-lyssavirus antibody, or the antigen-binding fragment thereof, for use according to any one of items 1-5, wherein the antibody, or the antigen-binding fragment thereof, is administered for the first time after symptoms of lyssavirus infection occur.
7. The anti-lyssavirus antibody, or the antigen-binding fragment thereof, for use according to any one of items 1-6, wherein the peripheral administration of the antibody, or the antigen-binding fragment thereof, and the administration of the antibody, or the antigen-binding fragment thereof, into the CNS is performed at about the same time (simultaneously).
8. The anti-lyssavirus antibody, or the antigen-binding fragment thereof, for use according to any one of items 1-6, wherein the peripheral administration of the antibody, or the antigen-binding fragment thereof, and the administration of the antibody, or the antigen-binding fragment thereof, into the CNS is performed consecutively (sequentially).
9. The anti-lyssavirus antibody, or the antigen-binding fragment thereof, for use according to item 8, wherein the time between the peripheral administration of the antibody, or the antigen-binding fragment thereof, and the administration of the antibody, or the antigen-binding fragment thereof, into the CNS is no more than one week.
10. The anti-lyssavirus antibody, or the antigen-binding fragment thereof, for use according to item 9, wherein the time between the peripheral administration of the antibody, or the antigen-binding fragment thereof, and the administration of the antibody, or the antigen-binding fragment thereof, into the CNS is no more than 3 days.
11. The anti-Lyssavirus antibody, or the antigen-binding fragment thereof, for use according to item 10, wherein the time between the peripheral administration of the antibody, or the antigen-binding fragment thereof, and the administration of the antibody, or the antigen-binding fragment thereof, into the CNS is no more than 2 days.
12. The anti-lyssavirus antibody, or the antigen-binding fragment thereof, for use according to item 11, wherein the time between the peripheral administration of the antibody, or the antigen-binding fragment thereof, and the administration of the antibody, or the antigen-binding fragment thereof, into the CNS is no more than 24 h.
13. The anti-lyssavirus antibody, or the antigen-binding fragment thereof, for use according to any one of items 1-12, wherein the antibody, or the antigen-binding fragment thereof, is administered peripherally while the antibody, or the antigen-binding fragment thereof, is administered into the CNS.
14. The anti-lyssavirus antibody, or the antigen-binding fragment thereof, for use according to any one of items 1-13, wherein the first peripheral administration of the antibody, or the antigen-binding fragment thereof, and the first administration of the antibody, or the antigen-binding fragment thereof, into the CNS occur on the same day.
15. The anti-lyssavirus antibody, or the antigen-binding fragment thereof, for use according to any one of items 1-14, comprising a single peripheral administration of the antibody, or the antigen-binding fragment thereof.
16. The anti-lyssavirus antibody, or the antigen-binding fragment thereof, for use according to any one of items 1-14, wherein the antibody, or the antigen-binding fragment thereof, is repeatedly administered peripherally.
17. The anti-lyssavirus antibody, or the antigen-binding fragment thereof, for use according to any one of items 1-16, wherein the antibody, or the antigen-binding fragment thereof, is administered into the CNS continuously for at least 15 min.
18. The anti-lyssavirus antibody, or the antigen-binding fragment thereof, for use according to any one of items 1-16, wherein the antibody, or the antigen-binding fragment thereof, is administered into the CNS continuously for or at least 30 min.
19. The anti-lyssavirus antibody, or the antigen-binding fragment thereof, for use according to any one of items 1-16, wherein the antibody, or the antigen-binding fragment thereof, is administered into the CNS continuously for at least 1 h.
20. The anti-lyssavirus antibody, or the antigen-binding fragment thereof, for use according to any one of items 1-16, wherein the antibody, or the antigen-binding fragment thereof, is administered into the CNS continuously for at least 6 h.
21. The anti-lyssavirus antibody, or the antigen-binding fragment thereof, for use according to any one of items 1-16, wherein the antibody, or the antigen-binding fragment thereof, is administered into the CNS continuously for at least 12 h.
22. The anti-lyssavirus antibody, or the antigen-binding fragment thereof, for use according to any one of items 1-16, wherein the antibody, or the antigen-binding fragment thereof, is administered into the CNS continuously for at least 24 hours.
23. The anti-lyssavirus antibody, or the antigen-binding fragment thereof, for use according to any one of items 1-16, wherein the antibody, or the antigen-binding fragment thereof, is administered into the CNS continuously for at least 2 days.
24. The anti-lyssavirus antibody, or the antigen-binding fragment thereof, for use according to any one of items 1-16, wherein the antibody, or the antigen-binding fragment thereof, is administered into the CNS continuously for at least 3 days.
25. The anti-lyssavirus antibody, or the antigen-binding fragment thereof, for use according to any one of items 1-16, wherein the antibody, or the antigen-binding fragment thereof, is administered into the CNS continuously for at least 4 days.
26. The anti-lyssavirus antibody, or the antigen-binding fragment thereof, for use according to any one of items 1-16, wherein the antibody, or the antigen-binding fragment thereof, is administered into the CNS continuously for at least 5 days.
27. The anti-lyssavirus antibody, or the antigen-binding fragment thereof, for use according to any one of items 1-26, wherein the antibody, or the antigen-binding fragment thereof, is administered into the CNS daily or every second day for at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days.
28. The anti-lyssavirus antibody, or the antigen-binding fragment thereof, for use according to any one of items 1-27, wherein the antibody, or the antigen-binding fragment thereof, administered into the CNS is administered into brain tissue.
29. The anti-lyssavirus antibody, or the antigen-binding fragment thereof, for use according to any one of items 1-27, wherein the antibody, or the antigen-binding fragment thereof, administered into the CNS is administered into spinal cord tissue.
30. The anti-lyssavirus antibody, or the antigen-binding fragment thereof, for use according to any one of items 1-27, wherein the antibody, or the antigen-binding fragment thereof, administered into the CNS is administered into cerebro-spinal fluid (intra-CSF).
31. The anti-lyssavirus antibody, or the antigen-binding fragment thereof, for use according to any one of items 1-27, wherein administration into the CNS is selected from intrathecal, intracerebroventricular, intracerebral, epidural, transnasal, intranasal, and perispinal administration.
32. The anti-lyssavirus antibody, or the antigen-binding fragment thereof, for use according to item 31, wherein the antibody, or the antigen-binding fragment thereof, administered into the CNS is administered intrathecally.
33. The anti-lyssavirus antibody, or the antigen-binding fragment thereof, for use according to item 31, wherein the antibody, or the antigen-binding fragment thereof, administered into the CNS is administered intracerebroventricularly.
34. The anti-lyssavirus antibody, or the antigen-binding fragment thereof, for use according to item 31, wherein the antibody, or the antigen-binding fragment thereof, administered into the CNS is administered intracerebrally.
35. The anti-lyssavirus antibody, or the antigen-binding fragment thereof, for use according to item 31, wherein the antibody, or the antigen-binding fragment thereof, administered into the CNS is administered epidurally.
36. The anti-lyssavirus antibody, or the antigen-binding fragment thereof, for use according to item 31, wherein the antibody, or the antigen-binding fragment thereof, administered into the CNS is administered transnasally.
37. The anti-lyssavirus antibody, or the antigen-binding fragment thereof, for use according to item 31, wherein the antibody, or the antigen-binding fragment thereof, administered into the CNS is administered intranasally.
38. The anti-lyssavirus antibody, or the antigen-binding fragment thereof, for use according to item 31, wherein the antibody, or the antigen-binding fragment thereof, administered into the CNS is administered perispinally.
39. The anti-lyssavirus antibody, or the antigen-binding fragment thereof, for use according to any one of items 1-27, wherein the antibody, or the antigen-binding fragment thereof, administered into the CNS is administered transocularly.
40. The anti-lyssavirus antibody, or the antigen-binding fragment thereof, for use according to any one of items 1-39, wherein the antibody, or the antigen-binding fragment thereof, is administered into the CNS by injection.
41. The anti-lyssavirus antibody, or the antigen-binding fragment thereof, for use according to any one of items 1-40, wherein the antibody, or the antigen-binding fragment thereof, is administered into the CNS by infusion.
42. The anti-lyssavirus antibody, or the antigen-binding fragment thereof, for use according to any one of items 1-39, wherein the antibody, or the antigen-binding fragment thereof, is administered into the CNS by nasal devices.
43. The anti-lyssavirus antibody, or the antigen-binding fragment thereof, for use according to item 42, wherein the antibody, or the antigen-binding fragment thereof, is administered into the CNS by spray.
44. The anti-lyssavirus antibody, or the antigen-binding fragment thereof, for use according to item 42, wherein the antibody, or the antigen-binding fragment thereof, is administered into the CNS by nose droppers.
45. The anti-lyssavirus antibody, or the antigen-binding fragment thereof, for use according to item 42, wherein the antibody, or the antigen-binding fragment thereof, is administered into the CNS by a needleless syringe.
46. The anti-lyssavirus antibody, or the antigen-binding fragment thereof, for use according to item 42, wherein the antibody, or the antigen-binding fragment thereof, is administered into the CNS by a nasal device designed to deposit the nasally applied formulation specifically to olfactory region.
47. The anti-lyssavirus antibody, or the antigen-binding fragment thereof, for use according to any one of items 1-46, wherein the peripherally administered antibody, or the antigen-binding fragment thereof, is administered systemically.
48. The anti-Lyssavirus antibody, or the antigen-binding fragment thereof, for use according to any one of items 1-46, wherein the peripherally administered antibody, or the antigen-binding fragment thereof, is administered locally.
49. The anti-lyssavirus antibody, or the antigen-binding fragment thereof, for use according to any one of items 1-46, wherein the peripherally administered antibody, or the antigen-binding fragment thereof, is administered enterally.
50. The anti-lyssavirus antibody, or the antigen-binding fragment thereof, for use according to any one of items 1-46, wherein the peripherally administered antibody, or the antigen-binding fragment thereof, is administered parenterally.
51. The anti-lyssavirus antibody, or the antigen-binding fragment thereof, for use according to item 49, wherein the peripherally administered antibody, or the antigen-binding fragment thereof, is administered orally.
52. The anti-lyssavirus antibody, or the antigen-binding fragment thereof, for use according to item 49, wherein the peripherally administered antibody, or the antigen-binding fragment thereof, is administered rectally.
53. The anti-lyssavirus antibody, or the antigen-binding fragment thereof, for use according to item 50, wherein the peripherally administered antibody, or the antigen-binding fragment thereof, is administered intravenously.
54. The anti-lyssavirus antibody, or the antigen-binding fragment thereof, for use according to item 50, wherein the peripherally administered antibody, or the antigen-binding fragment thereof, is administered intramuscularly.
55. The anti-lyssavirus antibody, or the antigen-binding fragment thereof, for use according to item 50, wherein the peripherally administered antibody, or the antigen-binding fragment thereof, is administered subcutaneously.
56. The anti-lyssavirus antibody, or the antigen-binding fragment thereof, for use according to item 50, wherein the peripherally administered antibody, or the antigen-binding fragment thereof, is administered intradermally.
57. The anti-lyssavirus antibody, or the antigen-binding fragment thereof, for use according to item 50, wherein the peripherally administered antibody, or the antigen-binding fragment thereof, is administered intraarterially.
58. The anti-lyssavirus antibody, or the antigen-binding fragment thereof, for use according to item 50, wherein the peripherally administered antibody, or the antigen-binding fragment thereof, is administered transdermally.
59. The anti-lyssavirus antibody, or the antigen-binding fragment thereof, for use according to item 50, wherein the peripherally administered antibody, or the antigen-binding fragment thereof, is administered intraperitoneally.
60. The anti-lyssavirus antibody, or the antigen-binding fragment thereof, for use according to any one of items 1-59, wherein the peripherally administered antibody, or the antigen-binding fragment thereof, is administered at the site of infliction.
61. The anti-lyssavirus antibody, or the antigen-binding fragment thereof, for use according to item 60, wherein the peripherally administered antibody, or the antigen-binding fragment thereof, is administered in the same muscle or the corresponding skin where the bite occurred.
62. The anti-lyssavirus antibody, or the antigen-binding fragment thereof, for use according to any one of items 1-48, 60 and 61, wherein the peripherally administered antibody, or the antigen-binding fragment thereof, is administered intramuscularly, intravenously, intradermally, or subcutaneously.
63. The anti-lyssavirus antibody, or the antigen-binding fragment thereof, for use according to any one of items 1-62, wherein the same antibody, or antigen-binding fragment thereof, is administered peripherally and into the CNS.
64. The anti-lyssavirus antibody, or the antigen-binding fragment thereof, for use according to any one of items 1-62, wherein distinct antibodies, or antigen-binding fragments thereof, are administered peripherally and into the CNS.
65. The anti-lyssavirus antibody, or the antigen-binding fragment thereof, for use according to any one of items 1-64, wherein the lyssavirus infection to be treated is rabies and the anti-lyssavirus antibody, or an antigen-binding fragment thereof, is an anti-RABV antibody or an antigen-binding fragment thereof.
66. The anti-lyssavirus antibody, or the antigen-binding fragment thereof, for use according to any one of items 1-65, wherein the antibody, or the antigen-binding fragment thereof, binds to lyssavirus glycoprotein G.
67. The anti-lyssavirus antibody, or the antigen-binding fragment thereof, for use according to item 66, wherein the antibody, or the antigen-binding fragment thereof, binds to glycoprotein G of RABV.
68. The anti-lyssavirus antibody, or the antigen-binding fragment thereof, for use according to item 67, wherein the antibody, or the antigen-binding fragment thereof, binds to antigenic site I or antigenic site III of glycoprotein G of RABV.
69. The anti-lyssavirus antibody, or the antigen-binding fragment thereof, for use according to item 68, wherein the antibody, or the antigen-binding fragment thereof, binds to antigenic site I of glycoprotein G of RABV.
70. The anti-lyssavirus antibody, or the antigen-binding fragment thereof, for use according to item 68, wherein the antibody, or the antigen-binding fragment thereof, binds to antigenic site III of glycoprotein G of RABV.
71. The anti-lyssavirus antibody, or the antigen-binding fragment thereof, for use according to item 70, wherein the antibody, or the antigen-binding fragment thereof, binds to an epitope, which at least partially overlaps with antigenic site III of glycoprotein G of RABV.
72. The anti-lyssavirus antibody, or the antigen-binding fragment thereof, for use according to any one of items 1-71, wherein the antibody, or the antigen-binding fragment thereof, is administered in combination with a further anti-lyssavirus antibody, or an antigen-binding fragment thereof.
73. The anti-lyssavirus antibody, or the antigen-binding fragment thereof, for use according to item 72, wherein the two anti-lyssavirus antibodies, or the antigen-binding fragments thereof, administered in combination are administered in the same pharmaceutical composition.
74. The anti-lyssavirus antibody, or the antigen-binding fragment thereof, for use according to item 72, wherein the two anti-lyssavirus antibodies, or the antigen-binding fragments thereof, administered in combination are administered in distinct pharmaceutical compositions.
75. The anti-lyssavirus antibody, or the antigen-binding fragment thereof, for use according to any one of items 72-74, wherein the two anti-Lyssavirus antibodies, or the antigen-binding fragments thereof, administered in combination into the CNS are the same antibodies, or the antigen-binding fragments thereof, as the two anti-lyssavirus antibodies, or the antigen-binding fragments thereof, administered in combination peripherally.
76. The anti-lyssavirus antibody, or the antigen-binding fragment thereof, for use according to any one of items 72-74, wherein the combination of two anti-lyssavirus antibodies, or the antigen-binding fragments thereof, administered into the CNS is distinct from the combination of two anti-lyssavirus antibodies, or the antigen-binding fragments thereof, administered peripherally.
77. The anti-lyssavirus antibody, or the antigen-binding fragment thereof, for use according to item 76, wherein the two anti-lyssavirus antibodies, or the antigen-binding fragments thereof, administered into the CNS are distinct from the two anti-lyssavirus antibodies, or the antigen-binding fragments thereof, administered peripherally.
78. The anti-lyssavirus antibody, or the antigen-binding fragment thereof, for use according to any one of items 72-77, wherein the two anti-lyssavirus antibodies, or the antigen-binding fragments thereof, administered in combination bind specifically to different epitopes on the glycoprotein G of RABV.
79. The anti-lyssavirus antibody, or the antigen-binding fragment thereof, for use according to item 78, wherein one of the two anti-lyssavirus antibodies, or the antigen-binding fragments thereof, binds to antigenic site I of glycoprotein G of RABV and the other of the two anti-lyssavirus antibodies, or the antigen-binding fragments thereof, binds to antigenic site III of glycoprotein G of RABV.
80. The anti-Lyssavirus antibody, or the antigen-binding fragment thereof, for use according to any one of items 72-79, wherein the two anti-lyssavirus antibodies, or the antigen-binding fragments thereof, administered in combination are administered at equimolar amounts.
81. The anti-lyssavirus antibody, or the antigen-binding fragment thereof, for use according to any one of items 1-80, wherein the antibody, or the antigen-binding fragment thereof, is a monoclonal antibody and/or a human antibody.
82. The anti-Lyssavirus antibody, or the antigen-binding fragment thereof, for use according to item 81, wherein the antibody, or the antigen-binding fragment thereof, is a monoclonal antibody.
83. The anti-Lyssavirus antibody, or the antigen-binding fragment thereof, for use according to item 81, wherein the antibody, or the antigen-binding fragment thereof, is a human antibody.
84. The anti-lyssavirus antibody, or the antigen-binding fragment thereof, for use according to any one of items 1-82, wherein the antibody, or the antigen-binding fragment thereof, is a humanized antibody.
85. The anti-lyssavirus antibody, or the antigen-binding fragment thereof, for use according to any one of items 1-82, wherein the antibody, or the antigen-binding fragment thereof, is a chimeric antibody.
86. The anti-lyssavirus antibody, or the antigen-binding fragment thereof, for use according to any one of items 1-85, wherein the antibody, or the antigen-binding fragment thereof, is a recombinant antibody.
87. The anti-lyssavirus antibody, or the antigen-binding fragment thereof, for use according to item 86, wherein the antibody, or the antigen-binding fragment thereof, is a genetically engineered antibody.
88. The anti-lyssavirus antibody, or the antigen-binding fragment thereof, for use according to any one of items 1-87, wherein the antibody, or the antigen-binding fragment thereof, is a purified antibody.
89. The anti-lyssavirus antibody, or the antigen-binding fragment thereof, according to any one of items 1-88, wherein the antibody, or the antigen-binding fragment thereof, is a single chain antibody.
90. The anti-lyssavirus antibody, or the antigen-binding fragment thereof, according to any one of items 1-88, wherein the antibody, or the antigen-binding fragment thereof, is a Fab.
91. The anti-lyssavirus antibody, or the antigen-binding fragment thereof, according to any one of items 1-88, wherein the antibody, or the antigen-binding fragment thereof, is a Fab′.
92. The anti-lyssavirus antibody, or the antigen-binding fragment thereof, according to any one of items 1-88, wherein the antibody, or the antigen-binding fragment thereof, is a F(ab′)2.
93. The anti-lyssavirus antibody, or the antigen-binding fragment thereof, according to any one of items 1-88, wherein the antibody, or the antigen-binding fragment thereof, is a Fv.
94. The anti-lyssavirus antibody, or the antigen-binding fragment thereof, according to any one of items 1-88, wherein the antibody, or the antigen-binding fragment thereof, is a scFv.
95. The anti-lyssavirus antibody, or the antigen-binding fragment thereof, according to any one of items 1-88, wherein the antibody, or the antigen-binding fragment thereof, is an IgG antibody.
96. The anti-lyssavirus antibody, or the antigen-binding fragment thereof, according to any one of items 1-88, wherein the antibody, or the antigen-binding fragment thereof, is an IgM antibody.
97. The anti-lyssavirus antibody, or the antigen-binding fragment thereof, according to any one of items 1-88, wherein the antibody, or the antigen-binding fragment thereof, is an IgA antibody.
98. The anti-lyssavirus antibody, or the antigen-binding fragment thereof, according to item 95, wherein the antibody, or the antigen-binding fragment thereof, is an IgG1 antibody.
99. The anti-lyssavirus antibody, or the antigen-binding fragment thereof, according to item 95, wherein the antibody, or the antigen-binding fragment thereof, is an IgG2 antibody.
100. The anti-lyssavirus antibody, or the antigen-binding fragment thereof, according to item 95, wherein the antibody, or the antigen-binding fragment thereof, is an IgG3 antibody.
101. The anti-lyssavirus antibody, or the antigen-binding fragment thereof, according to item 95, wherein the antibody, or the antigen-binding fragment thereof, is an IgG4 antibody.
102. The anti-lyssavirus antibody, or the antigen-binding fragment thereof, for use according to any one of items 1-101, wherein the antibody, or the antigen-binding fragment thereof, neutralizes lyssavirus infection by (i) RABV and (ii) at least 50% of non-RABV lyssaviruses selected from the group consisting of DUVV, EBLV-1, EBLV-2, ABLY, IRKV, KHUV, ARAV, LBV, MOK, SHIV, BBLV, WCBV and IKOV with an IC₅₀ of less than 10000 ng/ml.
103. The antibody, or the antigen binding fragment thereof, according to item 102, characterized in that the antibody or antigen binding fragment neutralizes lyssavirus infection by (i) RABV and (ii) at least 50% of all isolates of non-RABV lyssaviruses selected from the group consisting of ABLV/Australia/bat/9810AUS-1998/V1039-2011/ABLV, 98010/ABLV, 1301 Bokeloh bat lyssavirus/BBLV, 86132SA/DUVV, DUVV/SouthAfrica/human/96132SA-1971/RS639-2012/DUVV, EBLV1a/France/bat/122938-2002/V3951-2009/EBLV-1, EBLV1b/France/bat/8918-1989/EBLV-1, EBLV2/UK/bat/RV1332-2002/V3951-2009/EBLV-2, 94112/EBLV-2, 02053/EBLV-2, 8619/LBV, MOK/MOK, Shimoni bat Virus/SHIV, West Caucasian bat Virus/WCBV, Australian bat lyssavirus/RV634/ABLV, Aravan Virus/ARAV, Duvenhage Virus RSA2006/DUVV, Duvenhage Virus ZIM86-RV 131/DUVV, European bat lyssavirus 1.RV20/EBLV-1, European bat lyssavirus 1.RV9/EBLV-1, EBLV 1a/France/bat/122938-2002/V3951-2009/EBLV-1, EBLV2/UK/bat/RV1332-2002/V3951-2009/EBLV-2, European bat lyssavirus 2.RV1787/EBLV-2, European bat lyssavirus 2.RV628/EBLV-2, Irkut Virus/IRKV, Khujand Virus/KHUV, 8619/LBV, Lagos Bat Virus NIG56-RV1/LBV, Lagos Bat Virus SA2004/LBV, Mokola Virus NIG68.RV4/MOK, Mokola Virus 98/071 RA36/MOK and Ikoma lyssavirus/IKOV with an IC₅₀ of less than 10000 ng/ml for ABLV/Australia/bat/9810AUS-1998/V1039-2011/ABLV, 98010/ABLV, 1301 Bokeloh bat lyssavirus/BBLV, 86132SA/DUVV, DUVV/SouthAfrica/human/96132SA-1971/RS639-2012/DUVV, EBLV1a/France/bat/122938-2002/V3951-2009/EBLV-1, EBLV1b/France/bat/8918-1989/EBLV-1, EBLV2/UK/bat/RV1332-2002/V3951-2009/EBLV-2, 94112/EBLV-2, 02053/EBLV-2, 8619/LBV, MOK/MOK tested as infectious viruses and with an IC₅₀ of less than 10000 ng/ml for Shimoni bat Virus/SHIV, West Caucasian bat Virus/WCBV, Australian bat lyssavirus/RV634/ABLV, Aravan Virus/ARAV, Duvenhage Virus RSA2006/DUVV, Duvenhage Virus ZIM86-RV 131/DUVV, European bat lyssavirus 1.RV20/EBLV-1, European bat Lyssavirus 1.RV9/EBLV-1, EBLV 1a/France/bat/122938-2002/V3951-2009/EBLV-1, EBLV2/UK/bat/RV1332-2002/V3951-2009/EBLV-2, European bat lyssavirus 2.RV1787/EBLV-2, European bat lyssavirus 2.RV628/EBLV-2, Irkut Virus/IRKV, Khujand Virus/KHUV, 8619/LBV, Lagos Bat Virus NIG56-RV1/LBV, Lagos Bat Virus SA2004/LBV, Mokola Virus NIG68.RV4/MOK, Mokola Virus 98/071 RA36/MOK and Ikoma lyssavirus/IKOV tested as pseudotyped viruses.
104. The antibody, or the antigen binding fragment thereof, according to item 102 or 103, characterized in that the antibody or antigen binding fragment neutralizes lyssavirus infection by at least 70% of non-RABV phylogroup I lyssaviruses selected from the group consisting of DUVV, EBLV-1, EBLV-2, ABLV, IRKV, KHUV, ARAV, and BBLV with an IC₅₀ of less than 10000 ng/ml.
105. The antibody, or the antigen binding fragment thereof, according to item 102 or 103, characterized in that the antibody or antigen binding fragment neutralizes lyssavirus infection by at least 70% of non-RABV phylogroup I lyssaviruses selected from the group consisting of DUVV, EBLV-1, EBLV-2, ABLV, IRKV, KHUV, and ARAV, with an IC₅₀ of less than 10000 ng/ml.
106. The antibody, or the antigen binding fragment thereof, according to item 105, characterized in that the antibody or antigen binding fragment neutralizes lyssavirus infection by at least 75% of non-RABV phylogroup I lyssaviruses selected from the group consisting of DUVV, EBLV-1, EBLV-2, ABLY, IRKV, KHUV, and ARAV, with an IC₅₀ of less than 10000 ng/ml.
107. The antibody, or the antigen binding fragment thereof, according to item 106, characterized in that the antibody or antigen binding fragment neutralizes lyssavirus infection by at least 80% of non-RABV phylogroup I lyssaviruses selected from the group consisting of DUVV, EBLV-1, EBLV-2, ABLY, IRKV, KHUV, and ARAV, with an IC₅₀ of less than 10000 ng/ml.
108. The antibody, or the antigen binding fragment thereof, according to item 107, characterized in that the antibody or antigen binding fragment neutralizes lyssavirus infection by at least 82% of non-RABV phylogroup I lyssaviruses selected from the group consisting of DUVV, EBLV-1, EBLV-2, ABLY, IRKV, KHUV, and ARAV, with an IC₅₀ of less than 10000 ng/ml.
109. The antibody, or the antigen binding fragment thereof, according to any of items 1-108, characterized in that the antibody or antigen binding fragment neutralizes lyssavirus infection by at least 70% of the isolates of non-RABV phylogroup I lyssaviruses selected from the group consisting of ABLV/Australia/bat/9810AUS-1998/V1039-2011/ABLV, 98010/ABLV, 1301 Bokeloh bat lyssavirus/BBLV, 86132SA/DUVV, DUVV/SouthAfrica/human/96132SA-1971/RS639-2012/DUVV, EBLV1a/France/bat/122938-2002/V3951-2009/EBLV-1, EBLV1b/France/bat/8918-1989/EBLV-1, EBLV2/UK/bat/RV1332-2002/V3951-2009/EBLV-2, 94112/EBLV-2, 02053/EBLV-2, Australian bat lyssavirus/RV634/ABLV, Aravan Virus/ARAV, Duvenhage Virus RSA2006/DUVV, Duvenhage Virus ZIM86-RV 131/DUVV, European bat lyssavirus 1.RV20/EBLV-1, European bat lyssavirus 1.RV9/EBLV-1, EBLV 1a/France/bat/122938-2002/V3951-2009/EBLV-1, EBLV2/UK/bat/RV1332-2002/V3951-2009/EBLV-2, European bat lyssavirus 2.RV1787/EBLV-2 and European bat lyssavirus 2.RV628/EBLV-2, Irkut Virus/IRKV, Khujand Virus/KHUV, with an IC₅₀ of less than 10000 ng/ml for ABLV/Australia/bat/9810AUS-1998/V1039-2011/ABLV, 98010/ABLV, 1301 Bokeloh bat lyssavirus/BBLV, 86132SA/DUVV, DUVV/SouthAfrica/human/96132SA-1971/RS639-2012/DUVV, EBLV1a/France/bat/122938-2002/V3951-2009/EBLV-1, EBLV1b/France/bat/8918-1989/EBLV-1, EBLV2/UK/bat/RV1332-2002/V3951-2009/EBLV-2, 94112/EBLV-2 and 02053/EBLV-2, tested as infectious viruses and with an IC₉₀ of less than 10000 ng/ml for Australian bat lyssavirus/RV634/ABLV, Aravan Virus/ARAV, Duvenhage Virus RSA2006/DUVV, Duvenhage Virus ZIM86-RV 131/DUVV, European bat lyssavirus 1.RV20/EBLV-1, European bat lyssavirus 1.RV9/EBLV-1, EBLV 1a/France/bat/122938-2002/V3951-2009/EBLV-1, EBLV2/UK/bat/RV1332-2002/V3951-2009/EBLV-2, European bat lyssavirus 2.RV1787/EBLV-2, European bat lyssavirus 2. RV628/EB LV-2, Irkut Virus/IRKV and Khujand Virus/KHUV tested as pseudotyped viruses.
110. The antibody, or the antigen binding fragment thereof, according to any of items 1-109, characterized in that the antibody or antigen binding fragment neutralizes infection by EBLV-1.
111. The anti-lyssavirus antibody, or the antigen-binding fragment thereof, for use according to any one of items 1-110, wherein the antibody, or the antigen-binding fragment thereof, neutralizes infection by RABV CVS-11 with an IC₉₀ of 400 ng/ml or less.
112. The anti-lyssavirus antibody, or the antigen-binding fragment thereof, for use according to any one of items 1-111, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 201-205 and 207 or to the amino acid sequences of SEQ ID NOs: 201-204 and 206-207, respectively.
113. The anti-lyssavirus antibody, or the antigen-binding fragment thereof, for use according to item 112, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 201-205 and 207 or to the amino acid sequences of SEQ ID NOs: 201-204 and 206-207, respectively.
114. The anti-lyssavirus antibody, or the antigen-binding fragment thereof, for use according to item 113, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 201-205 and 207 or to the amino acid sequences of SEQ ID NOs: 201-204 and 206-207, respectively.
115. The anti-lyssavirus antibody, or the antigen-binding fragment thereof, for use according to item 114, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 201-205 and 207 or to the amino acid sequences of SEQ ID NOs: 201-204 and 206-207, respectively.
116. The anti-Lyssavirus antibody, or the antigen-binding fragment thereof, for use according to item 115, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 201-205 and 207 or to the amino acid sequences of SEQ ID NOs: 201-204 and 206-207, respectively.
117. The anti-lyssavirus antibody, or the antigen-binding fragment thereof, for use according to item 116, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 201-205 and 207 or to the amino acid sequences of SEQ ID NOs: 201-204 and 206-207, respectively.
118. The anti-lyssavirus antibody, or the antigen-binding fragment thereof, for use according to item 117, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 201-205 and 207 or to the amino acid sequences of SEQ ID NOs: 201-204 and 206-207, respectively.
119. The anti-lyssavirus antibody, or the antigen-binding fragment thereof, for use according to item 118, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 201-205 and 207 or to the amino acid sequences of SEQ ID NOs: 201-204 and 206-207, respectively.
120. The anti-Lyssavirus antibody, or the antigen-binding fragment thereof, for use according to item 119, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 201-205 and 207 or to the amino acid sequences of SEQ ID NOs: 201-204 and 206-207, respectively.
121. The anti-lyssavirus antibody, or the antigen-binding fragment thereof, for use according to item 120, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 201-205 and 207 or to the amino acid sequences of SEQ ID NOs: 201-204 and 206-207, respectively.
122. The anti-lyssavirus antibody, or the antigen-binding fragment thereof, for use according to item 121, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 201-205 and 207 or to the amino acid sequences of SEQ ID NOs: 201-204 and 206-207, respectively.
123. The anti-lyssavirus antibody, or the antigen-binding fragment thereof, for use according to item 122, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 201-205 and 207 or to the amino acid sequences of SEQ ID NOs: 201-204 and 206-207, respectively.
124. The anti-lyssavirus antibody, or the antigen-binding fragment thereof, for use according to item 123, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences as set forth in SEQ ID NOs: 1-5 and 7 or in SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences as set forth in SEQ ID NOs: 19-23 and 25 or in SEQ ID NOs: 19-22 and 24-25, respectively; (v) heavy chain CDRH1, CDRH2, and CDRH3 amino acid sequences and light chain CDRL1, CDRL2, and CDRL3 amino acid sequences as set forth in SEQ ID NOs: 37-41 and 43 or in SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences as set forth in SEQ ID NOs: 55-59 and 61 or in SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences as set forth in SEQ ID NOs: 75-79 and 81 or in SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences as set forth in SEQ ID NOs: 111-115 and 117 or in SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences as set forth in SEQ ID NOs: 129-133 and 135 or in SEQ ID NOs: 129-132 and 134-135, respectively; (x) heavy chain CDRH1, CDRH2, and CDRH3 amino acid sequences and light chain CDRL1, CDRL2, and CDRL3 amino acid sequences as set forth in SEQ ID NOs: 147-151 and 153 or in SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences as set forth in SEQ ID NOs: 183-187 and 189 or in SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences as set forth in SEQ ID NOs: 201-205 and 207 or in SEQ ID NOs: 201-204 and 206-207, respectively.
125. The anti-lyssavirus antibody, or the antigen-binding fragment thereof, for use according to item 112, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 93-97 and 99 or to the amino acid sequences of SEQ ID NOs: 93-96 and 98-99, respectively.
126. The anti-lyssavirus antibody, or the antigen-binding fragment thereof, for use according to item 125, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 93-97 and 99 or to the amino acid sequences of SEQ ID NOs: 93-96 and 98-99, respectively.
127. The anti-lyssavirus antibody, or the antigen-binding fragment thereof, for use according to item 126, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively.
128. The anti-lyssavirus antibody, or the antigen-binding fragment thereof, for use according to item 127, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 93-97 and 99 or to the amino acid sequences of SEQ ID NOs: 93-96 and 98-99, respectively.
129. The anti-lyssavirus antibody, or the antigen-binding fragment thereof, for use according to item 128, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 93-97 and 99 or to the amino acid sequences of SEQ ID NOs: 93-96 and 98-99, respectively.
130. The anti-lyssavirus antibody, or the antigen-binding fragment thereof, for use according to item 129, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 93-97 and 99 or to the amino acid sequences of SEQ ID NOs: 93-96 and 98-99, respectively.
131. The anti-lyssavirus antibody, or the antigen-binding fragment thereof, for use according to item 130, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 93-97 and 99 or to the amino acid sequences of SEQ ID NOs: 93-96 and 98-99, respectively.
132. The anti-lyssavirus antibody, or the antigen-binding fragment thereof, for use according to item 131, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 93-97 and 99 or to the amino acid sequences of SEQ ID NOs: 93-96 and 98-99, respectively.
133. The anti-lyssavirus antibody, or the antigen-binding fragment thereof, for use according to item 132, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 93-97 and 99 or to the amino acid sequences of SEQ ID NOs: 93-96 and 98-99, respectively.
134. The anti-Lyssavirus antibody, or the antigen-binding fragment thereof, for use according to item 133, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 93-97 and 99 or to the amino acid sequences of SEQ ID NOs: 93-96 and 98-99, respectively.
135. The anti-lyssavirus antibody, or the antigen-binding fragment thereof, for use according to item 134, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 93-97 and 99 or to the amino acid sequences of SEQ ID NOs: 93-96 and 98-99, respectively.
136. The anti-lyssavirus antibody, or the antigen-binding fragment thereof, for use according to item 135, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 93-97 and 99 or to the amino acid sequences of SEQ ID NOs: 93-96 and 98-99, respectively.
137. The anti-Lyssavirus antibody, or the antigen-binding fragment thereof, for use according to item 135, wherein the antibody, or the antigen-binding fragment thereof, comprises heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, respectively.
138. The anti-Lyssavirus antibody, or the antigen-binding fragment thereof, for use according to item 112, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 165-169 and 171 or to the amino acid sequences of SEQ ID NOs: 165-168 and 170-171, respectively.
139. The anti-lyssavirus antibody, or the antigen-binding fragment thereof, for use according to item 138, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 165-169 and 171 or to the amino acid sequences of SEQ ID NOs: 165-168 and 170-171, respectively.
140. The anti-lyssavirus antibody, or the antigen-binding fragment thereof, for use according to item 139, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively.
141. The anti-lyssavirus antibody, or the antigen-binding fragment thereof, for use according to item 141, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 165-169 and 171 or to the amino acid sequences of SEQ ID NOs: 165-168 and 170-171, respectively.
142. The anti-lyssavirus antibody, or the antigen-binding fragment thereof, for use according to item 141, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 165-169 and 171 or to the amino acid sequences of SEQ ID NOs: 165-168 and 170-171, respectively.
143. The anti-Lyssavirus antibody, or the antigen-binding fragment thereof, for use according to item 142, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 165-169 and 171 or to the amino acid sequences of SEQ ID NOs: 165-168 and 170-171, respectively.
144. The anti-lyssavirus antibody, or the antigen-binding fragment thereof, for use according to item 143, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 165-169 and 171 or to the amino acid sequences of SEQ ID NOs: 165-168 and 170-171, respectively.
145. The anti-lyssavirus antibody, or the antigen-binding fragment thereof, for use according to item 144, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 165-169 and 171 or to the amino acid sequences of SEQ ID NOs: 165-168 and 170-171, respectively.
146. The anti-lyssavirus antibody, or the antigen-binding fragment thereof, for use according to item 145, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 165-169 and 171 or to the amino acid sequences of SEQ ID NOs: 165-168 and 170-171, respectively.
147. The anti-lyssavirus antibody, or the antigen-binding fragment thereof, for use according to item 146, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 165-169 and 171 or to the amino acid sequences of SEQ ID NOs: 165-168 and 170-171, respectively.
148. The anti-lyssavirus antibody, or the antigen-binding fragment thereof, for use according to item 147, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 165-169 and 171 or to the amino acid sequences of SEQ ID NOs: 165-168 and 170-171, respectively.
149. The anti-lyssavirus antibody, or the antigen-binding fragment thereof, for use according to item 148, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 165-169 and 171 or to the amino acid sequences of SEQ ID NOs: 165-168 and 170-171, respectively.
150. The anti-lyssavirus antibody, or the antigen-binding fragment thereof, for use according to item 112, wherein the antibody, or the antigen-binding fragment thereof, comprises heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, respectively.
151. The anti-lyssavirus antibody, or the antigen-binding fragment thereof, for use according to any one of items 1-124, wherein the antibody, or the antigen-binding fragment thereof, comprises: (i) a heavy chain variable region having at least 70% sequence identity to the amino acid sequence of SEQ ID NO: 107 and a light chain variable region having at least 70% sequence identity to the amino acid sequence of SEQ ID NO: 108; or (ii) a heavy chain variable region having at least 70% sequence identity to the amino acid sequence of SEQ ID NO: 179 and a light chain variable region having at least 70% sequence identity to the amino acid sequence of SEQ ID NO: 180; or (iii) a heavy chain variable region having at least 70% sequence identity to the amino acid sequence of SEQ ID NO: 15 and a light chain variable region having at least 70% sequence identity to the amino acid sequence of SEQ ID NO: 16; or (iv) a heavy chain variable region having at least 70% sequence identity to the amino acid sequence of SEQ ID NO: 33 and a light chain variable region having at least 70% sequence identity to the amino acid sequence of SEQ ID NO: 34; or (v) a heavy chain variable region having at least 70% sequence identity to the amino acid sequence of SEQ ID NO: 51 and a light chain variable region having at least 70% sequence identity to the amino acid sequence of SEQ ID NO: 52; or (vi) a heavy chain variable region having at least 70% sequence identity to the amino acid sequence of SEQ ID NO: 69 and a light chain variable region having at least 70% sequence identity to the amino acid sequence of SEQ ID NO: 71; or (vii) a heavy chain variable region having at least 70% sequence identity to the amino acid sequence of SEQ ID NO: 70 and a light chain variable region having at least 70% sequence identity to the amino acid sequence of SEQ ID NO: 71; or (viii) a heavy chain variable region having at least 70% sequence identity to the amino acid sequence of SEQ ID NO: 89 and a light chain variable region having at least 70% sequence identity to the amino acid sequence of SEQ ID NO: 90; or (ix) a heavy chain variable region having at least 70% sequence identity to the amino acid sequence of SEQ ID NO: 125 and a light chain variable region having at least 70% sequence identity to the amino acid sequence of SEQ ID NO: 126; or (x) a heavy chain variable region having at least 70% sequence identity to the amino acid sequence of SEQ ID NO: 143 and a light chain variable region having at least 70% sequence identity to the amino acid sequence of SEQ ID NO: 144; or (xi) a heavy chain variable region having at least 70% sequence identity to the amino acid sequence of SEQ ID NO: 161 and a light chain variable region having at least 70% sequence identity to the amino acid sequence of SEQ ID NO: 162; or (xii) a heavy chain variable region having at least 70% sequence identity to the amino acid sequence of SEQ ID NO: 197 and a light chain variable region having at least 70% sequence identity to the amino acid sequence of SEQ ID NO: 198; or (xiii) a heavy chain variable region having at least 70% sequence identity to the amino acid sequence of SEQ ID NO: 215 and a light chain variable region having at least 70% sequence identity to the amino acid sequence of SEQ ID NO: 216.
152. The anti-lyssavirus antibody, or the antigen-binding fragment thereof, for use according to item 152, wherein the antibody, or the antigen-binding fragment thereof, comprises: (i) a heavy chain variable region having at least 75% sequence identity to the amino acid sequence of SEQ ID NO: 107 and a light chain variable region having at least 75% sequence identity to the amino acid sequence of SEQ ID NO: 108; or (ii) a heavy chain variable region having at least 75% sequence identity to the amino acid sequence of SEQ ID NO: 179 and a light chain variable region having at least 75% sequence identity to the amino acid sequence of SEQ ID NO: 180; or (iii) a heavy chain variable region having at least 75% sequence identity to the amino acid sequence of SEQ ID NO: 15 and a light chain variable region having at least 75% sequence identity to the amino acid sequence of SEQ ID NO: 16; or (iv) a heavy chain variable region having at least 75% sequence identity to the amino acid sequence of SEQ ID NO: 33 and a light chain variable region having at least 75% sequence identity to the amino acid sequence of SEQ ID NO: 34; or (v) a heavy chain variable region having at least 75% sequence identity to the amino acid sequence of SEQ ID NO: 51 and a light chain variable region having at least 75% sequence identity to the amino acid sequence of SEQ ID NO: 52; or (vi) a heavy chain variable region having at least 75% sequence identity to the amino acid sequence of SEQ ID NO: 69 and a light chain variable region having at least 75% sequence identity to the amino acid sequence of SEQ ID NO: 71; or (vii) a heavy chain variable region having at least 75% sequence identity to the amino acid sequence of SEQ ID NO: 70 and a light chain variable region having at least 75% sequence identity to the amino acid sequence of SEQ ID NO: 71; or (viii) a heavy chain variable region having at least 75% sequence identity to the amino acid sequence of SEQ ID NO: 89 and a light chain variable region having at least 75% sequence identity to the amino acid sequence of SEQ ID NO: 90; or (ix) a heavy chain variable region having at least 75% sequence identity to the amino acid sequence of SEQ ID NO: 125 and a light chain variable region having at least 75% sequence identity to the amino acid sequence of SEQ ID NO: 126; or (x) a heavy chain variable region having at least 75% sequence identity to the amino acid sequence of SEQ ID NO: 143 and a light chain variable region having at least 75% sequence identity to the amino acid sequence of SEQ ID NO: 144; or (xi) a heavy chain variable region having at least 75% sequence identity to the amino acid sequence of SEQ ID NO: 161 and a light chain variable region having at least 75% sequence identity to the amino acid sequence of SEQ ID NO: 162; or (xii) a heavy chain variable region having at least 75% sequence identity to the amino acid sequence of SEQ ID NO: 197 and a light chain variable region having at least 75% sequence identity to the amino acid sequence of SEQ ID NO: 198; or (xiii) a heavy chain variable region having at least 75% sequence identity to the amino acid sequence of SEQ ID NO: 215 and a light chain variable region having at least 75% sequence identity to the amino acid sequence of SEQ ID NO: 216.
153. The anti-lyssavirus antibody, or the antigen-binding fragment thereof, for use according to item 152, wherein the antibody, or the antigen-binding fragment thereof, comprises: (i) a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108; or (ii) a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180; or (iii) a heavy chain variable region having at least 80%, or at least 90%, sequence identity to the amino acid sequence of SEQ ID NO: 15 and a light chain variable region having at least 80%, or at least 90%, sequence identity to the amino acid sequence of SEQ ID NO: 16; or (iv) a heavy chain variable region having at least 80%, or at least 90%, sequence identity to the amino acid sequence of SEQ ID NO: 33 and a light chain variable region having at least 80%, or at least 90%, sequence identity to the amino acid sequence of SEQ ID NO: 34; or (v) a heavy chain variable region having at least 80%, or at least 90%, sequence identity to the amino acid sequence of SEQ ID NO: 51 and a light chain variable region having at least 80%, or at least 90%, sequence identity to the amino acid sequence of SEQ ID NO: 52; or (vi) a heavy chain variable region having at least 80%, or at least 90%, sequence identity to the amino acid sequence of SEQ ID NO: 69 and a light chain variable region having at least 80%, or at least 90%, sequence identity to the amino acid sequence of SEQ ID NO: 71; or (vii) a heavy chain variable region having at least 80%, or at least 90%, sequence identity to the amino acid sequence of SEQ ID NO: 70 and a light chain variable region having at least 80%, or at least 90%, sequence identity to the amino acid sequence of SEQ ID NO: 71; or (viii) a heavy chain variable region having at least 80%, or at least 90%, sequence identity to the amino acid sequence of SEQ ID NO: 89 and a light chain variable region having at least 80%, or at least 90%, sequence identity to the amino acid sequence of SEQ ID NO: 90; or (ix) a heavy chain variable region having at least 80%, or at least 90%, sequence identity to the amino acid sequence of SEQ ID NO: 125 and a light chain variable region having at least 80%, or at least 90%, sequence identity to the amino acid sequence of SEQ ID NO: 126; or (x) a heavy chain variable region having at least 80%, or at least 90%, sequence identity to the amino acid sequence of SEQ ID NO: 143 and a light chain variable region having at least 80%, or at least 90%, sequence identity to the amino acid sequence of SEQ ID NO: 144; or (xi) a heavy chain variable region having at least 80%, or at least 90%, sequence identity to the amino acid sequence of SEQ ID NO: 161 and a light chain variable region having at least 80%, or at least 90%, sequence identity to the amino acid sequence of SEQ ID NO: 162; or (xii) a heavy chain variable region having at least 80%, or at least 90%, sequence identity to the amino acid sequence of SEQ ID NO: 197 and a light chain variable region having at least 80%, or at least 90%, sequence identity to the amino acid sequence of SEQ ID NO: 198; or (xiii) a heavy chain variable region having at least 80%, or at least 90%, sequence identity to the amino acid sequence of SEQ ID NO: 215 and a light chain variable region having at least 80%, or at least 90%, sequence identity to the amino acid sequence of SEQ ID NO: 216.
154. The anti-lyssavirus antibody, or the antigen-binding fragment thereof, for use according to item 153, wherein the antibody, or the antigen-binding fragment thereof, comprises: (i) a heavy chain variable region having at least 80% sequence identity to the amino acid sequence of SEQ ID NO: 107 and a light chain variable region having at least 80% sequence identity to the amino acid sequence of SEQ ID NO: 108; or (ii) a heavy chain variable region having at least 80% sequence identity to the amino acid sequence of SEQ ID NO: 179 and a light chain variable region having at least 80% sequence identity to the amino acid sequence of SEQ ID NO: 180; or (iii) a heavy chain variable region having at least 80% sequence identity to the amino acid sequence of SEQ ID NO: 15 and a light chain variable region having at least 80% sequence identity to the amino acid sequence of SEQ ID NO: 16; or (iv) a heavy chain variable region having at least 80% sequence identity to the amino acid sequence of SEQ ID NO: 33 and a light chain variable region having at least 80% sequence identity to the amino acid sequence of SEQ ID NO: 34; or (v) a heavy chain variable region having at least 80% sequence identity to the amino acid sequence of SEQ ID NO: 51 and a light chain variable region having at least 80% sequence identity to the amino acid sequence of SEQ ID NO: 52; or (vi) a heavy chain variable region having at least 80% sequence identity to the amino acid sequence of SEQ ID NO: 69 and a light chain variable region having at least 80% sequence identity to the amino acid sequence of SEQ ID NO: 71; or (vii) a heavy chain variable region having at least 80% sequence identity to the amino acid sequence of SEQ ID NO: 70 and a light chain variable region having at least 80% sequence identity to the amino acid sequence of SEQ ID NO: 71; or (viii) a heavy chain variable region having at least 80% sequence identity to the amino acid sequence of SEQ ID NO: 89 and a light chain variable region having at least 80% sequence identity to the amino acid sequence of SEQ ID NO: 90; or (ix) a heavy chain variable region having at least 80% sequence identity to the amino acid sequence of SEQ ID NO: 125 and a light chain variable region having at least 80% sequence identity to the amino acid sequence of SEQ ID NO: 126; or (x) a heavy chain variable region having at least 80% sequence identity to the amino acid sequence of SEQ ID NO: 143 and a light chain variable region having at least 80% sequence identity to the amino acid sequence of SEQ ID NO: 144; or (xi) a heavy chain variable region having at least 80% sequence identity to the amino acid sequence of SEQ ID NO: 161 and a light chain variable region having at least 80% sequence identity to the amino acid sequence of SEQ ID NO: 162; or (xii) a heavy chain variable region having at least 80% sequence identity to the amino acid sequence of SEQ ID NO: 197 and a light chain variable region having at least 80% sequence identity to the amino acid sequence of SEQ ID NO: 198; or (xiii) a heavy chain variable region having at least 80% sequence identity to the amino acid sequence of SEQ ID NO: 215 and a light chain variable region having at least 80% sequence identity to the amino acid sequence of SEQ ID NO: 216.
155. The anti-lyssavirus antibody, or the antigen-binding fragment thereof, for use according to item 154, wherein the antibody, or the antigen-binding fragment thereof, comprises: (i) a heavy chain variable region having at least 85% sequence identity to the amino acid sequence of SEQ ID NO: 107 and a light chain variable region having at least 85% sequence identity to the amino acid sequence of SEQ ID NO: 108; or (ii) a heavy chain variable region having at least 85% sequence identity to the amino acid sequence of SEQ ID NO: 179 and a light chain variable region having at least 85% sequence identity to the amino acid sequence of SEQ ID NO: 180; or (iii) a heavy chain variable region having at least 85% sequence identity to the amino acid sequence of SEQ ID NO: 15 and a light chain variable region having at least 85% sequence identity to the amino acid sequence of SEQ ID NO: 16; or (iv) a heavy chain variable region having at least 85% sequence identity to the amino acid sequence of SEQ ID NO: 33 and a light chain variable region having at least 85% sequence identity to the amino acid sequence of SEQ ID NO: 34; or (v) a heavy chain variable region having at least 85% sequence identity to the amino acid sequence of SEQ ID NO: 51 and a light chain variable region having at least 85% sequence identity to the amino acid sequence of SEQ ID NO: 52; or (vi) a heavy chain variable region having at least 85% sequence identity to the amino acid sequence of SEQ ID NO: 69 and a light chain variable region having at least 85% sequence identity to the amino acid sequence of SEQ ID NO: 71; or (vii) a heavy chain variable region having at least 85% sequence identity to the amino acid sequence of SEQ ID NO: 70 and a light chain variable region having at least 85% sequence identity to the amino acid sequence of SEQ ID NO: 71; or (viii) a heavy chain variable region having at least 85% sequence identity to the amino acid sequence of SEQ ID NO: 89 and a light chain variable region having at least 85% sequence identity to the amino acid sequence of SEQ ID NO: 90; or (ix) a heavy chain variable region having at least 85% sequence identity to the amino acid sequence of SEQ ID NO: 125 and a light chain variable region having at least 85% sequence identity to the amino acid sequence of SEQ ID NO: 126; or (x) a heavy chain variable region having at least 85% sequence identity to the amino acid sequence of SEQ ID NO: 143 and a light chain variable region having at least 85% sequence identity to the amino acid sequence of SEQ ID NO: 144; or (xi) a heavy chain variable region having at least 85% sequence identity to the amino acid sequence of SEQ ID NO: 161 and a light chain variable region having at least 85% sequence identity to the amino acid sequence of SEQ ID NO: 162; or (xii) a heavy chain variable region having at least 85% sequence identity to the amino acid sequence of SEQ ID NO: 197 and a light chain variable region having at least 85% sequence identity to the amino acid sequence of SEQ ID NO: 198; or (xiii) a heavy chain variable region having at least 85% sequence identity to the amino acid sequence of SEQ ID NO: 215 and a light chain variable region having at least 85% sequence identity to the amino acid sequence of SEQ ID NO: 216.
156. The anti-lyssavirus antibody, or the antigen-binding fragment thereof, for use according to item 155, wherein the antibody, or the antigen-binding fragment thereof, comprises: (i) a heavy chain variable region having at least 88% sequence identity to the amino acid sequence of SEQ ID NO: 107 and a light chain variable region having at least 88% sequence identity to the amino acid sequence of SEQ ID NO: 108; or (ii) a heavy chain variable region having at least 88% sequence identity to the amino acid sequence of SEQ ID NO: 179 and a light chain variable region having at least 88% sequence identity to the amino acid sequence of SEQ ID NO: 180; or (iii) a heavy chain variable region having at least 88% sequence identity to the amino acid sequence of SEQ ID NO: 15 and a light chain variable region having at least 88% sequence identity to the amino acid sequence of SEQ ID NO: 16; or (iv) a heavy chain variable region having at least 88% sequence identity to the amino acid sequence of SEQ ID NO: 33 and a light chain variable region having at least 88% sequence identity to the amino acid sequence of SEQ ID NO: 34; or (v) a heavy chain variable region having at least 88% sequence identity to the amino acid sequence of SEQ ID NO: 51 and a light chain variable region having at least 88% sequence identity to the amino acid sequence of SEQ ID NO: 52; or (vi) a heavy chain variable region having at least 88% sequence identity to the amino acid sequence of SEQ ID NO: 69 and a light chain variable region having at least 88% sequence identity to the amino acid sequence of SEQ ID NO: 71; or (vii) a heavy chain variable region having at least 88% sequence identity to the amino acid sequence of SEQ ID NO: 70 and a light chain variable region having at least 88% sequence identity to the amino acid sequence of SEQ ID NO: 71; or (viii) a heavy chain variable region having at least 88% sequence identity to the amino acid sequence of SEQ ID NO: 89 and a light chain variable region having at least 88% sequence identity to the amino acid sequence of SEQ ID NO: 90; or (ix) a heavy chain variable region having at least 88% sequence identity to the amino acid sequence of SEQ ID NO: 125 and a light chain variable region having at least 88% sequence identity to the amino acid sequence of SEQ ID NO: 126; or (x) a heavy chain variable region having at least 88% sequence identity to the amino acid sequence of SEQ ID NO: 143 and a light chain variable region having at least 88% sequence identity to the amino acid sequence of SEQ ID NO: 144; or (xi) a heavy chain variable region having at least 88% sequence identity to the amino acid sequence of SEQ ID NO: 161 and a light chain variable region having at least 88% sequence identity to the amino acid sequence of SEQ ID NO: 162; or (xii) a heavy chain variable region having at least 88% sequence identity to the amino acid sequence of SEQ ID NO: 197 and a light chain variable region having at least 88% sequence identity to the amino acid sequence of SEQ ID NO: 198; or (xi ii) a heavy chain variable region having at least 88% sequence identity to the amino acid sequence of SEQ ID NO: 215 and a light chain variable region having at least 88% sequence identity to the amino acid sequence of SEQ ID NO: 216.
157. The anti-lyssavirus antibody, or the antigen-binding fragment thereof, for use according to item 156, wherein the antibody, or the antigen-binding fragment thereof, comprises: (i) a heavy chain variable region having at least 90% sequence identity to the amino acid sequence of SEQ ID NO: 107 and a light chain variable region having at least 90% sequence identity to the amino acid sequence of SEQ ID NO: 108; or (ii) a heavy chain variable region having at least 90% sequence identity to the amino acid sequence of SEQ ID NO: 179 and a light chain variable region having at least 90% sequence identity to the amino acid sequence of SEQ ID NO: 180; or (iii) a heavy chain variable region having at least 90% sequence identity to the amino acid sequence of SEQ ID NO: 15 and a light chain variable region having at least 90% sequence identity to the amino acid sequence of SEQ ID NO: 16; or (iv) a heavy chain variable region having at least 90% sequence identity to the amino acid sequence of SEQ ID NO: 33 and a light chain variable region having at least 90% sequence identity to the amino acid sequence of SEQ ID NO: 34; or (v) a heavy chain variable region having at least 90% sequence identity to the amino acid sequence of SEQ ID NO: 51 and a light chain variable region having at least 90% sequence identity to the amino acid sequence of SEQ ID NO: 52; or (vi) a heavy chain variable region having at least 90% sequence identity to the amino acid sequence of SEQ ID NO: 69 and a light chain variable region having at least 90% sequence identity to the amino acid sequence of SEQ ID NO: 71; or (vii) a heavy chain variable region having at least 90% sequence identity to the amino acid sequence of SEQ ID NO: 70 and a light chain variable region having at least 90% sequence identity to the amino acid sequence of SEQ ID NO: 71; or (viii) a heavy chain variable region having at least 90% sequence identity to the amino acid sequence of SEQ ID NO: 89 and a light chain variable region having at least 90% sequence identity to the amino acid sequence of SEQ ID NO: 90; or (ix) a heavy chain variable region having at least 90% sequence identity to the amino acid sequence of SEQ ID NO: 125 and a light chain variable region having at least 90% sequence identity to the amino acid sequence of SEQ ID NO: 126; or (x) a heavy chain variable region having at least 90% sequence identity to the amino acid sequence of SEQ ID NO: 143 and a light chain variable region having at least 90% sequence identity to the amino acid sequence of SEQ ID NO: 144; or (xi) a heavy chain variable region having at least 90% sequence identity to the amino acid sequence of SEQ ID NO: 161 and a light chain variable region having at least 90% sequence identity to the amino acid sequence of SEQ ID NO: 162; or (xii) a heavy chain variable region having at least 90% sequence identity to the amino acid sequence of SEQ ID NO: 197 and a light chain variable region having at least 90% sequence identity to the amino acid sequence of SEQ ID NO: 198; or (xiii) a heavy chain variable region having at least 90% sequence identity to the amino acid sequence of SEQ ID NO: 215 and a light chain variable region having at least 90% sequence identity to the amino acid sequence of SEQ ID NO: 216.
158. The anti-lyssavirus antibody, or the antigen-binding fragment thereof, for use according to item 157, wherein the antibody, or the antigen-binding fragment thereof, comprises: (i) a heavy chain variable region having at least 92% sequence identity to the amino acid sequence of SEQ ID NO: 107 and a light chain variable region having at least 92% sequence identity to the amino acid sequence of SEQ ID NO: 108; or (ii) a heavy chain variable region having at least 92% sequence identity to the amino acid sequence of SEQ ID NO: 179 and a light chain variable region having at least 92% sequence identity to the amino acid sequence of SEQ ID NO: 180; or (iii) a heavy chain variable region having at least 92% sequence identity to the amino acid sequence of SEQ ID NO: 15 and a light chain variable region having at least 92% sequence identity to the amino acid sequence of SEQ ID NO: 16; or (iv) a heavy chain variable region having at least 92% sequence identity to the amino acid sequence of SEQ ID NO: 33 and a light chain variable region having at least 92% sequence identity to the amino acid sequence of SEQ ID NO: 34; or (v) a heavy chain variable region having at least 92% sequence identity to the amino acid sequence of SEQ ID NO: 51 and a light chain variable region having at least 92% sequence identity to the amino acid sequence of SEQ ID NO: 52; or (vi) a heavy chain variable region having at least 92% sequence identity to the amino acid sequence of SEQ ID NO: 69 and a light chain variable region having at least 92% sequence identity to the amino acid sequence of SEQ ID NO: 71; or (vii) a heavy chain variable region having at least 92% sequence identity to the amino acid sequence of SEQ ID NO: 70 and a light chain variable region having at least 92% sequence identity to the amino acid sequence of SEQ ID NO: 71; or (viii) a heavy chain variable region having at least 92% sequence identity to the amino acid sequence of SEQ ID NO: 89 and a light chain variable region having at least 92% sequence identity to the amino acid sequence of SEQ ID NO: 90; or (ix) a heavy chain variable region having at least 92% sequence identity to the amino acid sequence of SEQ ID NO: 125 and a light chain variable region having at least 92% sequence identity to the amino acid sequence of SEQ ID NO: 126; or (x) a heavy chain variable region having at least 92% sequence identity to the amino acid sequence of SEQ ID NO: 143 and a light chain variable region having at least 92% sequence identity to the amino acid sequence of SEQ ID NO: 144; or (xi) a heavy chain variable region having at least 92% sequence identity to the amino acid sequence of SEQ ID NO: 161 and a light chain variable region having at least 92% sequence identity to the amino acid sequence of SEQ ID NO: 162; or (xii) a heavy chain variable region having at least 92% sequence identity to the amino acid sequence of SEQ ID NO: 197 and a light chain variable region having at least 92% sequence identity to the amino acid sequence of SEQ ID NO: 198; or (xiii) a heavy chain variable region having at least 92% sequence identity to the amino acid sequence of SEQ ID NO: 215 and a light chain variable region having at least 92% sequence identity to the amino acid sequence of SEQ ID NO: 216.
159. The anti-lyssavirus antibody, or the antigen-binding fragment thereof, for use according to item 158, wherein the antibody, or the antigen-binding fragment thereof, comprises: (i) a heavy chain variable region having at least 95% sequence identity to the amino acid sequence of SEQ ID NO: 107 and a light chain variable region having at least 95% sequence identity to the amino acid sequence of SEQ ID NO: 108; or (ii) a heavy chain variable region having at least 95% sequence identity to the amino acid sequence of SEQ ID NO: 179 and a light chain variable region having at least 95% sequence identity to the amino acid sequence of SEQ ID NO: 180; or (iii) a heavy chain variable region having at least 95% sequence identity to the amino acid sequence of SEQ ID NO: 15 and a light chain variable region having at least 95% sequence identity to the amino acid sequence of SEQ ID NO: 16; or (iv) a heavy chain variable region having at least 95% sequence identity to the amino acid sequence of SEQ ID NO: 33 and a light chain variable region having at least 95% sequence identity to the amino acid sequence of SEQ ID NO: 34; or (v) a heavy chain variable region having at least 95% sequence identity to the amino acid sequence of SEQ ID NO: 51 and a light chain variable region having at least 95% sequence identity to the amino acid sequence of SEQ ID NO: 52; or (vi) a heavy chain variable region having at least 95% sequence identity to the amino acid sequence of SEQ ID NO: 69 and a light chain variable region having at least 95% sequence identity to the amino acid sequence of SEQ ID NO: 71; or (vii) a heavy chain variable region having at least 95% sequence identity to the amino acid sequence of SEQ ID NO: 70 and a light chain variable region having at least 95% sequence identity to the amino acid sequence of SEQ ID NO: 71; or (viii) a heavy chain variable region having at least 95% sequence identity to the amino acid sequence of SEQ ID NO: 89 and a light chain variable region having at least 95% sequence identity to the amino acid sequence of SEQ ID NO: 90; or (ix) a heavy chain variable region having at least 95% sequence identity to the amino acid sequence of SEQ ID NO: 125 and a light chain variable region having at least 95% sequence identity to the amino acid sequence of SEQ ID NO: 126; or (x) a heavy chain variable region having at least 95% sequence identity to the amino acid sequence of SEQ ID NO: 143 and a light chain variable region having at least 95% sequence identity to the amino acid sequence of SEQ ID NO: 144; or (xi) a heavy chain variable region having at least 95% sequence identity to the amino acid sequence of SEQ ID NO: 161 and a light chain variable region having at least 95% sequence identity to the amino acid sequence of SEQ ID NO: 162; or (xii) a heavy chain variable region having at least 95% sequence identity to the amino acid sequence of SEQ ID NO: 197 and a light chain variable region having at least 95% sequence identity to the amino acid sequence of SEQ ID NO: 198; or (xi ii) a heavy chain variable region having at least 95% sequence identity to the amino acid sequence of SEQ ID NO: 215 and a light chain variable region having at least 95% sequence identity to the amino acid sequence of SEQ ID NO: 216.
160. The anti-lyssavirus antibody, or the antigen-binding fragment thereof, for use according to item 159, wherein the antibody, or the antigen-binding fragment thereof, comprises: (i) a heavy chain variable region having at least 97% sequence identity to the amino acid sequence of SEQ ID NO: 107 and a light chain variable region having at least 97% sequence identity to the amino acid sequence of SEQ ID NO: 108; or (ii) a heavy chain variable region having at least 97% sequence identity to the amino acid sequence of SEQ ID NO: 179 and a light chain variable region having at least 97% sequence identity to the amino acid sequence of SEQ ID NO: 180; or (iii) a heavy chain variable region having at least 97% sequence identity to the amino acid sequence of SEQ ID NO: 15 and a light chain variable region having at least 97% sequence identity to the amino acid sequence of SEQ ID NO: 16; or (iv) a heavy chain variable region having at least 97% sequence identity to the amino acid sequence of SEQ ID NO: 33 and a light chain variable region having at least 97% sequence identity to the amino acid sequence of SEQ ID NO: 34; or (v) a heavy chain variable region having at least 97% sequence identity to the amino acid sequence of SEQ ID NO: 51 and a light chain variable region having at least 97% sequence identity to the amino acid sequence of SEQ ID NO: 52; or (vi) a heavy chain variable region having at least 97% sequence identity to the amino acid sequence of SEQ ID NO: 69 and a light chain variable region having at least 97% sequence identity to the amino acid sequence of SEQ ID NO: 71; or (vii) a heavy chain variable region having at least 97% sequence identity to the amino acid sequence of SEQ ID NO: 70 and a light chain variable region having at least 97% sequence identity to the amino acid sequence of SEQ ID NO: 71; or (viii) a heavy chain variable region having at least 97% sequence identity to the amino acid sequence of SEQ ID NO: 89 and a light chain variable region having at least 97% sequence identity to the amino acid sequence of SEQ ID NO: 90; or (ix) a heavy chain variable region having at least 97% sequence identity to the amino acid sequence of SEQ ID NO: 125 and a light chain variable region having at least 97% sequence identity to the amino acid sequence of SEQ ID NO: 126; or (x) a heavy chain variable region having at least 97% sequence identity to the amino acid sequence of SEQ ID NO: 143 and a light chain variable region having at least 97% sequence identity to the amino acid sequence of SEQ ID NO: 144; or (xi) a heavy chain variable region having at least 97% sequence identity to the amino acid sequence of SEQ ID NO: 161 and a light chain variable region having at least 97% sequence identity to the amino acid sequence of SEQ ID NO: 162; or (xii) a heavy chain variable region having at least 97% sequence identity to the amino acid sequence of SEQ ID NO: 197 and a light chain variable region having at least 97% sequence identity to the amino acid sequence of SEQ ID NO: 198; or (xiii) a heavy chain variable region having at least 97% sequence identity to the amino acid sequence of SEQ ID NO: 215 and a light chain variable region having at least 97% sequence identity to the amino acid sequence of SEQ ID NO: 216.
161. The anti-Lyssavirus antibody, or the antigen-binding fragment thereof, for use according to item 160, wherein the antibody, or the antigen-binding fragment thereof, comprises: (i) a heavy chain variable region having at least 98% sequence identity to the amino acid sequence of SEQ ID NO: 107 and a light chain variable region having at least 98% sequence identity to the amino acid sequence of SEQ ID NO: 108; or (ii) a heavy chain variable region having at least 98% sequence identity to the amino acid sequence of SEQ ID NO: 179 and a light chain variable region having at least 98% sequence identity to the amino acid sequence of SEQ ID NO: 180; or (iii) a heavy chain variable region having at least 98% sequence identity to the amino acid sequence of SEQ ID NO: 15 and a light chain variable region having at least 98% sequence identity to the amino acid sequence of SEQ ID NO: 16; or (iv) a heavy chain variable region having at least 98% sequence identity to the amino acid sequence of SEQ ID NO: 33 and a light chain variable region having at least 98% sequence identity to the amino acid sequence of SEQ ID NO: 34; or (v) a heavy chain variable region having at least 98% sequence identity to the amino acid sequence of SEQ ID NO: 51 and a light chain variable region having at least 98% sequence identity to the amino acid sequence of SEQ ID NO: 52; or (vi) a heavy chain variable region having at least 98% sequence identity to the amino acid sequence of SEQ ID NO: 69 and a light chain variable region having at least 98% sequence identity to the amino acid sequence of SEQ ID NO: 71; or (vii) a heavy chain variable region having at least 98% sequence identity to the amino acid sequence of SEQ ID NO: 70 and a light chain variable region having at least 98% sequence identity to the amino acid sequence of SEQ ID NO: 71; or (viii) a heavy chain variable region having at least 98% sequence identity to the amino acid sequence of SEQ ID NO: 89 and a light chain variable region having at least 98% sequence identity to the amino acid sequence of SEQ ID NO: 90; or (ix) a heavy chain variable region having at least 98% sequence identity to the amino acid sequence of SEQ ID NO: 125 and a light chain variable region having at least 98% sequence identity to the amino acid sequence of SEQ ID NO: 126; or (x) a heavy chain variable region having at least 98% sequence identity to the amino acid sequence of SEQ ID NO: 143 and a light chain variable region having at least 98% sequence identity to the amino acid sequence of SEQ ID NO: 144; or (xi) a heavy chain variable region having at least 98% sequence identity to the amino acid sequence of SEQ ID NO: 161 and a light chain variable region having at least 98% sequence identity to the amino acid sequence of SEQ ID NO: 162; or (xii) a heavy chain variable region having at least 98% sequence identity to the amino acid sequence of SEQ ID NO: 197 and a light chain variable region having at least 98% sequence identity to the amino acid sequence of SEQ ID NO: 198; or (xiii) a heavy chain variable region having at least 98% sequence identity to the amino acid sequence of SEQ ID NO: 215 and a light chain variable region having at least 98% sequence identity to the amino acid sequence of SEQ ID NO: 216.
162. The anti-lyssavirus antibody, or the antigen-binding fragment thereof, for use according to item 161, wherein the antibody, or the antigen-binding fragment thereof, comprises: (i) a heavy chain variable region having at least 99% sequence identity to the amino acid sequence of SEQ ID NO: 107 and a light chain variable region having at least 99% sequence identity to the amino acid sequence of SEQ ID NO: 108; or (ii) a heavy chain variable region having at least 99% sequence identity to the amino acid sequence of SEQ ID NO: 179 and a light chain variable region having at least 99% sequence identity to the amino acid sequence of SEQ ID NO: 180; or (iii) a heavy chain variable region having at least 99% sequence identity to the amino acid sequence of SEQ ID NO: 15 and a light chain variable region having at least 99% sequence identity to the amino acid sequence of SEQ ID NO: 16; or (iv) a heavy chain variable region having at least 99% sequence identity to the amino acid sequence of SEQ ID NO: 33 and a light chain variable region having at least 99% sequence identity to the amino acid sequence of SEQ ID NO: 34; or (v) a heavy chain variable region having at least 99% sequence identity to the amino acid sequence of SEQ ID NO: 51 and a light chain variable region having at least 99% sequence identity to the amino acid sequence of SEQ ID NO: 52; or (vi) a heavy chain variable region having at least 99% sequence identity to the amino acid sequence of SEQ ID NO: 69 and a light chain variable region having at least 99% sequence identity to the amino acid sequence of SEQ ID NO: 71; or (vii) a heavy chain variable region having at least 99% sequence identity to the amino acid sequence of SEQ ID NO: 70 and a light chain variable region having at least 99% sequence identity to the amino acid sequence of SEQ ID NO: 71; or (viii) a heavy chain variable region having at least 99% sequence identity to the amino acid sequence of SEQ ID NO: 89 and a light chain variable region having at least 99% sequence identity to the amino acid sequence of SEQ ID NO: 90; or (ix) a heavy chain variable region having at least 99% sequence identity to the amino acid sequence of SEQ ID NO: 125 and a light chain variable region having at least 99% sequence identity to the amino acid sequence of SEQ ID NO: 126; or (x) a heavy chain variable region having at least 99% sequence identity to the amino acid sequence of SEQ ID NO: 143 and a light chain variable region having at least 99% sequence identity to the amino acid sequence of SEQ ID NO: 144; or (xi) a heavy chain variable region having at least 99% sequence identity to the amino acid sequence of SEQ ID NO: 161 and a light chain variable region having at least 99% sequence identity to the amino acid sequence of SEQ ID NO: 162; or (xii) a heavy chain variable region having at least 99% sequence identity to the amino acid sequence of SEQ ID NO: 197 and a light chain variable region having at least 99% sequence identity to the amino acid sequence of SEQ ID NO: 198; or (xiii) a heavy chain variable region having at least 99% sequence identity to the amino acid sequence of SEQ ID NO: 215 and a light chain variable region having at least 99% sequence identity to the amino acid sequence of SEQ ID NO: 216.
163. The anti-lyssavirus antibody, or the antigen-binding fragment thereof, for use according to item 162, wherein the antibody, or the antigen-binding fragment thereof, comprises: (i) a heavy chain variable region as set forth in SEQ ID NO: 107 and a light chain variable region as set forth in SEQ ID NO: 108; or (ii) a heavy chain variable region as set forth in SEQ ID NO: 179 and a light chain variable region as set forth in SEQ ID NO: 180; or (iii) a heavy chain variable region as set forth in SEQ ID NO: 15 and a light chain variable region as set forth in SEQ ID NO: 16; or (iv) a heavy chain variable region as set forth in SEQ ID NO: 33 and a light chain variable region as set forth in SEQ ID NO: 34; or (v) a heavy chain variable region as set forth in SEQ ID NO: 51 and a light chain variable region as set forth in SEQ ID NO: 52; or (vi) a heavy chain variable region as set forth in SEQ ID NO: 69 and a light chain variable region as set forth in SEQ ID NO: 71; or (vii) a heavy chain variable region as set forth in SEQ ID NO: 70 and a light chain variable region as set forth in SEQ ID NO: 71; or (viii) a heavy chain variable region as set forth in SEQ ID NO: 89 and a light chain variable region as set forth in SEQ ID NO: 90; or (ix) a heavy chain variable region as set forth in SEQ ID NO: 125 and a light chain variable region as set forth in SEQ ID NO: 126; or (x) a heavy chain variable region as set forth in SEQ ID NO: 143 and a light chain variable region as set forth in SEQ ID NO: 144; or (xi) a heavy chain variable region as set forth in SEQ ID NO: 161 and a light chain variable region as set forth in SEQ ID NO: 162; or (xii) a heavy chain variable region as set forth in SEQ ID NO: 197 and a light chain variable region as set forth in SEQ ID NO: 198; or (xiii) a heavy chain variable region as set forth in SEQ ID NO: 215 and a light chain variable region as set forth in SEQ ID NO: 216.
164. The anti-lyssavirus antibody, or the antigen-binding fragment thereof, for use according to item 151, wherein the antibody, or the antigen-binding fragment thereof, comprises a heavy chain variable region having at least 70% sequence identity to the amino acid sequence of SEQ ID NO: 107 and a light chain variable region having at least 70% sequence identity to the amino acid sequence of SEQ ID NO: 108.
165. The anti-lyssavirus antibody, or the antigen-binding fragment thereof, for use according to item 164, wherein the antibody, or the antigen-binding fragment thereof, comprises a heavy chain variable region having at least 75% sequence identity to the amino acid sequence of SEQ ID NO: 107 and a light chain variable region having at least 75% sequence identity to the amino acid sequence of SEQ ID NO: 108.
166. The anti-lyssavirus antibody, or the antigen-binding fragment thereof, for use according to item 165, wherein the antibody, or the antigen-binding fragment thereof, comprises a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108.
167. The anti-lyssavirus antibody, or the antigen-binding fragment thereof, for use according to item 166, wherein the antibody, or the antigen-binding fragment thereof, comprises a heavy chain variable region having at least 80% sequence identity to the amino acid sequence of SEQ ID NO: 107 and a light chain variable region having at least 80% sequence identity to the amino acid sequence of SEQ ID NO: 108.
168. The anti-lyssavirus antibody, or the antigen-binding fragment thereof, for use according to item 167, wherein the antibody, or the antigen-binding fragment thereof, comprises a heavy chain variable region having at least 85% sequence identity to the amino acid sequence of SEQ ID NO: 107 and a light chain variable region having at least 85% sequence identity to the amino acid sequence of SEQ ID NO: 108.
169. The anti-lyssavirus antibody, or the antigen-binding fragment thereof, for use according to item 168, wherein the antibody, or the antigen-binding fragment thereof, comprises a heavy chain variable region having at least 88% sequence identity to the amino acid sequence of SEQ ID NO: 107 and a light chain variable region having at least 88% sequence identity to the amino acid sequence of SEQ ID NO: 108.
170. The anti-lyssavirus antibody, or the antigen-binding fragment thereof, for use according to item 169, wherein the antibody, or the antigen-binding fragment thereof, comprises a heavy chain variable region having at least 90% sequence identity to the amino acid sequence of SEQ ID NO: 107 and a light chain variable region having at least 90% sequence identity to the amino acid sequence of SEQ ID NO: 108.
171. The anti-lyssavirus antibody, or the antigen-binding fragment thereof, for use according to item 170, wherein the antibody, or the antigen-binding fragment thereof, comprises a heavy chain variable region having at least 92% sequence identity to the amino acid sequence of SEQ ID NO: 107 and a light chain variable region having at least 92% sequence identity to the amino acid sequence of SEQ ID NO: 108.
172. The anti-lyssavirus antibody, or the antigen-binding fragment thereof, for use according to item 171, wherein the antibody, or the antigen-binding fragment thereof, comprises a heavy chain variable region having at least 95% sequence identity to the amino acid sequence of SEQ ID NO: 107 and a light chain variable region having at least 95% sequence identity to the amino acid sequence of SEQ ID NO: 108.
173. The anti-lyssavirus antibody, or the antigen-binding fragment thereof, for use according to item 172, wherein the antibody, or the antigen-binding fragment thereof, comprises a heavy chain variable region having at least 97% sequence identity to the amino acid sequence of SEQ ID NO: 107 and a light chain variable region having at least 97% sequence identity to the amino acid sequence of SEQ ID NO: 108.
174. The anti-lyssavirus antibody, or the antigen-binding fragment thereof, for use according to item 173, wherein the antibody, or the antigen-binding fragment thereof, comprises a heavy chain variable region having at least 98% sequence identity to the amino acid sequence of SEQ ID NO: 107 and a light chain variable region having at least 98% sequence identity to the amino acid sequence of SEQ ID NO: 108.
175. The anti-lyssavirus antibody, or the antigen-binding fragment thereof, for use according to item 174, wherein the antibody, or the antigen-binding fragment thereof, comprises a heavy chain variable region having at least 99% sequence identity to the amino acid sequence of SEQ ID NO: 107 and a light chain variable region having at least 99% sequence identity to the amino acid sequence of SEQ ID NO: 108.
176. The anti-Lyssavirus antibody, or the antigen-binding fragment thereof, for use according to item 175, wherein the antibody, or the antigen-binding fragment thereof, comprises a heavy chain variable region as set forth in SEQ ID NO: 107 and a light chain variable region as set forth in SEQ ID NO: 108.
177. The anti-lyssavirus antibody, or the antigen-binding fragment thereof, for use according to item 151, wherein the antibody, or the antigen-binding fragment thereof, comprises a heavy chain variable region having at least 70% sequence identity to the amino acid sequence of SEQ ID NO: 179 and a light chain variable region having at least 70% sequence identity to the amino acid sequence of SEQ ID NO: 180.
178. The anti-lyssavirus antibody, or the antigen-binding fragment thereof, for use according to item 177, wherein the antibody, or the antigen-binding fragment thereof, comprises a heavy chain variable region having at least 75% sequence identity to the amino acid sequence of SEQ ID NO: 179 and a light chain variable region having at least 75% sequence identity to the amino acid sequence of SEQ ID NO: 180.
179. The anti-lyssavirus antibody, or the antigen-binding fragment thereof, for use according to item 178, wherein the antibody, or the antigen-binding fragment thereof, comprises a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180.
180. The anti-Lyssavirus antibody, or the antigen-binding fragment thereof, for use according to item 179, wherein the antibody, or the antigen-binding fragment thereof, comprises a heavy chain variable region having at least 80% sequence identity to the amino acid sequence of SEQ ID NO: 179 and a light chain variable region having at least 80% sequence identity to the amino acid sequence of SEQ ID NO: 180.
181. The anti-lyssavirus antibody, or the antigen-binding fragment thereof, for use according to item 180, wherein the antibody, or the antigen-binding fragment thereof, comprises a heavy chain variable region having at least 85% sequence identity to the amino acid sequence of SEQ ID NO: 179 and a light chain variable region having at least 85% sequence identity to the amino acid sequence of SEQ ID NO: 180.
182. The anti-lyssavirus antibody, or the antigen-binding fragment thereof, for use according to item 181, wherein the antibody, or the antigen-binding fragment thereof, comprises a heavy chain variable region having at least 88% sequence identity to the amino acid sequence of SEQ ID NO: 179 and a light chain variable region having at least 88% sequence identity to the amino acid sequence of SEQ ID NO: 180.
183. The anti-lyssavirus antibody, or the antigen-binding fragment thereof, for use according to item 182, wherein the antibody, or the antigen-binding fragment thereof, comprises a heavy chain variable region having at least 90% sequence identity to the amino acid sequence of SEQ ID NO: 179 and a light chain variable region having at least 90% sequence identity to the amino acid sequence of SEQ ID NO: 180.
184. The anti-lyssavirus antibody, or the antigen-binding fragment thereof, for use according to item 183, wherein the antibody, or the antigen-binding fragment thereof, comprises a heavy chain variable region having at least 92% sequence identity to the amino acid sequence of SEQ ID NO: 179 and a light chain variable region having at least 92% sequence identity to the amino acid sequence of SEQ ID NO: 180.
185. The anti-lyssavirus antibody, or the antigen-binding fragment thereof, for use according to item 184, wherein the antibody, or the antigen-binding fragment thereof, comprises a heavy chain variable region having at least 95% sequence identity to the amino acid sequence of SEQ ID NO: 179 and a light chain variable region having at least 95% sequence identity to the amino acid sequence of SEQ ID NO: 180.
186. The anti-lyssavirus antibody, or the antigen-binding fragment thereof, for use according to item 185, wherein the antibody, or the antigen-binding fragment thereof, comprises a heavy chain variable region having at least 97% sequence identity to the amino acid sequence of SEQ ID NO: 179 and a light chain variable region having at least 97% sequence identity to the amino acid sequence of SEQ ID NO: 180.
187. The anti-lyssavirus antibody, or the antigen-binding fragment thereof, for use according to item 186, wherein the antibody, or the antigen-binding fragment thereof, comprises a heavy chain variable region having at least 98% sequence identity to the amino acid sequence of SEQ ID NO: 179 and a light chain variable region having at least 98% sequence identity to the amino acid sequence of SEQ ID NO: 180.
188. The anti-lyssavirus antibody, or the antigen-binding fragment thereof, for use according to item 187, wherein the antibody, or the antigen-binding fragment thereof, comprises a heavy chain variable region having at least 99% sequence identity to the amino acid sequence of SEQ ID NO: 179 and a light chain variable region having at least 99% sequence identity to the amino acid sequence of SEQ ID NO: 180.
189. The anti-lyssavirus antibody, or the antigen-binding fragment thereof, for use according to item 188, wherein the antibody, or the antigen-binding fragment thereof, comprises a heavy chain variable region as set forth in SEQ ID NO: 179 and a light chain variable region as set forth in SEQ ID NO: 180.
190. The anti-lyssavirus antibody, or the antigen-binding fragment thereof, for use according to any one of items 1-190, wherein the antibody, or the antigen-binding fragment thereof, is RVC20, RVC58, RVA122, RVA144, RVB185, RVB492, RVC3, RVC21, RVC38, RVC44, RVC68, or RVC111.
191. The anti-lyssavirus antibody, or the antigen-binding fragment thereof, for use according to any one of items 1-190, wherein the antibody, or the antigen-binding fragment thereof, is RVC20 or RVC58.
192. The anti-lyssavirus antibody, or the antigen-binding fragment thereof, for use according to any one of items 1-191, wherein the antibody, or the antigen-binding fragment thereof, is RVC20.
193. The anti-lyssavirus antibody, or the antigen-binding fragment thereof, for use according to any one of items 1-191, wherein the antibody, or the antigen-binding fragment thereof, is RVC58.
194. The anti-lyssavirus antibody, or the antigen-binding fragment thereof, for use according to item 72-80, wherein at least one of the two anti-lyssavirus antibodies, or the antigen-binding fragments thereof, administered in combination, is as defined in any one of items 112-193.
195. The anti-lyssavirus antibody, or the antigen-binding fragment thereof, for use according to item 72-80, wherein both anti-lyssavirus antibodies, or the antigen-binding fragments thereof, administered in combination, are as defined in any one of items 112-193.
196. The anti-lyssavirus antibody, or the antigen-binding fragment thereof, for use according to any one of items 72-80 and 194-195, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 70% identical to the amino acid sequences of SEQ ID NOs: 165-169 and 171 or to the amino acid sequences of SEQ ID NOs: 165-168 and 170-171, respectively.
197. The anti-lyssavirus antibody, or the antigen-binding fragment thereof, for use according to item 196, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 75% identical to the amino acid sequences of SEQ ID NOs: 165-169 and 171 or to the amino acid sequences of SEQ ID NOs: 165-168 and 170-171, respectively.
198. The anti-lyssavirus antibody, or the antigen-binding fragment thereof, for use according to item 197, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively.
199. The anti-lyssavirus antibody, or the antigen-binding fragment thereof, for use according to item 198, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 80% identical to the amino acid sequences of SEQ ID NOs: 165-169 and 171 or to the amino acid sequences of SEQ ID NOs: 165-168 and 170-171, respectively.
200. The anti-lyssavirus antibody, or the antigen-binding fragment thereof, for use according to item 199, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 85% identical to the amino acid sequences of SEQ ID NOs: 165-169 and 171 or to the amino acid sequences of SEQ ID NOs: 165-168 and 170-171, respectively.
201. The anti-lyssavirus antibody, or the antigen-binding fragment thereof, for use according to item 200, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 88% identical to the amino acid sequences of SEQ ID NOs: 165-169 and 171 or to the amino acid sequences of SEQ ID NOs: 165-168 and 170-171, respectively.
202. The anti-lyssavirus antibody, or the antigen-binding fragment thereof, for use according to item 201, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 90% identical to the amino acid sequences of SEQ ID NOs: 165-169 and 171 or to the amino acid sequences of SEQ ID NOs: 165-168 and 170-171, respectively.
203. The anti-Lyssavirus antibody, or the antigen-binding fragment thereof, for use according to item 202, wherein one of the two anti-Lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 92% identical to the amino acid sequences of SEQ ID NOs: 165-169 and 171 or to the amino acid sequences of SEQ ID NOs: 165-168 and 170-171, respectively.
204. The anti-lyssavirus antibody, or the antigen-binding fragment thereof, for use according to item 203, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 95% identical to the amino acid sequences of SEQ ID NOs: 165-169 and 171 or to the amino acid sequences of SEQ ID NOs: 165-168 and 170-171, respectively.
205. The anti-lyssavirus antibody, or the antigen-binding fragment thereof, for use according to item 204, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 97% identical to the amino acid sequences of SEQ ID NOs: 165-169 and 171 or to the amino acid sequences of SEQ ID NOs: 165-168 and 170-171, respectively.
206. The anti-lyssavirus antibody, or the antigen-binding fragment thereof, for use according to item 205, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 93-97 and 99 or to the amino acid sequences of SEQ ID NOs: 93-96 and 98-99, respectively; and the other of the two anti-Lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 98% identical to the amino acid sequences of SEQ ID NOs: 165-169 and 171 or to the amino acid sequences of SEQ ID NOs: 165-168 and 170-171, respectively.
207. The anti-lyssavirus antibody, or the antigen-binding fragment thereof, for use according to item 206, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 99% identical to the amino acid sequences of SEQ ID NOs: 165-169 and 171 or to the amino acid sequences of SEQ ID NOs: 165-168 and 170-171, respectively.
208. The anti-lyssavirus antibody, or the antigen-binding fragment thereof, for use according to item 207, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences as set forth in SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences as set forth in SEQ ID NOs: 165-169 and 171 or to the amino acid sequences of SEQ ID NOs: 165-168 and 170-171, respectively.
209. The anti-Lyssavirus antibody, or the antigen-binding fragment thereof, for use according to item 196, wherein one of the two anti-lyssavirus antibodies, or the antigen-binding fragments thereof, administered in combination comprises a heavy chain variable region having at least 70% sequence identity to the amino acid sequence of SEQ ID NO: 107 and a light chain variable region having at least 70% sequence identity to the amino acid sequence of SEQ ID NO: 108; and the other of the two anti-lyssavirus antibodies, or the antigen-binding fragments thereof, administered in combination comprises a heavy chain variable region having at least 70% sequence identity to the amino acid sequence of SEQ ID NO: 179 and a light chain variable region having at least 70% sequence identity to the amino acid sequence of SEQ ID NO: 180.
210. The anti-Lyssavirus antibody, or the antigen-binding fragment thereof, for use according to item 209, wherein one of the two anti-lyssavirus antibodies, or the antigen-binding fragments thereof, administered in combination comprises a heavy chain variable region having at least 75% sequence identity to the amino acid sequence of SEQ ID NO: 107 and a light chain variable region having at least 75% sequence identity to the amino acid sequence of SEQ ID NO: 108; and the other of the two anti-lyssavirus antibodies, or the antigen-binding fragments thereof, administered in combination comprises a heavy chain variable region having at least 75% sequence identity to the amino acid sequence of SEQ ID NO: 179 and a light chain variable region having at least 75% sequence identity to the amino acid sequence of SEQ ID NO: 180.
211. The anti-lyssavirus antibody, or the antigen-binding fragment thereof, for use according to item 210, wherein one of the two anti-lyssavirus antibodies, or the antigen-binding fragments thereof, administered in combination comprises a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108; and the other of the two anti-lyssavirus antibodies, or the antigen-binding fragments thereof, administered in combination comprises a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180.
212. The anti-lyssavirus antibody, or the antigen-binding fragment thereof, for use according to item 211, wherein one of the two anti-lyssavirus antibodies, or the antigen-binding fragments thereof, administered in combination comprises a heavy chain variable region having at least 80% sequence identity to the amino acid sequence of SEQ ID NO: 107 and a light chain variable region having at least 80% sequence identity to the amino acid sequence of SEQ ID NO: 108; and the other of the two anti-lyssavirus antibodies, or the antigen-binding fragments thereof, administered in combination comprises a heavy chain variable region having at least 80% sequence identity to the amino acid sequence of SEQ ID NO: 179 and a light chain variable region having at least 80% sequence identity to the amino acid sequence of SEQ ID NO: 180.
213. The anti-lyssavirus antibody, or the antigen-binding fragment thereof, for use according to item 212, wherein one of the two anti-lyssavirus antibodies, or the antigen-binding fragments thereof, administered in combination comprises a heavy chain variable region having at least 85% sequence identity to the amino acid sequence of SEQ ID NO: 107 and a light chain variable region having at least 85% sequence identity to the amino acid sequence of SEQ ID NO: 108; and the other of the two anti-lyssavirus antibodies, or the antigen-binding fragments thereof, administered in combination comprises a heavy chain variable region having at least 85% sequence identity to the amino acid sequence of SEQ ID NO: 179 and a light chain variable region having at least 85% sequence identity to the amino acid sequence of SEQ ID NO: 180.
214. The anti-lyssavirus antibody, or the antigen-binding fragment thereof, for use according to item 213, wherein one of the two anti-Lyssavirus antibodies, or the antigen-binding fragments thereof, administered in combination comprises a heavy chain variable region having at least 88% sequence identity to the amino acid sequence of SEQ ID NO: 107 and a light chain variable region having at least 88% sequence identity to the amino acid sequence of SEQ ID NO: 108; and the other of the two anti-lyssavirus antibodies, or the antigen-binding fragments thereof, administered in combination comprises a heavy chain variable region having at least 88% sequence identity to the amino acid sequence of SEQ ID NO: 179 and a light chain variable region having at least 88% sequence identity to the amino acid sequence of SEQ ID NO: 180.
215. The anti-lyssavirus antibody, or the antigen-binding fragment thereof, for use according to item 214, wherein one of the two anti-lyssavirus antibodies, or the antigen-binding fragments thereof, administered in combination comprises a heavy chain variable region having at least 90% sequence identity to the amino acid sequence of SEQ ID NO: 107 and a light chain variable region having at least 90% sequence identity to the amino acid sequence of SEQ ID NO: 108; and the other of the two anti-lyssavirus antibodies, or the antigen-binding fragments thereof, administered in combination comprises a heavy chain variable region having at least 90% sequence identity to the amino acid sequence of SEQ ID NO: 179 and a light chain variable region having at least 90% sequence identity to the amino acid sequence of SEQ ID NO: 180.
216. The anti-lyssavirus antibody, or the antigen-binding fragment thereof, for use according to item 215, wherein one of the two anti-lyssavirus antibodies, or the antigen-binding fragments thereof, administered in combination comprises a heavy chain variable region having at least 92% sequence identity to the amino acid sequence of SEQ ID NO: 107 and a light chain variable region having at least 92% sequence identity to the amino acid sequence of SEQ ID NO: 108; and the other of the two anti-lyssavirus antibodies, or the antigen-binding fragments thereof, administered in combination comprises a heavy chain variable region having at least 92% sequence identity to the amino acid sequence of SEQ ID NO: 179 and a light chain variable region having at least 92% sequence identity to the amino acid sequence of SEQ ID NO: 180.
217. The anti-lyssavirus antibody, or the antigen-binding fragment thereof, for use according to item 216, wherein one of the two anti-lyssavirus antibodies, or the antigen-binding fragments thereof, administered in combination comprises a heavy chain variable region having at least 95% sequence identity to the amino acid sequence of SEQ ID NO: 107 and a light chain variable region having at least 95% sequence identity to the amino acid sequence of SEQ ID NO: 108; and the other of the two anti-lyssavirus antibodies, or the antigen-binding fragments thereof, administered in combination comprises a heavy chain variable region having at least 95% sequence identity to the amino acid sequence of SEQ ID NO: 179 and a light chain variable region having at least 95% sequence identity to the amino acid sequence of SEQ ID NO: 180.
218. The anti-lyssavirus antibody, or the antigen-binding fragment thereof, for use according to item 217, wherein one of the two anti-lyssavirus antibodies, or the antigen-binding fragments thereof, administered in combination comprises a heavy chain variable region having at least 97% sequence identity to the amino acid sequence of SEQ ID NO: 107 and a light chain variable region having at least 97% sequence identity to the amino acid sequence of SEQ ID NO: 108; and the other of the two anti-lyssavirus antibodies, or the antigen-binding fragments thereof, administered in combination comprises a heavy chain variable region having at least 97% sequence identity to the amino acid sequence of SEQ ID NO: 179 and a light chain variable region having at least 97% sequence identity to the amino acid sequence of SEQ ID NO: 180.
219. The anti-lyssavirus antibody, or the antigen-binding fragment thereof, for use according to item 218, wherein one of the two anti-lyssavirus antibodies, or the antigen-binding fragments thereof, administered in combination comprises a heavy chain variable region having at least 98% sequence identity to the amino acid sequence of SEQ ID NO: 107 and a light chain variable region having at least 98% sequence identity to the amino acid sequence of SEQ ID NO: 108; and the other of the two anti-lyssavirus antibodies, or the antigen-binding fragments thereof, administered in combination comprises a heavy chain variable region having at least 98% sequence identity to the amino acid sequence of SEQ ID NO: 179 and a light chain variable region having at least 98% sequence identity to the amino acid sequence of SEQ ID NO: 180.
220. The anti-lyssavirus antibody, or the antigen-binding fragment thereof, for use according to item 219, wherein one of the two anti-lyssavirus antibodies, or the antigen-binding fragments thereof, administered in combination comprises a heavy chain variable region having at least 99% sequence identity to the amino acid sequence of SEQ ID NO: 107 and a light chain variable region having at least 99% sequence identity to the amino acid sequence of SEQ ID NO: 108; and the other of the two anti-lyssavirus antibodies, or the antigen-binding fragments thereof, administered in combination comprises a heavy chain variable region having at least 99% sequence identity to the amino acid sequence of SEQ ID NO: 179 and a light chain variable region having at least 99% sequence identity to the amino acid sequence of SEQ ID NO: 180.
221. The anti-lyssavirus antibody, or the antigen-binding fragment thereof, for use according to item 220, wherein one of the two anti-lyssavirus antibodies, or the antigen-binding fragments thereof, administered in combination comprises a heavy chain variable region as set forth in SEQ ID NO: 107 and a light chain variable region as set forth in SEQ ID NO: 108; and the other of the two anti-lyssavirus antibodies, or the antigen-binding fragments thereof, administered in combination comprises a heavy chain variable region as set forth in SEQ ID NO: 179 and a light chain variable region as set forth in SEQ ID NO: 180.
222. The anti-lyssavirus antibody, or the antigen-binding fragment thereof, for use according to any one of items 112-123, 125-136, 138-149, 151-162, 164-175, 177-188, 196-207, and 209-220, wherein the amino acid sequence comprises a deletion of one or more amino acids in comparison to the reference sequence.
223. The anti-lyssavirus antibody, or the antigen-binding fragment thereof, for use according to any one of items 112-123, 125-136, 138-149, 151-162, 164-175, 177-188, 196-207, 209-220, and 222, wherein the amino acid sequence comprises an insertion of one or more amino acids in comparison to the reference sequence.
224. The anti-lyssavirus antibody, or the antigen-binding fragment thereof, for use according to any one of items 112-123, 125-136, 138-149, 151-162, 164-175, 177-188, 196-207, 209-220, 222, and 223, wherein the amino acid sequence comprises a substitution of one or more amino acids in comparison to the reference sequence.
225. The anti-lyssavirus antibody, or the antigen-binding fragment thereof, for use according to item 224, wherein the substitution is a conservative substitution.
226. The anti-lyssavirus antibody, or the antigen-binding fragment thereof, for use according to item 224 or 225, wherein the substitution is a non-conservative substitution.
227. The anti-lyssavirus antibody, or the antigen-binding fragment thereof, for use according to any one of items 112-123, 125-136, 138-149, 151-162, 164-175, 177-188, 196-207, 209-220, and 222-226, wherein the alteration(s) in the amino acid sequence in comparison to the reference sequence does not abolish functionality.
228. The anti-lyssavirus antibody, or the antigen-binding fragment thereof, for use according to item 227, wherein the functionality is binding to the same epitope as the reference sequence.
229. The anti-lyssavirus antibody, or the antigen-binding fragment thereof, for use according to item 227 or 228, wherein the functionality is neutralizing infection of a lyssavirus.
230. The anti-lyssavirus antibody, or the antigen-binding fragment thereof, for use according to item 229, wherein the lyssavirus is RABV.
231. The anti-lyssavirus antibody, or the antigen-binding fragment thereof, for use according to item 229, wherein the lyssavirus is EBLV-1.
232. The anti-lyssavirus antibody, or the antigen-binding fragment thereof, for use according to any one of items 1-231, wherein the antibody, or the antigen-binding fragment thereof, is comprised in a pharmaceutical composition.
233. A nucleic acid molecule comprising a polynucleotide encoding an anti-lyssavirus antibody, or an antigen-binding fragment thereof, for use in the treatment of lyssavirus infection, wherein the nucleic acid molecule is administered
   (i) into the central nervous system (CNS); and
   (ii) peripherally.
234. The nucleic acid molecule for use according to item 233, wherein the nucleic acid molecule is administered as defined in in any one of items 2-80.
235. The nucleic acid molecule for use according to item 233 or 234, wherein the encoded anti-lyssavirus antibody, or the antigen-binding fragment thereof, is as defined in any one of items 81-231.
236. The nucleic acid molecule for use according to any one of items 233-235, wherein the nucleic acid is DNA.
237. The nucleic acid molecule for use according to any one of items 233-235, wherein the nucleic acid is RNA.
238. A combination of at least two distinct nucleic acid molecules encoding an anti-lyssavirus antibody, or an antigen-binding fragment thereof, wherein one nucleic acid molecule encodes at least a CDRH1, CDRH2 and CDRH3 of a heavy chain and another nucleic acid molecule encodes at least a CDRL1, CDRL2 and CDRL3 of a corresponding light chain of the antilyssavirus antibody, for use in the treatment of lyssavirus infection, wherein the nucleic acid molecule is administered
   (i) into the central nervous system (CNS); and
   (ii) peripherally.
239. The combination for use according to item 238, wherein one nucleic acid molecule encodes at least a variable region of a heavy chain (VH) and another nucleic acid molecule encodes at least a corresponding variable region of a light chain (VL) of the antilyssavirus antibody.
240. The combination for use according to item 238, wherein one nucleic acid molecule encodes a heavy chain and another nucleic acid molecule encodes a corresponding light chain of the antilyssavirus antibody.
241. The combination for use according to any one of items 238-240, wherein the encoded anti-lyssavirus antibody, or the antigen-binding fragment thereof, is as defined in any one of items 80-231.
242. The combination for use according to any one of items 238-241, wherein the combination is administered as defined in in any one of items 2-80.
243. The combination for use according to any one of items 238-242, wherein the nucleic acid is DNA.
244. The combination for use according to any one of items 238-242, wherein the nucleic acid is RNA.
245. A pharmaceutical composition comprising (as an active ingredient) an anti-lyssavirus antibody, or an antigen-binding fragment thereof, for use in the treatment of lyssavirus infection, wherein the pharmaceutical composition is administered
   (i) into the central nervous system (CNS); and
   (ii) peripherally.
246. The pharmaceutical composition for use according to item 245, wherein the pharmaceutical composition comprises a pharmaceutically acceptable carrier, diluent or excipient.
247. The pharmaceutical composition for use according to item 246, wherein the pharmaceutical composition comprises a pharmaceutically acceptable carrier.
248. The pharmaceutical composition for use according to item 246 or 247, wherein the pharmaceutical composition comprises a pharmaceutically acceptable diluent.
249. The pharmaceutical composition for use according to any one of items 246-248, wherein the pharmaceutical composition comprises a pharmaceutically acceptable excipient.
250. The pharmaceutical composition for use according to any one of items 246-249, wherein the pharmaceutical composition comprises saline.
251. The pharmaceutical composition for use according to any one of items 246-249, wherein the pharmaceutical composition comprises a buffer.
252. The pharmaceutical composition for use according to any one of items 246-249, wherein the pharmaceutical composition comprises phosphate-buffered saline (PBS).
253. The pharmaceutical composition for use according to any one of items 246-252, wherein the pharmaceutical composition has a pH between 5.5 and 8.5.
254. The pharmaceutical composition for use according to item 253, wherein the pharmaceutical composition has a pH between 6 and 8.
255. The pharmaceutical composition for use according to item 254, wherein the pharmaceutical composition has a pH between 6.5 and 7.5.
256. The pharmaceutical composition for use according to any one of items 245-255, wherein the pharmaceutical composition is administered as defined in any one of items 2-80.
257. The pharmaceutical composition for use according to any one of items 245-256, wherein the anti-lyssavirus antibody, or the antigen-binding fragment thereof, is as defined in any one of items 81-231.
258. The pharmaceutical composition for use according to any one of items 245-257, wherein the pharmaceutical composition comprises at least two distinct anti-lyssavirus antibodies, or antigen-binding fragments thereof, as defined in any one of items 81-231.
259. The pharmaceutical composition for use according to item 258, wherein the at least two antibodies, or antigen-binding fragments thereof, specifically bind to different epitopes on the glycoprotein G of RABV.
260. The pharmaceutical composition for use according to item 259, wherein the at least two antibodies, or antigen-binding fragments thereof, specifically bind to antigenic site I and to antigenic site III of glycoprotein G of RABV.
261. The pharmaceutical composition for use according to any one of items 245-260, wherein one of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively.
262. The pharmaceutical composition for use according to item 261, wherein one of the two anti-lyssavirus antibodies, or the antigen-binding fragments thereof, comprises a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108; and the other of the two anti-lyssavirus antibodies, or the antigen-binding fragments thereof, comprises a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180.
263. The pharmaceutical composition for use according to any one of items 245-262, wherein the at least two distinct anti-lyssavirus antibodies, or antigen-binding fragments thereof, are comprised in the pharmaceutical composition in equimolar amounts.
264. The anti-lyssavirus antibody, or the antigen-binding fragment thereof, for use according to any one of items 1-232, the nucleic acid molecule for use according to any one of items 233-237, the combination for use according to any one of items 238-244, or the pharmaceutical composition for use according to any one of items 245-263, wherein the antibody, or the antigen-binding fragment thereof, the nucleic acid molecule, or the pharmaceutical composition is administered in combination with an anti-lyssavirus vaccine, an antiviral agent, interferon-alpha and/or ketamine.
265. The anti-lyssavirus antibody, or the antigen-binding fragment thereof, or the pharmaceutical composition for use according to item 264, wherein the anti-lyssavirus vaccine is a rabies vaccine.
266. The anti-lyssavirus antibody, or the antigen-binding fragment thereof, or the pharmaceutical composition for use according to item 264 or 265, wherein the antiviral agent is ribavirin.
267. The anti-lyssavirus antibody, or the antigen-binding fragment thereof, for use according to any one of items 1-232, the nucleic acid molecule for use according to any one of items 233-237, the combination for use according to any one of items 238-244, or the pharmaceutical composition for use according to any one of items 245-263, wherein the antibody, or the antigen-binding fragment thereof, or the pharmaceutical composition is administered without concomitant and/or subsequent administration of an anti-lyssavirus vaccine.
268. A kit of parts comprising at least one antibody, or antigen binding fragment thereof, as defined in any of items 65 to 232, at least one nucleic acid molecule as defined in any one of items 233-237, the combination as defined in any one of items 238-244, or at least one pharmaceutical composition according to any of items 245 to 263 for use in the treatment of lyssavirus infection, wherein the antibody, or the antigen-binding fragment thereof, or the pharmaceutical composition is administered as defined in any one of items 1-62.
269. The kit of parts for use according to item 268, wherein the kit of parts comprises at least two distinct anti-lyssavirus antibodies, or antigen-binding fragments thereof, as defined in any one of items 65-232.
270. The kit of parts for use according to item 269, wherein the at least two antibodies, or antigen-binding fragments thereof, specifically bind to different epitopes on the glycoprotein G of RABV.
271. The kit of parts for use according to item 270, wherein the at least two antibodies, or antigen-binding fragments thereof, specifically bind to antigenic site I and to antigenic site Ill of glycoprotein G of RABV.
272. The kit of parts for use according to any one of items 269-271, wherein one of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively.
273. The kit of parts for use according to item 272, wherein one of the two anti-lyssavirus antibodies, or the antigen-binding fragments thereof, comprises a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108; and the other of the two anti-lyssavirus antibodies, or the antigen-binding fragments thereof, comprises a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180.
274. Method of treating or attenuating a lyssavirus infection in a subject, wherein the method comprises administering to a subject in need thereof the antibody, or the antigen binding fragment thereof, as defined in any one of items 1-232, at least one nucleic acid molecule as defined in any one of items 233-237, the combination as defined in any one of items 238-244, or the pharmaceutical composition according to any one of items 245-263, wherein the antibody, or the antigen-binding fragment thereof, or the pharmaceutical composition is administered
   (i) into the central nervous system (CNS); and
   (ii) peripherally.
275. The method of item 274, wherein the subject is a human subject.
276. The method of item 274, wherein the subject is an animal subject.
277. The method of item 276, wherein the subject is selected from the group consisting of cows, dogs, cats, horses, goats, sheep, pigs, and rabbits.
278. An anti-lyssavirus antibody, or an antigen-binding fragment thereof, comprising an Fc moiety comprising a CH2 domain and a CH2 L4A mutation and/or a CH2 L5A mutation.
279. The anti-lyssavirus antibody, or an antigen-binding fragment thereof, according to item 278, wherein the Fc moiety further comprises a hinge domain.
280. The anti-lyssavirus antibody, or an antigen-binding fragment thereof, according to item 278 or 279, wherein the Fc moiety further comprises a CH3 domain.
281. The anti-lyssavirus antibody, or an antigen-binding fragment thereof, according to any one of items 278-280 comprising an Fc region.
282. The anti-lyssavirus antibody, or an antigen-binding fragment thereof, according to any one of items 278-281, wherein the Fc moiety is a monomer.
283. The anti-lyssavirus antibody, or an antigen-binding fragment thereof, according to any one of items 278-282, wherein the Fc moiety is a dimer.
284. The anti-lyssavirus antibody, or an antigen-binding fragment thereof, according to item 283, wherein the Fc moiety is a homodimer.
285. The anti-lyssavirus antibody, or an antigen-binding fragment thereof, according to item 283, wherein the Fc moiety is a heterodimer.
286. The anti-lyssavirus antibody, or an antigen-binding fragment thereof, according to any one of items 278-285, wherein the Fc moiety comprises an amino acid sequence derived from a human immunoglobulin sequence.
287. The anti-lyssavirus antibody, or an antigen-binding fragment thereof, according to item 286, wherein the Fc moiety comprises a human immunoglobulin sequence.
288. The anti-Lyssavirus antibody, or an antigen-binding fragment thereof, according to any one of items 278-287, wherein the Fc moiety is human.
289. The anti-lyssavirus antibody, or an antigen-binding fragment thereof, according to any one of items 278-288 comprising an Fc region derived from human IgG1.
290. The anti-Lyssavirus antibody, or an antigen-binding fragment thereof, according to any one of items 278-289 comprising a human IgG1 Fc region.
291. The anti-lyssavirus antibody, or an antigen-binding fragment thereof, according to any one of items 278-290 comprising a CH1 region.
292. The anti-lyssavirus antibody, or an antigen-binding fragment thereof, according to any one of items 278-291 comprising IgG CH1-CH2-CH3.
293. The anti-Lyssavirus antibody, or an antigen-binding fragment thereof, according to item 292 comprising human IgG1 CH1-CH2-CH3.
294. The anti-Lyssavirus antibody, or an antigen-binding fragment thereof, according to any one of items 278-293 comprising an amino acid sequence that is at least 70% identical to SEQ ID NO: 219.
295. The anti-lyssavirus antibody, or an antigen-binding fragment thereof, according to any one of items 278-293 comprising an amino acid sequence that is at least 75% identical to SEQ ID NO: 219.
296. The anti-lyssavirus antibody, or an antigen-binding fragment thereof, according to any one of items 278-293 comprising an amino acid sequence that is at least 80% identical to SEQ ID NO: 219.
297. The anti-lyssavirus antibody, or an antigen-binding fragment thereof, according to any one of items 278-293 comprising an amino acid sequence that is at least 85% identical to SEQ ID NO: 219.
298. The anti-lyssavirus antibody, or an antigen-binding fragment thereof, according to any one of items 278-293 comprising an amino acid sequence that is at least 88% identical to SEQ ID NO: 219.
299. The anti-lyssavirus antibody, or an antigen-binding fragment thereof, according to any one of items 278-293 comprising an amino acid sequence that is at least 90% identical to SEQ ID NO: 219.
300. The anti-lyssavirus antibody, or an antigen-binding fragment thereof, according to any one of items 278-293 comprising an amino acid sequence that is at least 92% identical to SEQ ID NO: 219.
301. The anti-lyssavirus antibody, or an antigen-binding fragment thereof, according to any one of items 278-293 comprising an amino acid sequence that is at least 95% identical to SEQ ID NO: 219.
302. The anti-lyssavirus antibody, or an antigen-binding fragment thereof, according to any one of items 278-293 comprising an amino acid sequence that is at least 98% identical to SEQ ID NO: 219.
303. The anti-lyssavirus antibody, or an antigen-binding fragment thereof, according to any one of items 278-293 comprising an amino acid sequence that is at least 99% identical to SEQ ID NO: 219.
304. The anti-lyssavirus antibody, or an antigen-binding fragment thereof, according to any one of items 278-293 comprising an amino acid sequence as set forth in SEQ ID NO: 219.
305. The anti-lyssavirus antibody, or an antigen-binding fragment thereof, according to any one of items 72-304, wherein the anti-lyssavirus antibody, or an antigen-binding fragment thereof, is an anti-RABV antibody or an antigen-binding fragment thereof.
306. The anti-lyssavirus antibody, or the antigen-binding fragment thereof, according to any one of items 278-305, wherein the antibody, or the antigen-binding fragment thereof, binds to lyssavirus glycoprotein G.
307. The anti-lyssavirus antibody, or the antigen-binding fragment thereof, according to item 306, wherein the antibody, or the antigen-binding fragment thereof, binds to glycoprotein G of RABV.
308. The anti-lyssavirus antibody, or the antigen-binding fragment thereof, according to item 307, wherein the antibody, or the antigen-binding fragment thereof, binds to antigenic site I or antigenic site III of glycoprotein G of RABV.
309. The anti-lyssavirus antibody, or the antigen-binding fragment thereof, according to any one of items 278-308, wherein the antibody, or the antigen-binding fragment thereof, is a monoclonal antibody and/or a human antibody.
310. The anti-lyssavirus antibody, or the antigen-binding fragment thereof, according to any one of items 278-309, wherein the antibody, or the antigen-binding fragment thereof, neutralizes lyssavirus infection by (i) RABV and (ii) at least 50% of non-RABV lyssaviruses selected from the group consisting of DUVV, EBLV-1, EBLV-2, ABLY, IRKV, KHUV, ARAV, LBV, MOK, SHIV, BBLV and WCBV, with an IC₅₀ of less than 10000 ng/ml.
311. The anti-lyssavirus antibody, or the antigen-binding fragment thereof, according to any one of items 278-310, wherein the antibody, or the antigen-binding fragment thereof, neutralizes infection by RABV CVS-11 with an IC₉₀ of 400 ng/ml or less.
312. The anti-lyssavirus antibody, or the antigen-binding fragment thereof, according to any one of items 278-311, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 201-205 and 207 or to the amino acid sequences of SEQ ID NOs: 201-204 and 206-207, respectively.
313. The anti-lyssavirus antibody, or the antigen-binding fragment thereof, according to item 278-312, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively.
314. The anti-lyssavirus antibody, or the antigen-binding fragment thereof, according to item 278-312, wherein the antibody, or the antigen-binding fragment thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively.
315. The anti-Lyssavirus antibody, or the antigen-binding fragment thereof, according to any one of items 278-314, wherein the antibody, or the antigen-binding fragment thereof, comprises: (i) a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108; or (ii) a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180; or (iii) a heavy chain variable region having at least 80%, or at least 90%, sequence identity to the amino acid sequence of SEQ ID NO: 15 and a light chain variable region having at least 80%, or at least 90%, sequence identity to the amino acid sequence of SEQ ID NO: 16; or (iv) a heavy chain variable region having at least 80%, or at least 90%, sequence identity to the amino acid sequence of SEQ ID NO: 33 and a light chain variable region having at least 80%, or at least 90%, sequence identity to the amino acid sequence of SEQ ID NO: 34; or (v) a heavy chain variable region having at least 80%, or at least 90%, sequence identity to the amino acid sequence of SEQ ID NO: 51 and a light chain variable region having at least 80%, or at least 90%, sequence identity to the amino acid sequence of SEQ ID NO: 52; or (vi) a heavy chain variable region having at least 80%, or at least 90%, sequence identity to the amino acid sequence of SEQ ID NO: 69 and a light chain variable region having at least 80%, or at least 90%, sequence identity to the amino acid sequence of SEQ ID NO: 71; or (vii) a heavy chain variable region having at least 80%, or at least 90%, sequence identity to the amino acid sequence of SEQ ID NO: 70 and a light chain variable region having at least 80%, or at least 90%, sequence identity to the amino acid sequence of SEQ ID NO: 71; or (viii) a heavy chain variable region having at least 80%, or at least 90%, sequence identity to the amino acid sequence of SEQ ID NO: 89 and a light chain variable region having at least 80%, or at least 90%, sequence identity to the amino acid sequence of SEQ ID NO: 90; or (ix) a heavy chain variable region having at least 80%, or at least 90%, sequence identity to the amino acid sequence of SEQ ID NO: 125 and a light chain variable region having at least 80%, or at least 90%, sequence identity to the amino acid sequence of SEQ ID NO: 126; or (x) a heavy chain variable region having at least 80%, or at least 90%, sequence identity to the amino acid sequence of SEQ ID NO: 143 and a light chain variable region having at least 80%, or at least 90%, sequence identity to the amino acid sequence of SEQ ID NO: 144; or (xi) a heavy chain variable region having at least 80%, or at least 90%, sequence identity to the amino acid sequence of SEQ ID NO: 161 and a light chain variable region having at least 80%, or at least 90%, sequence identity to the amino acid sequence of SEQ ID NO: 162; or (xii) a heavy chain variable region having at least 80%, or at least 90%, sequence identity to the amino acid sequence of SEQ ID NO: 197 and a light chain variable region having at least 80%, or at least 90%, sequence identity to the amino acid sequence of SEQ ID NO: 198; or (xiii) a heavy chain variable region having at least 80%, or at least 90%, sequence identity to the amino acid sequence of SEQ ID NO: 215 and a light chain variable region having at least 80%, or at least 90%, sequence identity to the amino acid sequence of SEQ ID NO: 216.
316. The anti-Lyssavirus antibody, or the antigen-binding fragment thereof, according to item 315, wherein the antibody, or the antigen-binding fragment thereof, comprises a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108.
317. The anti-lyssavirus antibody, or the antigen-binding fragment thereof, according to item 278-316, wherein the antibody, or the antigen-binding fragment thereof, comprises a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180.
318. The anti-lyssavirus antibody, or the antigen-binding fragment thereof, according to any one of items 278-317, wherein the antibody, or the antigen-binding fragment thereof, is RVC20, RVC58, RVA122, RVA144, RVB185, RVB492, RVC3, RVC21, RVC38, RVC44, RVC68, or RVC111.
319. The anti-lyssavirus antibody, or the antigen-binding fragment thereof, according to any one of items 278-318, wherein the antibody, or the antigen-binding fragment thereof, is RVC20 or RVC58.
320. The anti-Lyssavirus antibody, or the antigen-binding fragment thereof, according to any one of items 278-319, wherein the antibody, or the antigen-binding fragment thereof, is a purified antibody.
321. The anti-lyssavirus antibody, or the antigen-binding fragment thereof, according to any one of items 278-320, wherein the antibody, or the antigen-binding fragment thereof, is a single chain antibody.
322. The anti-lyssavirus antibody, or the antigen-binding fragment thereof, according to any one of items 278-321, wherein the antibody, or the antigen-binding fragment thereof, is a Fab.
323. The anti-lyssavirus antibody, or the antigen-binding fragment thereof, according to any one of items 278-321, wherein the antibody, or the antigen-binding fragment thereof, is a Fab′.
324. The anti-lyssavirus antibody, or the antigen-binding fragment thereof, according to any one of items 278-321, wherein the antibody, or the antigen-binding fragment thereof, is a F(ab′)2.
325. The anti-lyssavirus antibody, or the antigen-binding fragment thereof, according to any one of items 278-321, wherein the antibody, or the antigen-binding fragment thereof, is a Fv.
326. The anti-lyssavirus antibody, or the antigen-binding fragment thereof, according to any one of items 278-321, wherein the antibody, or the antigen-binding fragment thereof, is a scFv.
327. The anti-lyssavirus antibody, or the antigen-binding fragment thereof, according to any one of items 278-321, wherein the antibody, or the antigen-binding fragment thereof, is an IgG antibody.
328. The anti-lyssavirus antibody, or the antigen-binding fragment thereof, according to any one of items 278-321, wherein the antibody, or the antigen-binding fragment thereof, is an IgM antibody.
329. The anti-lyssavirus antibody, or the antigen-binding fragment thereof, according to any one of items 278-321, wherein the antibody, or the antigen-binding fragment thereof, is an IgA antibody.
330. The anti-lyssavirus antibody, or the antigen-binding fragment thereof, according to item 327, wherein the antibody, or the antigen-binding fragment thereof, is an IgG1 antibody.
331. The anti-lyssavirus antibody, or the antigen-binding fragment thereof, according to item 327, wherein the antibody, or the antigen-binding fragment thereof, is an IgG2 antibody.
332. The anti-lyssavirus antibody, or the antigen-binding fragment thereof, according to item 327, wherein the antibody, or the antigen-binding fragment thereof, is an IgG3 antibody.
333. The anti-lyssavirus antibody, or the antigen-binding fragment thereof, according to item 327, wherein the antibody, or the antigen-binding fragment thereof, is an IgG4 antibody.
334. The anti-lyssavirus antibody, or the antigen-binding fragment thereof, according to any one of items 278-333, for use in the prevention and/or treatment of lyssavirus infection.
335. The anti-lyssavirus antibody, or the antigen-binding fragment thereof, according to item 334, wherein the antibody or the antigen-binding fragment thereof, is administered at a dose of 0.005 to 100 mg/kg.
336. The anti-lyssavirus antibody, or the antigen-binding fragment thereof, according to item 334, wherein the antibody or the antigen-binding fragment thereof, is administered at a dose of 0.0075 to 50 mg/kg.
337. The anti-lyssavirus antibody, or the antigen-binding fragment thereof, according to item 334, wherein the antibody or the antigen-binding fragment thereof, is administered at a dose of 0.01 to 10 mg/kg.
338. The anti-lyssavirus antibody, or the antigen-binding fragment thereof, according to item 334, wherein the antibody or the antigen-binding fragment thereof, is administered at a dose of 0.01 to 1 mg/kg.
339. The anti-lyssavirus antibody, or the antigen-binding fragment thereof, according to item 334, wherein the antibody or the antigen-binding fragment thereof, is administered at a dose of 0.01 to 0.1 mg/kg.
340. The anti-lyssavirus antibody, or the antigen-binding fragment thereof, according to item 334, wherein the antibody or the antigen-binding fragment thereof, is administered at a dose of 0.01 to 100 mg/kg.
341. The anti-lyssavirus antibody, or the antigen-binding fragment thereof, according to item 334, wherein the antibody or the antigen-binding fragment thereof, is administered at a dose of 0.1 to 75 mg/kg.
342. The anti-lyssavirus antibody, or the antigen-binding fragment thereof, according to item 334, wherein the antibody or the antigen-binding fragment thereof, is administered at a dose of 1 to 60 mg/kg.
343. The anti-lyssavirus antibody, or the antigen-binding fragment thereof, according to item 334, wherein the antibody or the antigen-binding fragment thereof, is administered at a dose of 10 to 50 mg/kg.
344. The anti-lyssavirus antibody, or the antigen-binding fragment thereof, according for use according to any one of items 334-343, wherein the antibody, or the antigen-binding fragment thereof, is administered in combination with a further anti-lyssavirus antibody, or an antigen-binding fragment thereof.
345. The anti-lyssavirus antibody, or the antigen-binding fragment thereof, for use according to item 344, wherein the two anti-lyssavirus antibodies, or the antigen-binding fragments thereof, administered in combination bind specifically to different epitopes on the glycoprotein G of RABV.
346. The anti-lyssavirus antibody, or the antigen-binding fragment thereof, for use according to item 345, wherein one of the two anti-lyssavirus antibodies, or the antigen-binding fragments thereof, binds to antigenic site I of glycoprotein G of RABV and the other of the two anti-lyssavirus antibodies, or the antigen-binding fragments thereof, binds to antigenic site III of glycoprotein G of RABV.
347. The anti-lyssavirus antibody, or the antigen-binding fragment thereof, for use according to any one of items 344-346, wherein one of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively.
348. The anti-lyssavirus antibody, or the antigen-binding fragment thereof, for use according to item 347, wherein one of the two anti-lyssavirus antibodies, or the antigen-binding fragments thereof, administered in combination comprises a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108; and the other of the two anti-lyssavirus antibodies, or the antigen-binding fragments thereof, administered in combination comprises a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180.
349. The anti-lyssavirus antibody, or the antigen-binding fragment thereof, for use according to any one of items 344-348, wherein the two anti-lyssavirus antibodies, or the antigen-binding fragments thereof, administered in combination are administered at equimolar amounts.
350. The anti-lyssavirus antibody, or the antigen-binding fragment thereof, for use according to any one of items 334-349, wherein the antibody, or the antigen-binding fragment thereof, is administered as defined in items 1-232.
351. A nucleic acid molecule comprising a polynucleotide encoding the antibody, or the antigen binding fragment thereof, according to any one of items 278-333.
352. The nucleic acid molecule according to item 351, wherein the nucleic acid is DNA.
353. The nucleic acid molecule according to item 351, wherein the nucleic acid is cDNA.
354. The nucleic acid molecule according to item 351, wherein the nucleic acid is RNA.
355. The nucleic acid molecule according to item 351, wherein the nucleic acid is mRNA.
356. The nucleic acid molecule according to item 351, wherein the nucleic acid is rRNA.
357. The nucleic acid molecule according to item 351, wherein the nucleic acid is miRNA.
358. The nucleic acid molecule according to item 351, wherein the nucleic acid is siRNA.
359. The nucleic acid molecule according to item 351, wherein the nucleic acid is tRNA.
360. The nucleic acid molecule according to any one of items 351-359, wherein the polynucleotide sequence is at least 75% identical to the nucleic acid sequence of any one of SEQ ID NOs: 8-14, 17, 18, 26-32, 35, 36, 44-50, 53, 54, 62-68, 72-74, 82-88, 91, 92, 100-106, 109, 110, 118-124, 127, 128, 136-142, 145, 146, 154-160, 163, 164, 172-178, 181, 182, 190-196, 199, 200, 208-214, 217 and 218.
361. A combination of at least two distinct nucleic acid molecules encoding an anti-lyssavirus antibody, or an antigen-binding fragment thereof, according to any one of items 72-88, wherein one nucleic acid molecule encodes at least a CDRH1, CDRH2 and CDRH3 of a heavy chain and another nucleic acid molecule encodes at least a CDRL1, CDRL2 and CDRL3 of a corresponding light chain of the antilyssavirus antibody or antigen-binding fragment.
362. The combination according to item 361, wherein one nucleic acid molecule encodes at least a variable region of a heavy chain (VH) and another nucleic acid molecule encodes at least a corresponding variable region of a light chain (VL) of the antilyssavirus antibody.
363. The combination according to item 362, wherein one nucleic acid molecule encodes a heavy chain and another nucleic acid molecule encodes a corresponding light chain of the antilyssavirus antibody.
364. The combination according to any one of items 361-363, wherein the nucleic acid is DNA.
365. The combination according to item 364, wherein the nucleic acid is cDNA.
366. The combination according to any one of items 361-363, wherein the nucleic acid is RNA.
367. The combination according to item 366, wherein the nucleic acid is mRNA.
368. The combination according to item 366, wherein the nucleic acid is rRNA.
369. The combination according to item 366, wherein the nucleic acid is miRNA.
370. The combination according to item 366, wherein the nucleic acid is siRNA.
371. The combination according to item 366, wherein the nucleic acid is tRNA.
372. A vector comprising the nucleic acid molecule according to any one of items 351-360.
373. A vector comprising the combination of nucleic acid molecules according to any one of items 361-371.
374. The vector according to item 373, wherein the vector is bicistronic.
375. The vector according to any one of items 373-375, wherein the vector is an expression vector.
376. The vector according to any one of items 373-375, wherein the vector is a storage vector.
377. The vector according to any one of items 373-375, wherein the vector is a cloning vector.
378. The vector according to any one of items 373-375, wherein the vector is a transfer vector.
379. A cell expressing the antibody, or the antigen binding fragment thereof, according to any of items 278-333; or comprising the vector according to any one of items 372-378.
380. The cell according to item 379, wherein the cell is a mammalian cell.
381. A pharmaceutical composition comprising the antibody, or the antigen binding fragment thereof, according to any of items 278-333, the nucleic acid according to any one of items 351-360, the combination according to any one of items 361-371, the vector according to any one of items 372-378, or the cell according to item 379
380, and a pharmaceutically acceptable excipient, diluent or carrier.
382. The pharmaceutical composition according to item 381, wherein the pharmaceutical composition comprises at least two distinct anti-lyssavirus antibodies, or antigen-binding fragments thereof, according to any of items 278-333.
383. The pharmaceutical composition according to item 382, wherein the at least two antibodies, or antigen-binding fragments thereof, specifically bind to different epitopes on the glycoprotein G of RABV.
384. The pharmaceutical composition according to item 383, wherein the at least two antibodies, or antigen-binding fragments thereof, specifically bind to antigenic site I and to antigenic site Ill of glycoprotein G of RABV.
385. The pharmaceutical composition according to any one of items 382-384, wherein one of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively.
386. The pharmaceutical composition according to item 385, wherein one of the two anti-lyssavirus antibodies, or the antigen-binding fragments thereof, comprises a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108; and the other of the two anti-lyssavirus antibodies, or the antigen-binding fragments thereof, comprises a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180.
387. The pharmaceutical composition according to any one of items 382-386, wherein the at least two distinct anti-lyssavirus antibodies, or antigen-binding fragments thereof, are comprised in the pharmaceutical composition in equimolar amounts.
388. The for use in (i) prophylaxis, in particular post-exposure prophylaxis, treatment.
389. The antibody, or an antigen binding fragment thereof, according to any one of items 278-333, the nucleic acid according to any one of items 351-360, the combination according to any one of items 361-371, the vector according to any one of items 372-378, the cell according to any one of items 379-380, or the pharmaceutical composition according to any one of items 381-387 for use in (i) prophylaxis, in particular post-exposure prophylaxis, treatment or attenuation of RABV and/or non-RABV lyssavirus infection; in (ii) vaccination against RABV and/or non-RABV lyssavirus infection; or in (iii) diagnosis of RABV and/or other lyssavirus infection.
390. The antibody, or an antigen binding fragment thereof, the nucleic acid, the combination, the vector, the cell, or the pharmaceutical composition for use according to item 389, wherein the pharmaceutical composition is administered as defined in items 1-64.
391. The antibody, or the antigen binding fragment thereof, according to any of items 278-333, the nucleic acid according to any one of items 351-360, the combination according to any one of items 361-371, the vector according to any one of items 372-378, the cell according to any one of items 379-380, or the pharmaceutical composition according to any of items 381-387 for use in post-exposure prophylaxis or attenuation of lyssavirus infection.
392. The antibody, or the antigen binding fragment thereof, the nucleic acid, the combination, the vector, the cell, or the pharmaceutical composition for use according to item 391, wherein the antibody, or the antigen binding fragment thereof, or the pharmaceutical composition is administered up to seven days, or up to five days, after infection.
393. The antibody, or the antigen binding fragment thereof, the nucleic acid, the combination, the vector, the cell, or the pharmaceutical composition for use according to item 391 or 392, wherein the antibody, or the antigen binding fragment thereof, or the pharmaceutical composition is administered in combination with a vaccine, an antiviral, interferon-alpha and/or ketamine.
394. The antibody, or the antigen binding fragment thereof, the nucleic acid, the combination, the vector, the cell, or the pharmaceutical composition for use according to item 393, wherein the vaccine is a rabies vaccine.
395. The antibody, or the antigen binding fragment thereof, the nucleic acid, the combination, the vector, the cell, or the pharmaceutical composition for use according to item 393 or 394, wherein the antiviral is ribavirin.
396. The antibody, or the antigen binding fragment thereof, the nucleic acid, the combination, the vector, the cell, or the pharmaceutical composition for use according to any one of items 391-395, wherein the antibody, or the antigen binding fragment thereof, or the pharmaceutical composition is administered in a standard PEP scheme.
397. The antibody, or the antigen binding fragment thereof, the nucleic acid, the combination, the vector, the cell, or the pharmaceutical composition for use according to item 396, wherein the antibody, or the antigen binding fragment thereof, or the pharmaceutical composition is administered in a standard PEP scheme in combination with a vaccine.
398. The antibody, or the antigen binding fragment thereof, the nucleic acid, the combination, the vector, the cell, or the pharmaceutical composition for use according to item 396 or 397, wherein the antibody, or the antigen binding fragment thereof, or the pharmaceutical composition is administered in the first treatment of the standard PEP scheme only.
399. The antibody, or the antigen binding fragment thereof, the nucleic acid, the combination, the vector, the cell, or the pharmaceutical composition for use according to any one of items 391-398, wherein the antibody, or the antigen binding fragment thereof, or the pharmaceutical composition is administered from 1 to 6 days, or from 2 to 5 days, after infection.
400. The antibody, or the antigen binding fragment thereof, the nucleic acid, the combination, the vector, the cell, or the pharmaceutical composition for use according to any one of items 391-398, wherein the antibody, or the antigen binding fragment thereof, or the pharmaceutical composition is administered without concomitant and/or subsequent administration of a vaccine.
401. A kit of parts comprising at least one antibody, or antigen binding fragment thereof, according to any of items 278-333, the nucleic acid according to any one of items 351-360, the combination according to any one of items 361-371, the vector according to any one of items 372-378, the cell according to any one of items 379-380, or the pharmaceutical composition according to any of items 381-387.
402. The kit of parts according to item 401, wherein the kit of parts comprises at least two distinct anti-lyssavirus antibodies, or antigen-binding fragments thereof, as defined in any one of items 278-333.
403. The kit of parts according to item 402, wherein the at least two antibodies, or antigen-binding fragments thereof, specifically bind to different epitopes on the glycoprotein G of RABV.
404. The kit of parts according to item 403, wherein the at least two antibodies, or antigen-binding fragments thereof, specifically bind to antigenic site I and to antigenic site III of glycoprotein G of RABV.
405. The kit of parts according to any one of items 402-404, wherein one of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences that are at least 80%, or at least 90%, identical to the amino acid sequences of SEQ ID NOs: 165-169 and 171 or to the amino acid sequences of SEQ ID NOs: 165-168 and 170-171, respectively.
406. The kit of parts according to item 405, wherein one of the two anti-lyssavirus antibodies, or the antigen-binding fragments thereof, comprises a heavy chain variable region having at least 80%, or at least 90%, sequence identity to the amino acid sequence of SEQ ID NO: 107 and a light chain variable region having at least 80%, or at least 90%, sequence identity to the amino acid sequence of SEQ ID NO: 108; and the other of the two anti-lyssavirus antibodies, or the antigen-binding fragments thereof, comprises a heavy chain variable region having at least 80%, or at least 90%, sequence identity to the amino acid sequence of SEQ ID NO: 179 and a light chain variable region having at least 80%, or at least 90%, sequence identity to the amino acid sequence of SEQ ID NO: 180.
407. Method of treating or attenuating a lyssavirus infection in a subject, wherein the method comprises administering to a subject in need thereof the antibody, or the antigen binding fragment thereof, as defined in any one of items 278-333, the nucleic acid according to any one of items 351-360, the combination according to any one of items 361-371, the vector according to any one of items 372-378, the cell according to any one of items 379-380,or the pharmaceutical composition according to any one of items 381-387.
408. The method according to item 407, wherein the antibody, or the antigen-binding fragment thereof, or the pharmaceutical composition is administered
   (i) into the central nervous system (CNS); and
   (ii) peripherally.
409. The method of item 407 or 408, wherein the subject is a human subject.
410. The method of item 407 or 408, wherein the subject is an animal subject.
411. The method of item 410, wherein the subject is selected from the group consisting of cows, dogs, cats, horses, goats, sheep, pigs, and rabbits.

The invention, and in particular the items outlined above, are described in more detail below.

### DETAILED DESCRIPTION

Although the invention is described in detail below, it is to be understood that this invention is not limited to the particular methodologies, protocols and reagents described herein as these may vary. It is also to be understood that the terminology used herein is not intended to limit the scope of the invention which will be limited only by the appended claims. Unless defined otherwise, all technical and scientific terms used herein have the same meanings as commonly understood by one of ordinary skill in the art.

In the following, the elements of the invention will be described. These elements are listed with specific embodiments, however, it should be understood that they may be combined in any manner and in any number to create additional embodiments. The variously described examples and embodiments should not be construed to limit the invention to only the explicitly described embodiments. This description should be understood to support and encompass embodiments which combine the explicitly described embodiments with any number of the disclosed and/or aspects. Furthermore, any permutations and combinations of all described elements in this application should be considered disclosed by the description of the application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention (reducing the occurrence), prophylaxis, attenuation, amelioration and therapy. The terms “subject” or “patient” are used interchangeably herein to mean all mammals, in particular humans. Examples of subjects include humans, cows, dogs, cats, horses, goats, sheep, pigs, and rabbits. For example, the patient is a human.

As used herein, the terms “antigen binding fragment,” “fragment,” and “antibody fragment” are used interchangeably to refer to any fragment of an antibody that retains the antigen-binding activity of the antibody. Examples of antibody fragments include, but are not limited to, a single chain antibody, Fab, Fab′, F(ab′)₂, Fv or scFv. Further, the term “antibody” as used herein includes both antibodies and antigen binding fragments thereof.

As used herein, the term “antibody” encompasses various forms of antibodies including, without being limited to, whole antibodies, antibody fragments, in particular antigen binding fragments, human antibodies, chimeric antibodies, humanized antibodies, recombinant antibodies and genetically engineered antibodies (variant or mutant antibodies) as long as the characteristic properties according to the invention are retained. For example, the antibody is a human antibody and/or a monoclonal antibody, in particular a recombinant human monoclonal antibody. For example, the antibody may be a monoclonal antibody. This includes administration of more than one, such as two, monoclonal antibodies.

Human antibodies are well-known in the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). Typically, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. The term “human antibody” as used herein also comprises such antibodies which are modified, e.g. in a constant and/or variable region, to generate the properties according to the invention as described herein. As used herein, the term “variable region” (variable region of a light chain (V_{L}), variable region of a heavy chain (V_{H})) denotes each of the pair of light and heavy chains which is involved directly in binding the antibody to the antigen.

Antibodies for use according to the invention can be of any isotype (e.g., IgA, IgG, IgM i.e. an α, γ or μ heavy chain), such as IgG. Within the IgG isotype, antibodies may be IgG1, IgG2, IgG3 or IgG4 subclass, such as IgG1. Antibodies may have a κ or a λ light chain.

For example, the antibody for use according the invention, or the antigen binding fragment thereof, is a purified antibody, a single chain antibody, Fab, Fab′, F(ab′)2, Fv or scFv.

The antibodies for use according the invention may, for example, be human antibodies, monoclonal antibodies, human monoclonal antibodies, recombinant antibodies or purified antibodies. Fragments of the antibodies typically retain the antigen-binding activity of the antibodies. Such fragments include, but are not limited to, single chain antibodies, Fab, Fab′, F(ab′)2, Fv or scFv. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

Fragments of the antibodies for use according the invention can be obtained from the antibodies by methods that include digestion with enzymes, such as pepsin or papain, and/or by cleavage of disulfide bonds by chemical reduction. Alternatively, fragments of the antibodies can be obtained by cloning and expression of part of the sequences of the heavy or light chains. Antibody “fragments” include Fab, Fab′, F(ab′)2 and Fv fragments. Single-chain Fv fragments (scFv) derived from the heavy and light chains of an antibody for use according to the invention are also encompassed. For example, a scFv comprising the CDRs from an antibody for use according to the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker.

Antibody fragments for use according to the invention may impart monovalent or multivalent interactions and be contained in a variety of structures as described above. For instance, scFv molecules may be synthesized to create a trivalent “triabody” or a tetravalent “tetrabody.” The scFv molecules may include a domain of the Fc region resulting in bivalent minibodies. In addition, the CDR or variable region sequences may be a component of multispecific molecules in which the CDR or variable region sequences target epitopes and other regions of the molecule bind to other targets. Exemplary molecules include, but are not limited to, bispecific Fab2, trispecific Fab3, bispecific scFv, and diabodies (Holliger and Hudson, 2005, Nature Biotechnology 9: 1126-1136).

Antibodies may be provided in purified form. Typically, the antibody will be in a composition that is substantially free of other polypeptides. As used herein, “substantially free of other polypeptides” means that less than 90% (by weight), usually less than 60%, such as less than 50% of the composition is made up of other polypeptides.

Antibodies may be immunogenic in human and/or in non-human (or heterologous) hosts e.g., in mice. For example, the antibodies may have an idiotope that is immunogenic in non-human hosts, but not in a human host. Antibodies for human use include those that cannot be easily isolated from hosts such as mice, goats, rabbits, rats, non-primate mammals, etc. and cannot generally be obtained by humanization or from xeno-mice.

As used herein, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used alone, or in combination, as prophylactic or therapeutic agents upon appropriate formulation, in association with active vaccination, as a diagnostic tool, or as a production tool as described herein.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The term “specifically binding” and similar reference does not encompass non-specific sticking.

The term “vaccine” as used herein is typically understood to be a prophylactic or therapeutic material providing at least one antigen, such as an immunogen. The antigen or immunogen may be derived from any material that is suitable for vaccination. For example, the antigen or immunogen may be derived from a pathogen, such as from bacteria or virus particles etc., or from a tumor or cancerous tissue. The antigen or immunogen stimulates the body's adaptive immune system to provide an adaptive immune response. In particular, an “antigen” or an “immunogen” refers typically to a substance which may be recognized by the immune system, for example, by the adaptive immune system, and which is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells.

As used herein, “sequence variant” (also referred to as “variant”) refers to any alteration in a reference sequence, whereby a reference sequence is any of the sequences listed in the “Tables of Sequences and SEQ ID Numbers” (sequence listing), i.e. SEQ ID NO: 1 to SEQ ID NO: 224. Thus, the term “sequence variant” includes nucleotide sequence variants and amino acid sequence variants. In particular, the sequence variants referred to herein are functional sequence variants, i.e. sequence variants maintaining the biological function of, for example, the antibody. In the one context of the invention such a maintained biological function is, for example, the neutralization of lyssavirus infection and/or the binding of the antibody to (a specific epitope of) the glycoprotein G of lyssavirus (such as RABV). As used herein, a sequence variant has, for example, at least 70% sequence identity to the respective reference sequence. For example, a sequence variant has at least 75% sequence identity to the respective reference sequence. For example, a sequence variant has at least 80% sequence identity to the respective reference sequence. For example, a sequence variant has at least 85% sequence identity to the respective reference sequence. For example, a sequence variant has at least 88% sequence identity to the respective reference sequence. For example, a sequence variant has at least 90% sequence identity to the respective reference sequence. For example, a sequence variant has at least 92% sequence identity to the respective reference sequence. For example, a sequence variant has at least 95% sequence identity to the respective reference sequence. For example, a sequence variant has at least 96% sequence identity to the respective reference sequence. For example, a sequence variant has at least 97% sequence identity to the respective reference sequence. For example, a sequence variant has at least 98% sequence identity or at least 99% sequence identity to the respective reference sequence.

The term “sequence variant” includes in particular such variants that comprise mutations and/or substitutions in comparison to the reference sequence. Exemplary variants of an Fc moiety sequence include, but are not limited to, those that have an L to A substitution at position CH2 4, CH2 5, or both.

Sequence identity is usually calculated with regard to the full length of the reference sequence (i.e. the sequence recited in the application). Percentage identity, as referred to herein, can be determined, for example, using BLAST using the default parameters specified by the NCBI (the National Center for Biotechnology information; http://www.ncbi.nlm.nih.gov/) [Blosum 62 matrix; gap open penalty=11 and gap extension penalty=1].

As used herein, a “nucleotide sequence variant” has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “nucleotide sequence variant” can either result in a change in the respective reference amino acid sequence, i.e. in an “amino acid sequence variant” or not. Example sequence variants are such nucleotide sequence variants, which do not result in amino acid sequence variants (silent mutations), but other non-silent mutations are within the scope as well, in particular mutant nucleotide sequences, which result in an amino acid sequence, which is at least 80%, or at least 90% sequence identical to the reference sequence, such as at least 95% or 99% sequence identical. It should be understood that in all instances used herein “at least 80% identical” includes at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and 100% identical. Likewise, “at least 90% identical” includes at least 90%, at least 91%, at least 92%, at least 93%, at least 94%, at least 95%, at least 96%, at least 97%, at least 98%, at least 99%, and 100% identical.

An “amino acid sequence variant” has an altered sequence in which one or more of the amino acids in the reference sequence is deleted or substituted, or one or more amino acids are inserted into the sequence of the reference amino acid sequence. As a result of the alterations, the amino acid sequence variant has an amino acid sequence which is at least 80% or at least 90% identical to the reference sequence, such as at least 95% or at least 99% identical to the reference sequence. Variant sequences which are at least 90% identical have no more than 10 alterations, i.e. any combination of deletions, insertions or substitutions, per 100 amino acids of the reference sequence. It should be understood that in all instances used herein “at least 80% identical” includes at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and 100% identical. Likewise, “at least 90% identical” includes at least 90%, at least 91%, at least 92%, at least 93%, at least 94%, at least 95%, at least 96%, at least 97%, at least 98%, at least 99%, and 100% identical.

For example, the amino acid substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another. However, non-conservative amino acid substitutions are also possible.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

In some cases, the alterations in the sequence variants do not abolish the functionality of the respective reference sequence, in the present case, e.g., the functionality of a sequence of an antibody, or antigen binding fragment thereof, to bind to the same epitope and/or to sufficiently neutralize infection of lyssavirus, such as RABV. Guidance in determining which nucleotides and amino acid residues, respectively, may be substituted, inserted or deleted without abolishing such functionality are found by using computer programs well known in the art.

As used herein, a nucleic acid sequence or an amino acid sequence “derived from” a designated nucleic acid, peptide, polypeptide or protein refers to the origin of the nucleic acid, peptide, polypeptide or protein. For example, the nucleic acid sequence or amino acid sequence which is derived from a particular sequence has an amino acid sequence that is essentially identical to that sequence or a portion thereof, from which it is derived, whereby “essentially identical” includes sequence variants as defined above. For example, the nucleic acid sequence or amino acid sequence which is derived from a particular peptide or protein, is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins and nucleic acids are thus easily identifiable to one of ordinary skill in the art. Likewise, sequences “derived from” other sequence are usually easily identifiable to one of ordinary skill in the art as having its origin in the sequence.

For example, a nucleic acid sequence or an amino acid sequence derived from another nucleic acid, peptide, polypeptide or protein may be identical to the starting nucleic acid, peptide, polypeptide or protein (from which it is derived). However, a nucleic acid sequence or an amino acid sequence derived from another nucleic acid, peptide, polypeptide or protein may also have one or more mutations relative to the starting nucleic acid, peptide, polypeptide or protein (from which it is derived), in particular a nucleic acid sequence or an amino acid sequence derived from another nucleic acid, peptide, polypeptide or protein may be a functional sequence variant as described above of the starting nucleic acid, peptide, polypeptide or protein (from which it is derived). For example, in a peptide/protein one or more amino acid residues may be substituted with other amino acid residues or one or more amino acid residue insertions or deletions may occur.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

As used herein, “rabies” refers to rabies disease. Rabies is caused by a number of lyssaviruses including rabies virus and other lyssaviruses, for example European bat lyssavirus.

Lyssaviruses have helical symmetry, with a length of about 180 nm and a cross-section of about 75 nm. These viruses are enveloped and have a single-stranded RNA genome with negative sense. The genetic information is packed as a ribonucleoprotein complex in which RNA is tightly bound by the viral nucleoprotein. The RNA genome of the virus encodes five genes whose order is highly conserved: nucleoprotein (N), phosphoprotein (P), matrix protein (M), glycoprotein (G), and the viral RNA polymerase (L).

The Lyssavirus genus is subdivided into four phylogroups, 14 species and 4 tentative new species. Phylogroup I includes the species Rabies virus (RABV), European bat lyssavirus type 1 (EBLV-1) and type 2 (EBLV-2), Duvenhage virus (DUVV), Australian bat lyssavirus (ABLY), Aravan virus (ARAV), Khujand virus (KHUV), Bokeloh bat lyssavirus (BBLV) and Irkut virus (IRKV). Phylogroup II includes Lagos bat virus (LBV), Mokola virus (MOKV), and Shimoni bat virus (SHIV or SHIBV). The remaining viruses, West Caucasian bat virus (WCBV) and Ikoma lyssavirus (IKOV) cannot be included in either of these two phylogroups and are classified as phylogroups III and IV, respectively (Bourhy, H., et al. Journal of Clinical Microbiology 30, 2419-2426,1992; Bourhy, H., et al. Virology 194, 70-81, 1993; Amengual, B., et al. J Gen. Virol 78, 2319-2328, 1997; Kuzmin, I. V. et al. Virus Res 149, 197-210, 2010; Badrane, H., et al. J Virol 75, 3268-3276, 2001; Marston, D. A. et al. Emerg Infect Dis 18, 664-667, 2012; Delmas O, Holmes E C, Talbi C, Larrous F, Dacheux L, Bouchier C, Bourhy H. Genomic diversity and evolution of the lyssaviruses. PLoS One. 2008 Apr. 30; 3(4):e2057). Importantly, all of these species of lyssaviruses have caused human and/or animal deaths in nature (Badrane, H., et al. J Virol 75, 3268-3276, 2001; Fooks A R, Cliquet F, Finke S, Freuling C, Hemachudha T, Mani R S, Müller T, Nadin-Davis S, Picard-Meyer E, Wilde H, Banyard A C. Rabies. Nat Rev Dis Primers. 2017 Nov. 30; 3:17091).

Rabies virus (RABV) was the first of the fourteen lyssavirus genotypes to be identified. The rabies virus is a large bullet-shaped, enveloped, single stranded RNA virus classified and the genome of rabies virus codes for five viral proteins: RNA-dependent RNA polymerase (L); a nucleoprotein (N); a phosphorylated protein (P); a matrix protein (M) located on the inner side of the viral envelope; and an external surface glycoprotein (G). The G protein (62-67 kDa) is a type-I glycoprotein composed of 505 amino acids that has two to four potential N glycosylation sites. The G protein covers the outer surface of the virion envelope and is the only target antigen, which can induce virus-neutralizing antibodies.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the invention which will be limited only by the appended claims. Unless defined otherwise, all technical and scientific terms used herein have the same meanings as commonly understood by one of ordinary skill in the art.

Anti-Lyssavirus Antibody Administered into the CNS and Peripherally

In a first aspect, an anti-lyssavirus antibody, or an antigen-binding fragment thereof, is provided for use in the treatment of lyssavirus infection, wherein the antibody, or the antigen-binding fragment thereof, is administered

(i) into the central nervous system (CNS); and
(ii) peripherally.

Disclosed herein is that symptomatic lyssavirus infection can be treated, even after symptoms of lyssavirus infection appeared, when anti-lyssavirus antibodies are administered (i) into the CNS; and (ii) peripherally (outside the CNS), as shown by the appended Examples. Without being bound to any theory, a role of passively administered anti-lyssavirus antibodies may be to inactivate the virus in the periphery, thereby limiting its entry into nerve endings. Once the lyssavirus has reached central nervous system (CNS) tissues, circulating anti-lyssavirus antibodies could have a lesser effect due to their limited ability to cross the blood-brain barrier. On the other hand, without being bound to any theory, the presence of anti-lyssavirus antibodies in the CNS only (i.e. without antibodies in the periphery) may not be sufficient for a successful therapeutic treatment of symptomatic rabies.

Anti-lyssavirus antibodies, and antigen-binding fragments thereof, are known in the art. For example, an overview is provided by Ilina E N, Larina M V, Aliev T K, Dolgikh D A, Kirpichnikov M P. Recombinant Monoclonal Antibodies for Rabies Post-exposure Prophylaxis. Biochemistry (Mosc). 2018 January; 83(1):1-12. doi: 10.1134/S0006297918010017. Examples of anti-lyssavirus antibodies, and antigen-binding fragments thereof, are described in WO 2016/078761 and in De Benedictis et al., 2016 (De Benedictis P, Minola A, Rota Nodari E, Aiello R, Zecchin B, Salomoni A, Foglierini M, Agatic G, Vanzetta F, Lavenir R, Lepelletier A, Bentley E, Weiss R, Cattoli G, Capua I, Sallusto F, Wright E, Lanzavecchia A, Bourhy H, Corti D. Development of broad-spectrum human monoclonal antibodies for rabies post-exposure prophylaxis. EMBO Mol Med. 2016 Apr. 1; 8(4):407-21. doi:10.15252/emmm.201505986), which are incorporated herein by reference.

According to the invention, an anti-lyssavirus antibody, or an antigen-binding fragment thereof, is administered (i) into the central nervous system (CNS) and (ii) peripherally. In general, the anti-lyssavirus antibody administered into the central nervous system (CNS) and the anti-lyssavirus antibody administered peripherally may be the same anti-lyssavirus antibody or distinct anti-lyssavirus antibodies (or the same or distinct combination(s) of two or more anti-lyssavirus antibodies). For example, the same anti-lyssavirus antibody (or the same combination of two or more anti-lyssavirus antibodies) is administered into the central nervous system (CNS) and peripherally.

As used herein, the term “peripherally” used in the context of a route of administration (e.g., peripheral administration) refers to any administration route outside the central nervous system (CNS) and blood-brain barrier (BBB). Accordingly, the terms “peripherally”, “peripheral” and “periphery”, as used herein, are to be understood in respect to the CNS/BBB and generally refer to those parts of the body “outside” the BBB and other than the CNS.

As used herein, the expression “into the central nervous system” used in the context of a route of administration (e.g., administration into the CNS) includes any administration route (in)to brain and spinal cord tissue and into cerebrospinal fluid (CSF) (i.e., anything “inside” the BBB).

In general, the central nervous system (also referred to as “CNS”) is the part of the nervous system, which consists of the brain and the spinal cord. The retina, the optic nerve (cranial nerve II), the olfactory nerves (cranial nerve I) and the olfactory epithelium are parts of the brain. The olfactory epithelium is the only central nervous tissue in direct contact with the environment.

The CNS and its extracellular fluid is separated from the circulating blood by a highly selective semipermeable membrane, the blood-brain barrier (also referred to as “BBB”). The blood brain barrier is formed by endothelial cells, which restrict the diffusion of microscopic objects (e.g., bacteria) and large molecules into the cerebrospinal fluid (CSF), while allowing the passage of water, some gases, and lipid-soluble (hydrophobic) molecules (O₂, CO₂, hormones) by passive diffusion, as well as the selective transport of molecules such as glucose and amino acids that are crucial to neural function. Larger hydrophilic molecules, such as antibodies, however, are typically prevented from crossing the BBB.

As antibodies typically cannot cross the blood-brain barrier, the invention comprises administration of antibodies (i) peripherally (i.e., to any part of the body outside the CNS/BBB) and (ii) into the CNS (i.e., to any part of the body “inside” the CNS/BBB). In this way, the anti-lyssavirus antibody can exert its effects on both sides of the BBB.

Administration into the CNS generally includes administration into brain tissue or spinal cord tissue as well as administration into the cerebrospinal fluid (CSF). Examples of administration routes into the CNS are described, for example, in Pathan S A, Iqbal Z, Zaidi S M, Talegaonkar S, Vohra D, Jain G K, Azeem A, Jain N, Lalani J R, Khar R K, Ahmad F J. CNS drug delivery systems: novel approaches. Recent Pat Drug Deliv Formul. 2009 January; 3(1):71-89, which is incorporated herein by reference.

In general, administration into the CNS may be performed by injection or infusion, e.g. by using a suitable needle or catheter. For repeated or continuous administration, reservoirs and/or minipumps may be used. The route of administration into the CNS may be selected from intrathecal, epidural, intracerebroventricular, intracerebral, transnasal, transocular, intranasal, and perispinal administration.

For example, the anti-lyssavirus antibody is administered into the cerebrospinal fluid (intra-CSF administration). Cerebrospinal fluid (CSF) is a clear, colorless body fluid found in the brain and spinal cord. CSF occupies the subarachnoid space (between the arachnoid mater and the pia mater) and the ventricular system around and inside the brain and spinal cord. It fills the ventricles of the brain, cisterns, and sulci, as well as the central canal of the spinal cord.

Examples of intra-CSF administration routes include intrathecal, intracerebroventricular, and epidural administration. For example, the anti-lyssavirus antibody may be delivered into the CSF in the spinal cord by intrathecal or epidural administration. For example, the anti-lyssavirus antibody may be delivered into the CSF in the brain by intracerebroventricular administration. For repeated or continuous administration, reservoirs and/or minipumps may be used. For example, the Ommaya reservoir may be used for intracerebroventricular (ICV) administration (Ommaya A K. Subcutaneous reservoir and pump for sterile access to ventricular cerebrospinal fluid. Lancet. 1963 Nov. 9; 2(7315):983-4). The Ommaya reservoir comprises a mushroom-shaped dome made of silicone rubber, which is connected to a catheter inserted into a lateral cerebral ventricle. The Ommaya reservoir is implanted subcutaneously and is a compressible pump.

The anti-Lyssavirus antibodies may also be directly administered into the brain tissue (intracerebral administration). For example, the anti-lyssavirus antibodies may be administered locally to a selected brain region, for example such that an afflicted brain region can be directly targeted.

Another option of administration into the CNS is intranasal administration, for example as described in U.S. Pat. No. 5,624,898 A, WO 00/33813, and in Dhuria S V, Hanson L R, Frey W H 2nd. Intranasal delivery to the central nervous system: mechanisms and experimental considerations. J Pharm Sci. 2010 April; 99(4):1654-73. doi: 10.1002/jps.21924. Intranasal delivery may be achieved by nasal delivery devices, such as sprays, nose droppers or needleless syringes. For example, a nasal delivery device is used, which is designed to deposit the nasally applied formulation specifically to the olfactory region (olfactory epithelium), such as the Bi-Directional Technology™ (OptiNose), the POD device (Impel NeuroPharma) or the Controlled Particle Dispersion (CPD™) Technology, e.g., the ViaNase™ device (Kurve Technology).

A further option of administration into the CNS is transnasal or transocular administration, for example as described in CA 2560798 A1. Thereby, a substance is administered through the olfactory nerve or the optical nerve. Further nasal and ocular administration ways are described in Pathan S A, Iqbal Z, Zaidi S M, Talegaonkar S, Vohra D, Jain G K, Azeem A, Jain N, Lalani J R, Khar R K, Ahmad F J. CNS drug delivery systems: novel approaches. Recent Pat Drug Deliv Formul. 2009 January; 3(1):71-89.

Another option of administration into the CNS is perispinal administration, for example as described in Tobinick E L. Perispinal Delivery of CNS Drugs. CNS Drugs. 2016 June; 30(6):469-80. doi: 10.1007/s40263-016-0339-2. Perispinal administration is optionally performed by perispinal injection. Perispinal administration is designed to use the cerebrospinal venous system (CSVS) and delivers a substance into the anatomic area posterior to the ligamentum flavum, an anatomic region drained by the external vertebral venous plexus (EVVP), a division of the CSVS.

According to the invention, the anti-lyssavirus antibody, or the antigen-binding fragment thereof, is also administered (in addition to administration into the CNS) peripherally, i.e. in the body “outside” the central nervous system (CNS) and blood-brain barrier (BBB). Accordingly, any administration route, which does not target the CNS and/or bypass the BBB, may be used for peripheral administration. It is understood that the peripherally administered antibody, or the antigen-binding fragment thereof, is not administered into the CNS (as described above).

Typically, the peripherally administered antibody, or the antigen-binding fragment thereof, is administered systemically or locally (e.g., in the periphery). Routes for systemic administration in general include enteral and parenteral routes of administration. Examples of enteral administration include oral and rectal administration. Examples of parenteral administration include intravenous, intramuscular, intraarterial, subcutaneous, intradermal, transdermal, and intraperitoneal routes. Routes for local administration in general include, for example, topical administration routes but, in particular, also intradermal, transdermal, subcutaneous, or intramuscular administration.

In a particular embodiment, the peripherally administered anti-lyssavirus antibody, or the antigen-binding fragment thereof, is administered intravenously (i.v.), intramuscularly (i.m.), subcutaneously (s.c.), or intradermally. For example, the peripherally administered anti-lyssavirus antibody, or the antigen-binding fragment thereof, is administered subcutaneously. For example, the peripherally administered anti-lyssavirus antibody, or the antigen-binding fragment thereof, is administered intradermally. For example, the peripherally administered anti-lyssavirus antibody, or the antigen-binding fragment thereof, is administered intramuscularly. For example, the peripherally administered anti-lyssavirus antibody, or the antigen-binding fragment thereof, is administered intravenously. For example, the peripherally administered anti-lyssavirus antibody, or the antigen-binding fragment thereof, is administered intravenously (i.v.) or intramuscularly (i.m.). The anti-lyssavirus antibody is therefore formulated, for example, in liquid (or sometimes in solid) form.

For example, the peripherally administered antibody, or the antigen-binding fragment thereof, is administered at the site of infliction. The “site of infliction” is typically the site of the body, where the lyssavirus entered the body, for example the site of a (dog or bat) bite. It is understood that administration at the site of infliction does not necessarily mean that the anti-lyssavirus antibody must be administered into a bite wound itself (which may disturb wound healing), but administration in close vicinity is usually sufficient. For example, the same muscle (in which the bite occurred) may be targeted for administration at the site of infliction. For example, the anti-lyssavirus antibody may be administered intramuscularly, subcutaneously or intradermally at the site of infliction (e.g., the same muscle or the corresponding skin, where the bite occurred).

According to the invention, the anti-lyssavirus antibody, or the antigen-binding fragment thereof, is generally used for treatment of lyssavirus infection. As used herein, the term “treatment” refers to prevention, prophylaxis (such as post-exposure prophylaxis), therapy, curative healing, amelioration, and alleviation of lyssavirus infection (and/or its symptoms). The anti-lyssavirus antibody administration according to the invention is very effective before and after symptoms of lyssavirus infection occur.

However, as peripheral administration is usually more convenient for a patient than administration into the CNS, the anti-lyssavirus antibody administration according to the invention is optionally used in more serious situations, for example after (assumed or proven) lyssavirus infection, in particular if (classical) post-exposure therapy is ineffective or insufficient, or after symptoms of lyssavirus infection occur. Accordingly, the antibody, or the antigen-binding fragment thereof, is administered, for example, for the first time after (the first) symptoms of lyssavirus infection occur. Early symptoms include fever, anorexia, nausea, headache, general weakness or discomfort, and prickling, itching or local pain at the site of bite. These symptoms progress, in particular within days, to one or more of the following symptoms, which are related to cerebral dysfunction: violent movements, uncontrolled excitement, fear of water, aerophobia, confusion, hyperactivity, an inability to move parts of the body, paralysis, and loss of consciousness. For example, for rabies two different types of the disease may develop after the virus attacks the CNS: furious rabies with symptoms including insomnia, anxiety, confusion, agitation, hallucinations, excess salivation, problems of swallowing and fear of water; and paralytic rabies wherein infected patients slowly become paralyzed and eventually slip into a coma. For example, the anti-lyssavirus antibody is administered according to the invention (for the first time) as soon as possible after (the first) symptoms of lyssavirus infection occur (e.g., were observed).

For example, the antibody, or the antigen-binding fragment thereof, is administered for the first time, e.g., at least five or at least six days after exposure to a lyssavirus. For example, the antibody, or the antigen-binding fragment thereof, may be administered for the first time, e.g., at least seven or at least eight days after exposure to a lyssavirus. This time frame typically coincides with the occurrence of the first symptoms of lyssavirus infection as described above. In other examples, the antibody, or antigen binding fragment thereof, is administered for the first time, e.g., the same day as exposure to a lyssavirus, or at least one day after exposure to a lyssavirus. For example, the antibody, or antigen-binding fragment thereof, may be administered for the first time, e.g., at least 0, 1, 2, 3, or 4 days after exposure to a lyssavirus. In some embodiments, the antibody, or the antigen-binding fragment thereof, is administered for the first time at least 5 days after exposure to a lyssavirus.

As described above, the antibody, or the antigen binding fragment thereof, for use according to the invention, or the pharmaceutical composition for use according to the invention may be administered after the first symptoms occur, i.e. after onset of symptoms. In some embodiments, the antibody, or the antigen binding fragment thereof, for use according to the invention, or the pharmaceutical composition for use according to the invention may be administered not more than one week after onset of symptoms. For example, the antibody, or the antigen binding fragment thereof, for use according to the invention, or the pharmaceutical composition for use according to the invention may be administered no more than 6 or 5 days after onset of symptoms, e.g. from 1 to 6 days or from 2 to 5 days after onset of symptoms. In some embodiments, antibody, or the antigen binding fragment thereof, for use according to the invention, or the pharmaceutical composition for use according to the invention may be administered no more than 20, 19, 18, 17, 16, 15, 14, 13, 12, 11, 10, 9, 8, 7, 6, 5, 4, 3, 2 or 1 day(s) after onset of symptoms.

In general, the anti-lyssavirus antibody, or the antigen-binding fragment thereof, may be administered into the CNS and peripherally either at about the same time (concomitantly) or consecutively (sequentially) as described herein. Moreover, the anti-lyssavirus antibody, or the antigen-binding fragment thereof, may be administered into the CNS and peripherally in the same pharmaceutical form (same type of formulation) or in distinct pharmaceutical forms (distinct types of formulations, e.g. one formulation adapted for administration into the CNS and the other adapted for peripheral administration).

For example, peripheral administration and administration into the CNS may be performed consecutively (sequentially). This means that peripheral administration is performed before and/or after administration into the CNS. In consecutive administration, the time between the two types of administration (peripherally and into the CNS) is, for example, no more than one week or no more than 3 days, such as no more than 2 days or no more than 24 h. For example, both, peripheral administration and administration into the CNS, are performed at the same day, and, e.g., the time between administration of the first component and administration of the second component is no more than 6 hours or no more than 3 hours, such as no more than 2 hours or no more than 1 h.

In some embodiments, the peripheral administration of the antibody, or the antigen-binding fragment thereof, and the administration of the antibody, or the antigen-binding fragment thereof, into the CNS is performed at about the same time (concomitantly).

“At about the same time”, as used herein, means in particular simultaneous administration or that directly after administration into the CNS, the antibody is administered peripherally or directly after peripheral administration the antibody is administered into the CNS. The skilled person understands that “directly after” includes the time necessary to prepare the second administration in particular the time necessary for exposing and disinfecting the location for the second administration as well as appropriate preparation of the “administration device” (e.g., syringe, pump, etc.). Simultaneous administration also includes if the periods of peripheral administration and administration into the CNS overlap or if, for example, one type of administration (e.g., into the CNS or peripherally) occurs over a longer period of time, such as 30 min, 1 h, 2 h or even more, e.g. by infusion, and the other type of administration is performed at some time during such a long period, e.g. by injection.

In particular, the antibody, or the antigen-binding fragment thereof, is administered peripherally while the antibody, or the antigen-binding fragment thereof, is administered into the CNS. In other words, the antibody, or the antigen-binding fragment thereof, is administered peripherally for example during administration of the antibody, or the antigen-binding fragment thereof, into the CNS. For example, the antibody, or the antigen-binding fragment thereof, may be administered peripherally by (e.g., a single) injection (e.g., i.v., i.m., s.c. or intradermally), during administration of the antibody, or the antigen-binding fragment thereof into the CNS by (continuous) infusion.

For example, the anti-lyssavirus antibody, or the antigen-binding fragment thereof, may be administered peripherally once (single peripheral administration) or repeatedly, for example by injection (e.g., i.v., i.m., s.c. or intradermally). Accordingly, in some embodiments the anti-lyssavirus antibody, or the antigen-binding fragment thereof, is administered peripherally once (single peripheral administration). In another embodiment the anti-lyssavirus antibody, or the antigen-binding fragment thereof, is repeatedly administered peripherally.

For example, the first peripheral administration of the antibody, or the antigen-binding fragment thereof, and the first administration of the antibody, or the antigen-binding fragment thereof, into the CNS occur on the same day. As used herein, the “first” administration (peripherally or into the CNS) refers either (i) to the first administration of repeated administrations (e.g., the first of 2, 3, 4, 5, 6, 7, 8, 9, 10 or more administrations); or (ii) to the only administration if there is only one single administration (e.g., only one single peripheral administration or only one single administration into the CNS). In other words, the term “first”, as used in this context, does not imply that there were necessarily repeated administrations. For example, the first peripheral administration of the antibody, or the antigen-binding fragment thereof, and the first administration of the antibody, or the antigen-binding fragment thereof, into the CNS occur on the same day, e.g. at about the same time. For example, the antibody, or the antigen-binding fragment thereof, may be administered peripherally, while the antibody, or the antigen-binding fragment thereof is administered into the CNS, as described above.

In some embodiments, the anti-lyssavirus antibody, or the antigen-binding fragment thereof, is administered into the CNS by continuous administration, for example for at least 15 min or for at least 30 min, such as for at least 1 h or for 6 h, (e.g., for at least 12 h). In some embodiments, the antibody, or the antigen-binding fragment thereof, is administered into the CNS continuously for at least 24 hours or at least 2 days, such as at least 3 days or at least 4 days (e.g., at least 5 days).

Continuous administration typically continues for at least 5 min or at least 10 min, for example at least 15 min. In particular, continuous administration provides a steady delivery of the anti-lyssavirus antibody, or the antigen-binding fragment thereof. For example, continuous administration is performed for time periods of hours (e.g., 1, 2, 3, 4, 5, 6, 7, 8, 9, 10, 11, 12, 13, 14, 15, 16, 17, 18, 19, 20, 21, 22, 23 or 24 hours) or days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days). Continuous administration is achieved, e.g., by infusion. For example, suitable administration devices may be used, which may include, for example, reservoirs and/or minipumps.

For example, the antibody, or the antigen-binding fragment thereof, is administered into the CNS daily or every second day, for example for at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days. Daily administration may be achieved either by continuous administration as described above or by repeated intermittent administration (one administration, e.g. single injection, per day).

Moreover, the (monoclonal) antibody, or the antigen binding fragment thereof, for use according to the invention, or comprised in the pharmaceutical composition for use according to the invention, may be administered at a dose of 0.01 to 100 mg/kg or at a dose of 0.1 to 75 mg/kg, such as at a dose of 0.5 to 60 mg/kg or at a dose of 1 to 50 mg/kg. For example for peripheral administration, the minimum dose may be 2 or 4 mg/kg, e.g. a dose range of 2-45 mg/kg or 4-40 mg/kg may be used. In some embodiments, the minimum dose may be 10 or 20 mg/kg, such as 40 mg/kg, in particular for peripheral administration. With regard to the administration into the CNS, the minimum dose may be 0.2 or 0.4 mg/kg, e.g. a dose range of 0.2-20 mg/kg or 0.4-10 mg/kg may be used. In some embodiments, the minimum dose may be 1 or 2 mg/kg, such as 4 mg/kg, in particular for administration into the CNS.

It is understood that the above doses relate to single doses, such as the dose of a single administration (e.g. a single injection) or, if continuous administration is applied, to the dose administered per day (singly day dose). Moreover, if a combination of (monoclonal) antibodies is applied, as described herein, the above doses represent a “sum” (i.e., the total amount of anti-lyssavirus antibody, or antigen-binding fragment thereof, as described herein). In such cases, the amount of antibody may be equally distributed, for example, if two distinct anti-lyssavirus antibodies as described herein are used, each antibody may be administered at half of the above-described doses.

In general, “higher” doses are in particular useful if the exposure was severe and/or if treatment is initiated later than one or two days after exposure. As a general rule, the later the treatment is initiated after exposure or after the first symptoms occur, the higher the dose of the antibody, or the antigen binding fragment thereof, which may be used.

The lyssavirus infection to be treated by the invention may be infection with any one of the lyssaviruses. The skilled person will select the anti-lyssavirus antibody according to the lyssavirus infection to be treated. Moreover, broadly neutralizing anti-lyssavirus antibodies, which neutralize infection with rabies virus (RABV) and a large number of non-RABV lyssaviruses are known in the art. Such broadly neutralizing anti-lyssavirus antibodies are described, for example in WO 2016/078761 and in De Benedictis et al. (De Benedictis P, Minola A, Rota Nodari E, Aiello R, Zecchin B, Salomoni A, Foglierini M, Agatic G, Vanzetta F, Lavenir R, Lepelletier A, Bentley E, Weiss R, Cattoli G, Capua I, Sallusto F, Wright E, Lanzavecchia A, Bourhy H, Corti D. 2016. Development of broad-spectrum human monoclonal antibodies for rabies post-exposure prophylaxis. EMBO Mol Med 8:407-421), which are incorporated herein by reference. For example, the lyssavirus infection to be treated is rabies and the anti-lyssavirus antibody, or an antigen-binding fragment thereof, is an anti-RABV antibody or an antigen-binding fragment thereof.

Typically, the anti-lyssavirus antibody, or the antigen-binding fragment thereof, binds to a lyssavirus glycoprotein G (also referred to as “G protein”), such as the G protein (glycoprotein G) of RABV. For example, the antibody, or the antigen binding fragment thereof, according to the invention binds to the G protein (glycoprotein G) of RABV and/or to the G protein (glycoprotein G) of non-RABV lyssaviruses.

The RNA genome of a lyssavirus codes for five viral proteins: RNA-dependent RNA polymerase (L); a nucleoprotein (N); a phosphorylated protein (P); a matrix protein (M) located on the inner side of the viral envelope; and an external surface glycoprotein (G). The G protein covers the outer surface of the virion envelope and can induce virus-neutralizing antibodies. The G protein (62-67 kDa) is a type-I glycoprotein composed of 505 amino acids that has two to four potential N glycosylation sites. Sequences of lyssavirus glycoprotein G are known in the art, for example, as described in Buthelezi S G, Dirr H W, Chakauya E, Chikwamba R, Martens L, Tsekoa T L, Stoychev S H, Vandermarliere E. The Lyssavirus glycoprotein: A key to cross-immunity. Virology. 2016 November; 498:250-256. doi: 10.1016/j.virol.2016.08.034 (in particular Supplemental Material of Buthelezi et al., which provides a large number of lyssavirus glycoprotein G sequences).

In general, the epitopes to which the antibodies bind may be linear (continuous) or conformational (discontinuous). In one embodiment, the antibodies and antibody fragments thereof bind a conformational epitope. The conformational epitope is, for example, present only under non-reducing conditions.

In some embodiments, the anti-lyssavirus antibody, or the antigen-binding fragment thereof, binds to a glycoprotein G of RABV, for example to antigenic site I or antigenic site III (or to an epitope which at least partially overlaps with antigenic site III) of glycoprotein G of RABV. Antigenic sites I and III of glycoprotein G of RABV are well-known and described in the art (for example Buthelezi S G, Dirr H W, Chakauya E, Chikwamba R, Martens L, Tsekoa T L, Stoychev S H, Vandermarliere E. The Lyssavirus glycoprotein: A key to cross-immunity. Virology. 2016 November; 498:250-256. doi: 10.1016/j.virol.2016.08.034).

Interestingly, human anti-rabies antibodies most often recognize antigenic sites I or III on the glycoprotein G of RABV, whereas for example mouse anti-rabies antibodies most often recognize antigenic site II on the glycoprotein G of RABV. It is assumed that the immunogenic dominance of antigenic site II is lower in humans than in mice (Kramer R A, Marissen W E, Goudsmit J, Visser T J, Clijsters-Van der Horst M, Bakker A Q, de Jong M, Jongeneelen M, Thijsse S, Backus H H, Rice A B, Weldon W C, Rupprecht C E, Dietzschold B, Bakker A B, de Kruif J (2005) The human antibody repertoire specific for rabies virus glycoprotein as selected from immune libraries. Eur J Immunol. 35(7):2131-45). Accordingly, antibodies recognizing antigenic sites I and III on the glycoprotein G of RABV are believed to be more effective in humans than antibodies recognizing antigenic site II on the glycoprotein G of RABV.

Examples of antibodies binding to antigenic sites I and III (or to an epitope which at least partially overlaps with antigenic site III) on the glycoprotein G of RABV are well-known in the art. For example, the two anti-RABV antibodies CR57 and CR4098 were previously shown to recognize RABV G protein antigenic sites I and III, respectively (Bakker, A. B. H. et al., J Virol 79, 9062-9068, 2005). Moreover, WO 2016/078761 describes a panel of broadly neutralizing human anti-lyssavirus antibodies, which include antibodies RVA125, RVC3, RVC20 and RVD74, which bind to the antigenic site I, and antibodies RVA122, RVA144, RVB492, RVC4, RVC69, RVC38 and RVC58, which bind to the antigenic site III.

For example, the antibody, or the antigen-binding fragment thereof, is administered in combination with a further anti-lyssavirus antibody, or an antigen-binding fragment thereof, for example as described herein. It is understood that the two anti-lyssavirus antibodies, or the antigen-binding fragments thereof, which are administered in combination, are distinct. For example, the two anti-lyssavirus antibodies, or the antigen-binding fragments thereof, which are administered in combination, can bind specifically to different epitopes of a lyssavirus glycoprotein G, in particular on the glycoprotein G of RABV. Targeting different epitopes (different antigenic sites) avoids appearance of resistant virus strains and prevents the escape of resistant variants of the virus (viral escape mutants). Accordingly, the combination of two anti-lyssavirus antibodies, which bind to different epitopes of the lyssavirus G protein represents a treatment with an unprecedented breadth of reactivity and with reduced risk of escape mutant selection. In particular, a combination of two or more monoclonal antibodies binding to different epitopes or sites on the lyssavirus (e.g., RABV) G protein increases the protective effect and prevents the escape of resistant variants of the virus. Whether or not two or more antibodies bind to the same or different epitopes on the lyssavirus G protein may be easily determined by the person skilled in the art, for example by use of any competition study, for example as described in Example 3 of WO 2016/078761, which is incorporated herein by reference.

One of the two anti-lyssavirus antibodies, or the antigen-binding fragments thereof, which are administered in combination, may specifically bind to antigenic site I of glycoprotein G of a lyssavirus (e.g., RABV), whereas the other of the two anti-lyssavirus antibodies, or the antigen-binding fragments thereof, which are administered in combination, may specifically bind to antigenic site III (or to an epitope which at least partially overlaps with antigenic site III) of glycoprotein G of a lyssavirus (e.g., RABV).

The two anti-lyssavirus antibodies, or the antigen-binding fragments thereof, which are administered in combination, may be comprised in the same or distinct pharmaceutical composition. In other words, the two anti-lyssavirus antibodies, or the antigen-binding fragments thereof, which are administered in combination, may be administered separately, for example in separate pharmaceutical compositions, or together, i.e. as antibody “cocktail”, for example in the same pharmaceutical composition. For example, the two anti-lyssavirus antibodies, or the antigen-binding fragments thereof, which are administered in combination, are comprised in the same pharmaceutical composition.

For example, the antibody, or the antigen binding fragment thereof, for use according to the invention as described herein and the other antibody, which is administered in combination, are administered at equimolar amounts. If they are comprised in the same pharmaceutical composition, the (at least) two antibodies are in particular present (in the pharmaceutical composition) at equimolar amounts, for example as an equimolar mixture.

For example, the antibody, or the antigen-binding fragment thereof, neutralizes lyssavirus infection by (i) RABV and (ii) at least 50% of non-RABV lyssaviruses selected from the group consisting of DUVV, EBLV-1, EBLV-2, ABLY, IRKV, KHUV, ARAV, LBV, MOK, SHIV, BBLV, WCBV and IKOV with an IC₅₀ of less than 10000 ng/ml. In other words, the concentration of the antibody, or the antigen binding fragment thereof, required for 50% neutralization (IC₅₀) of the RABV and at least 50% of the above-mentioned non-RABV lyssaviruses is, for example, less than 10000 ng/ml.

Thereby, the antibodies according to the invention have a particular high or strong affinity for RABV and non-RABV lyssaviruses and are therefore particularly suitable for counteracting and/or at least in part preventing (reducing the occurrence) a RABV- and/or non-RABV-lyssavirus-infection and/or adverse effects of a RABV- and/or non-RABV-lyssavirus infection. In particular, antibodies with IC₅₀ values of more than 10000 ng/ml are unlikely to be effective in vivo.

“At least 50% of non-RABV lyssaviruses selected from the group consisting of DUVV, EBLV-1, EBLV-2, ABLY, IRKV, KHUV, ARAV, LBV, MOK, SHIV, BBLV, WCBV and IKOV” refers to at least 50% of the species Duvenhage virus (DUVV), European bat lyssavirus type 1 (EBLV-1) and type 2 (EBLV-2), Australian bat lyssavirus (ABLY), Irkut virus (IRKV), Khujand virus (KHUV), Aravan virus (ARAV), Lagos bat virus (LBV), Mokola virus (MOKV), Shimoni bat virus (SHIBV), Bokeloh bat lyssavirus (BBLV), West Caucasian bat virus (WCBV) and Ikoma lyssavirus (IKOV) i.e. at least 7 species among the above mentioned 13 species.

Each single lyssavirus species is considered as being neutralized with an IC₅₀ of less than 10000 ng/ml, whenever at least one isolate of any such lyssavirus species is neutralized with an IC₅₀ of less than 10000 ng/ml. For example, at least two isolates of any such lyssavirus species are neutralized with an IC₅₀ of less than 10000 ng/ml.

For example, the IC₅₀ of less than 10000 ng/ml is achieved with infectious viruses, i.e. in particular not with pseudotyped viruses.

Thereby, the antibodies, antibody variants and antigen binding fragments thereof, for use according to the invention are able to neutralize a broad spectrum of lyssaviruses.

For example, the antibody, or the antigen binding fragment thereof, neutralizes lyssavirus infection of at least 55% or at least 60% of the non-RABV lyssaviruses selected from the group consisting of DUVV, EBLV-1, EBLV-2, ABLV, IRKV, KHUV, ARAV, LBV, MOK, SHIV, BBLV, WCBV and IKOV with an IC₅₀ below 10000 ng/ml, such as at least 65%, at least 68% or at least 70% of the non-RABV lyssaviruses selected from the group consisting of DUVV, EBLV-1, EBLV-2, ABLV, IRKV, KHUV, ARAV, LBV, MOK, SHIV, BBLV, WCBV and IKOV with an IC₅₀ below 10000 ng/ml.

For example, the isolated antibody, antibody variants and antigen binding fragments thereof, neutralize lyssavirus infection by (i) RABV and (ii) at least 50% of all isolates of non-RABV lyssaviruses selected from the group consisting of DUVV, EBLV-1, EBLV-2, ABLV, IRKV, KHUV, ARAV, LBV, MOK, SHIV, BBLV, WCBV and IKOV, with an IC₅₀ of less than 10000 ng/ml.

Thereby, “at least 50% of all isolates of non-RABV lyssaviruses selected from the group consisting of DUVV, EBLV-1, EBLV-2, ABLV, IRKV, KHUV, ARAV, LBV, MOK, SHIV, BBLV, WCBV and IKOV” refers to all isolates of the above 13 species considered (i.e. all isolates considered represent 100% and the number of isolates neutralized with an IC₅₀ of less than 10000 ng/ml represents the respective percentage). In particular, all 32 isolates shown in Table 1 are considered to reflect 100% isolates:

TABLE 1
non-RABV lyssavirus isolates

| Isolate name | Viral species | Phylogroup |
|---|---|---|
| ABLV/Australia/bat/9810AUS-1998/ | ABLV | I |
| V1039-2011 | | |
| 98010 | ABLV | I |
| 1301 Bokeloh bat lyssavirus | BBLV | I |
| 86132SA | DUVV | I |
| DUW/ SouthAfrica/human/96132SA-1971/ | DUVV | I |
| RS639-2012 | | |
| EBLV1a/France/bat/ 122938-2002/V3951-2009 | EBLV-1 | I |
| EBLV1b/France/ bat/8918-1989 | EBLV-1 | I |
| EBLV2/UK/bat/RV1332-2002/V3951-2009 | EBLV-2 | I |
| 94112 | EBLV-2 | I |
| 02053 | EBLV-2 | I |
| 8619 | LBV | II |
| MOK | MOK | II |
| Shimoni bat Virus | SHIV | II |
| West Caucasian bat Virus | WCBV | III |
| Australian bat lyssavirus/RV634 | ABLV | I |
| Aravan Virus | ARAV | I |
| Duvenhage Virus RSA2006 | DUVV | I |
| Duvenhage Virus ZIM86-RV 131 | DUVV | I |
| European bat lyssavirus 1.RV20 | EBLV-1 | I |
| European bat lyssavirus 1.RV9 | EBLV-1 | I |
| EBLV 1a/France/bat/ 122938-2002/ | EBLV-1 | I |
| V3951-2009* | | |
| EBLV2/ UK/bat/RV1332-2002/V3951-2009* | EBLV-2 | I |
| European bat lyssavirus 2.RV1787 | EBLV-2 | I |
| European bat lyssavirus 2.RV628 | EBLV-2 | I |
| Irkut Virus | IRKV | I |
| Khujand Virus | KHUV | I |
| 8619* | LBV | II |
| Lagos Bat Virus NIG56-RV1 | LBV | II |
| Lagos Bat Virus SA2004 | LBV | II |
| Mokola Virus NIG68.RV4 | MOK | II |
| Mokola Virus 98/071 RA36 | MOK | II |
| Ikoma lyssavirus | IKOV | IV |

*same isolate tested as pseudovirus and infectious virus.

A more detailed description of the non-RABV lyssavirus isolates shown in Table 1 (as well as of various RABV isolates) is shown in FIG. 1 of WO 2016/078761, which is incorporated herein by reference. This includes—in addition to isolate name, viral species and phylogroup (as shown in Table 1)—host species, country and year of origin, lineage and the GenBank accession number of the amino acid and/or nucleotide sequence of the glycoprotein G of that isolate, if available (cf. FIG. 1 of WO 2016/078761).

Accordingly, if at least 16 of the 32 isolates specified in Table 1 are neutralized by the antibody, or antigen binding fragment thereof, with an IC₅₀ of less than 10000 ng/ml, the antibody, or antigen binding fragment thereof neutralizes infection of at least 50% of isolates of non-RABV lyssaviruses selected from the group consisting of DUVV, EBLV-1, EBLV-2, ABLY, IRKV, KHUV, ARAV, LBV, MOK, SHIV, BBLV, WCBV and IKOV with an IC₅₀ of less than 10000 ng/ml.

In some embodiments, the antibody, or antigen binding fragments thereof, neutralizes lyssavirus infection of at least 55%, in one embodiment at least 60%, in one embodiment at least 65%, in one embodiment at least 68% and in one embodiment at least 70% of all isolates of the non-RABV lyssaviruses selected from the group consisting of DUVV, EBLV-1, EBLV-2, ABLY, IRKV, KHUV, ARAV, LBV, MOK, SHIV, BBLV, WCBV and IKOV with an IC₅₀ below 10000 ng/ml.

For example, the first 12 isolates mentioned in Table 1 (i.e. from ABLV/Australia/bat/9810AUS-1998/V1039-2011 to MOK/MOK) are tested as infectious viruses, whereas the other isolates mentioned in Table 1 (i.e. from Shimoni bat Virus/SHIV to Ikoma lyssavirus/IKOV) are, for example, tested as pseudotyped viruses. Thereby, an isolate is considered as neutralizing a certain virus for infectious viruses, for example, if the IC₅₀ is less than 10000 ng/ml and for pseudotyped viruses, for example if the IC₉₀ is less than 10000 ng/ml.

For example, the antibody, or antigen binding fragments thereof, neutralizes lyssavirus infection by (i) RABV and (ii) at least 50% of all isolates of non-RABV lyssaviruses selected from the group consisting of ABLV/Australia/bat/9810AUS-1998/V1039-2011/ABLV, 98010/ABLV, 1301 Bokeloh bat lyssavirus/BBLV, 86132SA/DUVV, DUVV/SouthAfrica/human/961325A-1971/RS639-2012/DUVV, EBLV1a/France/bat/122938-2002/V3951-2009/EBLV-1, EBLV1b/France/bat/8918-1989/EBLV-1, EBLV2/UK/bat/RV1332-2002/V3951-2009/EBLV-2, 94112/EBLV-2, 02053/EBLV-2, 8619/LBV, MOK/MOK, Shimoni bat Virus/SHIV, West Caucasian bat Virus/WCBV, Australian bat lyssavirus/RV634/ABLV, Aravan Virus/ARAV, Duvenhage Virus RSA2006/DUVV, Duvenhage Virus ZIM86-RV 131/DUVV, European bat lyssavirus 1.RV20/EBLV-1, European bat lyssavirus 1.RV9/EBLV-1, EBLV 1a/France/bat/122938-2002/V3951-2009/EBLV-1, EBLV2/UK/bat/RV1332-2002/V3951-2009/EBLV-2, European bat lyssavirus 2.RV1787/EBLV-2, European bat lyssavirus 2.RV628/EBLV-2, Irkut Virus/IRKV, Khujand Virus/KHUV, 8619/LBV, Lagos Bat Virus N1G56-RV1/LBV, Lagos Bat Virus SA2004/LBV, Mokola Virus NIG68.RV4/MOK, Mokola Virus 98/071 RA36/MOK and Ikoma lyssavirus/IKOV with an IC₅₀ of less than 10000 ng/ml for ABLV/Australia/bat/9810AUS-1998/V1039-2011/ABLV, 98010/ABLV, 1301 Bokeloh bat lyssavirus/BBLV, 86132SA/DUVV, DUVV/SouthAfrica/human/96132SA-1971/RS639-2012/DUVV, EBLV1a/France/bat/122938-2002/V3951-2009/EBLV-1, EBLV1b/France/bat/8918-1989/EBLV-1, EBLV2/UK/bat/RV1332-2002/V3951-2009/EBLV-2, 94112/EBLV-2, 02053/EBLV-2, 8619/LBV, MOK/MOK tested as infectious viruses and with an IC₉₀ of less than 10000 ng/ml for Shimoni bat Virus/SHIV, West Caucasian bat Virus/WCBV, Australian bat lyssavirus/RV634/ABLV, Aravan Virus/ARAV, Duvenhage Virus RSA2006/DUVV, Duvenhage Virus ZIM86-RV 131/DUVV, European bat lyssavirus 1.RV20/EBLV-1, European bat lyssavirus 1.RV9/EBLV-1, EBLV 1a/France/bat/122938-2002/V3951-2009/EBLV-1, EBLV2/UK/bat/RV1332-2002/V3951-2009/EBLV-2, European bat lyssavirus 2.RV1787/EBLV-2, European bat lyssavirus 2.RV628/EBLV-2, Irkut Virus/IRKV, Khujand Virus/KHUV, 8619/LBV, Lagos Bat Virus NIG56-RV1/LBV, Lagos Bat Virus SA2004/LBV, Mokola Virus N1G68.RV4/MOK, Mokola Virus 98/071 RA36/MOK and Ikoma lyssavirus/IKOV tested as pseudotyped viruses.

In order to determine the IC₅₀ value in a neutralization assay, pseudoviruses and/or infectious viruses may be used. Respective neutralization assays are known to the person skilled in the art. For example, the neutralization assay according to Wright, E. et al., Vaccine 27, 7178-7186, 2009, which is incorporated by reference herein, is used for assessing pseudoviruses (PV). For infectious viruses, for example the “fluorescent-antibody virus neutralization test” (FAVN) according to Cliquet, F., et al., J. Immunol Methods 212, 79-87, 1998, which is incorporated by reference herein, or “the rapid fluorescent focus inhibition test” (REFIT) according to Smith, J. S., et al., Bull. World Health Organ. 48, 535-541, 1973, which is also incorporated by reference herein, is used.

In general, a neutralization assay typically measures the loss of infectivity of the virus through reaction of the virus with specific antibodies. Typically, a loss of infectivity is caused by interference by the bound antibody with any of the virus replication steps including binding to target cells, entry, and/or viral release. In the following a non-limiting example of a neutralization assay is given to illustrate the principle: a given amount of a virus, e.g. 50-100 TCDID50 (50% tissue culture infective dose), and different concentrations of the antibodies are mixed under appropriate conditions, e.g. for 1 hour at room temperature, and then inoculated into an appropriate target cell culture, e.g. Hep-2 cells or BHK-21 (baby hamster kidney 21) cells. Values may be typically provided per ml cell culture. The presence of unneutralized virus is detected for example after a predetermined amount of time, e.g. 1, 2, 3, 4, 5, 6, or 7 days, by measuring the cytopathic effect of the (unneutralized) virus on target cells, e.g. by using a colorimetric assay for the quantification of cellular viability, like for instance the WST-1 reagent. The more target cells are rescued from cell death or are measured to be viable, the more virus was neutralized by the antibodies. The effects measured are usually dose-dependent: The higher the antibody titer, the more cells are rescued. Depending on the neutralizing character of the antibody, the TCID50 values vary, e.g. an antibody of significant neutralizing character will require lower amounts (of the antibody) to be added (for, e.g., achieving the same amount of “rescued” target cells in the assay, i.e. cells measured to be viable) than another antibody of less pronounced neutralizing character.

For an anti-lyssavirus antibody for use according to the invention the IC₅₀ value (i.e. 50% neutralization) in an infectious virus neutralization assay as described above is, for example, less than 10000 ng/ml, i.e. regarding infectious viruses. For the same antibody the IC₉₀ value (i.e. 90% neutralization) in a pseudovirus neutralization assay as described above is, for example, less than 10000 ng/ml, i.e. regarding pseudoviruses.

Moreover, for example, the antibody, or the antigen binding fragment thereof, neutralizes lyssavirus infection by at least 70% of non-RABV phylogroup I lyssaviruses selected from the group consisting of DUVV, EBLV-1, EBLV-2, ABLY, IRKV, KHUV, ARAV and BBLV with an IC₅₀ of less than 10000 ng/ml. Thereby, “at least 70%” of the above mentioned species means at least 6 out of the 8 species. Infection of one lyssavirus species is considered as neutralized with an IC₅₀ of less than 10000 ng/ml, if infection of at least one isolate of this lyssavirus species is neutralized with an IC₅₀ of less than 10000 ng/ml.

For example, the antibody, or antigen binding fragment thereof, neutralizes lyssavirus infection of at least 75% or at least 80%, such as at least 82%, of the non-RABV phylogroup I lyssaviruses selected from the group consisting of DUVV, EBLV-1, EBLV-2, ABLY, IRKV, KHUV, ARAV and BBLV with an IC₅₀ of less than 10000 ng/ml.

According to another embodiment, the antibody, or the antigen binding fragment thereof, neutralizes lyssavirus infection by at least 70% of isolates of non-RABV phylogroup I lyssaviruses selected from the group consisting of DUVV, EBLV-1, EBLV-2, ABLY, IRKV, KHUV, ARAV and BBLV with an IC₅₀ of less than 10000 ng/ml. Thereby, “at least 70% of isolates of non-RABV lyssaviruses selected from the group consisting of DUVV, EBLV-1, EBLV-2, ABLY, IRKV, KHUV ARAV and BBLV” refers to all isolates of the above 8 species considered (i.e. all isolates considered represent 100% and the number of isolates neutralized with an IC₅₀ of less than 10000 ng/ml represents the respective percentage). In particular, the 22 isolates mentioned in Table 1 regarding phylogroup I species are considered to calculate the percentage. Accordingly, if at least 16 out of these 22 isolates mentioned in Table 1 regarding phylogroup 1 species are neutralized by the antibody, antibody variant or antigen binding fragment thereof, with an IC₅₀ of less than 10000 ng/ml, the antibody, antibody variant or antigen binding fragment thereof neutralizes infection of at least 70% of isolates of non-RABV phylogroup I lyssaviruses selected from the group consisting of DUVV, EBLV-1, EBLV-2, ABLY, IRKV, KHUV, ARAV and, e.g BBLV, with an IC₅₀ of less than 10000 ng/ml. For example, the isolated antibody, antibody variants and antigen binding fragments thereof neutralizes lyssavirus infection of at least 75% or at least 80%, such as at least 82%, of isolates of the non-RABV phylogroup I lyssaviruses selected from the group consisting of DUVV, EBLV-1, EBLV-2, ABLY, IRKV, KHUV, ARAV and, e.g., BBLV with an IC₅₀ of less than 10000 ng/ml.

In some embodiments, the antibody, or antigen binding fragment thereof, neutralizes lyssavirus infection by at least 70% of the isolates of non-RABV phylogroup I lyssaviruses selected from the group consisting of ABLV/Australia/bat/9810AUS-1998/V1039-2011/ABLV, 98010/ABLV, 1301 Bokeloh bat lyssavirus/BBLV, 86132SA/DUVV, DUVV/SouthAfrica/human/96132SA-1971/RS639-2012/DUVV, EBLV1a/France/bat/122938-2002/V3951-2009/EBLV-1, EBLV1b/France/bat/8918-1989/EBLV-1, EBLV2/UK/bat/RV1332-2002/V3951-2009/EBLV-2, 94112/EBLV-2, 02053/EBLV-2, Australian bat lyssavirus/RV634/ABLV, Aravan Virus/ARAV, Duvenhage Virus RSA2006/DUVV, Duvenhage Virus ZIM86-RV 131/DUVV, European bat lyssavirus 1.RV20/EBLV-1, European bat lyssavirus 1.RV9/EBLV-1, EBLV 1a/France/bat/122938-2002/V3951-2009/EBLV-1, EBLV2/UK/bat/RV1332-2002/V3951-2009/EBLV-2, European bat lyssavirus 2.RV1787/EBLV-2 and European bat lyssavirus 2.RV628/EBLV-2, Irkut Virus/IRKV, Khujand Virus/KHUV, with an IC₅₀ of less than 10000 ng/ml for ABLV/Australia/bat/9810AUS-1998/V1039-2011/ABLV, 98010/ABLV, 1301 Bokeloh bat lyssavirus/BBLV, 86132SA/DUVV, DUVV/SouthAfrica/human/96132SA-1971/RS639-2012/DUVV, EBLV1a/France/bat/122938-2002/V3951-2009/EBLV-1, EBLV1b/France/bat/8918-1989/EBLV-1, EBLV2/UK/bat/RV1332-2002/V3951-2009/EBLV-2, 94112/EBLV-2 and 02053/EBLV-2, tested as infectious viruses and with an IC %) of less than 10000 ng/ml for Australian bat lyssavirus/RV634/ABLV, Aravan Virus/ARAV, Duvenhage Virus RSA2006/DUVV, Duvenhage Virus ZIM86-RV 131/DUVV, European bat lyssavirus 1.RV20/EBLV-1, European bat lyssavirus 1.RV9/EBLV-1, EBLV 1a/France/bat/122938-2002/V3951-2009/EBLV-1, EBLV2/UK/bat/RV1332-2002/V3951-2009/EBLV-2, European bat lyssavirus 2.RV1787/EBLV-2, European bat lyssavirus 2.RV628/EBLV-2, Irkut Virus/IRKV and Khujand Virus/KHUV tested as pseudotyped viruses.

Among the lyssaviruses, and in particular the phylogroup I lyssaviruses, the antibody, or the antigen binding fragment thereof, neutralizes, for example, infection of EBLV-1. Rabies due to European Bat Lyssavirus type 1 is present in many European countries and bats are listed as protected species across Europe. The disease is fatal in humans and has been described in Europe following a bat bite. Rabies pre-exposure vaccination and post-exposure treatment is recommended for occupationally exposed persons and treatment of international travellers after bat bites is also recommended (Stantic-Pavlinic M. (2005) Eurosurveillance, Volume 10, Issue 11). In particular, the antibody, or the antigen binding fragment thereof, neutralizes infection of at least one isolate of EBLV-1, such as of at least two or three EBLV-1 isolates, e.g. the EBLV-1 isolates mentioned in Table 1, with an IC₅₀ below 10000 ng/ml. Thereby, the one or more EBLV-1 isolates are, for example, neutralized in both, in a pseudovirus neutralization assay as described above and in an infectious virus neutralization assay as described above. For example, the IC₅₀ value in the infectious virus neutralization assay is below 10000 ng/ml and the IC₉₀ value in the pseudovirus neutralization assay is below 10000 ng/ml.

For example, the antibody, or the antigen binding fragment thereof, neutralizes infection by RABV CVS-11 with an IC₉₀ of 400 ng/ml or less or with an IC₉₀ of 100 ng/ml or less, e.g., with an IC₉₀ of 50 ng/ml or less or with an IC₉₀ of 1 ng/ml or less. In other words, the concentration of the antibody, or the antigen binding fragment thereof, required for 90% neutralization (IC₉₀) of the RABV isolate CVS-11 (challenge virus strain 11) is, for example, 400 ng/ml or less or 100 ng/ml or less, such as 50 ng/ml or less or 1 ng/ml or less, for example in a pseudovirus neutralization assay as described above, and, optionally, also in a REFIT infectious virus neutralization assay as described above.

In general, the anti-lyssavirus antibody, or the antigen binding fragment thereof, typically comprises (at least) three CDRs on the heavy chain and (at least) three CDRs on the light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Antigen receptors are typically composed of two variable domains (on two different polypeptide chains, i.e. heavy and light chain). Accordingly, the antibody comprises in particular six CDRs for each antigen receptor (heavy chain: CDRH1, CDRH2, and CDRH3; light chain: CDRL1, CDRL2, and CDRL3). For example, a single antibody molecule has two antigen receptors and therefore contains twelve CDRs. The position of the CDR amino acids are usually defined according to the IMGT numbering system (IMGT: http://www.imgt.org cf. Lefranc, M.-P. et al. (2009) Nucleic Acids Res. 37, D1006-D1012). The CDRs of the antibody heavy chains are also referred to as CDRH1, CDRH2 and CDRH3, respectively. Similarly, the CDRs of the antibody light chains are also referred to as CDRL1, CDRL2 and CDRL3, respectively. The CDRs on the heavy and/or light chain may be separated by framework regions, whereby a framework region (FR) is a region in the variable domain which is less “variable” than the CDR. For example, a chain (or each chain, respectively) may be composed of four framework regions, separated by three CDR.

In one embodiment, the CDRs, in particular at least CDRH3, of the anti-lyssavirus antibody are derived from an antibody developed in a human. In particular, the CDRs, in particular at least the CDRH3, of the anti-lyssavirus antibody are of human origin or sequence variants thereof. In general, sequence variants are in particular functional sequence variants, e.g. wherein the binding of the antibody to a lyssavirus glycoprotein G, such as the RABV (and/or non-RABV) G protein, is maintained.

In the context of the invention, examples of anti-lyssavirus antibodies or antigen-binding fragments thereof, are described in WO 2016/078761, which is incorporated herein by reference.

For example, the anti-lyssavirus antibody, or the antigen binding fragment thereof, comprises a heavy chain comprising CDRH1, CDRH2 and CDRH3 and a light chain comprising CDRL1, CDRL2 and CDRL3, wherein the heavy chain CDRH3 comprises an amino acid sequence that is at least 80% or at least 90% identical to any one of SEQ ID NOs: 3, 21, 39, 57, 77, 95, 113, 131, 149, 167, 185, or 203, for example amino acid sequences that are at least 80% or at least 90% identical to SEQ ID NOs: 95 or 167. In particular, the heavy chain CDRH3 of the antibody, or of the antigen binding fragment thereof, comprises the amino acid sequence of SEQ ID NOs: 3, 21, 39, 57, 77, 95, 113, 131, 149, 167, 185, or 203, or a sequence variant thereof, e.g., of SEQ ID NOs: 95 or 167, or a sequence variant thereof. In particular, the antibody, or an antigen binding fragment thereof, may comprise a heavy chain comprising CDRH1, CDRH2 and CDRH3 and a light chain comprising CDRL1, CDRL2 and CDRL3, wherein the heavy chain CDRH3 comprises an amino acid sequence variant to SEQ ID NOs: 3, 21, 39, 57, 77, 95, 113, 131, 149, 167, 185, or 203, or a sequence variant thereof, such as amino acid sequence variants according to SEQ ID NOs: 95 or 167, or a sequence variant thereof. For example, the heavy chain comprises at least two CDRH3 with one heavy chain CDRH3 comprising an amino acid sequence that is at least 80% or at least 90% identical to SEQ ID NO: 95 and one heavy chain CDRH3 comprising an amino acid sequence that is at least 80% or at least 90% identical to SEQ ID NO: 167.

In some embodiments, the antibody, or the antigen binding fragment thereof, comprises a heavy chain comprising CDR1, CDR2 and CDR3 and a light chain comprising CDR1, CDR2 and CDR3, wherein the heavy chain CDR3 comprises an amino acid sequence that is at least 90%, for example, 90%, 91%, 92%, 93%, 94%, 95%, 96%, 97%, 98%, 99% or 100% identical to SEQ ID NOs: 3, 21, 39, 57, 77, 95, 113, 131, 149, 167, 185, or 203, such as SEQ ID NOs: 95 or 167. For example, the heavy chain comprises at least two CDRH3 with one heavy chain CDRH3 comprising an amino acid sequence of SEQ ID NO: 95 and one heavy chain CDRH3 comprising an amino acid sequence of SEQ ID NO: 167.

Table 2 provides the SEQ ID numbers for the amino acid sequences of the six CDRs of the heavy and light chains, respectively, of examples, without being limited, of anti-lyssavirus antibodies that may be used in invention.

TABLE 2
SEQ ID Numbers for CDR Polypeptides
of examples of anti-lyssavirus antibodies.

| | SEQ ID NOs. for CDR Polypeptides | | | | | |
|---|---|---|---|---|---|---|
| | CDRH1 | CDRH2 | CDRH3 | CDRL1 | CDRL2 | CDRL3 |
| RVA122 | 1 | 2 | 3 | 4 | 5/6 | 7 |
| RVA144 | 19 | 20 | 21 | 22 | 23/24 | 25 |
| RVB185 | 37 | 38 | 39 | 40 | 41/42 | 43 |
| RVB492 | 55 | 56 | 57 | 58 | 59/60 | 61 |
| RVC3 | 75 | 76 | 77 | 78 | 79/80 | 81 |
| RVC20 | 93 | 94 | 95 | 96 | 97/98 | 99 |
| RVC21 | 111 | 112 | 113 | 114 | 115/116 | 117 |
| RVC38 | 129 | 130 | 131 | 132 | 133/134 | 135 |
| RVC44 | 147 | 148 | 149 | 150 | 151/152 | 153 |
| RVC58 | 165 | 166 | 167 | 168 | 169/170 | 171 |
| RVC68 | 183 | 184 | 185 | 186 | 187/188 | 189 |
| RVC111 | 201 | 202 | 203 | 204 | 205/206 | 207 |

In some embodiments, the anti-lyssavirus antibody, or the antigen-binding fragment thereof, is a sequence variant of the antibodies described herein or known in the prior art. Such sequence variants are in particular functional, e.g. wherein the binding of the antibody to the anti-lyssavirus glycoprotein G, such as RABV (and/or non-RABV) G protein, is maintained. Such variants include natural variants generated by somatic mutation in vivo during the immune response or in vitro, for example upon culture of immortalized B cell clones. Alternatively, variants may arise due to the degeneracy of the genetic code or may be produced due to errors in transcription or translation.

Further sequence variants of the antibody sequences, e.g. having improved affinity and/or potency, may be obtained using methods known in the art. For example, amino acid substitutions may be used. Alternatively, codon optimization of the nucleotide sequence may be used, e.g. to improve the efficiency of translation in expression systems for the production of the antibody. Further, polynucleotides comprising a sequence optimized for antibody specificity or neutralizing activity by the application of a directed evolution method to any of the nucleic acid sequences of the invention are also within the scope of the invention.

As described above, sequence variants of antibodies have, for example, at least 70% or 75% sequence identity to the respective reference sequence, such as at least 80% or 85% sequence identity. For example, a sequence variant has at least 88% sequence identity to the respective reference sequence. For example, a sequence variant has at least 90% sequence identity to the respective reference sequence. For example, a sequence variant has at least 92% sequence identity to the respective reference sequence. For example, a sequence variant has at least 95% sequence identity to the respective reference sequence. For example, a sequence variant has at least 96% sequence identity to the respective reference sequence. For example, a sequence variant has at least 97% sequence identity to the respective reference sequence. For example, a sequence variant has at least 98% or at least 99% sequence identity to the respective reference sequence. For example, a sequence variants of a complete antibody (chain) has a greater homology to the sequences listed herein in the CDRs of the heavy chain variable region (V_{H}) and light chain variable region (V_{L}) than in the framework region. Accordingly, the sequence variant of the antibody, or an antigen binding fragment thereof, comprises the variation in the framework region(s) of the antibody or in the nucleic acid residues that encode the framework region(s) of the antibody.

In the (variant) antibody the number of somatic mutations is, for example, reduced (e.g.,“germlined” antibodies; “germlined” meaning reverted back to the “germline” configuration). Germline sequences of antibodies may be determined, for example, with reference to IMGT database (e.g., according to the IMGT VDJ and Vi assignments and rearrangement interpretation: http://www.imgt.org/; cf. Lefranc, M.-P. et al. (2009) Nucleic Acids Res. 37, D1006-D1012) and “germlined” antibody variants may be produced, for example, by gene synthesis or by site-directed mutagenesis. A low level of somatic mutations reduces the potential risk of antibody immunogenicity. For example, the number of somatic mutations is reduced in the framework regions (FR) (i.e. “framework regions germlined” antibodies, also referred to herein as FR-GL variants). (Variant) antibodies, or an antigen binding fragment thereof, and FR-GL variants, respectively, without any somatic mutations in the framework regions (FR) are, for example, used. In some embodiments such (variant) antibodies, or an antigen binding fragment thereof, and FR-GL variants, respectively, with as few somatic mutations as possible, whereby on the other hand the neutralizing activity is not impaired (as compared to the reference antibody/fragment containing (more) somatic mutations) are used. Such antibodies are on the one hand not impaired in their neutralizing activities, thus showing a very high potency and breadth. On the other hand, a potential risk of antibody immunogenicity is significantly reduced.

In an embodiment, the antibody or antibody fragment comprises at least one CDR with a sequence that has at least 95% sequence identity to any one of SEQ ID NOs: 1-7, 19-25, 37-43, 55-61, 75-81, 93-99, 111-117, 129-135, 147-153, 165-171, 183-189, or 201-207. The amino acid sequences of the heavy and light chain variable regions of the antibodies as well as the nucleic acid sequences that encode them are provided in the Table of Sequences and SEQ ID Numbers below. The amino acid residues corresponding to the six CDRs and the nucleic acid residues that encode them are highlighted in bold text.

In some embodiments, the antibody or antigen binding fragment thereof, comprises a heavy chain CDR1, CDR2 and CDR3 and light chain CDR1, CDR2, and CDR3 amino acid sequences that are at least 80%, for example, 80%, 81%, 82%, 83%, 84%, 85%, 86%, 87%, 88%, 89%, 90%, 91%, 92%, 93%, 94%, 95%, 96%, 97%, 98%, 99% or 100% identical to the amino acid sequences of SEQ ID NOs: 1-7, 19-25, 37-43, 55-61, 75-81, 93-99, 111-117, 129-135, 147-153, 165-171, 183-189, and 201-207, respectively.

In some embodiments, the antibody, or antigen binding fragment thereof, comprises more than one CDR with a sequence that has at least 95% sequence identity to any one of SEQ ID NOs: 1-7, 19-25, 37-43, 55-61, 75-81, 93-99, 111-117, 129-135, 147-153, 165-171, 183-189, and 201-207.

In some embodiments, the antibody, or antigen binding fragment thereof, comprises two CDRs with a sequence that has at least 95% sequence identity to any one of SEQ ID NOs: 1-7, 19-25, 37-43, 55-61, 75-81, 93-99, 111-117, 129-135, 147-153, 165-171, 183-189, or 201-207. For example, the antibody, or antigen binding fragment thereof, comprises (i) a CDRH1 that has at least 95% sequence identity to any one of SEQ ID NOs: 1, 19, 37, 55, 75, 93, 111, 129, 147, 165, 183 or 201 and a CDRL1 that has at least 95% sequence identity to any one of SEQ ID NOs: 4, 22, 40, 58, 78, 96, 114, 132, 150, 168, 186, or 204; (ii) a CDRH2 that has at least 95% sequence identity to any one of SEQ ID NOs: 2, 20, 38, 56, 76, 94, 112, 130, 148, 166, 184, or 202, and a CDRL2 that has at least 95% sequence identity to any one of SEQ ID NOs: 5, 6, 23, 24, 41, 42, 59, 60, 79, 80, 97, 98, 115, 116, 133, 134, 151, 152, 169, 170, 187, 188, 205, or 206; or (iii) a CDRH3 that has at least 95% sequence identity to any one of SEQ ID NOs: 3, 21, 39, 57, 77, 95, 113, 131, 149, 167, 185, or 203, and a CDRL3 that has at least 95% sequence identity to any one of SEQ ID NOs: 7, 25, 43, 61, 81, 99, 117, 135, 153, 171, 189, or 201.

For example, the antibody, or antigen binding fragment thereof, comprises three CDRs with a sequence that has at least 95% sequence identity to any one of SEQ ID NOs: 1-7, 19-25, 37-43, 55-61, 75-81, 93-99, 111-117, 129-135, 147-153, 165-171, 183-189, and 201-207. For example, the antibody, or antigen binding fragment thereof, comprises (i) a CDRH1 that has at least 95% sequence identity to any one of SEQ ID NOs: 1, 19, 37, 55, 75, 93, 111, 129, 147, 165, 183 or 201, a CDRH2 that has at least 95% sequence identity to any one of SEQ ID NOs: 2, 20, 38, 56, 76, 94, 112, 130, 148, 166, 184, or 202, and a CDRH3 that has at least 95% sequence identity to any one of SEQ ID NOs: 3, 21, 39, 57, 77, 95, 113, 131, 149, 167, 185, or 203; or (ii) a CDRL1 that has at least 95% sequence identity to any one of SEQ ID NOs: 4, 22, 40, 58, 78, 96, 114, 132, 150, 168, 186, or 204, a CDRL2 that has at least 95% sequence identity to any one of SEQ ID NOs: 5, 6, 23, 24, 41, 42, 59, 60, 79, 80, 97, 98, 115, 116, 133, 134, 151, 152, 169, 170, 187, 188, 205, or 206, and a CDRL3 that has at least 95% sequence identity to any one of SEQ ID NOs: 7, 25, 43, 61, 81, 99, 117, 135, 153, 171, 189, or 201.

For example, the antibody, or antigen binding fragment thereof, comprises four CDRs with a sequence that has at least 95% sequence identity to any one of SEQ ID NOs: 1-7, 19-25, 37-43, 55-61, 75-81, 93-99, 111-117, 129-135, 147-153, 165-171, 183-189, and 201-207. For example, the antibody, or antigen binding fragment thereof, comprises (i) a CDRH1 that has at least 95% sequence identity to any one of SEQ ID NOs: 1, 19, 37, 55, 75, 93, 111, 129, 147, 165, 183 or 201, a CDRH2 that has at least 95% sequence identity to any one of SEQ ID NOs: 2, 20, 38, 56, 76, 94, 112, 130, 148, 166, 184, or 202, a CDRH3 that has at least 95% sequence identity to any one of SEQ ID NOs: 3, 21, 39, 57, 77, 95, 113, 131, 149, 167, 185, or 203, and a CDRL that has at least 95% sequence identity to any one of SEQ ID NOs: 4-6, 20-22, 36-38, or 52-54; (ii) a CDRL1 that has at least 95% sequence identity to any one of SEQ ID NOs: 4, 22, 40, 58, 78, 96, 114, 132, 150, 168, 186, or 204, a CDRL2 that has at least 95% sequence identity to any one of SEQ ID NOs: 5, 6, 23, 24, 41, 42, 59, 60, 79, 80, 97, 98, 115, 116, 133, 134, 151, 152, 169, 170, 187, 188, 205, or 206, a CDRL3 that has at least 95% sequence identity to any one of SEQ ID NOs: 7, 25, 43, 61, 81, 99, 117, 135, 153, 171, 189, or 201, and a CDRH that has at least 95% sequence identity to any one of SEQ ID NOs: 1-3, 19-21, 37-39, 55-57, 75-77, 93-95, 111-113, 129-131, 147-149, 165-167, 183-185 or 201-203, whereby a CDRH3 that has at least 95% sequence identity to any one of SEQ ID NOs: 3, 21, 39, 57, 77, 95, 113, 131, 149, 167, 185, or 203 is an example; (iii) a CDRH1 that has at least 95% sequence identity to any one of SEQ ID NOs: 1, 19, 37, 55, 75, 93, 111, 129, 147, 165, 183 or 201, a CDRL1 that has at least 95% sequence identity to any one of SEQ ID NOs: 4, 22, 40, 58, 78, 96, 114, 132, 150, 168, 186, or 204, a CDRH2 that has at least 95% sequence identity to any one of SEQ ID NOs: 2, 20, 38, 56, 76, 94, 112, 130, 148, 166, 184, or 202, and a CDRL2 that has at least 95% sequence identity to any one of SEQ ID NOs: 5, 6, 23, 24, 41, 42, 59, 60, 79, 80, 97, 98, 115, 116, 133, 134, 151, 152, 169, 170, 187, 188, 205, or 206; (iv) a CDRH1 that has at least 95% sequence identity to any one of SEQ ID NOs: 1, 19, 37, 55, 75, 93, 111, 129, 147, 165, 183 or 201, a CDRL1 that has at least 95% sequence identity to any one of SEQ ID NOs: 4, 22, 40, 58, 78, 96, 114, 132, 150, 168, 186, or 204, a CDRH3 that has at least 95% sequence identity to any one of SEQ ID NOs: 3, 21, 39, 57, 77, 95, 113, 131, 149, 167, 185, or 203, and a CDRL3 that has at least 95% sequence identity to any one of SEQ ID NOs: 7, 25, 43, 61, 81, 99, 117, 135, 153, 171, 189, or 201; or (v) a CDRH2 that has at least 95% sequence identity to any one of SEQ ID NOs: 2, 20, 38, 56, 76, 94, 112, 130, 148, 166, 184, or 202, a CDRL2 that has at least 95% sequence identity to any one of SEQ ID NOs: 5, 6, 23, 24, 41, 42, 59, 60, 79, 80, 97, 98, 115, 116, 133, 134, 151, 152, 169, 170, 187, 188, 205, or 206, a CDRH3 that has at least 95% sequence identity to any one of SEQ ID NOs: 3, 21, 39, 57, 77, 95, 113, 131, 149, 167, 185, or 203, and a CDRL3 that has at least 95% sequence identity to any one of SEQ ID NOs: 7, 25, 43, 61, 81, 99, 117, 135, 153, 171, 189, or 201.

For example, the antibody, or antigen binding fragment thereof, comprises five CDRs with a sequence that has at least 95% sequence identity to any one of SEQ ID NOs: 1-7, 19-25, 37-43, 55-61, 75-81, 93-99, 111-117, 129-135, 147-153, 165-171, 183-189, and 201-207. For example, the antibody, or antigen binding fragment thereof, comprises five CDRs selected from the group of a CDRH1 that has at least 95% sequence identity to any one of SEQ ID NOs: 1, 19, 37, 55, 75, 93, 111, 129, 147, 165, 183 or 201, a CDRH2 that has at least 95% sequence identity to any one of SEQ ID NOs: 2, 20, 38, 56, 76, 94, 112, 130, 148, 166, 184, or 202, a CDRH3 that has at least 95% sequence identity to any one of SEQ ID NOs: 3, 21, 39, 57, 77, 95, 113, 131, 149, 167, 185, or 203, a CDRL1 that has at least 95% sequence identity to any one of SEQ ID NOs: 4, 22, 40, 58, 78, 96, 114, 132, 150, 168, 186, or 204, a CDRL2 that has at least 95% sequence identity to any one of SEQ ID NOs: 5, 6, 23, 24, 41, 42, 59, 60, 79, 80, 97, 98, 115, 116, 133, 134, 151, 152, 169, 170, 187, 188, 205, or 206, and a CDRL3 that has at least 95% sequence identity to any one of SEQ ID NOs: 7, 25, 43, 61, 81, 99, 117, 135, 153, 171, 189, or 201.

For example, the antibody, or antigen binding fragment thereof, comprises six CDRs with a sequence that has at least 95% sequence identity to any one of SEQ ID NOs: 1-7, 19-25, 37-43, 55-61, 75-81, 93-99, 111-117, 129-135, 147-153, 165-171, 183-189, and 201-207. For example, the antibody, or antigen binding fragment thereof, comprises six CDRs selected from the group of a CDRH1 that has at least 95% sequence identity to any one of SEQ ID NOs: 1, 19, 37, 55, 75, 93, 111, 129, 147, 165, 183 or 201, a CDRH2 that has at least 95% sequence identity to any one of SEQ ID NOs: 2, 20, 38, 56, 76, 94, 112, 130, 148, 166, 184, or 202, a CDRH3 that has at least 95% sequence identity to any one of SEQ ID NOs: 3, 21, 39, 57, 77, 95, 113, 131, 149, 167, 185, or 203, a CDRL1 that has at least 95% sequence identity to any one of SEQ ID NOs: 4, 22, 40, 58, 78, 96, 114, 132, 150, 168, 186, or 204, a CDRL2 that has at least 95% sequence identity to any one of SEQ ID NOs: 5, 6, 23, 24, 41, 42, 59, 60, 79, 80, 97, 98, 115, 116, 133, 134, 151, 152, 169, 170, 187, 188, 205, or 206, and a CDRL3 that has at least 95% sequence identity to any one of SEQ ID NOs: 7, 25, 43, 61, 81, 99, 117, 135, 153, 171, 189, or 201.

For example, the antibody, or antigen binding fragment thereof, comprises: (i) heavy chain CDRH1, CDRH2, and CDRH3 amino acid sequences and light chain CDRL1, CDRL2, and CDRL3 amino acid sequences as set forth in SEQ ID NOs: 1-5 and 7, or sequence variants thereof, or as set forth in SEQ ID NOs: 1-4 and 6-7, or sequence variants thereof; (ii) heavy chain CDRH1, CDRH2, and CDRH3 amino acid sequences and light chain CDRL1, CDRL2, and CDRL3 amino acid sequences as set forth in SEQ ID NOs: 19-23 and 25, or sequence variants thereof, or as set forth in SEQ ID NOs: 19-22 and 24-25, or sequence variants thereof; (iii) heavy chain CDRH1, CDRH2, and CDRH3 amino acid sequences and light chain CDRL1, CDRL2, and CDRL3 amino acid sequences as set forth in SEQ ID NOs: 37-41 and 43, or sequence variants thereof, or as set forth in SEQ ID NOs: 37-40 and 42-43, or sequence variants thereof; (iv) heavy chain CDRH1, CDRH2, and CDRH3 amino acid sequences and light chain CDRL1, CDRL2, and CDRL3 amino acid sequences as set forth in SEQ ID NOs: 55-59 and 61, or sequence variants thereof, or as set forth in SEQ ID NOs: 55-58 and 60-61, or sequence variants thereof; (v) heavy chain CDRH1, CDRH2, and CDRH3 amino acid sequences and light chain CDRL1, CDRL2, and CDRL3 amino acid sequences as set forth in SEQ ID NOs: 75-79 and 81, or sequence variants thereof, or as set forth in SEQ ID NOs: 75-78 and 80-81, or sequence variants thereof; (vi) heavy chain CDRH1, CDRH2, and CDRH3 amino acid sequences and light chain CDRL1, CDRL2, and CDRL3 amino acid sequences as set forth in SEQ ID NOs: 93-97 and 99, or sequence variants thereof, or as set forth in SEQ ID NOs: 93-96 and 98-99, or sequence variants thereof; (vii) heavy chain CDRH1, CDRH2, and CDRH3 amino acid sequences and light chain CDRL1, CDRL2, and CDRL3 amino acid sequences as set forth in SEQ ID NOs: 111-115 and 117, or sequence variants thereof, or as set forth in SEQ ID NOs: 111-114 and 116-117, or sequence variants thereof; (viii) heavy chain CDRH1, CDRH2, and CDRH3 amino acid sequences and light chain CDRL1, CDRL2, and CDRL3 amino acid sequences as set forth in SEQ ID NOs: 129-133 and 135, or sequence variants thereof, or as set forth in SEQ ID NOs: 129-132 and 134-135, or sequence variants thereof; (ix) heavy chain CDRH1, CDRH2, and CDRH3 amino acid sequences and light chain CDRL1, CDRL2, and CDRL3 amino acid sequences as set forth in SEQ ID NOs: 147-151 and 153, or sequence variants thereof, or as set forth in SEQ ID NOs: 147-150 and 152-153, or sequence variants thereof; (x) heavy chain CDRH1, CDRH2, and CDRH3 amino acid sequences and light chain CDRL1, CDRL2, and CDRL3 amino acid sequences as set forth in SEQ ID NOs: 165-169 and 171, or sequence variants thereof, or as set forth in SEQ ID NOs: 165-168 and 170-171, or sequence variants thereof; (xi) heavy chain CDRH1, CDRH2, and CDRH3 amino acid sequences and light chain CDRL1, CDRL2, and CDRL3 amino acid sequences as set forth in SEQ ID NOs: 183-187 and 189, or sequence variants thereof, or as set forth in SEQ ID NOs: 183-186 and 188-189, or sequence variants thereof; or (xii) heavy chain CDRH1, CDRH2, and CDRH3 amino acid sequences and light chain CDRL1, CDRL2, and CDRL3 amino acid sequences as set forth in SEQ ID NOs: 201-205 and 207, or sequence variants thereof, or as set forth in SEQ ID NOs: 201-204 and 206-207, or sequence variants thereof.

For example, the antibody, or antigen binding fragment thereof, comprises: (i) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 1-5 and 7 or to the amino acid sequences of SEQ ID NOs: 1-4 and 6-7, respectively; (ii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 19-23 and 25 or to the amino acid sequences of SEQ ID NOs: 19-22 and 24-25, respectively; (iii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 37-41 and 43 or to the amino acid sequences of SEQ ID NOs: 37-40 and 42-43, respectively; (iv) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 55-59 and 61 or to the amino acid sequences of SEQ ID NOs: 55-58 and 60-61, respectively; (v) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 75-79 and 81 or to the amino acid sequences of SEQ ID NOs: 75-78 and 80-81, respectively; (vi) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 93-97 and 99 or to the amino acid sequences of SEQ ID NOs: 93-96 and 98-99, respectively; (vii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 111-115 and 117 or to the amino acid sequences of SEQ ID NOs: 111-114 and 116-117, respectively; (viii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 129-133 and 135 or to the amino acid sequences of SEQ ID NOs: 129-132 and 134-135, respectively; (ix) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 147-151 and 153 or to the amino acid sequences of SEQ ID NOs: 147-150 and 152-153, respectively; (x) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 165-169 and 171 or to the amino acid sequences of SEQ ID NOs: 165-168 and 170-171, respectively; (xi) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 201-205 and 207 or to the amino acid sequences of SEQ ID NOs: 201-204 and 206-207, respectively.

In some embodiments, the antibody, or antigen binding fragment thereof, comprises heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 93-97 and 99 or to the amino acid sequences of SEQ ID NOs: 93-96 and 98-99, respectively. For example, the antibody, or antigen binding fragment thereof, comprises heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof.

In another embodiment, the antibody, or antigen binding fragment thereof, comprises heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 165-169 and 171 or to the amino acid sequences of SEQ ID NOs: 165-168 and 170-171, respectively. For example, the antibody, or antigen binding fragment thereof, comprises heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, or sequence variants thereof.

In some embodiments, the antibody or antigen binding fragment comprises a heavy chain CDR1 with the amino acid sequence of SEQ ID NOs: 1, 19, 37, 55, 75, 93, 111, 129, 147, 165, 183 or 201 or sequence variants thereof; a heavy chain CDR2 with the amino acid sequence of SEQ ID NOs: 2, 20, 38, 56, 76, 94, 112, 130, 148, 166, 184, or 202 or sequence variants thereof; and a heavy chain CDR3 with the amino acid sequence of SEQ ID NOs: 3, 21, 39, 57, 77, 95, 113, 131, 149, 167, 185, or 203 or sequence variants thereof. In certain embodiments, an antibody or antibody fragment as provided herein comprises a heavy chain comprising the amino acid sequence of (i) SEQ ID NO: 1 for CDRH1, SEQ ID NO: 2 for CDRH2 and SEQ ID NO: 3 for CDRH3, (ii) SEQ ID NO: 19 for CDRH1, SEQ ID NO: 20 for CDRH2, and SEQ ID NO: 21 for CDRH3, (iii) SEQ ID NO: 37 for CDRH1, SEQ ID NO: 38 for CDRH2, and SEQ ID NO: 39 for CDRH3, (iv) SEQ ID NO: 55 for CDRH1, SEQ ID NO: 56 for CDRH2, and SEQ ID NO: 57 for CDRH3; (v) SEQ ID NO: 75 for CDRH1, SEQ ID NO: 76 for CDRH2, and SEQ ID NO: 77 for CDRH3; (vi) SEQ ID NO: 93 for CDRH1, SEQ ID NO: 94 for CDRH2, and SEQ ID NO: 95 for CDRH3; (vii) SEQ ID NO: 111 for CDRH1, SEQ ID NO: 112 for CDRH2, and SEQ ID NO: 113 for CDRH3; (viii) SEQ ID NO: 129 for CDRH1, SEQ ID NO: 130 for CDRH2, and SEQ ID NO: 131 for CDRH3; (ix) SEQ ID NO: 147 for CDRH1, SEQ ID NO: 148 for CDRH2, and SEQ ID NO: 149 for CDRH3; (x) SEQ ID NO: 165 for CDRH1, SEQ ID NO: 166 for CDRH2, and SEQ ID NO: 167 for CDRH3; (xi) SEQ ID NO: 183 for CDRH1, SEQ ID NO: 184 for CDRH2, and SEQ ID NO: 185 for CDRH3; or (xii) SEQ ID NO: 201 for CDRH1, SEQ ID NO: 202 for CDRH2, and SEQ ID NO: 203 for CDRH3.

In some embodiments, the antibody or antigen binding fragment comprises a light chain CDR1 with the amino acid sequence of SEQ ID NOs: 4, 22, 40, 58, 78, 96, 114, 132, 150, 168, 186, or 204 or sequence variants thereof; a light chain CDR2 with the amino acid sequence of SEQ ID NOs: 5, 6, 23, 24, 41, 42, 59, 60, 79, 80, 97, 98, 115, 116, 133, 134, 151, 152, 169, 170, 187, 188, 205, or 206 or sequence variants thereof; and a light chain CDR3 with the amino acid sequence of SEQ ID NO: 7, 25, 43, 61, 81, 99, 117, 135, 153, 171, 189, or 201 or sequence variants thereof. In certain embodiments, an antibody or antibody fragment as provided herein comprises a light chain comprising the amino acid sequence of (i) SEQ ID NO: 4 for CDRL1, SEQ ID NO: 5 or 6 for CDRL2, and SEQ ID NO: 7 for CDRL3; (ii) SEQ ID NO: 22 for CDRL1, SEQ ID NO: 23 or 24 for CDRL2, and SEQ ID NO: 25 for CDRL3; (iii) SEQ ID NO: 40 for CDRL1, SEQ ID NO: 41 or 42 for CDRL2, and SEQ ID NO: 43 for CDRL3; (iv) SEQ ID NO: 58 for CDRL1, SEQ ID NO: 59 or 60 for CDRL2, and SEQ ID NO: 61 for CDRL3; (v) SEQ ID NO: 78 for CDRL1, SEQ ID NO: 79 or 80 for CDRL2, and SEQ ID NO: 81 for CDRL3; (vi) SEQ ID NO: 96 for CDRL1, SEQ ID NO: 97 or 98 for CDRL2, and SEQ ID NO: 99 for CDRL3; (vii) SEQ ID NO: 114 for CDRL1, SEQ ID NO: 115 or 116 for CDRL2, and SEQ ID NO: 117 for CDRL3; (viii) SEQ ID NO: 132 for CDRL1, SEQ ID NO: 133 or 134 for CDRL2, and SEQ ID NO: 135 for CDRL3; (ix) SEQ ID NO: 150 for CDRL1, SEQ ID NO: 151 or 152 for CDRL2, and SEQ ID NO: 152 for CDRL3; (x) SEQ ID NO: 168 for CDRL1, SEQ ID NO: 169 or 170 for CDRL2, and SEQ ID NO: 171 for CDRL3; (xi) SEQ ID NO: 186 for CDRL1, SEQ ID NO: 187 or 188 for CDRL2, and SEQ ID NO: 189 for CDRL3; or (xii) SEQ ID NO: 204 for CDRL1, SEQ ID NO: 205 or 206 for CDRL2, and SEQ ID NO: 207 for CDRL3.

For example, the antibody, or antigen binding fragment thereof, comprises: (i) heavy chain CDRH1, CDRH2, and CDRH3 amino acid sequences and light chain CDRL1, CDRL2, and CDRL3 amino acid sequences as set forth in SEQ ID NOs: 1-5 and 7 or in SEQ ID NOs: 1-4 and 6-7, respectively; (ii) heavy chain CDRH1, CDRH2, and CDRH3 amino acid sequences and light chain CDRL1, CDRL2, and CDRL3 amino acid sequences as set forth in SEQ ID NOs: 19-23 and 25 or in SEQ ID NOs: 19-22 and 24-25, respectively; (iii) heavy chain CDRH1, CDRH2, and CDRH3 amino acid sequences and light chain CDRL1, CDRL2, and CDRL3 amino acid sequences as set forth in SEQ ID NOs: 37-41 and 43 or in SEQ ID NOs: 37-40 and 42-43, respectively; (iv) heavy chain CDRH1, CDRH2, and CDRH3 amino acid sequences and light chain CDRL1, CDRL2, and CDRL3 amino acid sequences as set forth in SEQ ID NOs: 55-59 and 61 or in SEQ ID NOs: 55-58 and 60-61, respectively; (v) heavy chain CDRH1, CDRH2, and CDRH3 amino acid sequences and light chain CDRL1, CDRL2, and CDRL3 amino acid sequences as set forth in SEQ ID NOs: 75-79 and 81 or in SEQ ID NOs: 75-78 and 80-81, respectively; (vi) heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, respectively; (vii) heavy chain CDRH1, CDRH2, and CDRH3 amino acid sequences and light chain CDRL1, CDRL2, and CDRL3 amino acid sequences as set forth in SEQ ID NOs: 111-115 and 117 or in SEQ ID NOs: 111-114 and 116-117, respectively; (viii) heavy chain CDRH1, CDRH2, and CDRH3 amino acid sequences and light chain CDRL1, CDRL2, and CDRL3 amino acid sequences as set forth in SEQ ID NOs: 129-133 and 135 or in SEQ ID NOs: 129-132 and 134-135, respectively; (ix) heavy chain CDRH1, CDRH2, and CDRH3 amino acid sequences and light chain CDRL1, CDRL2, and CDRL3 amino acid sequences as set forth in SEQ ID NOs: 147-151 and 153 or in SEQ ID NOs: 147-150 and 152-153, respectively; (x) heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, respectively; (xi) heavy chain CDRH1, CDRH2, and CDRH3 amino acid sequences and light chain CDRL1, CDRL2, and CDRL3 amino acid sequences as set forth in SEQ ID NOs: 183-187 and 189 or in SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences as set forth in SEQ ID NOs: 201-205 and 207 or in SEQ ID NOs: 201-204 and 206-207, respectively.

In some embodiments, the antibody, or the antigen binding fragment thereof, is according to gRVA122, gRVA144, gRVB185, gRVB492, gRVC3, gRVC20, gRVC21, gRVC38, gRVC44, gRVC58, gRVC68, or gRVC111, in one embodiment it is according to gRVC20 or gRVC58. In one embodiment, the antibody, or the antigen binding fragment thereof, is RVA122, RVA144, RVB185, RVB492, RVC3, RVC20, RVC21, RVC38, RVC44, RVC58, RVC68, or RVC111, in certain aspects is RVC20 or RVC58.

Monoclonal antibodies (mAbs) RVA122, RVA144, RVB185, RVB492, RVC3, RVC20, RVC21, RVC38, RVC44, RVC58, RVC68, and RVC111 are described in WO 2016/078761 (e.g., WO 2016/078761, Examples 1 to 4). Based on the antibodies RVA122, RVA144, RVB185, RVB492, RVC3, RVC20, RVC21, RVC38, RVC44, RVC58, RVC68, and RVC111, in particular on the VH and VL genes of RVA122, RVA144, RVB185, RVB492, RVC3, RVC20, RVC21, RVC38, RVC44, RVC58, RVC68, and RVC111, the terms gRVA122, gRVA144, gRVB185, gRVB492, gRVC3, gRVC20, gRVC21, gRVC38, gRVC44, gRVC58, gRVC68, or gRVC111, as used herein, refer to respective “generic” antibodies, or antigen binding fragments thereof, having the specific amino acid sequences, encoded by the specific nucleotide sequences, as outlined below.

As used herein, “gRVA122” refers to an antibody, or antigen binding fragment thereof, having a CDRH1 amino acid sequence according to SEQ ID NO: 1, which is encoded by a CDRH1 nucleotide sequence according to SEQ ID NO: 8, a CDRH2 amino acid sequence according to SEQ ID NO: 2, which is encoded by a CDRH2 nucleotide sequence according to SEQ ID NO: 9, a CDRH3 amino acid sequence according to SEQ ID NO: 3, which is encoded by a CDRH3 nucleotide sequence according to SEQ ID NO: 10, a CDRL1 amino acid sequence according to SEQ ID NO: 4, which is encoded by a CDRL1 nucleotide sequence according to SEQ ID NO: 11, a CDRL2 amino acid sequence according to SEQ ID NO: 5 or 6, which is encoded by a CDRL2 nucleotide sequence according to SEQ ID NO: 12 or 13, and a CDRL3 amino acid sequence according to SEQ ID NO: 7, which is encoded by a CDRL3 nucleotide sequence according to SEQ ID NO: 14. The heavy chain variable region (V_{H}) of “gRVA122” has an amino acid sequence according to SEQ ID NO: 15, which is encoded by a nucleotide sequence according to SEQ ID NO: 17, and the light chain variable region (V_{L}) of “gRVA122” has an amino acid sequence according to SEQ ID NO: 16, which is encoded by a nucleotide sequence according to SEQ ID NO: 18.

As used herein, “gRVA144” refers to an antibody, or antigen binding fragment thereof, having a CDRH1 amino acid sequence according to SEQ ID NO: 19, which is encoded by a CDRH1 nucleotide sequence according to SEQ ID NO: 26, a CDRH2 amino acid sequence according to SEQ ID NO: 20, which is encoded by a CDRH2 nucleotide sequence according to SEQ ID NO: 27, a CDRH3 amino acid sequence according to SEQ ID NO: 21, which is encoded by a CDRH3 nucleotide sequence according to SEQ ID NO: 28, a CDRL1 amino acid sequence according to SEQ ID NO: 22, which is encoded by a CDRL1 nucleotide sequence according to SEQ ID NO: 29, a CDRL2 amino acid sequence according to SEQ ID NO: 23 or 24, which is encoded by a CDRL2 nucleotide sequence according to SEQ ID NO: 30 or 31, and a CDRL3 amino acid sequence according to SEQ ID NO: 25, which is encoded by a CDRL3 nucleotide sequence according to SEQ ID NO: 32. The heavy chain variable region (V_{H}) of “gRVA144” has an amino acid sequence according to SEQ ID NO: 33, which is encoded by a nucleotide sequence according to SEQ ID NO: 35, and the light chain variable region (V_{L}) of “gRVA144” has an amino acid sequence according to SEQ ID NO: 34, which is encoded by a nucleotide sequence according to SEQ ID NO: 36.

As used herein, “gRVB185” refers to an antibody, or antigen binding fragment thereof, having a CDRH1 amino acid sequence according to SEQ ID NO: 37, which is encoded by a CDRH1 nucleotide sequence according to SEQ ID NO: 44, a CDRH2 amino acid sequence according to SEQ ID NO: 38, which is encoded by a CDRH2 nucleotide sequence according to SEQ ID NO: 45, a CDRH3 amino acid sequence according to SEQ ID NO: 39, which is encoded by a CDRH3 nucleotide sequence according to SEQ ID NO: 46, a CDRL1 amino acid sequence according to SEQ ID NO: 40, which is encoded by a CDRL1 nucleotide sequence according to SEQ ID NO: 47, a CDRL2 amino acid sequence according to SEQ ID NO: 41 or 42, which is encoded by a CDRL2 nucleotide sequence according to SEQ ID NO: 48 or 49, and a CDRL3 amino acid sequence according to SEQ ID NO: 43, which is encoded by a CDRL3 nucleotide sequence according to SEQ ID NO: 50. The heavy chain variable region (V_{H}) of “gRVB185” has an amino acid sequence according to SEQ ID NO: 51, which is encoded by a nucleotide sequence according to SEQ ID NO: 53, and the light chain variable region (V_{L}) of “gRVB185” has an amino acid sequence according to SEQ ID NO: 52, which is encoded by a nucleotide sequence according to SEQ ID NO: 54.

As used herein, “gRVB492” refers to an antibody, or antigen binding fragment thereof, having a CDRH1 amino acid sequence according to SEQ ID NO: 55, which is encoded by a CDRH1 nucleotide sequence according to SEQ ID NO: 62, a CDRH2 amino acid sequence according to SEQ ID NO: 56, which is encoded by a CDRH2 nucleotide sequence according to SEQ ID NO: 63, a CDRH3 amino acid sequence according to SEQ ID NO: 57, which is encoded by a CDRH3 nucleotide sequence according to SEQ ID NO: 64, a CDRL1 amino acid sequence according to SEQ ID NO: 58, which is encoded by a CDRL1 nucleotide sequence according to SEQ ID NO: 65, a CDRL2 amino acid sequence according to SEQ ID NO: 59 or 60, which is encoded by a CDRL2 nucleotide sequence according to SEQ ID NO: 66 or 67, and a CDRL3 amino acid sequence according to SEQ ID NO: 61, which is encoded by a CDRL3 nucleotide sequence according to SEQ ID NO: 68. The heavy chain variable region (V_{H}) of “gRVB492” has an amino acid sequence according to SEQ ID NO: 69 or 70, which is encoded by a nucleotide sequence according to SEQ ID NO: 72 or 73, and the light chain variable region (V_{L}) of “gRVB492” has an amino acid sequence according to SEQ ID NO: 71, which is encoded by a nucleotide sequence according to SEQ ID NO: 74.

As used herein, “gRVC3” refers to an antibody, or antigen binding fragment thereof, having a CDRH1 amino acid sequence according to SEQ ID NO: 75, which is encoded by a CDRH1 nucleotide sequence according to SEQ ID NO: 82, a CDRH2 amino acid sequence according to SEQ ID NO: 76, which is encoded by a CDRH2 nucleotide sequence according to SEQ ID NO: 83, a CDRH3 amino acid sequence according to SEQ ID NO: 77, which is encoded by a CDRH3 nucleotide sequence according to SEQ ID NO: 84, a CDRL1 amino acid sequence according to SEQ ID NO: 78, which is encoded by a CDRL1 nucleotide sequence according to SEQ ID NO: 85, a CDRL2 amino acid sequence according to SEQ ID NO: 79 or 80, which is encoded by a CDRL2 nucleotide sequence according to SEQ ID NO: 86 or 87, and a CDRL3 amino acid sequence according to SEQ ID NO: 81, which is encoded by a CDRL3 nucleotide sequence according to SEQ ID NO: 88. The heavy chain variable region (V_{H}) of “gRVC3” has an amino acid sequence according to SEQ ID NO: 89, which is encoded by a nucleotide sequence according to SEQ ID NO: 91, and the light chain variable region (V_{L}) of “gRVC3” has an amino acid sequence according to SEQ ID NO: 90, which is encoded by a nucleotide sequence according to SEQ ID NO: 92.

As used herein, “gRVC20” refers to an antibody, or antigen binding fragment thereof, having a CDRH1 amino acid sequence according to SEQ ID NO: 93, which is encoded by a CDRH1 nucleotide sequence according to SEQ ID NO: 100, a CDRH2 amino acid sequence according to SEQ ID NO: 94, which is encoded by a CDRH2 nucleotide sequence according to SEQ ID NO: 101, a CDRH3 amino acid sequence according to SEQ ID NO: 95, which is encoded by a CDRH3 nucleotide sequence according to SEQ ID NO: 102, a CDRL1 amino acid sequence according to SEQ ID NO: 96, which is encoded by a CDRL1 nucleotide sequence according to SEQ ID NO: 103, a CDRL2 amino acid sequence according to SEQ ID NO: 97 or 98, which is encoded by a CDRL2 nucleotide sequence according to SEQ ID NO: 104 or 105, and a CDRL3 amino acid sequence according to SEQ ID NO: 99, which is encoded by a CDRL3 nucleotide sequence according to SEQ ID NO: 106. The heavy chain variable region (V_{H}) of “gRVC20” has an amino acid sequence according to SEQ ID NO: 107, which is encoded by a nucleotide sequence according to SEQ ID NO: 109, and the light chain variable region (V_{L}) of “gRVC20” has an amino acid sequence according to SEQ ID NO: 108, which is encoded by a nucleotide sequence according to SEQ ID NO: 110.

As used herein, “gRVC21” refers to an antibody, or antigen binding fragment thereof, having a CDRH1 amino acid sequence according to SEQ ID NO: 111, which is encoded by a CDRH1 nucleotide sequence according to SEQ ID NO: 118, a CDRH2 amino acid sequence according to SEQ ID NO: 112, which is encoded by a CDRH2 nucleotide sequence according to SEQ ID NO: 119, a CDRH3 amino acid sequence according to SEQ ID NO: 113, which is encoded by a CDRH3 nucleotide sequence according to SEQ ID NO: 120, a CDRL1 amino acid sequence according to SEQ ID NO: 114, which is encoded by a CDRL1 nucleotide sequence according to SEQ ID NO: 121, a CDRL2 amino acid sequence according to SEQ ID NO: 115 or 116, which is encoded by a CDRL2 nucleotide sequence according to SEQ ID NO: 122 or 123, and a CDRL3 amino acid sequence according to SEQ ID NO: 117, which is encoded by a CDRL3 nucleotide sequence according to SEQ ID NO: 124. The heavy chain variable region (V_{H}) of “gRVC21” has an amino acid sequence according to SEQ ID NO: 125, which is encoded by a nucleotide sequence according to SEQ ID NO: 127, and the light chain variable region (V_{L}) of “gRVC21” has an amino acid sequence according to SEQ ID NO: 126, which is encoded by a nucleotide sequence according to SEQ ID NO: 128.

As used herein, “gRVC38” refers to an antibody, or antigen binding fragment thereof, having a CDRH1 amino acid sequence according to SEQ ID NO: 129, which is encoded by a CDRH1 nucleotide sequence according to SEQ ID NO: 136, a CDRH2 amino acid sequence according to SEQ ID NO: 130, which is encoded by a CDRH2 nucleotide sequence according to SEQ ID NO: 137, a CDRH3 amino acid sequence according to SEQ ID NO: 131, which is encoded by a CDRH3 nucleotide sequence according to SEQ ID NO: 138, a CDRL1 amino acid sequence according to SEQ ID NO: 132, which is encoded by a CDRL1 nucleotide sequence according to SEQ ID NO: 139, a CDRL2 amino acid sequence according to SEQ ID NO: 133 or 134, which is encoded by a CDRL2 nucleotide sequence according to SEQ ID NO: 140 or 141, and a CDRL3 amino acid sequence according to SEQ ID NO: 135, which is encoded by a CDRL3 nucleotide sequence according to SEQ ID NO: 142. The heavy chain variable region (V_{H}) of “gRVC38” has an amino acid sequence according to SEQ ID NO: 143, which is encoded by a nucleotide sequence according to SEQ ID NO: 145, and the light chain variable region (V_{L}) of “gRVC38” has an amino acid sequence according to SEQ ID NO: 144, which is encoded by a nucleotide sequence according to SEQ ID NO: 146.

As used herein, “gRVC44” refers to an antibody, or antigen binding fragment thereof, having a CDRH1 amino acid sequence according to SEQ ID NO: 147, which is encoded by a CDRH1 nucleotide sequence according to SEQ ID NO: 154, a CDRH2 amino acid sequence according to SEQ ID NO: 148, which is encoded by a CDRH2 nucleotide sequence according to SEQ ID NO: 155, a CDRH3 amino acid sequence according to SEQ ID NO: 149, which is encoded by a CDRH3 nucleotide sequence according to SEQ ID NO: 156, a CDRL1 amino acid sequence according to SEQ ID NO: 150, which is encoded by a CDRL1 nucleotide sequence according to SEQ ID NO: 157, a CDRL2 amino acid sequence according to SEQ ID NO: 151 or 152, which is encoded by a CDRL2 nucleotide sequence according to SEQ ID NO: 158 or 159, and a CDRL3 amino acid sequence according to SEQ ID NO: 153, which is encoded by a CDRL3 nucleotide sequence according to SEQ ID NO: 160. The heavy chain variable region (V_{H}) of “gRVC44” has an amino acid sequence according to SEQ ID NO: 161, which is encoded by a nucleotide sequence according to SEQ ID NO: 163, and the light chain variable region (V_{L}) of “gRVC44” has an amino acid sequence according to SEQ ID NO: 162, which is encoded by a nucleotide sequence according to SEQ ID NO: 164.

As used herein, “gRVC58” refers to an antibody, or antigen binding fragment thereof, having a CDRH1 amino acid sequence according to SEQ ID NO: 165, which is encoded by a CDRH1 nucleotide sequence according to SEQ ID NO: 172, a CDRH2 amino acid sequence according to SEQ ID NO: 166, which is encoded by a CDRH2 nucleotide sequence according to SEQ ID NO: 173, a CDRH3 amino acid sequence according to SEQ ID NO: 167, which is encoded by a CDRH3 nucleotide sequence according to SEQ ID NO: 174, a CDRL1 amino acid sequence according to SEQ ID NO: 168, which is encoded by a CDRL1 nucleotide sequence according to SEQ ID NO: 175, a CDRL2 amino acid sequence according to SEQ ID NO: 169 or 170, which is encoded by a CDRL2 nucleotide sequence according to SEQ ID NO: 176 or 177, and a CDRL3 amino acid sequence according to SEQ ID NO: 171, which is encoded by a CDRL3 nucleotide sequence according to SEQ ID NO: 178. The heavy chain variable region (V_{H}) of “gRVC58” has an amino acid sequence according to SEQ ID NO: 179, which is encoded by a nucleotide sequence according to SEQ ID NO: 181, and the light chain variable region (V_{L}) of “gRVC58” has an amino acid sequence according to SEQ ID NO: 180, which is encoded by a nucleotide sequence according to SEQ ID NO: 182.

As used herein, “gRVC68” refers to an antibody, or antigen binding fragment thereof, having a CDRH1 amino acid sequence according to SEQ ID NO: 183, which is encoded by a CDRH1 nucleotide sequence according to SEQ ID NO: 190, a CDRH2 amino acid sequence according to SEQ ID NO: 184, which is encoded by a CDRH2 nucleotide sequence according to SEQ ID NO: 191, a CDRH3 amino acid sequence according to SEQ ID NO: 185, which is encoded by a CDRH3 nucleotide sequence according to SEQ ID NO: 192, a CDRL1 amino acid sequence according to SEQ ID NO: 186, which is encoded by a CDRL1 nucleotide sequence according to SEQ ID NO: 193, a CDRL2 amino acid sequence according to SEQ ID NO: 187 or 188, which is encoded by a CDRL2 nucleotide sequence according to SEQ ID NO: 194 or 195, and a CDRL3 amino acid sequence according to SEQ ID NO: 189, which is encoded by a CDRL3 nucleotide sequence according to SEQ ID NO: 196. The heavy chain variable region (V_{H}) of “gRVC68” has an amino acid sequence according to SEQ ID NO: 197, which is encoded by a nucleotide sequence according to SEQ ID NO: 199, and the light chain variable region (V_{L}) of “gRVC68” has an amino acid sequence according to SEQ ID NO: 198, which is encoded by a nucleotide sequence according to SEQ ID NO: 200.

As used herein, “gRVC111” refers to an antibody, or antigen binding fragment thereof, having a CDRH1 amino acid sequence according to SEQ ID NO: 201, which is encoded by a CDRH1 nucleotide sequence according to SEQ ID NO: 208, a CDRH2 amino acid sequence according to SEQ ID NO: 202, which is encoded by a CDRH2 nucleotide sequence according to SEQ ID NO: 209, a CDRH3 amino acid sequence according to SEQ ID NO: 203, which is encoded by a CDRH3 nucleotide sequence according to SEQ ID NO: 210, a CDRL1 amino acid sequence according to SEQ ID NO: 204, which is encoded by a CDRL1 nucleotide sequence according to SEQ ID NO: 211, a CDRL2 amino acid sequence according to SEQ ID NO: 205 or 206, which is encoded by a CDRL2 nucleotide sequence according to SEQ ID NO: 212 or 213, and a CDRL3 amino acid sequence according to SEQ ID NO: 207, which is encoded by a CDRL3 nucleotide sequence according to SEQ ID NO: 214. The heavy chain variable region (V_{H}) of “gRVC111” has an amino acid sequence according to SEQ ID NO: 215, which is encoded by a nucleotide sequence according to SEQ ID NO: 217, and the light chain variable region (V_{L}) of “gRVC111” has an amino acid sequence according to SEQ ID NO: 216, which is encoded by a nucleotide sequence according to SEQ ID NO: 218.

In some embodiments, the antibody according to any one of gRVA122, gRVA144, gRVB185, gRVB492, gRVC3, gRVC20, gRVC21, gRVC38, gRVC44, gRVC58, gRVC68, or gRVC111, is of the IgG1 type.

For example, the antibody or antigen binding fragment comprises a heavy chain comprising one or more (i.e., one, two or all three) heavy chain CDRs from gRVA122, gRVA144, gRVB185, gRVB492, gRVC3, gRVC20, gRVC21, gRVC38, gRVC44, gRVC58, gRVC68, or gRVC111, such as gRVC20 or gRVC58.

For example, the antibody or antigen binding fragment comprises light chain CDRs from gRVA122, gRVA144, gRVB185, gRVB492, gRVC3, gRVC20, gRVC21, gRVC38, gRVC44, gRVC58, gRVC68, or gRVC111, such as gRVC20 or gRVC58.

For example, the antibody or antigen binding fragment comprises the six CDRs (CDRH1, CDRH2, CDRH3, CDRL1, CDRL2, and CDRL3) of antibody RVC20 as listed in Table 2 the six CDRs (CDRH1, CDRH2, CDRH3, CDRL1, CDRL2, and CDRL3) of antibody RVC58 as listed in Table 2. Alternatively, the antibody or antigen binding fragment may comprise the six CDRs (CDRH1, CDRH2, CDRH3, CDRL1, CDRL2, and CDRL3) of antibody RVA122 as listed in Table 2. Alternatively, the antibody or antigen binding fragment may comprise the six CDRs (CDRH1, CDRH2, CDRH3, CDRL1, CDRL2, and CDRL3) of antibody RVA144 as listed in Table 2. Alternatively, the antibody or antigen binding fragment may comprise the six CDRs (CDRH1, CDRH2, CDRH3, CDRL1, CDRL2, and CDRL3) of antibody RVB185 as listed in Table 2. Alternatively, the antibody or antigen binding fragment may comprise the six CDRs (CDRH1, CDRH2, CDRH3, CDRL1, CDRL2, and CDRL3) of antibody RVB492 as listed in Table 2. Alternatively, the antibody or antigen binding fragment may comprise the six CDRs (CDRH1, CDRH2, CDRH3, CDRL1, CDRL2, and CDRL3) of antibody RVC3 as listed in Table 2. Alternatively, the antibody or antigen binding fragment may comprise the six CDRs (CDRH1, CDRH2, CDRH3, CDRL1, CDRL2, and CDRL3) of antibody RVC21 as listed in Table 2. Alternatively, the antibody or antigen binding fragment may comprise the six CDRs (CDRH1, CDRH2, CDRH3, CDRL1, CDRL2, and CDRL3) of antibody RVC38 as listed in Table 2. Alternatively, the antibody or antigen binding fragment may comprise the six CDRs (CDRH1, CDRH2, CDRH3, CDRL1, CDRL2, and CDRL3) of antibody RVC44 as listed in Table 2. Alternatively, the antibody or antigen binding fragment may comprise the six CDRs (CDRH1, CDRH2, CDRH3, CDRL1, CDRL2, and CDRL3) of antibody RVC68 as listed in Table 2. Alternatively, the antibody or antigen binding fragment may comprise the six CDRs (CDRH1, CDRH2, CDRH3, CDRL1, CDRL2, and CDRL3) of antibody RVC111 as listed in Table 2.

The SEQ ID numbers for the amino acid sequence for the heavy chain variable region (V_{H}) and the light chain variable region (V_{L}) of examples of anti-lyssavirus antibodies as well as the SEQ ID numbers for the nucleic acid sequences encoding them are listed in Table 3.

TABLE 3
SEQ ID Numbers for V_{H} and V_{L} amino acid and nucleic acid
residues for examples of anti-lyssavirus antibodies.

| | V_{H} amino | V_{L} amino | V_{H} nucleic | V_{L} nucleic |
|---|---|---|---|---|
| | acid | acid | acid | acid |
| RVA122 | 15 | 16 | 17 | 18 |
| RVA144 | 33 | 34 | 35 | 36 |
| RVB185 | 51 | 52 | 53 | 54 |
| RVB492 | 69 or 70 | 71 | 72 or 73 | 74 |
| RVC3 | 89 | 90 | 91 | 92 |
| RVC20 | 107 | 108 | 109 | 110 |
| RVC21 | 125 | 126 | 127 | 128 |
| RVC38 | 143 | 144 | 145 | 146 |
| RVC44 | 161 | 162 | 163 | 164 |
| RVC58 | 179 | 180 | 181 | 182 |
| RVC68 | 197 | 198 | 199 | 200 |
| RVC111 | 215 | 216 | 217 | 218 |

In some embodiments, the antibody or antigen binding fragment comprises a heavy chain variable region having an amino acid sequence that is about 70%, 75%, 80%, 85%, 88%, 90%, 92%, 95%, 96%, 97%, 98%, 99% or 100% identical to the sequence recited in any one of SEQ ID NOs: 15, 33, 51, 69, 70, 89, 107, 125, 143, 161, 179, 197, or 215. In some embodiments the antibody or antibody fragment comprises a light chain variable region having an amino acid sequence that is about 70%, 75%, 80%, 85%, 90%, 85%, 88%, 90%, 92%, 95%, 96%, 97%, 98%, 99% or 100% identical to the sequence recited in SEQ ID NOs: 16, 34, 52, 71, 90, 108, 126, 144, 162, 180, 198, or 216.

In some embodiments, the anti-lyssavirus antibody or antigen binding fragment comprises (i) a heavy chain variable region having at least 80%, for example, 85%, 88%, 90%, 92%, 95%, 96%, 97%, 98%, 99% or 100% sequence identity to the amino acid sequence of SEQ ID NO: 15 and a light chain variable region having at least 80%, for example, 85%, 88%, 90%, 92%, 95%, 96%, 97%, 98%, 99% or 100% sequence identity to the amino acid sequence of SEQ ID NO: 16; or (ii) a heavy chain variable region having at least 80%, for example, 85%, 88%, 90%, 92%, 95%, 96%, 97%, 98%, 99% or 100% sequence identity to the amino acid sequence of SEQ ID NO: 33 and a light chain variable region having at least 80%, for example, 85%, 88%, 90%, 92%, 95%, 96%, 97%, 98%, 99% or 100% sequence identity to the amino acid sequence of SEQ ID NO: 34; (iii) or a heavy chain variable region having at least 80%, for example, 85%, 88%, 90%, 92%, 95%, 96%, 97%, 98%, 99% or 100% sequence identity to the amino acid sequence of SEQ ID NO: 51 and a light chain variable region having at least 80%, for example, 85%, 88%, 90%, 92%, 95%, 96%, 97%, 98%, 99% or 100% sequence identity to the amino acid sequence of SEQ ID NO: 52; or (iv) a heavy chain variable region having at least 80%, for example, 85%, 88%, 90%, 92%, 95%, 96%, 97%, 98%, 99% or 100% sequence identity to the amino acid sequence of SEQ ID NO: 69 and a light chain variable region having at least 80%, for example, 85%, 88%, 90%, 92%, 95%, 96%, 97%, 98%, 99% or 100% sequence identity to the amino acid sequence of SEQ ID NO: 71; or (v) a heavy chain variable region having at least 80%, for example, 85%, 88%, 90%, 92%, 95%, 96%, 97%, 98%, 99% or 100% sequence identity to the amino acid sequence of SEQ ID NO: 70 and a light chain variable region having at least 80%, for example, 85%, 88%, 90%, 92%, 95%, 96%, 97%, 98%, 99% or 100% sequence identity to the amino acid sequence of SEQ ID NO: 71; or (vi) a heavy chain variable region having at least 80%, for example, 85%, 88%, 90%, 92%, 95%, 96%, 97%, 98%, 99% or 100% sequence identity to the amino acid sequence of SEQ ID NO: 89 and a light chain variable region having at least 80%, for example, 85%, 88%, 90%, 92%, 95%, 96%, 97%, 98%, 99% or 100% sequence identity to the amino acid sequence of SEQ ID NO: 90; or (vii) a heavy chain variable region having at least 80%, for example, 85%, 88%, 90%, 92%, 95%, 96%, 97%, 98%, 99% or 100% sequence identity to the amino acid sequence of SEQ ID NO: 107 and a light chain variable region having at least 80%, for example, 85%, 88%, 90%, 92%, 95%, 96%, 97%, 98%, 99% or 100% sequence identity to the amino acid sequence of SEQ ID NO: 108; or (viii) a heavy chain variable region having at least 80%, for example, 85%, 88%, 90%, 92%, 95%, 96%, 97%, 98%, 99% or 100% sequence identity to the amino acid sequence of SEQ ID NO: 125 and a light chain variable region having at least 80%, for example, 85%, 88%, 90%, 92%, 95%, 96%, 97%, 98%, 99% or 100% sequence identity to the amino acid sequence of SEQ ID NO: 126; or (ix) a heavy chain variable region having at least 80%, for example, 85%, 88%, 90%, 92%, 95%, 96%, 97%, 98%, 99% or 100% sequence identity to the amino acid sequence of SEQ ID NO: 143 and a light chain variable region having at least 80%, for example, 85%, 88%, 90%, 92%, 95%, 96%, 97%, 98%, 99% or 100% sequence identity to the amino acid sequence of SEQ ID NO: 144; or (x) a heavy chain variable region having at least 80%, for example, 85%, 88%, 90%, 92%, 95%, 96%, 97%, 98%, 99% or 100% sequence identity to the amino acid sequence of SEQ ID NO: 161 and a light chain variable region having at least 80%, for example, 85%, 88%, 90%, 92%, 95%, 96%, 97%, 98%, 99% or 100% sequence identity to the amino acid sequence of SEQ ID NO: 162; or (xi) a heavy chain variable region having at least 80%, for example, 85%, 88%, 90%, 92%, 95%, 96%, 97%, 98%, 99% or 100% sequence identity to the amino acid sequence of SEQ ID NO: 179 and a light chain variable region having at least 80%, for example, 85%, 88%, 90%, 92%, 95%, 96%, 97%, 98%, 99% or 100% sequence identity to the amino acid sequence of SEQ ID NO: 180; or (xii) a heavy chain variable region having at least 80%, for example, 85%, 88%, 90%, 92%, 95%, 96%, 97%, 98%, 99% or 100% sequence identity to the amino acid sequence of SEQ ID NO: 197 and a light chain variable region having at least 80%, for example, 85%, 88%, 90%, 92%, 95%, 96%, 97%, 98%, 99% or 100% sequence identity to the amino acid sequence of SEQ ID NO: 198; or (xiii) a heavy chain variable region having at least 80%, for example, 85%, 88%, 90%, 92%, 95%, 96%, 97%, 98%, 99% or 100% sequence identity to the amino acid sequence of SEQ ID NO: 215 and a light chain variable region having at least 80%, for example, 85%, 88%, 90%, 92%, 95%, 96%, 97%, 98%, 99% or 100% sequence identity to the amino acid sequence of SEQ ID NO: 216.

For example, the anti-lyssavirus antibody, or the antigen binding fragment thereof, comprises: (i) a heavy chain variable region comprising the amino acid sequence of SEQ ID NO: 15 and a light chain variable region comprising the amino acid sequence of SEQ ID NO: 16; or (ii) a heavy chain variable region comprising the amino acid sequence of SEQ ID NO: 33 and a light chain variable region comprising the amino acid sequence of SEQ ID NO: 34; or (iii) a heavy chain variable region comprising the amino acid sequence of SEQ ID NO: 51 and a light chain variable region comprising the amino acid sequence of SEQ ID NO: 52; or (iv) a heavy chain variable region comprising the amino acid sequence of SEQ ID NO: 69 and a light chain variable region comprising the amino acid sequence of SEQ ID NO: 71; or (v) a heavy chain variable region comprising the amino acid sequence of SEQ ID NO: 70 and a light chain variable region comprising the amino acid sequence of SEQ ID NO: 71; or (vi) a heavy chain variable region comprising the amino acid sequence of SEQ ID NO: 89 and a light chain variable region comprising the amino acid sequence of SEQ ID NO: 90; or (vii) a heavy chain variable region comprising the amino acid sequence of SEQ ID NO: 107 and a light chain variable region comprising the amino acid sequence of SEQ ID NO: 108; or (viii) a heavy chain variable region comprising the amino acid sequence of SEQ ID NO: 125 and a light chain variable region comprising the amino acid sequence of SEQ ID NO: 126; or (ix) a heavy chain variable region comprising the amino acid sequence of SEQ ID NO: 143 and a light chain variable region comprising the amino acid sequence of SEQ ID NO: 144; or (x) a heavy chain variable region comprising the amino acid sequence of SEQ ID NO: 161 and a light chain variable region comprising the amino acid sequence of SEQ ID NO: 162; or (xi) a heavy chain variable region comprising the amino acid sequence of SEQ ID NO: 179 and a light chain variable region comprising the amino acid sequence of SEQ ID NO: 180; or (xii) a heavy chain variable region comprising the amino acid sequence of SEQ ID NO: 197 and a light chain variable region comprising the amino acid sequence of SEQ ID NO: 198; or (xiii) a heavy chain variable region comprising the amino acid sequence of SEQ ID NO: 215 and a light chain variable region comprising the amino acid sequence of SEQ ID NO: 216.

For example, the anti-lyssavirus antibody, or the antigen-binding fragment thereof, comprises a heavy chain variable region having an amino acid sequence according to SEQ ID NO: 107 and a light chain variable region having an amino acid sequence according to SEQ ID NO: 108, or sequence variants thereof, such as a heavy chain variable region having at least 80% or at least 90% sequence identity to the amino acid sequence of SEQ ID NO: 107 and a light chain variable region having at least 80% or at least 90% sequence identity to the amino acid sequence of SEQ ID NO: 108. For instance, the anti-lyssavirus antibody, or the antigen-binding fragment thereof, comprises a heavy chain variable region having an amino acid sequence as set forth in SEQ ID NO: 107 and a light chain variable region having an amino acid sequence as set forth in SEQ ID NO: 108.

For example, the anti-lyssavirus antibody, or the antigen-binding fragment thereof, comprises a heavy chain variable region having an amino acid sequence according to SEQ ID NO: 179 and a light chain variable region having an amino acid sequence according to SEQ ID NO: 180, or sequence variants thereof, such as a heavy chain variable region having at least 80% or at least 90% sequence identity to the amino acid sequence of SEQ ID NO: 179 and a light chain variable region having at least 80% or at least 90% sequence identity to the amino acid sequence of SEQ ID NO: 180. For instance, the anti-lyssavirus antibody, or the antigen-binding fragment thereof, comprises a heavy chain variable region having an amino acid sequence as set forth in SEQ ID NO: 179 and a light chain variable region having an amino acid sequence as set forth in SEQ ID NO: 180.

Examples of antibodies include, but are not limited to, RVA122, RVA144, RVB185, RVB492, RVC3, RVC20, RVC21, RVC38, RVC44, RVC58, RVC68, or RVC111, such as antibodies RVC20 and RVC58.

Two anti-Lyssavirus antibodies as described above (e.g., having the CDRs as set forth in Table 2 or the variable regions as set forth in Table 3, or functional sequence variants thereof) may be administered in combination as described herein.

For example, one of the two anti-lyssavirus antibodies administered in combination as described herein comprises heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof, such as amino acid sequences that are at least 80% or at least 90% identical to the amino acid sequences of SEQ ID NOs: 93-97 and 99 or to the amino acid sequences of SEQ ID NOs: 93-96 and 98-99, respectively; and the other of the two anti-lyssavirus antibodies administered in combination as described herein comprises heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, or sequence variants thereof, such as amino acid sequences that are at least 80 or at least 90% identical to the amino acid sequences of SEQ ID NOs: 165-169 and 171 or to the amino acid sequences of SEQ ID NOs: 165-168 and 170-171, respectively. In particular, one of the two anti-lyssavirus antibodies administered in combination as described herein comprises heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, respectively; and the other of the two anti-lyssavirus antibodies administered in combination as described herein comprises heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, respectively.

For example, one of the two anti-lyssavirus antibodies administered in combination as described herein comprises a heavy chain variable region having an amino acid sequence according to SEQ ID NO: 107 and a light chain variable region having an amino acid sequence according to SEQ ID NO: 108, or sequence variants thereof, such as a heavy chain variable region having at least 80% or at least 90% sequence identity to the amino acid sequence of SEQ ID NO: 107 and a light chain variable region having at least 80% or at least 90% sequence identity to the amino acid sequence of SEQ ID NO: 108; and the other of the two anti-lyssavirus antibodies administered in combination as described herein comprises a heavy chain variable region having an amino acid sequence according to SEQ ID NO: 179 and a light chain variable region having an amino acid sequence according to SEQ ID NO: 180, or sequence variants thereof, such as a heavy chain variable region having at least 80% or at least 90% sequence identity to the amino acid sequence of SEQ ID NO: 179 and a light chain variable region having at least 80% or at least 90% sequence identity to the amino acid sequence of SEQ ID NO: 180. For example, one of the two anti-lyssavirus antibodies administered in combination as described herein comprises a heavy chain variable region having an amino acid sequence as set forth in SEQ ID NO: 107 and a light chain variable region having an amino acid sequence as set forth in SEQ ID NO: 108; and the other of the two anti-lyssavirus antibodies administered in combination as described herein comprises a heavy chain variable region having an amino acid sequence as set forth in SEQ ID NO: 179 and a light chain variable region having an amino acid sequence as set forth in SEQ ID NO: 180.

In one embodiment, anti-lyssavirus antibodies RVC20 and RVC58 (e.g., as described herein or in WO 2016/078761) are administered in combination as described herein.

Thereby, a combination of antibodies is provided for the prophylaxis, treatment or attenuation of infection by RABV and/or non-RABV lyssaviruses, for example, RABV and/or non-RABV phylogroup I lyssaviruses, such as RABV and/or EBLV-1, with at least one broadly neutralizing antibody, which also neutralizes EBLV-1.

In particular, the anti-lyssavirus antibody, or the antigen-binding fragment thereof, for use according to the invention, is comprised in a pharmaceutical composition.

### Pharmaceutical Compositions

In a further aspect, the invention also provides a pharmaceutical composition comprising (as an active ingredient) an anti-lyssavirus antibody, or an antigen-binding fragment thereof, for use in the treatment of lyssavirus infection, wherein the pharmaceutical composition is administered

(i) into the central nervous system (CNS); and
(ii) peripherally.

Typically, the pharmaceutical composition comprises an anti-lyssavirus antibody, or an antigen-binding fragment thereof, as described above. Accordingly, embodiments of the anti-lyssavirus antibody, or the antigen-binding fragment thereof, as described above correspond to some embodiments of the anti-lyssavirus antibody, or the antigen-binding fragment thereof comprised in the pharmaceutical composition.

Usually, the pharmaceutical composition is administered as described above for the administration of the anti-lyssavirus antibody, or the antigen-binding fragment thereof. Accordingly, embodiments of the administration of the anti-lyssavirus antibody, or the antigen-binding fragment thereof, correspond to embodiments of the administration of the pharmaceutical composition comprising an anti-lyssavirus antibody, or an antigen-binding fragment thereof.

The pharmaceutical composition may in some embodiments also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In particular, the pharmaceutically acceptable carrier in the pharmaceutical composition is not an active component in respect to lyssavirus infection.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions.

The pharmaceutical composition may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin™, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for nasal or ocular administration e.g., as drops or spray, using a fine powder or a liquid. The composition may be prepared as a suppository or pessary. The composition may be in kit form, designed such that a combined composition is reconstituted just prior to administration to a subject. For example, a lyophilized antibody may be provided in kit form with sterile water or a sterile buffer. Further examples, in particular for administration into the CNS, include saline and phosphate buffered saline (PBS).

Typically, the active ingredient in the composition is the anti-lyssavirus antibody, or an antigen-binding fragment thereof. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

The pharmaceutical composition may have a pH between 5.5 and 8.5, in particular between 6 and 8, for example about 7. The pH may be maintained by the use of a buffer. The composition may be sterile and/or pyrogen free. The composition may be isotonic with respect to humans. In one embodiment pharmaceutical compositions of the invention may be supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody molecule may be in dry form, for reconstitution before use with an appropriate sterile liquid. A vehicle is typically understood to be a material that is suitable for storing, transporting, and/or administering a compound, such as a pharmaceutically active compound, in particular the anti-lyssavirus antibodies. For example, the vehicle may be a physiologically acceptable liquid, which is suitable for storing, transporting, and/or administering a pharmaceutically active compound, in particular the anti-lyssavirus antibodies. Once formulated, the pharmaceutical composition can be administered directly to the subject. In one embodiment the pharmaceutical composition is adapted for administration to mammalian, e.g., human subjects.

The pharmaceutical composition may be prepared, for example, as hypospray. For oral administration the pharmaceutical composition may be prepared e.g. as tablets or capsules. For topical administration or as injectable the pharmaceutical composition may be prepared e.g. as liquid solutions or suspensions, such as an injectable. Solid forms suitable for solution in, or suspension in, liquid vehicles prior to injection are used in some embodiments, e.g. the pharmaceutical composition is in lyophilized form.

For injection or infusion the pharmaceutical composition may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Administration of the anti-lyssavirus antibody is typically in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition may be provided for example in a pre-filled syringe.

The pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the anti-lyssavirus antibody, is combined with emulsifying and suspending agents. If desired, certain sweetening, flavoring or coloring agents may also be added.

The pharmaceutical composition may also be administered topically, especially when the target of treatment includes areas or organs readily accessible by topical application. Suitable topical formulations are readily prepared for each of these areas or organs. For topical applications, the pharmaceutical composition may be formulated in a suitable ointment, containing the pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical composition can be formulated in a suitable lotion or cream. In the context of the invention, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. In particular, the pharmaceutical composition may be provided as single-dose product. In particular, the amount of the antibody in the pharmaceutical composition in particular if provided as single-dose product does not exceed 200 mg, for example, it does not exceed 100 mg or 50 mg.

In particular, a single dose, e.g. a daily, weekly or monthly dose, such as for a weekly dose, the amount of the antibody, or the antigen binding fragment thereof, in the pharmaceutical composition according to the invention, does not exceed 1 g or 500 mg, for example, it does not exceed 200 mg or 100 mg, such as 50 mg.

Pharmaceutical compositions typically include an “effective” amount of one or more antibodies of the invention, i.e. an amount that is sufficient to treat, ameliorate, attenuate or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. In one embodiment, the pharmaceutical composition may include anti-lyssavirus antibodies, wherein the antibodies may make up at least 50% by weight (e.g., 60%, 70%, 75%, 80%, 85%, 90%, 95%, 96%, 97%, 98%, 99% or more) of the total protein in the composition. In such a composition, the antibodies are typically in purified form.

Pharmaceutical compositions may include an antimicrobial particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions of the invention may also comprise one or more immunoregulatory agents.

The pharmaceutical composition may comprise at least two anti-lyssavirus antibodies or antigen binding fragments thereof, as described above, wherein the two anti-lyssavirus antibodies, or the antigen binding fragments thereof, specifically bind to different epitopes on the glycoprotein G of RABV. For example, the pharmaceutical composition according to the invention comprises a first anti-lyssavirus antibody or an antigen binding fragment thereof, and a second anti-lyssavirus antibody, or an antigen binding fragment thereof, wherein the first antibody, or the antigen binding fragment thereof, specifically binds to another epitope on the glycoprotein G of RABV than the second antibody or the second antigen binding fragment thereof.

In other words, the two anti-lyssavirus antibodies comprised in such a pharmaceutical composition bind specifically to the RABV G protein, but to different epitopes on the RABV G protein. For example, one antibody may specifically bind to antigenic site I on the RABV G protein and another antibody may specifically bind to antigenic site III or to an epitope on the glycoprotein G of RABV, which at least partially overlaps with antigenic site Ill on the glycoprotein G of RABV.

In particular, one antibody of the at least two anti-lyssavirus antibodies comprised in such a pharmaceutical composition binds (specifically) to antigenic site I on the glycoprotein G of RABV and another anti-lyssavirus antibody of the at least two antibodies comprised in such a pharmaceutical composition binds (specifically) to antigenic site III on the glycoprotein G of RABV.

For example, one of the at least two anti-lyssavirus antibodies or antigen binding fragments thereof comprised in the pharmaceutical composition comprises heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof, such as amino acid sequences, which are at least 80% or at least 90% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, or sequence variants thereof, such as amino acid sequences, which are at least 80% or at least 90% identical to the amino acid sequences of SEQ ID NOs: 165-169 and 171 or to the amino acid sequences of SEQ ID NOs: 165-168 and 170-171, respectively.

For example, the pharmaceutical composition comprises at least two anti-lyssavirus antibodies as described herein, wherein one of the at least two antibodies or antigen binding fragments thereof comprises a heavy chain variable region having an amino acid sequence as set forth in SEQ ID NO: 107 and a light chain variable region having an amino acid sequence as set forth in SEQ ID NO: 108, or sequence variants thereof, such as a heavy chain variable region having at least 80% or at least 90% sequence identity to the amino acid sequence of SEQ ID NO: 107 and a light chain variable region having at least 80% or at least 90% sequence identity to the amino acid sequence of SEQ ID NO: 108; and the other of the two anti-lyssavirus antibodies, or the antigen-binding fragments thereof, comprises a heavy chain variable region having an amino acid sequence as set forth in SEQ ID NO: 179 and a light chain variable region having an amino acid sequence as set forth in SEQ ID NO: 180, or sequence variants thereof, such as a heavy chain variable region having at least 80% or at least 90% sequence identity to the amino acid sequence of SEQ ID NO: 179 and a light chain variable region having at least 80% or at least 90% sequence identity to the amino acid sequence of SEQ ID NO: 180.

For example, the pharmaceutical composition comprises anti-lyssavirus antibodies RVC20 and RVC58, such as a cocktail of anti-lyssavirus antibodies RVC20 and RVC58.

Moreover, the pharmaceutical composition may also contain more than two, e.g. 3, 4, 5, 6, etc., anti-lyssavirus antibodies, whereby at least two or more than two, such as all antibodies contained, bind to different epitopes on the RABV G protein.

For example, the (at least) two antibodies are present in the pharmaceutical composition at equimolar amounts, such as an equimolar mixture. For example, the pharmaceutical composition comprises the antibodies RVC58 and RVC20 (or antigen binding fragments thereof), e.g., in equimolar amounts, and a pharmaceutically acceptable carrier.

The combination of two anti-lyssavirus antibodies, which bind to different epitopes of the RABV G protein, represents a treatment with an unprecedented breadth of reactivity and with reduced risk of escape mutant selection. In particular, a combination of two or more anti-lyssavirus antibodies, whereby the antibodies bind to a different epitopes or sites on the RABV G protein, increases the protective effect and prevents the escape of resistant variants of the virus.

As an alternative to delivering antibodies, it is possible to deliver nucleic acid (typically DNA or RNA) that encodes the anti-lyssavirus antibody, or an antigen-binding fragment thereof, (e.g., derived from a B cell or a cultured plasma cell) to a subject, such that the nucleic acid can be expressed in the subject in situ to provide a desired therapeutic effect. Suitable administration requirements, techniques and nucleic acid delivery vectors are known in the art. Accordingly, the invention also provides a nucleic acid molecule (e.g., DNA or RNA) comprising a polynucleotide encoding an anti-lyssavirus antibody (as described herein) or an antigen-binding fragment thereof (as described herein) for use in the treatment of lyssavirus infection (as described herein) comprising administration of the antibody or an antigen-binding fragment thereof into the CNS and peripherally (as described herein). Furthermore, the present invention also provides a combination of at least two distinct nucleic acid molecules encoding an anti-lyssavirus antibody, or an antigen-binding fragment thereof, wherein one nucleic acid molecule encodes at least a CDRH1, CDRH2 and CDRH3 of a heavy chain and another nucleic acid molecule encodes at least a CDRL1, CDRL2 and CDRL3 of a corresponding light chain of the antilyssavirus antibody, for use in the treatment of lyssavirus infection, wherein the nucleic acid molecule is administered

(i) into the central nervous system (CNS); and
(ii) peripherally.

In such a combination, for example, one nucleic acid molecule may encode at least a variable region of a heavy chain (VH) and another nucleic acid molecule may encode at least a corresponding variable region of a light chain (VL) of the antilyssavirus antibody. In some embodiments, one nucleic acid molecule encodes a heavy chain and another nucleic acid molecule encodes a corresponding light chain of the antilyssavirus antibody. In some embodiments, the encoded anti-lyssavirus antibody, or the antigen-binding fragment thereof, is as defined above. The combination may be administered as described above. In some embodiments, the nucleic acid is DNA. In some embodiments, the nucleic acid is RNA.

### Combination Therapy and Kits of Parts

The invention also provides a method of treating or attenuating a lyssavirus infection in a subject, wherein the method comprises administering to a subject in need thereof the anti-lyssavirus antibody, or the antigen binding fragment thereof, as described above or the pharmaceutical composition as described above, wherein the antibody, or the antigen-binding fragment thereof, or the pharmaceutical composition is administered

(i) into the central nervous system (CNS); and
(ii) peripherally
as described above. Embodiments of such administration is also described above.

In general, the treatment according to the invention with the anti-lyssavirus antibody, or the antigen-binding fragment thereof, or the pharmaceutical composition as described herein, may be used as stand-alone treatment or it may be combined with other known anti-lyssavirus therapies. Well-known anti-lyssavirus therapies include rabies vaccination, ribavirin (or other antivirals), interferon-alpha or ketamine. In other words, the anti-lyssavirus antibody, or the antigen-binding fragment thereof, or the pharmaceutical composition may be administered according to the invention in combination with an anti-lyssavirus vaccine, such as a rabies vaccine, an antiviral agent, such as ribavirin, interferon-alpha and/or ketamine.

Currently available rabies vaccines include the most widely used but highly risk-prone nerve tissue vaccines, or the safer but more costly cell culture and embryonated egg vaccines (CCEEVs). In Germany e.g. only two anti-rabies vaccines are on the market, Rabipur® and “Tollwut-Impfstoff (human diploid cell [HDC]) inaktiviert”. These vaccines contain inactivated rabies virus. Both vaccines are recommended for pre- and postexposure use. Another example of a rabies vaccine is Imovax (Sanofi-Pasteur), which is a commercial inactivated human diploid cell vaccine. Rabies vaccines are in general administered according to the information of the manufacturer. A typical post-exposure prophylaxis protocol includes, for example, administration of the vaccine at days 0, 3, 7, 14 and 28 after infection.

An antiviral refers to a class of medication used specifically for treating viral infections. Like antibiotics for bacteria, specific antivirals are used for specific viruses. Unlike most antibiotics, antiviral drugs do not destroy their target pathogen; instead they inhibit their development. For example, the antiviral is ribavirin.

However, in some embodiments, the anti-lyssavirus antibody, or the antigen-binding fragment thereof, or the pharmaceutical composition is administered without previous, concomitant and/or subsequent administration of an anti-lyssavirus vaccine.

Moreover, the anti-lyssavirus antibody, or the antigen-binding fragment thereof, or the pharmaceutical composition may be administered to those subjects who have previously shown no response, i.e., have been shown to be refractive to treatment of lyssavirus infection. Such treatment may include previous treatment with an anti-viral agent. This may be due to, for example, infection with an anti-viral resistant strain of RABV and/or non-RABV lyssaviruses.

In a further aspect, the invention provides a kit of parts comprising at least one anti-lyssavirus antibody, or antigen binding fragment thereof, as described above or at least one pharmaceutical composition as described above. In particular, the kit of parts may contain a leaflet with instructions to use, which contains details regarding the administration of the antibody, or the antigen-binding fragment thereof, as described above. The kit of parts can be used in the treatment of lyssavirus infection, in particular wherein the antibody, or the antigen-binding fragment thereof, or the pharmaceutical composition is administered as described above.

For example, such a kit of parts comprises at least two different anti-lyssavirus antibodies, or antigen binding fragments thereof, as described above. The at least two different anti-lyssavirus antibodies, or the antigen binding fragments thereof, specifically bind, for instance, to different epitopes on the glycoprotein G of RABV. Such a kit of parts is particularly useful for the combination of two antibodies as described herein. The at least two antibodies may be present in the kit of parts as separate entities or combined, e.g. as a mixture, for example if both antibodies are contained in the same pharmaceutical composition. For example, the at least two different antibodies are separate entities in the kit of parts, which may be mixed by the user if needed. For example, at least two different antibodies are combined, e.g. as a mixture, for example if both antibodies are contained in the same pharmaceutical composition, e.g. at equimolar amounts.

In particular, in such a kit of parts one antibody of the at least two antibodies binds to antigenic site I on the glycoprotein G of RABV and another antibody of the at least two antibodies binds to antigenic site III on the glycoprotein G of RABV.

For example, one of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof, such as amino acid sequences that are at least 80% or at least 90% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, comprises heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, or sequence variants thereof, such as amino acid sequences that are at least 80% or at least 90% identical to the amino acid sequences of SEQ ID NOs: 165-169 and 171 or to the amino acid sequences of SEQ ID NOs: 165-168 and 170-171, respectively.

For example, the kit of parts comprises two distinct anti-lyssavirus antibodies, wherein one of the two anti-lyssavirus antibodies, or the antigen-binding fragments thereof, comprises a heavy chain variable region having an amino acid sequence as set forth in SEQ ID NO: 107 and a light chain variable region having an amino acid sequence as set forth in SEQ ID NO: 108, or sequence variants thereof, such as amino acid sequences having at least 80% or at least 90% sequence identity to the amino acid sequence of SEQ ID NO: 107 and 108; and the other of the two anti-lyssavirus antibodies, or the antigen-binding fragments thereof, comprises a heavy chain variable region having an amino acid sequence as set forth in SEQ ID NO: 179 and a light chain variable region having an amino acid sequence as set forth in SEQ ID NO: 180, or sequence variants thereof, such as amino acid sequences having at least 80% or at least 90% sequence identity to the amino acid sequence of SEQ ID NO: 179 and 180.

### Anti-Lyssavirus Antibody Comprising a LALA Mutation in the Fc Moiety

In a further aspect, the invention also provides an anti-lyssavirus antibody, or an antigen-binding fragment thereof, comprising an Fc moiety comprising a CH2 domain and a CH2 L4A mutation and/or a CH2 L5A mutation.

Disclosed herein is that an effective treatment with anti-lyssavirus antibodies does not rely on an intact Fc region mediating antibody effector functions. This finding was unexpected, in particular since antibodies devoid of effector function were even administered at a time when most of the neuronal cells were already infected.

The CH2 L4A and CH2 L5A mutation in the CH2 domain of an Fc moiety of an antibody (for example L234A, L235A) is also often referred to as “LALA” mutation. The “LALA” mutation is known in antibodies against flaviviruses, such as dengue and zika virus, in order to prevent/inhibit antibody-dependent enhancement (ADE) in flavivirus infection (e.g., dengue and zika virus infection) (WO 2010/043977, WO 2018/011283, Beltramello M, Williams K L, Simmons C P, et al. The Human Immune Response to Dengue Virus Is Dominated by Highly Cross-Reactive Antibodies Endowed with Neutralizing and Enhancing Activity. Cell host & microbe. 2010; 8(3):10.1016/j.chom.2010.08.007. doi: 10.1016/j.chom.2010.08.007; Stettler K, Beltramello M, Espinosa D A, Graham V, Cassotta A, Bianchi S, et al. Specificity, cross-reactivity, and function of antibodies elicited by Zika virus infection. Science (2016) 353(6301):823-6.10.1126/science.aaf8505; Khandia R, Munjal A, Dhama K, et al. Modulation of Dengue/Zika Virus Pathogenicity by Antibody-Dependent Enhancement and Strategies to Protect Against Enhancement in Zika Virus Infection. Frontiers in Immunology. 2018; 9:597. doi:10.3389/fimmu.2018.00597).

As used herein, the term “Fc moiety” refers to a sequence derived from an Fc region of an immunoglobulin heavy chain or a portion thereof. Typically, the Fc region of an immunoglobulin heavy chain begins in the hinge region just upstream of the papain cleavage site (e.g., residue 216 in native IgG) and ends at the C-terminus of the immunoglobulin heavy chain. Accordingly, an Fc moiety may be a complete Fc region or a portion (e.g., a domain) thereof. A complete Fc region comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody. Each of the amino acid positions within an Fc moiety have been numbered according to the art-recognized EU numbering system of Kabat, see e.g., by Kabat et al., in “Sequences of Proteins of Immunological Interest”, U.S. Dept. Health and Human Services, 1983 and 1987.

In the context of the invention, the Fc moiety comprises at least a CH2 domain. In particular, the Fc moiety further comprises at least one of: a hinge (e.g., upper, middle, and/or lower hinge region) domain, a CH3 domain, or a variant, portion, or fragment thereof. In some embodiments, an Fc moiety further comprises at least a hinge domain or a CH3 domain, such as an Fc moiety comprising a complete Fc region.

In the context of the invention, the Fc moiety comprises a CH2 L4A mutation and/or a CH2 L5A mutation in the CH2 domain. In addition, the Fc moiety may also comprise one or more (additional) amino acid insertions, deletions, or substitutions relative to a naturally-occurring Fc moiety. For example, at least one of a hinge domain or CH3 domain (or portion thereof) may be deleted. For example, an Fc moiety may comprise or consist of: (i) hinge domain (or portion thereof) fused to a CH2 domain (or portion thereof), (ii) a CH2 domain (or portion thereof) fused to a CH3 domain (or portion thereof), or (iii) a CH2 domain (or portion thereof).

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include, for example, antibody half-life modulation, protein A binding, and protein G binding. The portions of naturally occurring Fc moieties, which are responsible and/or essential for such functions are well known by those skilled in the art.

For example, the Fc moiety is an Fc region. Accordingly, the antibody, or antigen binding fragment thereof, comprises an Fc region. As used herein, the term “Fc region” refers to the portion of an immunoglobulin heavy chain, which begins in the hinge region just upstream of the papain cleavage site (e.g., residue 216 in native IgG) and ends at the C-terminus of the immunoglobulin heavy chain.

The Fc region or Fc moiety may occur as monomer (scFc) or as dimer (dcFc). A “dimeric Fc region” or “dimeric Fc moiety” refers to the dimer formed by the Fc regions/Fc moieties of two separate immunoglobulin heavy chains. The Fc dimer may be a homodimer of two identical Fc moieties (e.g., an Fc region of a naturally occurring immunoglobulin) or a heterodimer of two non-identical Fc moieties/Fc regions.

The Fc regions or Fc moieties of an Fc dimer may be of the same or different class and/or subclass. For example, the Fc regions or Fc moieties may be derived from an immunoglobulin (e.g., a human immunoglobulin) of an IgG1, IgG2, IgG3 or IgG4 subclass. In particular, the Fc regions or Fc moieties of a dimer are of the same class and subclass. However, the Fc dimer may also be chimeric, whereby a chimeric Fc dimer may comprise Fc moieties derived from different immunoglobulin classes and/or subclasses. For example, at least two of the Fc moieties of a dimeric or single-chain Fc may be from different immunoglobulin classes and/or subclasses. Additionally or alternatively, the chimeric Fc dimers may comprise one or more chimeric Fc moieties.

For example, a chimeric Fc region or moiety may comprise one or more portions derived from an immunoglobulin of a first subclass (e.g., an IgG1, IgG2, or IgG3 subclass) while the remainder of the Fc region or moiety is of a different subclass. For example, an Fc region or moiety of an Fc polypeptide may comprise a CH2 and/or CH3 domain derived from an immunoglobulin of a first subclass (e.g., an IgG1, IgG2 or IgG4 subclass) and a hinge region from an immunoglobulin of a second subclass (e.g., an IgG3 subclass). For example, the Fc region or moiety may comprise a hinge and/or CH2 domain derived from an immunoglobulin of a first subclass (e.g., an IgG4 subclass) and a CH3 domain from an immunoglobulin of a second subclass (e.g., an IgG1, IgG2, or IgG3 subclass). For example, the chimeric Fc dimer may comprise an Fc region from an immunoglobulin for a first subclass (e.g., an IgG4 subclass) and an Fc region from an immunoglobulin of a second subclass (e.g., an IgG1, IgG2 or IgG3 subclass). For example, the Fc region or moiety may comprise a CH2 domain from an IgG4 immunoglobulin and a CH3 domain from an IgG1 immunoglobulin. For example, the Fc region or moiety may comprise a CH1 domain and a CH2 domain from an IgG4 molecule and a CH3 domain from an IgG1 molecule. For example, the Fc region or moiety may comprise a portion of a CH2 domain from a particular subclass of antibody, e.g., EU positions 292-340 of a CH2 domain. For example, an Fc region or moiety may comprise amino acids a positions 292-340 of CH2 derived from an IgG4 moiety and the remainder of CH2 derived from an IgG1 moiety (alternatively, 292-340 of CH2 may be derived from an IgG1 moiety and the remainder of CH2 derived from an IgG4 moiety).

Moreover, an Fc region or moiety may (additionally or alternatively) for example comprise a chimeric hinge region. For example, the chimeric hinge may be derived, e.g. in part, from an IgG1, IgG2, or IgG4 molecule (e.g., an upper and lower middle hinge sequence) and, in part, from an IgG3 molecule (e.g., a middle hinge sequence). In another example, an Fc region or moiety may comprise a chimeric hinge derived, in part, from an IgG1 molecule and, in part, from an IgG4 molecule. In another example, the chimeric hinge may comprise upper and lower hinge domains from an IgG4 molecule and a middle hinge domain from an IgG1 molecule. Such a chimeric hinge may be made, for example, by introducing a proline substitution (Ser228Pro) at EU position 228 in the middle hinge domain of an IgG4 hinge region. In another embodiment, the chimeric hinge can comprise amino acids at EU positions 233-236 are from an IgG2 antibody and/or the Ser228Pro mutation, wherein the remaining amino acids of the hinge are from an IgG4 antibody. Chimeric hinges, which may be used in the Fc moiety of the antibody according to the invention are described in US 2005/0163783 A1.

The Fc moiety, or the Fc region, may comprise or consist of an amino acid sequence derived from a human immunoglobulin sequence (e.g., from an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc moiety or in the Fc region. For example, the antibody according to the invention comprises a (complete) Fc region derived from human IgG1. For example, the antibody according to the invention comprises, in particular in addition to a (complete) Fc region derived from human IgG1 also all other parts of the constant regions of IgG, such as all other parts of the constant regions of IgG1 or all other parts of the constant regions of human IgG1.

Accordingly, the antibody according to the invention comprises, for example, a constant region of IgG, such as a constant region of IgG1, e.g., a constant region of human IgG1. In some embodiments, the sequences of constant regions are the amino acid sequences according to SEQ ID NOs: 219-221 (nucleic acid sequences according to SEQ ID NOs: 222-224). In particular, anti-lyssavirus antibody comprises an IgG1 CH1-CH2-CH3 with the “LALA” mutation as described above. In some embodiments, the amino acid sequence of IgG1 CH1-CH2-CH3 is according to SEQ ID NO: 219 or a functional sequence variant thereof, as described herein, wherein the “LALA” mutation is maintained.

The “LALA” mutation (CH2 L4A and CH2 L5A mutation) abolishes antibody binding to FcγRI, FcγRII and FcγRIIIa (Beltramello M, Williams K L, Simmons C P, et al. The Human Immune Response to Dengue Virus Is Dominated by Highly Cross-Reactive Antibodies Endowed with Neutralizing and Enhancing Activity. Cell host & microbe. 2010; 8(3):10.1016/j.chom.2010.08.007. doi:10.1016/j.chom.2010.08.007), but does not compromise interaction with FcRn. In general, the amino acid at positions 4 and 5 of CH2 of the wild-type IgG1 and IgG3 is a leucine (“L”). According to the invention, the anti-Lyssavirus antibody comprises an alanine (“A”) at position CH2 4, CH2 5, or both, instead of the naturally occurring leucine (L). In particular, the antibody according to the invention comprises both, a CH2 L4A and a CH2 L5A substitution. Such antibodies are referred to herein as a “LALA” variant. An exemplary amino acid sequence of IgG1 CH1-CH2-CH3 comprising the “LALA” mutation is set forth in SEQ ID NO: 219. Accordingly, the amino acid sequence of IgG1 CH1-CH2-CH3 is, for example, according to SEQ ID NO: 219 or a functional sequence variant thereof, as described herein, wherein the “LALA” mutation is maintained.

In general, the anti-lyssavirus antibody, or the antigen-binding fragment thereof, is described in detail above. Accordingly, embodiments of the anti-lyssavirus antibody, or the antigen-binding fragment thereof, for use according to the invention (in particular which is administered into the CNS and peripherally) as described above correspond to embodiments of the anti-lyssavirus antibody, or the antigen-binding fragment thereof, comprising an Fc moiety comprising a CH2 domain and a CH2 L4A mutation and/or a CH2 L5A mutation.

For example, the anti-lyssavirus antibody, or the antigen-binding fragment thereof, may be an anti-RABV antibody or an antigen-binding fragment thereof.

For example, the anti-lyssavirus antibody, or the antigen-binding fragment thereof, binds to lyssavirus glycoprotein G, in particular to glycoprotein G of RABV, as described above, e.g., to antigenic site I or antigenic site III of glycoprotein G of RABV, as described in detail above.

For example, the anti-lyssavirus antibody, or the antigen-binding fragment thereof, is a monoclonal antibody and/or a human antibody, as described above. For example, the anti-lyssavirus antibody, or the antigen-binding fragment thereof, is a purified antibody, a single chain antibody, Fab, Fab′, F(ab′)2, Fv or scFv, as described above.

As described above, the anti-lyssavirus antibody, or the antigen-binding fragment thereof, neutralizes, for instance, lyssavirus infection by (i) RABV and (ii) at least 50% of non-RABV lyssaviruses selected from the group consisting of DUVV, EBLV-1, EBLV-2, ABLY, IRKV, KHUV, ARAV, LBV, MOK, SHIV, BBLV and WCBV, with an IC₅₀ of less than 10000 ng/ml. Moreover, the anti-lyssavirus antibody, or the antigen-binding fragment thereof, neutralizes, for instance, infection by RABV CVS-11 with an IC₉₀ of 400 ng/ml or less, as described above.

For example, the antibody, or antigen binding fragment thereof, comprises: (i) heavy chain CDRH1, CDRH2, and CDRH3 amino acid sequences and light chain CDRL1, CDRL2, and CDRL3 amino acid sequences as set forth in SEQ ID NOs: 93-97 and 99, or sequence variants thereof, or as set forth in SEQ ID NOs: 93-96 and 98-99, or sequence variants thereof; (ii) heavy chain CDRH1, CDRH2, and CDRH3 amino acid sequences and light chain CDRL1, CDRL2, and CDRL3 amino acid sequences as set forth in SEQ ID NOs: 165-169 and 171, or sequence variants thereof, or as set forth in SEQ ID NOs: 165-168 and 170-171, or sequence variants thereof; (iii) heavy chain CDRH1, CDRH2, and CDRH3 amino acid sequences and light chain CDRL1, CDRL2, and CDRL3 amino acid sequences as set forth in SEQ ID NOs: 1-5 and 7, or sequence variants thereof, or as set forth in SEQ ID NOs: 1-4 and 6-7, or sequence variants thereof; (iv) heavy chain CDRH1, CDRH2, and CDRH3 amino acid sequences and light chain CDRL1, CDRL2, and CDRL3 amino acid sequences as set forth in SEQ ID NOs: 19-23 and 25, or sequence variants thereof, or as set forth in SEQ ID NOs: 19-22 and 24-25, or sequence variants thereof; (v) heavy chain CDRH1, CDRH2, and CDRH3 amino acid sequences and light chain CDRL1, CDRL2, and CDRL3 amino acid sequences as set forth in SEQ ID NOs: 37-41 and 43, or sequence variants thereof, or as set forth in SEQ ID NOs: 37-40 and 42-43, or sequence variants thereof; (vi) heavy chain CDRH1, CDRH2, and CDRH3 amino acid sequences and light chain CDRL1, CDRL2, and CDRL3 amino acid sequences as set forth in SEQ ID NOs: 55-59 and 61, or sequence variants thereof, or as set forth in SEQ ID NOs: 55-58 and 60-61, or sequence variants thereof; (vii) heavy chain CDRH1, CDRH2, and CDRH3 amino acid sequences and light chain CDRL1, CDRL2, and CDRL3 amino acid sequences as set forth in SEQ ID NOs: 75-79 and 81, or sequence variants thereof, or as set forth in SEQ ID NOs: 75-78 and 80-81, or sequence variants thereof; (viii) heavy chain CDRH1, CDRH2, and CDRH3 amino acid sequences and light chain CDRL1, CDRL2, and CDRL3 amino acid sequences as set forth in SEQ ID NOs: 111-115 and 117, or sequence variants thereof, or as set forth in SEQ ID NOs: 111-114 and 116-117, or sequence variants thereof; (ix) heavy chain CDRH1, CDRH2, and CDRH3 amino acid sequences and light chain CDRL1, CDRL2, and CDRL3 amino acid sequences as set forth in SEQ ID NOs: 129-133 and 135, or sequence variants thereof, or as set forth in SEQ ID NOs: 129-132 and 134-135, or sequence variants thereof; (x) heavy chain CDRH1, CDRH2, and CDRH3 amino acid sequences and light chain CDRL1, CDRL2, and CDRL3 amino acid sequences as set forth in SEQ ID NOs: 147-151 and 153, or sequence variants thereof, or as set forth in SEQ ID NOs: 147-150 and 152-153, or sequence variants thereof; (xi) heavy chain CDRH1, CDRH2, and CDRH3 amino acid sequences and light chain CDRL1, CDRL2, and CDRL3 amino acid sequences as set forth in SEQ ID NOs: 183-187 and 189, or sequence variants thereof, or as set forth in SEQ ID NOs: 183-186 and 188-189, or sequence variants thereof; or (xii) heavy chain CDRH1, CDRH2, and CDRH3 amino acid sequences and light chain CDRL1, CDRL2, and CDRL3 amino acid sequences as set forth in SEQ ID NOs: 201-205 and 207, or sequence variants thereof, or as set forth in SEQ ID NOs: 201-204 and 206-207, or sequence variants thereof.

For example, the anti-lyssavirus antibody, or the antigen-binding fragment thereof, comprises: (i) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 93-97 and 99 or to the amino acid sequences of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 165-169 and 171 or to the amino acid sequences of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 1-5 and 7 or to the amino acid sequences of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 19-23 and 25 or to the amino acid sequences of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 37-41 and 43 or to the amino acid sequences of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 55-59 and 61 or to the amino acid sequences of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 75-79 and 81 or to the amino acid sequences of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 111-115 and 117 or to the amino acid sequences of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 129-133 and 135 or to the amino acid sequences of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 147-151 and 153 or to the amino acid sequences of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 183-187 and 189 or to the amino acid sequences of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences that are at least 80% or at least 90% identical to the amino acid sequences of SEQ ID NOs: 201-205 and 207 or to the amino acid sequences of SEQ ID NOs: 201-204 and 206-207, respectively, as described above.

As described above, the anti-lyssavirus antibody, or the antigen-binding fragment thereof, comprises, for example, heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof, such as amino acid sequences that are at least 80% or at least 90% identical to the amino acid sequences of SEQ ID NOs: 93-97 and 99 or to the amino acid sequences of SEQ ID NOs: 93-96 and 98-99, respectively.

As described above, the anti-lyssavirus antibody, or the antigen-binding fragment thereof, comprises, for example, heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof, such as amino acid sequences that are at least 80% or at least 90% identical to the amino acid sequences of SEQ ID NOs: 165-169 and 171 or to the amino acid sequences of SEQ ID NOs: 165-168 and 170-171, respectively.

For example, the anti-lyssavirus antibody, or the antigen-binding fragment thereof, comprises: (i) a heavy chain variable region having an amino acid sequence according to SEQ ID NO: 107 and a light chain variable region having an amino acid sequence according to SEQ ID NO: 108, or sequence variants thereof, such as a heavy chain variable region having at least 80% or at least 90% sequence identity to the amino acid sequence of SEQ ID NO: 107 and a light chain variable region having at least 80% or at least 90% sequence identity to the amino acid sequence of SEQ ID NO: 108; or (ii) a heavy chain variable region having an amino acid sequence according to SEQ ID NO: 179 and a light chain variable region having an amino acid sequence according to SEQ ID NO: 180, or sequence variants thereof, such as a heavy chain variable region having at least 80% or at least 90% sequence identity to the amino acid sequence of SEQ ID NO: 179 and a light chain variable region having at least 80% or at least 90% sequence identity to the amino acid sequence of SEQ ID NO: 180; or (iii) a heavy chain variable region having an amino acid sequence according to SEQ ID NO: 15 and a light chain variable region having an amino acid sequence according to SEQ ID NO: 16, or sequence variants thereof, such as a heavy chain variable region having at least 80% or at least 90% sequence identity to the amino acid sequence of SEQ ID NO: 15 and a light chain variable region having at least 80% or at least 90% sequence identity to the amino acid sequence of SEQ ID NO: 16; or (iv) a heavy chain variable region having an amino acid sequence according to SEQ ID NO: 33 and a light chain variable region having an amino acid sequence according to SEQ ID NO: 34, or sequence variants thereof, such as a heavy chain variable region having at least 80% or at least 90% sequence identity to the amino acid sequence of SEQ ID NO: 33 and a light chain variable region having at least 80% or at least 90% sequence identity to the amino acid sequence of SEQ ID NO: 34; or (v) a heavy chain variable region having an amino acid sequence according to SEQ ID NO: 51 and a light chain variable region having an amino acid sequence according to SEQ ID NO: 52, or sequence variants thereof, such as a heavy chain variable region having at least 80% or at least 90% sequence identity to the amino acid sequence of SEQ ID NO: 51 and a light chain variable region having at least 80% or at least 90% sequence identity to the amino acid sequence of SEQ ID NO: 52; or (vi) a heavy chain variable region having an amino acid sequence according to SEQ ID NO: 69 and a light chain variable region having an amino acid sequence according to SEQ ID NO: 71, or sequence variants thereof, such as a heavy chain variable region having at least 80% or at least 90% sequence identity to the amino acid sequence of SEQ ID NO: 69 and a light chain variable region having at least 80% or at least 90% sequence identity to the amino acid sequence of SEQ ID NO: 71; or (vii) a heavy chain variable region having an amino acid sequence according to SEQ ID NO: 70 and a light chain variable region having an amino acid sequence according to SEQ ID NO: 71, or sequence variants thereof, such as a heavy chain variable region having at least 80% or at least 90% sequence identity to the amino acid sequence of SEQ ID NO: 70 and a light chain variable region having at least 80% or at least 90% sequence identity to the amino acid sequence of SEQ ID NO: 71; or (viii) a heavy chain variable region having an amino acid sequence according to SEQ ID NO: 89 and a light chain variable region having an amino acid sequence according to SEQ ID NO: 90, or sequence variants thereof, such as a heavy chain variable region having at least 80% or at least 90% sequence identity to the amino acid sequence of SEQ ID NO: 89 and a light chain variable region having at least 80% or at least 90% sequence identity to the amino acid sequence of SEQ ID NO: 90; or (ix) a heavy chain variable region having an amino acid sequence according to SEQ ID NO: 125 and a light chain variable region having an amino acid sequence according to SEQ ID NO: 126, or sequence variants thereof, such as a heavy chain variable region having at least 80% or at least 90% sequence identity to the amino acid sequence of SEQ ID NO: 125 and a light chain variable region having at least 80% or at least 90% sequence identity to the amino acid sequence of SEQ ID NO: 126; or (x) a heavy chain variable region having an amino acid sequence according to SEQ ID NO: 143 and a light chain variable region having an amino acid sequence according to SEQ ID NO: 144, or sequence variants thereof, such as a heavy chain variable region having at least 80% or at least 90% sequence identity to the amino acid sequence of SEQ ID NO: 143 and a light chain variable region having at least 80% or at least 90% sequence identity to the amino acid sequence of SEQ ID NO: 144; or (xi) a heavy chain variable region having an amino acid sequence according to SEQ ID NO: 161 and a light chain variable region having an amino acid sequence according to SEQ ID NO: 162, or sequence variants thereof, such as a heavy chain variable region having at least 80% or at least 90% sequence identity to the amino acid sequence of SEQ ID NO: 161 and a light chain variable region having at least 80% or at least 90% sequence identity to the amino acid sequence of SEQ ID NO: 162; or (xii) a heavy chain variable region having an amino acid sequence according to SEQ ID NO: 197 and a light chain variable region having an amino acid sequence according to SEQ ID NO: 198, or sequence variants thereof, such as a heavy chain variable region having at least 80% or at least 90% sequence identity to the amino acid sequence of SEQ ID NO: 197 and a light chain variable region having at least 80% or at least 90% sequence identity to the amino acid sequence of SEQ ID NO: 198; or (xiii) a heavy chain variable region having an amino acid sequence according to SEQ ID NO: 215 and a light chain variable region having an amino acid sequence according to SEQ ID NO: 216, or sequence variants thereof, such as a heavy chain variable region having at least 80% or at least 90% sequence identity to the amino acid sequence of SEQ ID NO: 215 and a light chain variable region having at least 80% or at least 90% sequence identity to the amino acid sequence of SEQ ID NO: 216.

As described above, the anti-lyssavirus antibody, or the antigen-binding fragment thereof, comprises, for example, a heavy chain variable region having an amino acid sequence according to SEQ ID NO: 107 and a light chain variable region having an amino acid sequence according to SEQ ID NO: 108, or sequence variants thereof, such as a heavy chain variable region having at least 80% or at least 90% sequence identity to the amino acid sequence of SEQ ID NO: 107 and a light chain variable region having at least 80% or at least 90% sequence identity to the amino acid sequence of SEQ ID NO: 108.

As described above, the anti-lyssavirus antibody, or the antigen-binding fragment thereof, comprises, for example, a heavy chain variable region having an amino acid sequence according to SEQ ID NO: 179 and a light chain variable region having an amino acid sequence according to SEQ ID NO: 180, or sequence variants thereof, such as a heavy chain variable region having at least 80% or at least 90% sequence identity to the amino acid sequence of SEQ ID NO: 179 and a light chain variable region having at least 80% or at least 90% sequence identity to the amino acid sequence of SEQ ID NO: 180.

For example, the anti-lyssavirus antibody, or the antigen-binding fragment thereof, may be RVC20, RVC58, RVA122, RVA144, RVB185, RVB492, RVC3, RVC21, RVC38, RVC44, RVC68, or RVC111 such as RVC20 or RVC58, as described above.

In another aspect, the invention also provides a nucleic acid molecule comprising a polynucleotide encoding the anti-lyssavirus antibody, or the antigen-binding fragment thereof, comprising an Fc moiety comprising a CH2 domain and a CH2 L4A mutation and/or a CH2 L5A mutation.

A nucleic acid molecule is a molecule comprising, typically consisting of, nucleic acid components. The term nucleic acid molecule typically refers to DNA or RNA molecules. It is typically used synonymous with the term “polynucleotide”. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules. Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA.

Nucleic acid sequences encode, in particular, part or all of the light and heavy chains and/or CDRs of the antibodies of the invention. In some embodiments, nucleic acid sequences encoding part or all of the light and heavy chains and CDRs of exemplary antibodies of the invention are used. The above table 3 provides the SEQ ID numbers for the nucleic acid sequences encoding the heavy chain and light chain variable regions of exemplified antibodies. Table 4 below provides the SEQ ID numbers for the nucleic acid sequences encoding the CDRs of exemplified antibodies. Due to the redundancy of the genetic code, variants of these nucleic acid sequences will exist that encode the same amino acid sequences.

Thus, the invention also comprises a nucleic acid molecule comprising a polynucleotide encoding the antibody, or the antigen binding fragment thereof, according to the invention.

The present invention also provides a combination of at least two distinct nucleic acid molecules encoding the anti-lyssavirus antibody, or an antigen-binding fragment thereof, as described herein comprising an Fc moiety comprising a CH2 domain and a CH2 L4A mutation and/or a CH2 L5A mutation, wherein one nucleic acid molecule encodes at least a CDRH1, CDRH2 and CDRH3 of a heavy chain and another nucleic acid molecule encodes at least a CDRL1, CDRL2 and CDRL3 of a corresponding light chain of the antilyssavirus antibody or antigen-binding fragment. For example, one nucleic acid molecule encodes at least a variable region of a heavy chain (VH) or a (complete) heavy chain; and another nucleic acid molecule encodes at least a corresponding variable region of a light chain (VL) or a (complete) light chain of the anti lyssavirus antibody.

TABLE 4
SEQ ID Numbers for CDR Polynucleotides
of exemplary antibodies according to the invention.

| | SEQ ID NOs. for CDR Polynucleotides | | | | | |
|---|---|---|---|---|---|---|
| | CDRH1 | CDRH2 | CDRH3 | CDRL1 | CDRL2 | CDRL3 |
| RVA122 | 8 | 9 | 10 | 11 | 12 or 13 | 14 |
| RVA144 | 26 | 27 | 28 | 29 | 30 or 31 | 32 |
| RVB185 | 44 | 45 | 46 | 47 | 48 or 49 | 50 |
| RVB492 | 62 | 63 | 64 | 65 | 66 or 67 | 68 |
| RVC3 | 82 | 83 | 84 | 85 | 86 or 87 | 88 |
| RVC20 | 100 | 101 | 102 | 103 | 104 or 105 | 106 |
| RVC21 | 118 | 119 | 120 | 121 | 122 or 123 | 124 |
| RVC38 | 136 | 137 | 138 | 139 | 140 or 141 | 142 |
| RVC44 | 154 | 155 | 156 | 157 | 158 or 159 | 160 |
| RVC58 | 172 | 173 | 174 | 175 | 176 or 177 | 178 |
| RVC68 | 190 | 191 | 192 | 193 | 194 or 195 | 196 |
| RVC111 | 208 | 209 | 210 | 211 | 212 or 213 | 214 |

For example, the polynucleotide sequence of the nucleic acid molecule according to the invention is at least 75% identical to the nucleic acid sequence of any one of SEQ ID NOs: 8-14, 17, 18, 26-32, 35, 36, 44-50, 53, 54, 62-68, 72-74, 82-88, 91, 92, 100-106, 109, 110, 118-124, 127, 128, 136-142, 145, 146, 154-160, 163, 164, 172-178, 181, 182, 190-196, 199, 200, 208-214, 217, 218 or 222-224. For example, the nucleotide sequence of the nucleic acid molecule according to the invention is according to any one of SEQ ID NOs: 8-14, 17, 18, 26-32, 35, 36, 44-50, 53, 54, 62-68, 72-74, 82-88, 91, 92, 100-106, 109, 110, 118-124, 127, 128, 136-142, 145, 146, 154-160, 163, 164, 172-178, 181, 182, 190-196, 199, 200, 208-214, 217, 218, or sequence variants thereof.

In particular, nucleic acid sequences according to the invention include nucleic acid sequences having at least 70%, at least 75%, at least 80%, at least 85%, at least 88%, at least 90%, at least 92%, at least 95%, at least 96%, at least 97%, at least 98% or at least 99% identity to the nucleic acid encoding the variable region of a heavy or light chain of an antibody of the invention. In another embodiment, a nucleic acid sequence of the invention has the sequence of a nucleic acid encoding a heavy or light chain CDR of an antibody of the invention. For example, a nucleic acid sequence according to the invention comprises a sequence that is at least 75%, at least 80%, at least 85%, at least 88%, at least 90%, at least 92%, at least 95%, at least 96%, at least 97%, at least 98% or at least 99% identical to the nucleic acid sequences of SEQ ID NOs: 8-14, 17, 18, 26-32, 35, 36, 44-50, 53, 54, 62-68, 72-74, 82-88, 91, 92, 100-106, 109, 110, 118-124, 127, 128, 136-142, 145, 146, 154-160, 163, 164, 172-178, 181, 182, 190-196, 199, 200, 208-214, 217, or 218.

For example, nucleic acid sequences according to the invention include nucleic acid sequences having at least 70%, at least 75%, at least 80%, at least 85%, at least 88%, at least 90%, at least 92%, at least 95%, at least 96%, at least 97%, at least 98% or at least 99% identity to the nucleic acid encoding a heavy or light chain of an antibody of the invention as described herein.

In some embodiments, the nucleic acid molecule comprises a polynucleotide encoding an anti-lyssavirus antibody as described herein, which comprises an IgG1 CH1-CH2-CH3 according to SEQ ID NO: 219, or a functional sequence variant thereof as described herein. In some embodiments, the nucleic acid molecule comprises a nucleotide sequence as set forth in SEQ ID NO: 222, or a functional sequence variant thereof as described herein.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Mutations can be introduced in specific sites or can be introduced at random, followed by selection (e.g., molecular evolution). For instance, a nucleic acid encoding an antibody of the invention can be randomly or directionally mutated to introduce different properties in the encoded amino acids. Such changes can be the result of an iterative process wherein initial changes are retained and new changes at other nucleotide positions are introduced. Further, changes achieved in independent steps may be combined. Different properties introduced into the encoded amino acids may include, but are not limited to, enhanced affinity.

Further included within the scope of the invention are vectors, for example, expression vectors, comprising a nucleic acid sequence according to the invention. Typically, a vector comprises a nucleic acid molecule according to the invention, for example a nucleic acid molecule as described above. In some embodiments, the vector may be bicistronic. For example, at least two distinct nucleic acid molecules encoding the anti-lyssavirus antibody, or an antigen-binding fragment thereof, as described herein comprising an Fc moiety comprising a CH2 domain and a CH2 L4A mutation and/or a CH2 L5A mutation, may be encoded in distinct cistrons. In this way, for example, the vector may comprise the above described combination of nucleic acid molecules, e.g. with each nucleic acid molecule in a distinct cistron. Accordingly, at least a CDRH1, CDRH2 and CDRH3 of a heavy chain may be encoded in one cistron and another cistron may encode at least a CDRL1, CDRL2 and CDRL3 of a corresponding light chain of the antilyssavirus antibody or antigen-binding fragment. For example, one cistron encodes at least a variable region of a heavy chain (VH) or a (complete) heavy chain; and another cistron encodes at least a corresponding variable region of a light chain (VL) or a (complete) light chain of the antilyssavirus antibody.

The term “vector” refers to a nucleic acid molecule, such as an artificial nucleic acid molecule, i.e. a nucleic acid molecule which does not occur in nature. A vector in the context of the invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a desired antibody or antibody fragment thereof according to the invention. An expression vector may be used for production of expression products such as RNA, e.g. mRNA, or peptides, polypeptides or proteins. For example, an expression vector may comprise sequences needed for transcription of a sequence stretch of the vector, such as a promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the invention may be, e.g., an RNA vector or a DNA vector. For example, a vector is a DNA molecule. For example, a vector in the sense of the present application comprises a cloning site, a selection marker, such as an antibiotic resistance factor, and a sequence suitable for multiplication of the vector, such as an origin of replication. For example, a vector in the context of the disclosure is a plasmid vector.

Cells transformed with such vectors are also included within the scope of the invention. Examples of such cells include but are not limited to, eukaryotic cells, e.g., yeast cells, animal cells or plant cells. In one embodiment the cells are mammalian, e.g., human, CHO, HEK293T, PER.C6, NS0, myeloma or hybridoma cells. Accordingly, the invention also relates to a cell expressing the antibody, or the antigen binding fragment thereof, according to the invention; or comprising the vector according to the invention.

In particular, the cell may be transfected with a vector according to the invention, such as an expression vector. The term “transfection” refers to the introduction of nucleic acid molecules, such as DNA or RNA (e.g. mRNA) molecules, into cells, such as eukaryotic cells. In the context of the invention, the term “transfection” encompasses any method known to the skilled person for introducing nucleic acid molecules into cells, such as eukaryotic cells, e.g., mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

In a further aspect, the invention also provides a pharmaceutical composition comprising the anti-lyssavirus antibody, or the antigen-binding fragment thereof, which comprises an Fc moiety comprising a CH2 domain and a CH2 L4A mutation and/or a CH2 L5A mutation, as described above.

In some embodiments of such a pharmaceutical composition correspond to embodiments of the pharmaceutical composition comprising the anti-lyssavirus antibody, or the antigen-binding fragment thereof, for use according to the invention (in particular, which is administered into the CNS and peripherally) as described above.

For example, the pharmaceutical composition comprises a pharmaceutically acceptable excipient, diluent or carrier as described above.

As described in detail above, the pharmaceutical composition comprises, for instance, at least two distinct anti-lyssavirus antibodies, or antigen-binding fragments thereof, as described herein, which comprise an Fc moiety comprising a CH2 domain and a CH2 L4A mutation and/or a CH2 L5A mutation. As described above, the at least two antibodies, or antigen-binding fragments thereof, bind specifically, for example, to different epitopes on the glycoprotein G of RABV, such as to antigenic site I and to antigenic site III. For example, at least two distinct anti-lyssavirus antibodies, or antigen-binding fragments thereof, are comprised in the pharmaceutical composition in equimolar amounts, as described above. For example, one of the two anti-Lyssavirus antibodies, or the antigen-binding fragments thereof, may comprise heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof, such as amino acid sequences that are at least 80% or at least 90% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, may comprise heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, or sequence variants thereof, such as amino acid sequences that are at least 80% or at least 90% identical to the amino acid sequences of SEQ ID NOs: 165-169 and 171 or to the amino acid sequences of SEQ ID NOs: 165-168 and 170-171, respectively, as described above. For example, one of the two anti-lyssavirus antibodies, or the antigen-binding fragments thereof, comprises a heavy chain variable region having an amino acid sequence according to SEQ ID NO: 107 and a light chain variable region having an amino acid sequence according to SEQ ID NO: 108, or sequence variants thereof, such as a heavy chain variable region having at least 80% or at least 90% sequence identity to the amino acid sequence of SEQ ID NO: 107 and a light chain variable region having at least 80% or at least 90% sequence identity to the amino acid sequence of SEQ ID NO: 108; and the other of the two anti-lyssavirus antibodies, or the antigen-binding fragments thereof, comprises a heavy chain variable region having an amino acid sequence according to SEQ ID NO: 179 and a light chain variable region having an amino acid sequence according to SEQ ID NO: 180, or sequence variants thereof, such as a heavy chain variable region having at least 80% or at least 90% sequence identity to the amino acid sequence of SEQ ID NO: 179 and a light chain variable region having at least 80% or at least 90% sequence identity to the amino acid sequence of SEQ ID NO: 180, as described above.

In a further aspect, the invention also provides a kit of parts comprising
the anti-lyssavirus antibody, or the antigen-binding fragment thereof, which comprises an Fc moiety comprising a CH2 domain and a CH2 L4A mutation and/or a CH2 L5A mutation, as described above; or
the pharmaceutical composition comprising the anti-Lyssavirus antibody, or the antigen-binding fragment thereof, which comprises an Fc moiety comprising a CH2 domain and a CH2 L4A mutation and/or a CH2 L5A mutation, as described above.

Embodiments of such a kit of parts correspond to embodiments of the kit of parts comprising the anti-lyssavirus antibody, or the antigen-binding fragment thereof, for use according to the invention (in particular, which is administered into the CNS and peripherally) as described above.

For example, the kit of parts comprises at least two distinct anti-lyssavirus antibodies, or antigen-binding fragments thereof, as described above, which comprise an Fc moiety comprising a CH2 domain and a CH2 L4A mutation and/or a CH2 L5A mutation. As described above, the at least two antibodies, or antigen-binding fragments thereof, bind specifically, for example, to different epitopes on the glycoprotein G of RABV, e.g., to antigenic site I and to antigenic site III. For example, one of the two anti-lyssavirus antibodies, or the antigen-binding fragments thereof, may comprise heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof, such as amino acid sequences that are at least 80% or at least 90% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, may comprise heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, or sequence variants thereof, such as amino acid sequences that are at least 80% or at least 90% identical to the amino acid sequences of SEQ ID NOs: 165-169 and 171 or to the amino acid sequences of SEQ ID NOs: 165-168 and 170-171, respectively, as described above. For example, one of the two anti-lyssavirus antibodies, or the antigen-binding fragments thereof, comprises a heavy chain variable region having an amino acid sequence according to SEQ ID NO: 107 and a light chain variable region having an amino acid sequence according to SEQ ID NO: 108, or sequence variants thereof, such as a heavy chain variable region having at least 80% or at least 90% sequence identity to the amino acid sequence of SEQ ID NO: 107 and a light chain variable region having at least 80%, or at least 90% sequence identity to the amino acid sequence of SEQ ID NO: 108; and the other of the two anti-lyssavirus antibodies, or the antigen-binding fragments thereof, comprises a heavy chain variable region having an amino acid sequence according to SEQ ID NO: 179 and a light chain variable region having an amino acid sequence according to SEQ ID NO: 180, or sequence variants thereof, such as a heavy chain variable region having at least 80% or at least 90% sequence identity to the amino acid sequence of SEQ ID NO: 179 and a light chain variable region having at least 80% or at least 90% sequence identity to the amino acid sequence of SEQ ID NO: 180, as described above.

### Medical Treatment and Uses

The anti-lyssavirus antibody, or the antigen-binding fragment thereof, according to the invention as described above, which comprises an Fc moiety comprising a CH2 domain and a CH2 L4A mutation and/or a CH2 L5A mutation, as described above, can be used in the prevention and/or treatment of lyssavirus infection.

In particular, the invention provides the use of such an antibody, or an antigen binding fragment thereof, according to the invention, the nucleic acid according to the invention, the vector according to the invention, the cell according to the invention, or the pharmaceutical composition according to the invention in (i) prophylaxis, in particular post-exposure prophylaxis, treatment or attenuation of RABV and/or non-RABV lyssavirus infection; in (ii) vaccination against RABV and/or non-RABV lyssavirus infection; or in (iii) diagnosis of RABV and/or other lyssavirus infection.

For example, the antibody, or the antigen-binding fragment thereof, is administered in combination with a further anti-lyssavirus antibody, or an antigen-binding fragment thereof, for example as described herein. It is understood that the two anti-lyssavirus antibodies, or the antigen-binding fragments thereof, which are administered in combination, are distinct. For example, the two anti-lyssavirus antibodies, or the antigen-binding fragments thereof, which are administered in combination, can bind specifically to different epitopes of a lyssavirus glycoprotein G, in particular on the glycoprotein G of RABV. Targeting different epitopes (different antigenic sites) avoids appearance of resistant virus strains and prevents the escape of resistant variants of the virus (viral escape mutants). Accordingly, the combination of two anti-lyssavirus antibodies, which bind to different epitopes of the lyssavirus G protein represents a treatment with an unprecedented breadth of reactivity and with reduced risk of escape mutant selection. In particular, a combination of two or more monoclonal antibodies binding to different epitopes or sites on the lyssavirus (e.g., RABV) G protein increases the protective effect and prevents the escape of resistant variants of the virus. Accordingly, the present invention provides in particular a combination of two or more monoclonal antibodies binding to different epitopes or sites on the lyssavirus (e.g., RABV) G protein. Whether or not two or more antibodies bind to the same or different epitopes on the lyssavirus G protein may be easily determined by the person skilled in the art, for example by use of any competition study, for example as described in Example 3 of WO 2016/078761, which is incorporated herein by reference.

For example, one of the two anti-lyssavirus antibodies, or the antigen-binding fragments thereof, which are administered in combination, may specifically bind to antigenic site I of glycoprotein G of a lyssavirus (e.g., RABV), whereas the other of the two anti-lyssavirus antibodies, or the antigen-binding fragments thereof, which are administered in combination, may specifically bind to antigenic site III (or to an epitope which at least partially overlaps with antigenic site III) of glycoprotein G of a lyssavirus (e.g., RABV). For example, one of the two anti-lyssavirus antibodies, or the antigen-binding fragments thereof, administered in combination may comprise heavy chain CDRH1, CDRH2, and CDRH3 amino acid sequences and light chain CDRL1, CDRL2, and CDRL3 amino acid sequences as set forth in SEQ ID NOs: 93-97 and 99 or in SEQ ID NOs: 93-96 and 98-99, or sequence variants thereof, such as amino acid sequences that are at least 80% or at least 90% identical to the amino acid sequences of SEQ ID NOs: 93-97 and 99 or to the amino acid sequences of SEQ ID NOs: 93-96 and 98-99, respectively; and the other of the two anti-lyssavirus antibodies, or the antigen-binding fragments thereof, administered in combination may comprise heavy chain CDRH1, CDRH2, and CDRH3 amino acid sequences and light chain CDRL1, CDRL2, and CDRL3 amino acid sequences as set forth in SEQ ID NOs: 165-169 and 171 or in SEQ ID NOs: 165-168 and 170-171, or sequence variants thereof, such as amino acid sequences that are at least 80% or at least 90% identical to the amino acid sequences of SEQ ID NOs: 165-169 and 171 or to the amino acid sequences of SEQ ID NOs: 165-168 and 170-171, respectively. For example, one of the two anti-lyssavirus antibodies, or the antigen-binding fragments thereof, administered in combination comprises a heavy chain variable region having an amino acid sequence according to SEQ ID NO: 107 and a light chain variable region having an amino acid sequence according to SEQ ID NO: 108, or sequence variants thereof, such as a heavy chain variable region having at least 80% or at least 90% sequence identity to the amino acid sequence of SEQ ID NO: 107 and a light chain variable region having at least 80% or at least 90% sequence identity to the amino acid sequence of SEQ ID NO: 108; and the other of the two anti-lyssavirus antibodies, or the antigen-binding fragments thereof, administered in combination comprises a heavy chain variable region having an amino acid sequence according to SEQ ID NO: 179 and a light chain variable region having an amino acid sequence according to SEQ ID NO: 180, or sequence variants thereof, such as a heavy chain variable region having at least 80% or at least 90% sequence identity to the amino acid sequence of SEQ ID NO: 179 and a light chain variable region having at least 80% or at least 90% sequence identity to the amino acid sequence of SEQ ID NO: 180. For example, the two anti-lyssavirus antibodies administered in combination are LALA-variants of RVC20 and RVC58 (anti-lyssavirus antibodies RVC20 and RVC58, each comprising the LALA mutation as described herein).

The two anti-lyssavirus antibodies, or the antigen-binding fragments thereof, which are administered in combination, may be comprised in the same or distinct pharmaceutical composition. In other words, the two anti-lyssavirus antibodies, or the antigen-binding fragments thereof, which are administered in combination, may be administered separately, for example in separate pharmaceutical compositions, or together, i.e. as antibody “cocktail”, for example in the same pharmaceutical composition. For example, the two anti-lyssavirus antibodies, or the antigen-binding fragments thereof, which are administered in combination, are comprised in the same pharmaceutical composition.

For instance, the antibody, or the antigen binding fragment thereof, for use according to the invention as described herein and the other antibody, which is administered in combination, are administered at equimolar amounts. If they are comprised in the same pharmaceutical composition, the (at least) two antibodies are present in the pharmaceutical composition at equimolar amounts, e.g., as an equimolar mixture.

The invention also provides the pharmaceutical composition according to the invention as described above, which comprises the anti-lyssavirus antibody, or the antigen-binding fragment thereof, comprising an Fc moiety comprising a CH2 domain and a CH2 L4A mutation and/or a CH2 L5A mutation, as described above, for use in the prevention and/or treatment of lyssavirus infection.

Accordingly, the invention also provides a method of treating or attenuating a lyssavirus infection in a subject, wherein the method comprises administering to a subject in need thereof the antibody, or the antigen binding fragment thereof, comprising an Fc moiety comprising a CH2 domain and a CH2 L4A mutation and/or a CH2 L5A mutation, as described above; or the pharmaceutical composition comprising the anti-lyssavirus antibody, or the antigen-binding fragment thereof, which comprises an Fc moiety comprising a CH2 domain and a CH2 L4A mutation and/or a CH2 L5A mutation, as described above.

For treatment of lyssavirus infection, the antibody, or the antigen-binding fragment thereof, according to the invention as described above, which comprises an Fc moiety comprising a CH2 domain and a CH2 L4A mutation and/or a CH2 L5A mutation, or the pharmaceutical composition according to the invention as described above, which comprises said antibody, may be administered

(i) into the central nervous system (CNS); and
(ii) peripherally
as described above.

In other words, the antibody, or the antigen-binding fragment thereof, according to the invention as described above, which comprises an Fc moiety comprising a CH2 domain and a CH2 L4A mutation and/or a CH2 L5A mutation, or the pharmaceutical composition according to the invention as described above, which comprises said antibody, is, for example, administered as described above. Thereby, details and embodiments described above for the inventive administration of the anti-lyssavirus antibody into the CNS and peripherally correspond to details and embodiments of treatment with the antibody, or the antigen-binding fragment thereof, according to the invention as described above, which comprises an Fc moiety comprising a CH2 domain and a CH2 L4A mutation and/or a CH2 L5A mutation, or the pharmaceutical composition according to the invention as described above, which comprises said antibody.

For example, the antibody, or the antigen-binding fragment thereof, according to the invention as described above, which comprises an Fc moiety comprising a CH2 domain and a CH2 L4A mutation and/or a CH2 L5A mutation, or the pharmaceutical composition according to the invention as described above, which comprises said antibody, may be used for the post-exposure prophylaxis or attenuation of lyssavirus infection.

The term “post-exposure prophylaxis” (also referred to as “PEP”) as used herein refers to a treatment protocol, which starts after exposure to the virus and before the first symptoms of rabies are detectable. Methods and treatment schedules for post-exposure prophylaxis are well-known to those skilled in the art.

In general, post-exposure prophylaxis is started as soon as possible after exposure or suspected exposure to the virus, e.g., within a few hours until up to 24 hours or up to 48 hours after exposure. In this limited time window, post-exposure prophylaxis is known to be most effective. For example, in post-exposure prophylaxis or attenuation the antibody, or the antigen binding fragment thereof, or the pharmaceutical composition is administered up to seven days or up to five days, after lyssavirus infection.

For example, the antibody, or the antigen binding fragment thereof, according to the invention or the pharmaceutical composition according to the invention is used in post-exposure prophylaxis, treatment or attenuation of RABV and/or non-RABV lyssavirus infection, wherein the antibody, or the antigen binding fragment thereof, or the pharmaceutical composition is administered in combination with a vaccine, such as a rabies vaccine, an antiviral, such as ribavirin, interferon-alpha and/or ketamine.

As described above, currently available rabies vaccines include the most widely used but highly risk-prone nerve tissue vaccines, or the safer but more costly cell culture and embryonated egg vaccines (CCEEVs). In Germany e.g. only two anti-rabies vaccines are on the market, Rabipur® and “Tollwut-Impfstoff (human diploid cell [HDC]) inaktiviert”. These vaccines contain inactivated rabies virus. Both vaccines are recommended for pre- and postexposure use. Another example of a rabies vaccine is Imovax (Sanofi-Pasteur), which is a commercial inactivated human diploid cell vaccine. Rabies vaccines are in general administered according to the information of the manufacturer, whereby a typical post-exposure prophylaxis protocol includes administration of the vaccine at days 0, 3, 7, 14 and 28 after infection.

An antiviral refers to a class of medication used specifically for treating viral infections. Like antibiotics for bacteria, specific antivirals are used for specific viruses. Unlike most antibiotics, antiviral drugs do not destroy their target pathogen; instead they inhibit their development. For example, the antiviral is ribavirin.

For example, the antibody, or the antigen binding fragment thereof, according to the invention, or the pharmaceutical composition according to the invention are administered in a standard PEP scheme, e.g., in combination with a vaccine, for example, in the first treatment of the standard PEP scheme only.

A “standard PEP scheme” typically refers to the post-exposure prophylaxis scheme as recommended by the WHO (cf. http://www.who.int/rabies/human/WHO_strategy_prepost_exposure/en/index1.html#, retrieved at Nov. 12, 2014), wherein the antibodies according to the invention replace the RIGs, i.e. HRIG or ERIG. Namely, a post-exposure vaccination is started as soon as possible after exposure with a rabies vaccine as described herein, which follows the protocol of the manufacturer, typically at least two injections. For example, a standard protocol includes injections of the vaccine at days 0, 3, 7, 14, and 28 after exposure. Concomitantly to the first injection, or as soon as possible afterwards, the only and single dose of the antibody is administered.

When used in in post-exposure prophylaxis, treatment or attenuation of lyssavirus infection, the antibody, or the antigen binding fragment thereof, according to the invention is, for example, administered at a dose of 0.005 to 100 mg/kg or at a dose of 0.0075 to 50 mg/kg, such as at a dose of 0.01 to 10 mg/kg or at a dose of 0.01 to 1 mg/kg, e.g., at a dose of 0.01 to 0.1 mg/kg.

For example, the antibody, or the antigen binding fragment thereof, according to the invention, or the pharmaceutical composition according to the invention for use in post-exposure prophylaxis, treatment or attenuation of lyssavirus infection, is administered from 1 to 6 days or from 2 to 5 days, after infection or onset of symptoms.

In another embodiment, which does not refer to the standard PEP scheme, the antibody, or the antigen binding fragment thereof, according to the invention, may be administered without concomitant and/or subsequent administration of a vaccine.

In addition to administration in combination with a vaccine, e.g. in a standard PEP scheme, the antibodies according to the invention are also effective when administered without a vaccine, e.g. if administered more than one or two days after exposure.

Moreover, the antibody, or the antigen binding fragment thereof, according to the invention, or comprised in the pharmaceutical composition according to the invention, is administered at a dose of 0.01 to 100 mg/kg or at a dose of 0.1 to 75 mg/kg, such as at a dose of 1 to 60 mg/kg or at a dose of 10 to 50 mg/kg. Such “higher” doses are in particular useful if the exposure was severe and/or if treatment is initiated later than one or two days after exposure.

In one embodiment, the antibody, antibody fragment, or pharmaceutical composition according to the invention may be administered to a subject in need of such treatment. Such a subject includes, but is not limited to, one who is particularly at risk of or susceptible to RABV and/or non-RABV lyssavirus infection.

The invention also provides the use of the antibody or an antigen-binding fragment thereof according to the invention, or the pharmaceutical composition of the invention in (i) the manufacture of a medicament for the treatment or attenuation of infection by RABV and/or non-RABV lyssavirus, (ii) a vaccine, or (iii) diagnosis of RABV and/or non-RABV lyssavirus infection.

Methods of diagnosis may include contacting an antibody or an antibody fragment with a sample. Such samples may be isolated tissue samples, for example, taken from nasal passages, sinus cavities, salivary glands, lung, liver, pancreas, kidney, ear, eye, placenta, alimentary tract, heart, ovaries, pituitary, adrenals, thyroid, brain or skin. The methods of diagnosis may also include the detection of an antigen/antibody complex.

Antibodies and fragments thereof as described in the invention may also be used in a kit for the diagnosis of RABV and/or non-RABV lyssavirus infection. Antibodies or antibody fragments, as described herein, may also be used in a kit for monitoring vaccine manufacture with the desired immunogenicity.

### BRIEF DESCRIPTION OF THE FIGURES

In the following a brief description of the appended figures will be given. The figures are intended to illustrate the invention in more detail. However, they are not intended to limit the subject matter of the invention in any way.

FIG. 1 Experimental model of rabies infection using Balb/c mice. (A) Survival rate of 8 weeks female Balb/c mice infected with 4000 FFU of a field RABV strain (isolate 8743THA). (B) Body weight of mice indicating a weight loss starting at 4 days post-infection in infected mice. (C) Clinical signs of infected mice, characterized as ‘sick’ (lethargy, ruffled fur) or ‘sick and paralyzed’. (D) Rabies virus quantification in the brainstem and in the cerebral cortex of infected mice. Each conditions corresponds to 5 mice.

FIG. 2 Intramuscular (IM) therapy using an experimental model of rabies infection. (A) Experimental plan of the intramuscular therapy at 2, 4, 6 or 8 days post-infection using two different doses of a cocktail of two monoclonal antibodies (RVC20+RVC58). (B, D, F) Survival rate of the mice from the three experimental groups: untreated (B), treated at 2+2 mg/kg (D) and treated at 20 mg/kg (F). (C, E, G) Body weight of the mice from the three experimental groups: untreated (C), treated at 2+2 mg/kg (E) and treated at 20 mg/kg (G). Each condition corresponds to 5 mice.

FIG. 3 Serological and molecular analyses of mice treated with the intramuscular therapy. (A-B) Mouse neutralizing antibodies (A) and human antibodies (B) detected in the mice from the different groups: untreated, treated at 2+2 mg/kg and treated at 20 mg/kg. (C) Rabies virus detection in the brain of mice from the different groups. (D-E). Relative quantification of the chemokines CCL-5 (D) and CXCL-10 (E) in the brain of mice from the different groups. Samples were collected either in the moment of the death (full squares) or at 100 days post-infection for the survivors (open squares).

FIG. 4 Intracerebroventricular (ICV) and intramuscular (IM) therapy in early rabies virus infection. (A) Experimental plan of the continuous ICV therapy alone or continuous ICV therapy coupled with one IM injection starting six days post-infection using a cocktail of two monoclonal antibodies (RVC20+RVC58). (B, D) Survival rate of the mice from the three experimental groups: untreated (B), treated with ICV alone or ICV+IM (D). (C, E) Body weight of the mice from the three experimental groups: untreated (C), treated with ICV alone or ICV+IM (E). Each condition corresponds to 5 mice.

FIG. 5 Serological and molecular analyses of mice treated with the intracerebroventricular (ICV) and intramuscular (IM) therapy in early rabies virus infection. (A-B) Mouse neutralizing antibodies (A) and human antibodies (B) detected in the mice from the different groups: untreated, treated with ICV alone or ICV+IM. (C) Rabies virus detection in the brain of mice from the different groups. (D-E). Relative quantification of the chemokines CCL-5 (D) and CXCL-10 (E) in the brain of mice from the different groups. Samples were collected either in the moment of the death (full squares) or at 100 days post-infection for the survivors (open squares).

FIG. 6 Intracerebroventricular (ICV) and intramuscular (IM) therapy in late rabies infection. (A) Experimental plan of the ICV therapy coupled with one IM injection starting seven or eight days post-infection using a cocktail of two monoclonal antibodies (RVC20+RVC58). (B, D) Survival rate of the mice from the three experimental groups: untreated (B), treated at day 7 post-infection or at day 8 post-infection (D). (C, E) Body weight of the mice from the three experimental groups: untreated (C), treated at day 7 post-infection or at day 8 post-infection (E). (F). Clinical signs profile of the infected mice treated at day 7 post-infection or at day 8 post-infection. Each condition corresponds to 5 mice.

FIG. 7 Serological and molecular analyses of mice treated with the intracerebroventricular (ICV) and intramuscular (IM) therapy in late rabies virus infection. (A-B) Mouse neutralizing antibodies (A) and human antibodies (B) detected in the mice from the different groups: untreated, treated at day 7 post-infection or treated at day 8 post-infection. (C) Rabies virus detection in the brain of mice from the different groups. (D-E). Relative quantification of the chemokines CCL-5 (D) and CXCL-10 (E) in the brain of mice from the different groups. Samples were collected either in the moment of the death (full squares) or at 61 days post-infection for the survivors (open squares).

FIG. 8 Intracerebroventricular (ICV) and intramuscular (IM) therapy in late rabies infection. (A) Experimental plan of the ICV therapy starting eight days post-infection, coupled with one or two IM injections, using a cocktail of two monoclonal antibodies (RVC20+RVC58). (B, D) Survival rate of the mice from the experimental groups: untreated (B), ICV coupled with one IM injection, ICV coupled with two IM injections, ICV coupled with one IM injection using LALA monoclonal antibodies, and ICV coupled with one IM injection using monoclonal antibodies against the Respiratory Syncytial Virus (D). (C, E) Body weight of the mice from the different experimental groups: untreated (C) and treated (E). (F). Clinical signs profile of the infected mice from the different experimental groups. Each condition corresponds to 5 mice.

FIG. 9 Serological and molecular analyses of mice treated with the intracerebroventricular (ICV) and intramuscular (IM) therapy in late rabies virus infection. (A-B) Mouse neutralizing antibodies (A) and human antibodies (B) detected in the mice from the different groups: untreated, ICV coupled with one IM injection, ICV coupled with two IM injections, ICV coupled with one IM injection using LALA monoclonal antibodies, and ICV coupled with one IM injection using monoclonal antibodies against the Respiratory Syncytial Virus. (C) Rabies virus detection in the brain of mice from the different groups. (D-J). Relative quantification of the chemokines, cytokines and antiviral mediators in the brain of mice from the different groups. Samples were collected either in the moment of the death (full squares) or at 61 days post-infection for the survivors (open squares). * indicates P<0.05, determined using group wise comparison (REST^{∘}-MCS).

FIG. 10 Histological analyses of mice treated with the intracerebroventricular (ICV) and intramuscular (IM) therapy in late rabies virus infection. Intensity of inflammation (measured by lba1+microglial cells detection) and presence of Rabies virus in the brain of mice from the different groups: uninfected (A), uninfected and treated (B), infected and non-treated (C), infected and treated at day 7 post-infection (p.i.) with ICV coupled with one IM injection (D), infected and treated at day 8 p.i. with ICV coupled with one IM injection (E), infected and treated at day 8 p.i. with ICV coupled with two IM injections (F), infected and treated at day 8 p.i. with ICV coupled with one IM injection using LALA monoclonal antibodies (G), and infected and treated at day 8 p.i. with ICV coupled with one IM. The number of animals in each condition is indicated in the graphs. Related to FIGS. 7-9.

### EXAMPLES

In the following, particular examples illustrating various embodiments and aspects of the invention are presented. However, the invention shall not to be limited in scope by the specific embodiments described herein. The following preparations and examples are given to enable those skilled in the art to more clearly understand and to practice the invention. The invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Characterization of Model of Establishing (Lethal) RABV Disease in Mice

To investigate whether anti-lyssavirus antibodies display neutralizing activity against a lethal RABV infection in vivo, a model of RABV infection in wild-type mice (BALB/c) was developed.

To this end, a RABV strain obtained from the brain of a human bitten by a rabid dog in Thailand (Tha) was used. Briefly, the Tha RABV strain (4000 FFU, focus forming units) was administered to wild-type mice (Balb/c) intramuscularly in a total volume of 100 μl into the gastrocnemius muscle of both hind legs (2 injections of 25 μl in each leg). Thereafter, animals were monitored and were euthanized when symptoms of clinical rabies occurred, such as paralysis, lethargy, ruffled fur.

After sacrifice, brains were removed to quantify the viral load in CNS tissues. Brains were removed and separated in two hemispheres; one hemisphere was fixed in 4% formalin and the other hemisphere was stored at −80° C. Total RNA was extracted from the brains stored at −80° C. using trizol and the phenol:chlorophorm method. RNA isolation was performed on the clear upper aqueous layer with the RNeasy Mini kit (74104, Qiagen) according to the manufacturer's instructions. Total RNAs were reverse transcribed to first strand cDNA using RT² First Strand (330401, Qiagen). qPCR was performed in a final volume of 20 μL per reaction in 96-well PCR plates using a thermocycler (7500t real time PCR system, Applied Biosystems) in triplicates. Briefly, 5 μL of cDNA (25 ng) was added to 15 μL of a master mix containing 10 μL of QuantiTect SYBR Green PCR Kit (204143, Qiagen) and 5 μL of nuclease-free water with primers at a final concentration of 0.5 μM. The amplification conditions were as follows: 95° C. for 15 min, 45 cycles of 94° C. for 15 s and 60° C. for 1 min; followed by a melt curve, from 60° C. to 95° C. The gene of the nucleoprotein was used to quantification of the viral load in the brains, which was assessed by linear regression using a standard curve of eight known quantities of plasmids containing the nucleoprotein sequence (ranging from 10⁸ to 10¹ copies). Primers used for the amplification were the following: forward primer 5′-CTG ACG TAG CAC TGG CAG AC-3′ (SEQ ID NO: 225) and reverse primer 5′-AGT CGA CCT CCG TTC ATC AT-3′ (SEQ ID NO: 226). The amplicons were 117 nucleotide long.

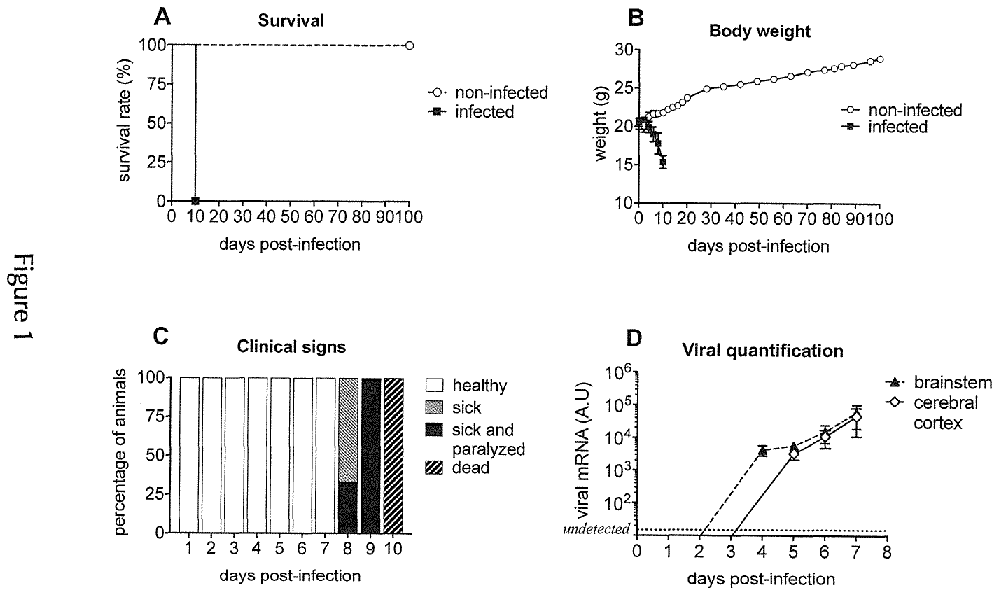

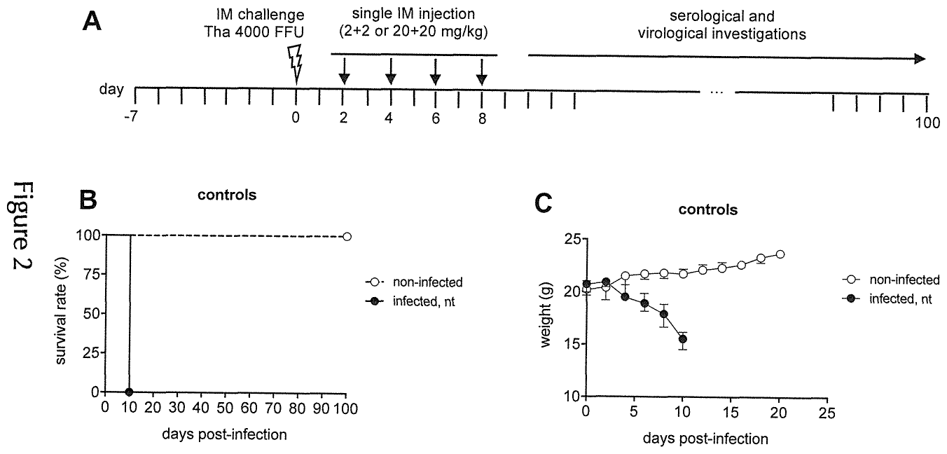

Results are shown in FIG. 1. After RABV injection, animals continuously lost body weight and all animals succumbed on day 10 after RABV exposure (FIG. 1A-B). The quantification of the virus in CNS tissues showed that the virus appears in the brainstem and cerebral cortex on day 4 and 5, respectively (FIG. 1C). The first evident symptoms of disease (paralysis, lethargy, ruffled fur) appeared on day 8 after RABV exposure (FIG. 1D).

### Example 2: Effects of Peripheral Delivery of Anti-Lyssavirus Antibodies on Established RABV Disease

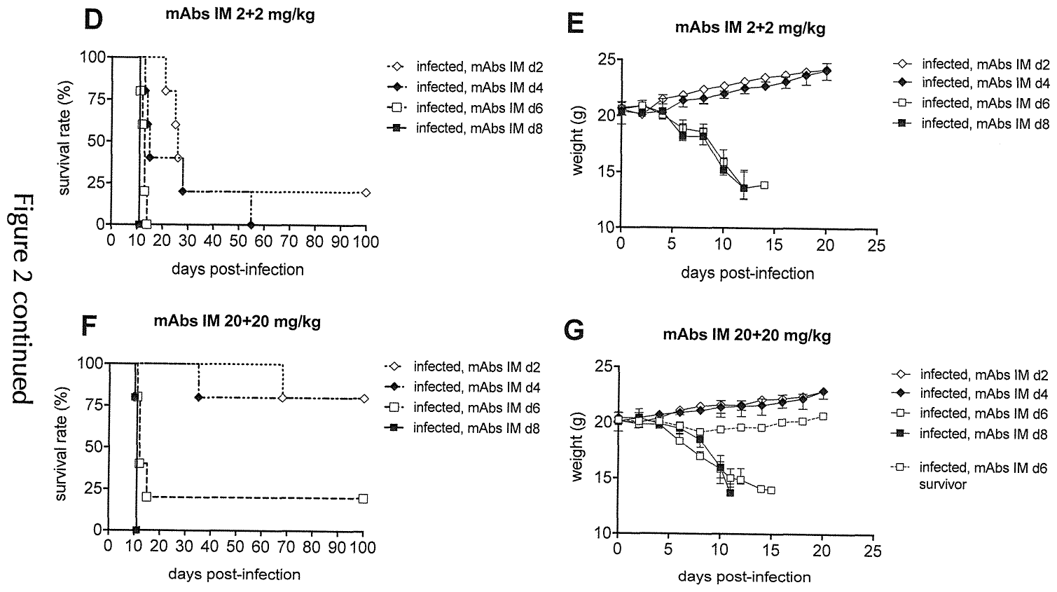

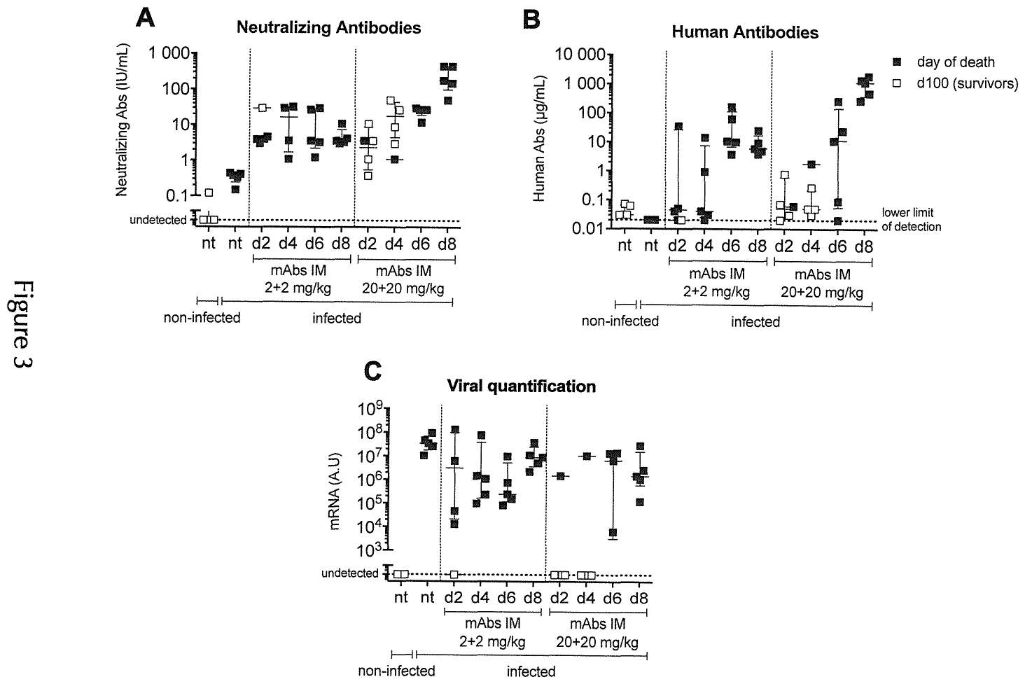

In the model described in Example 1, the therapeutic potential of intramuscular administration of anti-lyssavirus antibodies was assessed at different time points after RABV exposure. To this end, different groups of mice (n=5 per group) received a single i.m. dose (2+2 or 20+20 mg/kg, in two injections of 25 μl in each gastrocnemius muscle of both hind legs) of the 1:1 combination of RVC58+RVC20 anti-lyssavirus human monoclonal antibodies at different time points after RABV exposure, namely, on day 2, 4, 6 or 8 after injection with a lethal dose (4000 FFU) of Tha RABV strain (FIG. 2A). In addition, one control group was not exposed to RABV and another control group did not receive treatment with RVC58+RVC20 anti-Lyssavirus human monoclonal antibodies after RABV exposure. Body weight and survival rates were observed and recorded for all animals. After sacrifice, brains were removed and blood samples of each animal were collected.

Blood samples were collected by cardiac puncture and serum was recovered after coagulation and centrifugation at 2,000×g during 30 minutes at 4° C. Serum samples were stored at −20° C. until analyses. Mouse neutralising antibody levels were measured by an adaptation of the rapid fluorescent focus inhibition test (REFIT) for 96 well plates (Bourhy H, Sureau P (1990) Laboratory methods for rabies diagnosis. Paris: Institut Pasteur. pp. 191-3; Feyssaguet M, Dacheux L, Audry L, Compoint A, Morize J L, et al. (2007) Multicenter comparative study of a new ELISA, PLATELIA RABIES II, for the detection and titration of anti-rabies glycoprotein antibodies and comparison with the rapid fluorescent focus inhibition test (REFIT) on human samples from vaccinated and non-vaccinated people. Vaccine 25: 2244-51). Briefly, a constant dose of challenge virus standard (CVS) is incubated with diluted test sera. An in-house reference serum is calibrated against an international standard. Serum/virus mixtures are incubated, and BSR cells (a clone of BHK-21 cells) were added. After 24 hours incubation, the monolayer is acetone-fixed and stained with a fluorescent anti-nucleocapsid antibody (Chemicon). The result in IU/ml was the mean of independent duplicate tests.

A standard ELISA was used to determine binding of serum human antibodies to RABV G protein (CVS-11). Briefly, ELISA plates were coated with RABV G protein at 5 μg/ml, blocked with 10% FCS in PBS, incubated with sera or human antibodies, and washed. Bound antibodies were detected by incubation with AP-conjugated goat anti-human IgG (Southern Biotech). Plates were then washed, substrate (p-NPP, Sigma) was added and plates were read at 405 nm. Quantification was performed using an internal standard based on a human IgG1 monoclonal antibody.

Brains were removed and separated in two hemispheres; one hemisphere was fixed in 4% formalin and the other hemisphere was stored at −80° C. Total RNA was extracted from the brains stored at −80° C. using trizol and the phenol:chlorophorm method. RNA isolation was performed on the clear upper aqueous layer with the RNeasy Mini kit (74104, Qiagen) according to the manufacturer's instructions. Total RNAs were reverse transcribed to first strand cDNA using RT² First Strand (330401, Qiagen). qPCR was performed in a final volume of 20 μL per reaction in 96-well PCR plates using a thermocycler (7500t real time PCR system, Applied Biosystems) in triplicates. Briefly, 5 μL of cDNA (25 ng) was added to 15 μL of a master mix containing 10 μL. of QuantiTect SYBR Green PCR Kit (204143, Qiagen) and 5 μL of nuclease-free water with primers at a final concentration of 0.5 μM. The amplification conditions were as follows: 95° C. for 15 min, 45 cycles of 94° C. for 15 s and 60° C. for 1 min; followed by a melt curve, from 60° C. to 95° C. The gene of the nucleoprotein was used to quantification of the viral load in the brains, which was assessed by linear regression using a standard curve of eight known quantities of plasmids containing the nucleoprotein sequence (ranging from 10⁸ to 10¹ copies). Primers used for the amplification were the following: forward primer 5′-CTG ACG TAG CAC TGG CAG AC-3′ (SEQ ID NO: 225) and reverse primer 5′-AGT CGA CCT CCG TTC ATC AT-3′ (SEQ ID NO: 226). The amplicons were 117 nucleotide long. The mouse gene targets were selected for quantifying host inflammatory mediators transcripts in the brain (249900, Qiagen) using GAPDH gene as a reference.

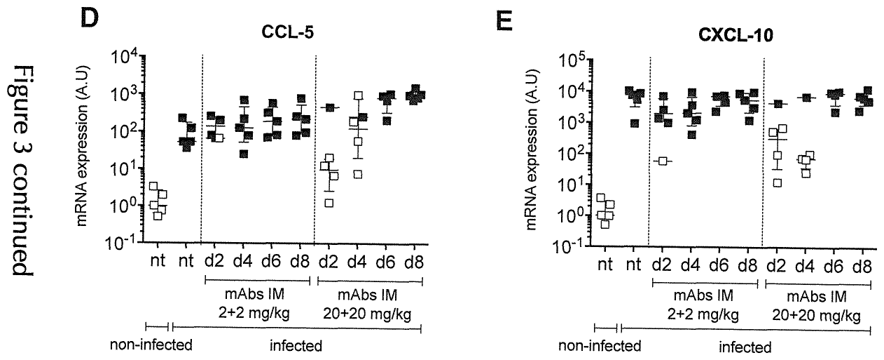

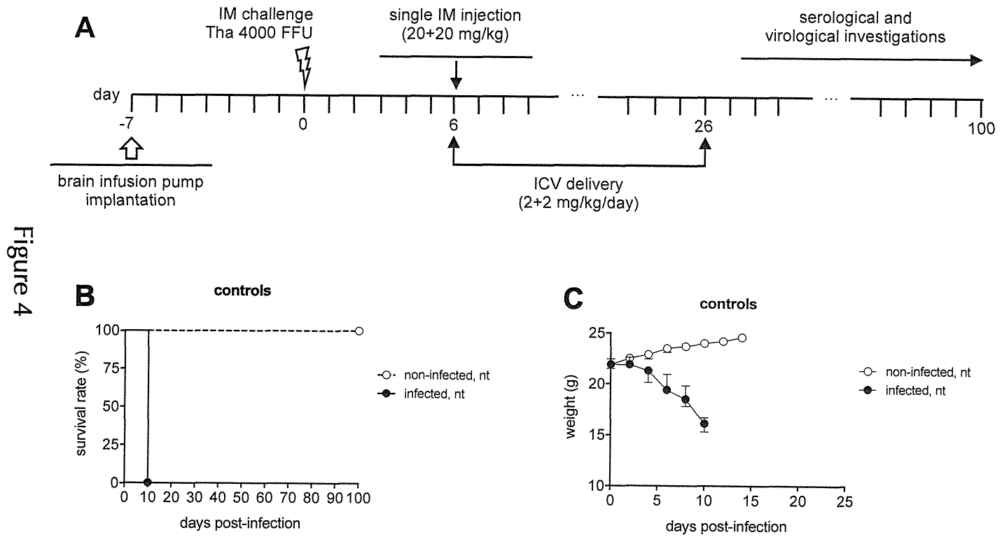

Results are shown in FIGS. 2 and 3. Similar to Example 1, all infected but untreated control animals succumbed by day 10 after infection with a lethal dose (4000 FFU) of Tha RABV strain (FIG. 2B-C). When administered at 2+2 mg/kg RVC58+RVC20 human monoclonal antibodies provided a modest effect when administered 2 or 4 days after RABV infection, while administration of antibodies on day 6 or 8 did not prevent morbidity and mortality (FIG. 2D-E). When administered at 20+20 mg/kg RVC58+RVC20 human monoclonal antibodies protected almost all animals from morbidity and mortality when administered 2 or 4 days after infection, and showed a modest effect when administered on day 6. However, administration of antibodies on day 8 did not prevent morbidity and mortality (FIG. 2F-G). Surviving animals were monitored until day 100 and did not further develop signs of disease. These data show that in animals treated for the first (and only) time at day 2 or 4 after RABV infection even a very low dose of antibodies resulted in at least modest treatment efficacy. When animals were treated on day 6 after RABV infection (for the first and only time), a higher dose of antibodies was required to achieve a modest treatment effect. However, neither the low nor the higher dose of antibodies was sufficient, if antibodies were administered only on day 8 after RABV infection.

In addition, the levels of neutralizing antibodies as well as the level of human antibodies were measured in serum of the animals at the day of sacrifice (death or at the end of the observation period for surviving animals, i.e. day 100; FIG. 3A-B). This high level of neutralizing antibodies found in peripheral blood was, however, not capable of preventing morbidity and mortality in animals receiving neutralizing antibodies on day 8.

The measurement of viral mRNA present in the brain clearly showed that no virus was detectable in the brain of surviving animals (FIG. 3C). In contrast, in all succumbing animals virus was detectable in the brain and only a partial reduction of RABV viral titers was observed in treated succumbing animals (FIG. 3C).

Finally, measurement CCL5 (Chemokine (C-C motif) ligand 5, also known as RANTES (regulated on activation, normal T cell expressed and secreted)) and CXCL10 (C-X-C motif chemokine 10, also known as Interferon gamma-induced protein 10 (IP-10)) mRNA levels in the brain revealed increased levels of CCL5 and CXCL10 mRNA levels in diseased animals, with highest levels observed in succumbed animals treated on day 6 or 8 (FIG. 3D-F). These results indicate that treatment with RVC58+RVC20 human monoclonal antibodies did not inhibit the activation of inflammation, as measured by CCL5 and CXCL10.

Taken together, these results show that even the presence of high concentrations of neutralizing antibodies in the blood is not sufficient to prevent from morbidity and mortality. In particular, in all succumbed animals, RABV was detectable in the CNS—whereas no RABV could be detected in surviving animals. However, without being bound to any theory, antibodies cannot cross the blood-brain barrier and, thus, the results suggest that peripherally administered neutralizing antibodies and neutralizing antibodies produced endogenously fail to “reach” (and neutralize) the virus in the CNS. However, in particular on days 6 and 8 after RABV injection the virus is detectable in the CNS (as shown in Example 1, FIG. 1C). Without being bound to any theory, antibodies administered at days 6 or 8 cannot neutralize the virus anymore, which is already located in the CNS and due to the blood-brain barrier “out of reach” for the antibodies outside the CNS.

### Example 3: Effects of Intra-CNS Delivery of Anti-Lyssavirus Antibodies on Established RABV Disease

In view of the results of Example 2, the effects of intra-CNS administration of anti-lyssavirus antibodies at different time points was investigated.

For intra-CNS administration, mice were equipped with mini pumps. Micro Infusion Pump REF SMP-300 (iPrecio, Japan) were used for the intracerebroventricular delivery. The ability to program the device to start, stop and deliver different doses at different time points or just deliver one continuous dose makes these pumps ideally suited to monitor the amount of drug delivery. Pumps were programmed using a PC based application software [iPrecio Management Software IMS-300, iPrecio, Japan]. The device comprises a microinfusion pump connected by a flexible tube to a cannula [Alzet Brain Infusion Kit 3, 0008851, Durect Corporation, United States of America], which was inserted by stereotaxic surgery in order to deliver the selected solution by the intracerebroventricular route. The pump is initiated just before surgery.

All surgical procedures and postoperative care were reviewed and approved by the Animal Care and Use Committee of Institut Pasteur (Paris, France), and were carried out in accordance with European Directive 86/609-2010/63/UE guidelines for animal experimentation. Female two-month old BALB/c mice were anesthetized with ketamine (100 mg/kg, i.p.) and xylazine (10 mg/kg, i.p.). Sedation was monitored using a gentle toe pinch withdraw reflex. Thermoregulation was provided through a thermostat-regulated heating pad. Head was shaved of fur and cleaned with iodine before incision. After skin incision (about 1 cm) and removal of all soft tissue from the surface of the skull, placement of the cannula was determined in relation to bregma. A 1 mm hole was drilled through the skull with a battery-operated driller designed for rodent surgery (Brenda M. Geiger, Lauren E. Frank, Angela D. Caldera-Siu, and Emmanuel N. Pothos. Survivable Stereotaxic Surgery in Rodents. J Vis Exp. 2008; (20): 880). Care was taken so that the drill bit does not penetrate through meningeal membranes or blood vessels. The stereotaxic coordinates were established as per Franklin and Paxinos, 1997 (The Mouse Brain in Stereotaxic Coordinates, Academic Press). A cannula having a diameter of 0.31 mm was inserted at the following stereotaxic coordinates: antero-posterior (in relation to bregma): −0.5 mm; medio-lateral: +1 mm; dorso-ventral (from brain surface): −2 mm and the implants were secured by cement. The pumps were implanted subcutaneously in dorsolateral site.

At the end of surgery a bolus of 0.9% saline is given (1 ml s.c. after fluids were warmed to normal body temperature) to prevent dehydration. Buprenorphine (0.1-0.5 mg/kg s.c.) is administered twice daily on the first day after surgery. Following surgery, animals were individually housed. At least one week was allowed for recovery before virus challenge. Solutions were administered at a flow rate of 1 μl/h for 20 days. The solution in the pump was changed every 4 days. Animals received PBS until the start of the experiment.

At the end of the experiment the animals were euthanized and brains were removed and separated in two hemispheres; one hemisphere was fixed in 4% formalin and the other hemisphere was stored at −80°.

### Intracerebroventricular Delivery of the Human Monoclonal Antibodies

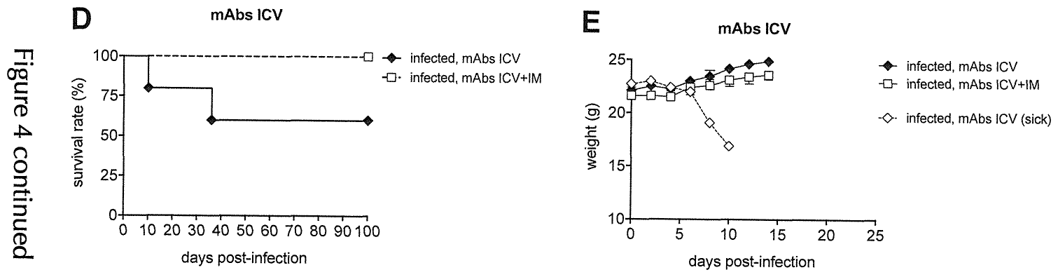

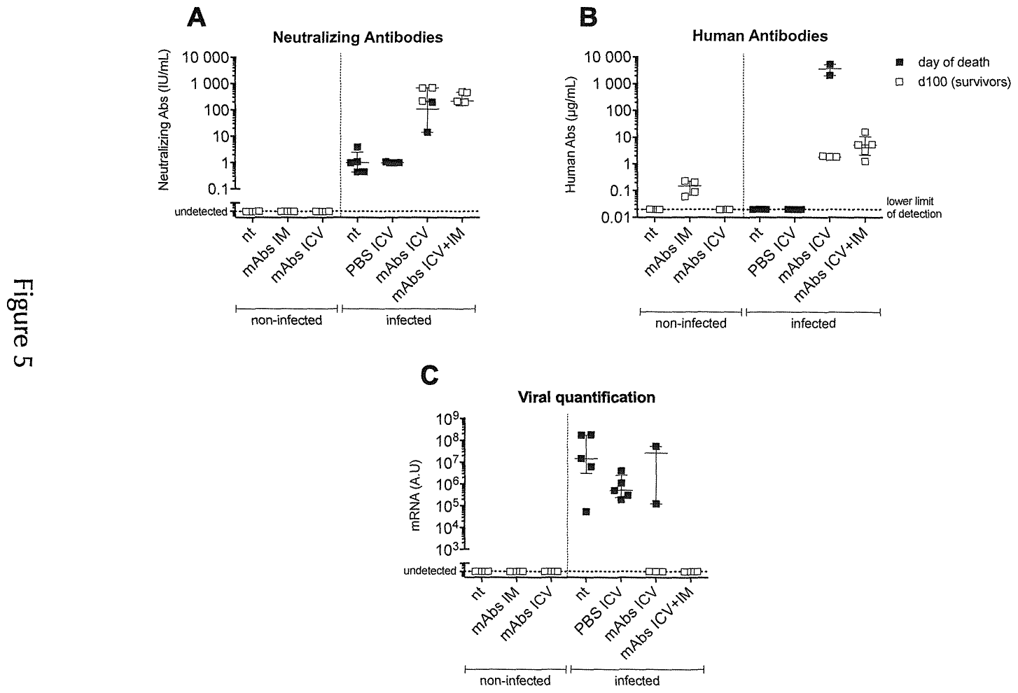

Brain infusion pump was implanted 7 days before intramuscular challenge with Tha RABV strain (4000 FFU; as described above) to allow for intracerebroventricular (icy) administration of antibodies. Six days after RABV infection (in particular before the appearance of the first symptoms) animals started receiving 2+2 mg/kg/day of RVC58+RVC20 human monoclonal antibodies or PBS (control animals) through the implanted pump for 20 days (FIG. 4A). As an additional control, the same experimental approach was also performed on pump-equipped non-infected animals to evaluate effect of intraventricular administration of antibodies or PBS.

Results are shown in FIG. 4. Infected mice receiving only PBS by icy administration did not survive (FIG. 4B-4C). However, despite delivery of the antibodies directly into the CNS, this treatment provided only a partial efficacy resulting in the survival of 3 out of 5 infected mice (FIG. 4D-E). These data suggest that a further delay in the start of the intracerebroventricular (icy) administration at the appearance of symptoms (e.g., clay 8 after RABV injection) could be even less effective.

### Example 4: Effects of Combined Intra-CNS and Peripheral Delivery of Anti-Lyssavirus Antibodies on Established RABV Disease

In view of the results of Examples 2 and 3, a combined approach of peripheral and intra-CNS delivery of anti-Lyssavirus antibodies was investigated.

To this end, brain infusion pumps were implanted 7 days before intramuscular challenge with Tha RABV strain (4000 FFU) as described in Example 3 to allow for the intracerebroventricular (ICV) administration of antibodies. Six days after RABV infection (in particular before the appearance of the first symptoms) animals started receiving 2+2 mg/kg/day of RVC58+RVC20 human monoclonal antibodies or PBS (control animals) through the implanted pump for 20 days (FIG. 4A). Animals were also administered with a single intramuscular (IM) injection of RVC58+RVC20 (at 20 mg/kg+20 mg/kg) on day 6.

Body weight and survival rates were observed and recorded for all animals. After sacrifice, brains were removed and blood samples of each animal were collected.

Blood samples were collected by cardiac puncture and serum was recovered after coagulation and centrifugation at 2,000×g during 30 minutes at 4° C. Serum samples were stored at −20° C. until analyses.

Mouse neutralising antibody levels were measured by an adaptation of the rapid fluorescent focus inhibition test (RFFIT) for 96 well plates (Bourhy H, Sureau P (1990) Laboratory methods for rabies diagnosis. Paris: Institut Pasteur. pp. 191-3; Feyssaguet M, Dacheux L, Audry L, Compoint A, Morize J L, et al. (2007) Multicenter comparative study of a new ELISA, PLATELIA RABIES II, for the detection and titration of anti-rabies glycoprotein antibodies and comparison with the rapid fluorescent focus inhibition test (RFFIT) on human samples from vaccinated and non-vaccinated people. Vaccine 25: 2244-51). Briefly, a constant dose of challenge virus standard (CVS) is incubated with diluted test sera. An in-house reference serum is calibrated against an international standard. Serum/virus mixtures are incubated, and BSR cells (a clone of BHK-21 cells) were added. After 24 hours incubation, the monolayer is acetone-fixed and stained with a fluorescent anti-nucleocapsid antibody (Chemicon). The result in IU/ml was the mean of independent duplicate tests.

A standard ELISA was used to determine binding of serum human antibodies to RABV G protein (CVS-11). Briefly, ELISA plates were coated with RABV G protein at 5 μg/ml, blocked with 10% FCS in PBS, incubated with sera or human antibodies, and washed. Bound antibodies were detected by incubation with AP-conjugated goat anti-human IgG (Southern Biotech). Plates were then washed, substrate (p-NPP, Sigma) was added and plates were read at 405 nm. Quantification was performed using an internal standard based on a human IgG1 monoclonal antibody.

Brains were removed and separated in two hemispheres; one hemisphere was fixed n 4% formalin and the other hemisphere was stored at −80° C. Total RNA was extracted from the brains stored at −80° C. using trizol and the phenol:chlorophorm method. RNA isolation was performed on the clear upper aqueous layer with the RNeasy Mini kit (74104, Qiagen) according to the manufacturer's instructions. Total RNAs were reverse transcribed to first strand cDNA using RTZ First Strand (330401, Qiagen). qPCR was performed in a final volume of 20 μL per reaction in 96-well PCR plates using a thermocycler (7500t real time PCR system, Applied Biosystems) in triplicates. Briefly, 5 μL of cDNA (25 ng) was added to 15 μL of a master mix containing 10 μL of QuantiTect SYBR Green PCR Kit (204143, Qiagen) and 5 of nuclease-free water with primers at a final concentration of 0.5 μM. The amplification conditions were as follows: 95° C. for 15 min, 45 cycles of 94° C. for 15 s and 60° C. for 1 min;

followed by a melt curve, from 60° C. to 95° C. The gene of the nucleoprotein was used to quantification of the viral load in the brains, which was assessed by linear regression using a standard curve of eight known quantities of plasmids containing the nucleoprotein sequence (ranging from 10⁸ to 10¹ copies). Primers used for the amplification were the following: forward primer 5′-CTG ACG TAG CAC TGG CAG AC-3′ (SEQ ID NO: 225) and reverse primer 5′-AGT CGA CCT CCG TTC ATC AT-3′ (SEQ ID NO: 226). The amplicons were 117 nucleotide long. The mouse gene targets were selected for quantifying host inflammatory mediators transcripts in the brain (249900, Qiagen) using GAPDH gene as a reference.

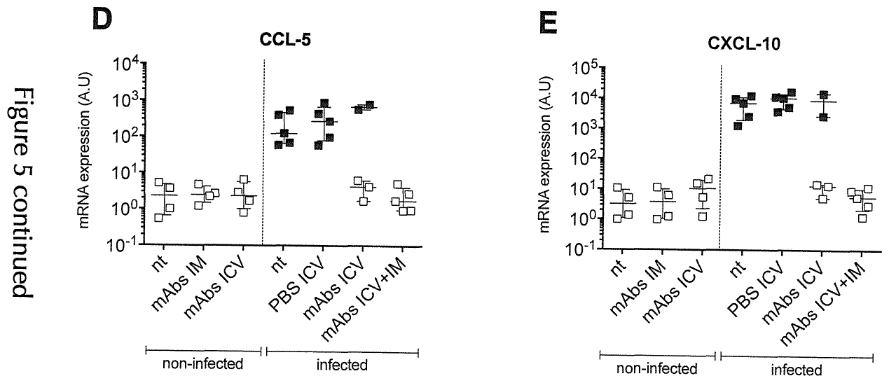

Results are shown in FIGS. 4 and 5. Surprisingly, the combined administration into the CNS and into the periphery provided protection from mortality (FIG. 4D) and morbidity (FIG. 4E) of all 5 out of 5 infected mice. Of note, these data show a pronounced effect of combined administration into the CNS and into the periphery as administration to the periphery only (on day 6 after RABV infection) protected only one out of five mice (Example 2, FIG. 2F) and administration to the CNS only protected only three out of five mice (Example 3, FIG. 4D). Again, the levels of neutralizing antibodies as well as the level of human antibodies were measured in serum of the animals at the day of sacrifice (death or at the end of the observation period for surviving animals, i.e. day 100; FIG. 5A-B). High levels of neutralizing antibodies were found in blood, which correspond to a combination of active production of mice antibodies and of persistence of human antibodies resulting from the ICV and IM injection of monoclonal antibodies.

Similar to Example 2, the measurement of viral mRNA showed that virus was not detected in the CNS of surviving animals at the end of the study (i.e. 100 days after infection) (FIG. 5C).

As measured on day 100 in antibody-treated surviving animals and as compared to control uninfected animals, there was no evidence of activation of inflammation, as measured by the CCL-5 and CXCL-10 mRNA levels (FIG. 5D-H). These results indicate that surviving animals did not show signs of CNS inflammation at the end of the study.

### Example 5: Effects of Combined Intra-CNS and Peripheral Delivery of Anti-Lyssavirus Antibodies on Established RABV Disease at Day 7 or 8 after RABV Infection

In view of the remarkable results of Example 4, the hypothesis was tested that the combined administration of anti-lyssavirus antibodies into CNS and periphery is also effective when started at later time points (after RABV infection).

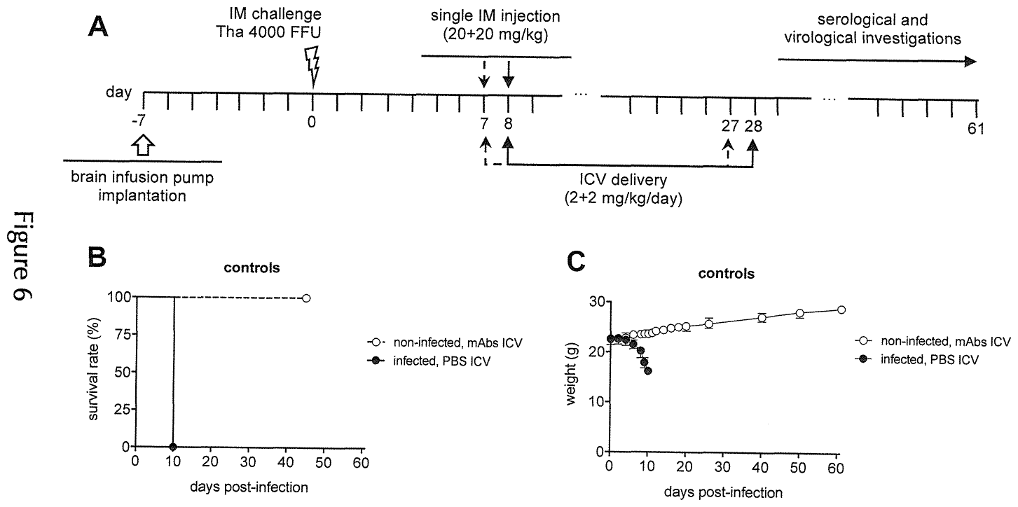

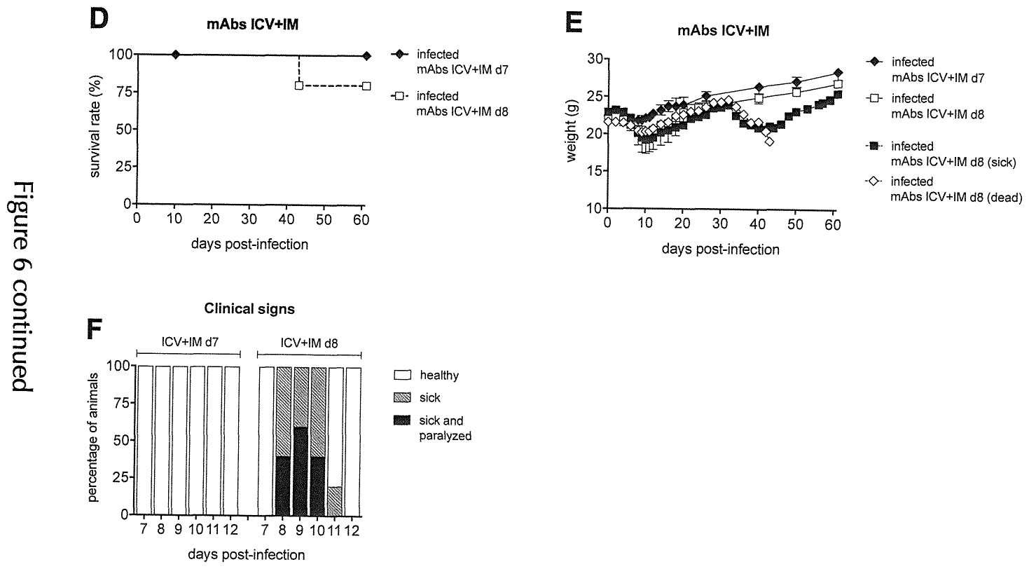

To this end, the experiment described in Example 4 was repeated (same conditions, same doses) with the only difference that icy administration started on day 7 or day 8 after RABV infection (for 20 days) and i.m. administration was performed by a single injection on the first day of icy administration (day 7 or day 8, respectively, after RABV infection) (FIG. 6A). Of note, on day 8 all animals developed clear symptoms of disease, i.e. paralysis and/or sickness (cf. Example 1, FIG. 1D).

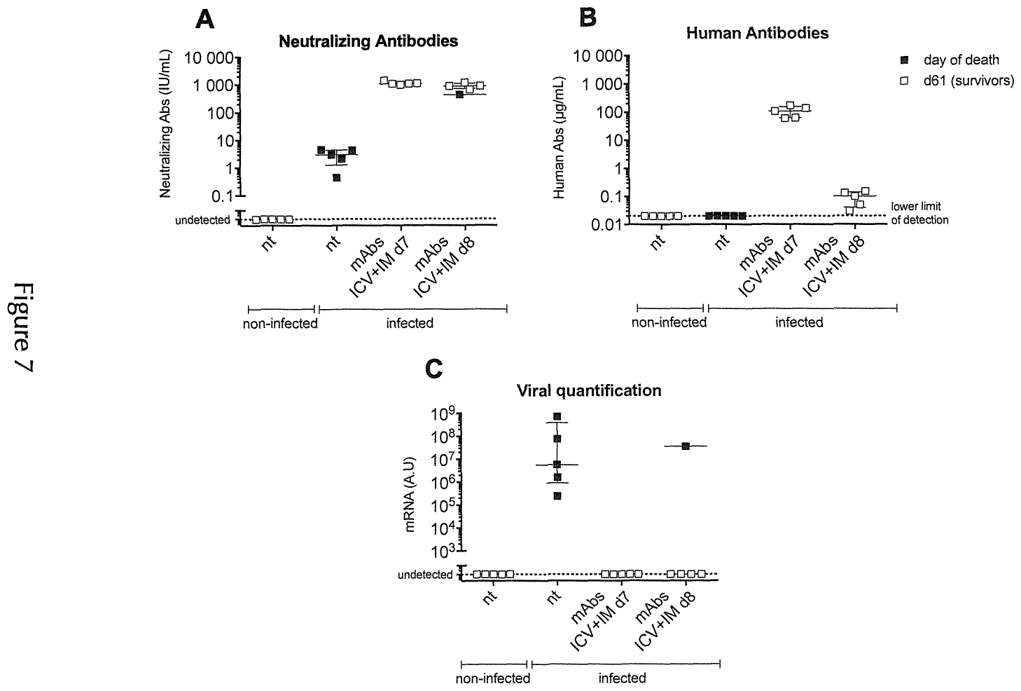

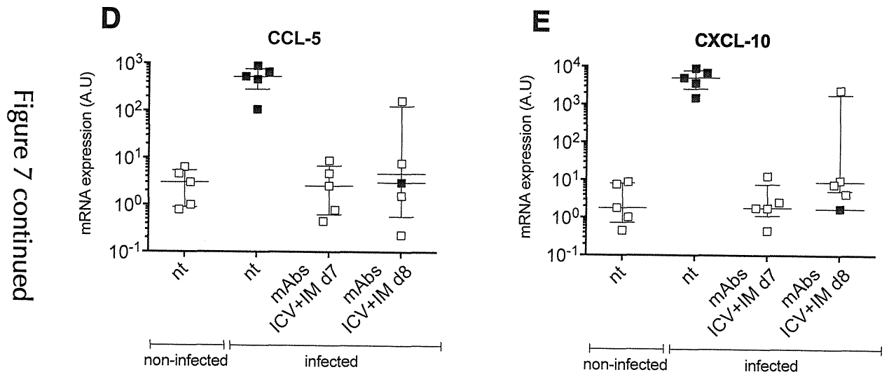

Results are shown in FIGS. 6 and 7. Surprisingly, all animals treated on day 7 survived. Moreover, all animals treated on day 8 survived until day 42 (when one animal succumbed), i.e. 4 out 5 animals of those treated on day 8 survived long-term (FIG. 6D-E). In the d-8 group, in surviving animals all rabies symptoms that were present on day 8 disappeared on day 12 (FIG. 6F).

The measurement of viral mRNA in the brain showed that virus was not detected in the CNS of surviving animals at the end of the study. In addition, in antibody-treated surviving animals and as compared to control uninfected animals, there was no evidence of activation of inflammation, as measured by the CCL-5 and CXCL10 mRNA levels (FIG. 7D-E). These results indicate that surviving animals did not show signs of CNS inflammation at the end of the study. The results of this experiment clearly indicate that combined administration of anti-lyssavirus antibodies into the CNS and into the periphery is effective in treating symptomatic lyssavirus infection, such as rabies disease.

### Example 6: Combined Intra-CNS and Peripheral Delivery of Anti-Lyssavirus Antibodies Devoid of Effector Functions on Established RABV Disease

To understand if the therapeutic activity of RVC58+RVC20 human monoclonal antibodies relies also on Fc-dependent effector functions (i.e. antibody-dependent cellular cytotoxicity, antibody-dependent cellular phagocytosis and/or complement-dependent cytotoxicity), the Fc region of IgG1 RVC58+RVC20 human monoclonal antibodies was engineered to introduce the “LALA” mutation (CH2 4, CH2 5; e.g., L234A, L235A) into the antibody's heavy chain. To obtain the LALA variant, each of the heavy chains was mutated at amino acids 4 and 5 of CH2 domain by substituting an alanine in place of the natural leucine using site-directed mutagenesis. The “LALA” mutation was shown to abrogate the binding to complement and Fc-gamma receptors. Moreover, this mutation is known to not alter the in vivo pharmacokinetics of human antibodies.

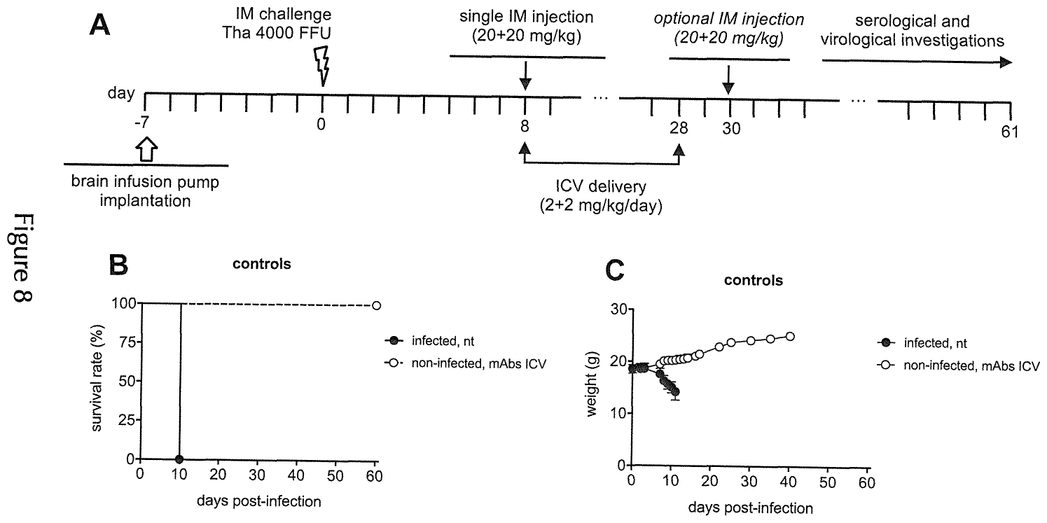

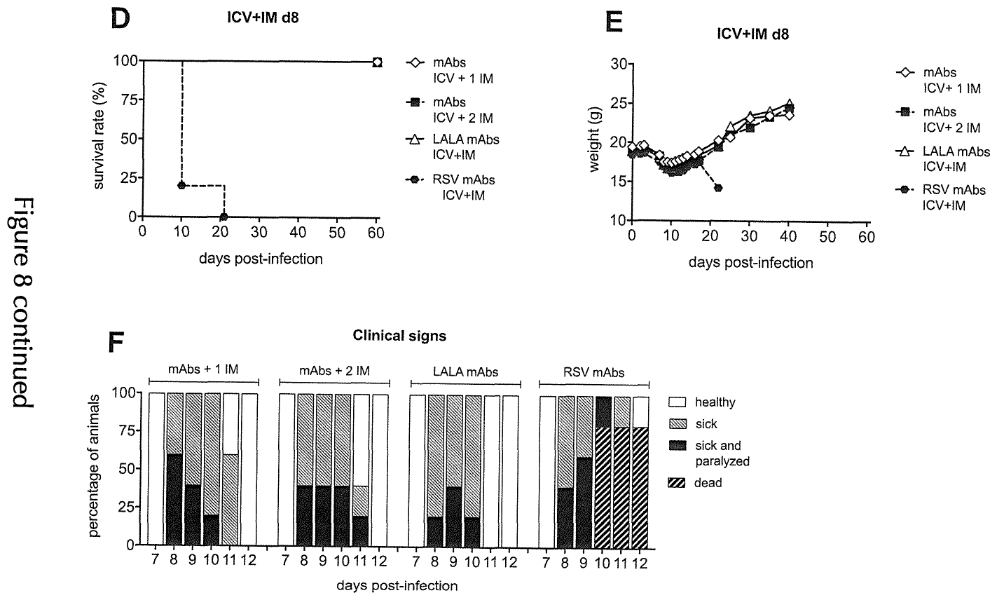

To this end, brain infusion pumps were implanted 7 days before intramuscular challenge with Tha RABV strain (4000 FFU) to allow for the intracerebroventricular (ICV) administration of antibodies as described above. Eight days after infection (i.e. when all animals developed symptoms of rabies disease), animals started receiving for 20 days through the implanted pump either 2+2 mg/kg/day of RVC58+RVC20 human monoclonal antibodies or their LALA variants (i.e., antibodies differing from RVC58 and RVC20 only in the “LALA” mutation in the Fc region of the heavy chain (L234A, L235A)). In addition, under the same conditions a control group of animals received 4 mg/kg of a control human monoclonal antibody specific for the Fusion protein of RSV (respiratory syncytial virus) (FIG. 8A). Animals were also administered with a single matched intramuscular (IM) injection of RVC58+RVC20 (at 20 mg/kg+20 mg/kg), RVC58-LALA+RVC20-LALA (at 20 mg/kg+20 mg/kg), or the control RSV human monoclonal antibody (at 40 mg/kg) on day 8. Another group of animals was also administered with an intramuscular (IM) injection of RVC58+RVC20 (at 20 mg/kg+20 mg/kg) on day 8 and another one on day 30 (FIG. 8A).

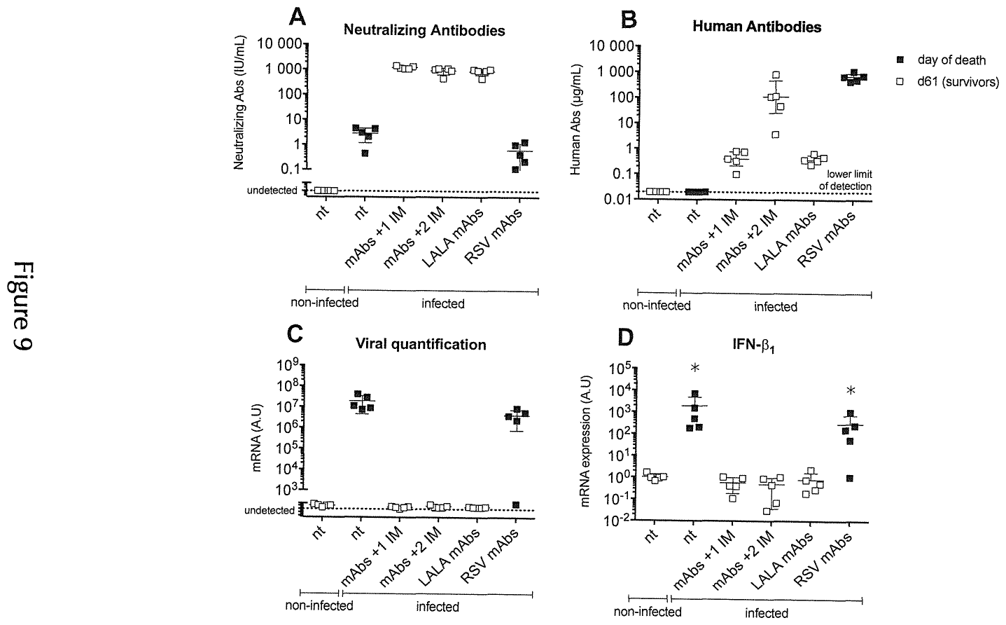

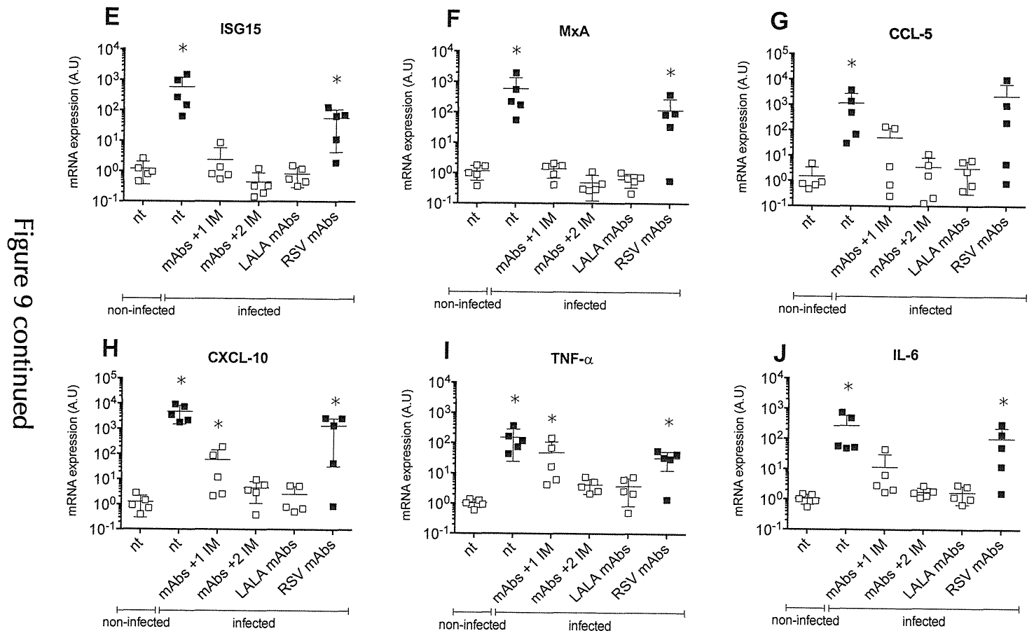

Results are shown in FIGS. 8 and 9. Confirming the results described above, the combined icv/i.m. administration with RVC58+RVC20 human monoclonal antibodies started on day 8 protected all (5 out of 5) animals from mortality and morbidity in the group receiving only one IM dose and all (5 out of 5) animals from mortality and morbidity in the group receiving two IM doses (FIG. 8D-E). In addition, again in surviving animals all symptoms that were present on day 8 disappeared on day 12 (FIG. 8F).

Surprisingly, the combined treatment (i.e. ICV+IM administrations) with antibodies with the “LALA” mutation (RVC58-LALA+RVC20-LALA human monoclonal antibodies; devoid of effector function) started on day 8 protected all 5 out 5 animals from mortality and morbidity (FIG. 8D-E). Of note, in surviving animals all symptoms that were present on day 8 disappeared on day 11, i.e. the resolution of symptoms appeared one day faster as compared to that observed with the Fc-effector functional RVC58+RVC20 human monoclonal antibodies (FIG. 8F).

These results indicate that the therapeutic activity of RVC58+RVC20 human monoclonal antibodies does not rely on antibody effector functions. This finding was unexpected, since antibodies were administered when most of the neuronal cells were already infected. Without being bound to any theory, a possible explanation could be that the therapeutic activity of these antibodies relies uniquely on the neutralization of viral entry. However, other mechanisms of action could also contribute to the observed in vivo efficacy.

In addition, the levels of neutralizing antibodies as well as the level of human antibodies were measured in serum of the animals at the day of sacrifice (death or at the end of the observation period for surviving animals, i.e. day 61; FIG. 9A-B). Mouse neutralising antibody levels were measured by an adaptation of the rapid fluorescent focus inhibition test (RFFIT) for 96 well plates (Bourhy H, Sureau P (1990) Laboratory methods for rabies diagnosis. Paris: Institut Pasteur. pp. 191-3; Feyssaguet M, Dacheux L, Audry L, Compoint A, Morize J L, et al. (2007) Multicenter comparative study of a new ELISA, PLATELIA RABIES II, for the detection and titration of anti-rabies glycoprotein antibodies and comparison with the rapid fluorescent focus inhibition test (RFFIT) on human samples from vaccinated and non-vaccinated people. Vaccine 25: 2244-51). Briefly, a constant dose of challenge virus standard (CVS) is incubated with diluted test sera. An in-house reference serum is calibrated against an international standard. Serum/virus mixtures are incubated, and BSR cells (a clone of BHK-21 cells) were added. After 24 hours incubation, the monolayer is acetone-fixed and stained with a fluorescent anti-nucleocapsid antibody (Chemicon). The result in Mimi was the mean of independent duplicate tests.

A standard ELISA was used to determine binding of serum human antibodies to RABV G protein (CVS-11). Briefly, ELISA plates were coated with RABV G protein at 5 μg/ml, blocked with 10% FCS in PBS, incubated with sera or human antibodies, and washed. Bound antibodies were detected by incubation with AP-conjugated goat anti-human IgG (Southern Biotech). Plates were then washed, substrate (p-NPP, Sigma) was added and plates were read at 405 nm. Quantification was performed using an internal standard based on a human IgG1 monoclonal antibody.

A high level of neutralizing antibodies found in peripheral blood is found in surviving animals indicating that a robust immunological response against the virus is generated by the infection. A high level of human antibodies was found in peripheral blood in surviving animals reflecting the long lasting presence of these antibodies more than 30 days after the last injection. Further, the antibodies injected by the IM route are also found in the circulating blood as demonstrated by the comparison between animals receiving one and 2 IM doses of RVC58+RVC20 human monoclonal antibodies (FIG. 9B).

The measurement of viral mRNA present in the brain clearly showed that no virus was detectable in the brain of surviving animals (FIG. 9C). In contrast, in all succumbing animals virus was detectable in the brain and only a partial reduction of RABV viral titers was observed in treated succumbing animals (FIG. 9C).

Finally, measurement of the IFN-beta, Interferon-stimulated gene 15 (ISG15), myxovirus resistance protein 1 (MxA, a key mediator of the interferon-induced antiviral response), CCL5, CXCL10, TNF-alpha, IL-1-beta and IL-6 (which are indicators of inflammation) mRNA levels in the brain revealed increased levels of IFN-beta, ISG15, MxA, CCL-5, CXCL-10, TNF-alpha, IL1-beta and IL-6 mRNA levels in diseased animals (FIG. 9D-K). However, the level of expression of these genes in treated mice are similar to control (non-infected) mice. These results indicate that treatment with RVC58+RVC20 human monoclonal antibodies inhibit the activation of inflammation in surviving animals. It is interesting to note that mice receiving one IM dose of RVC58-LALA+RVC20-LALA human monoclonal antibodies or 2 IM doses of RVC58+RVC20 human monoclonal antibodies show a particularly low level of activation of transcription of these genes compared to those receiving one IM dose of RVC58+RVC20 human monoclonal antibodies.

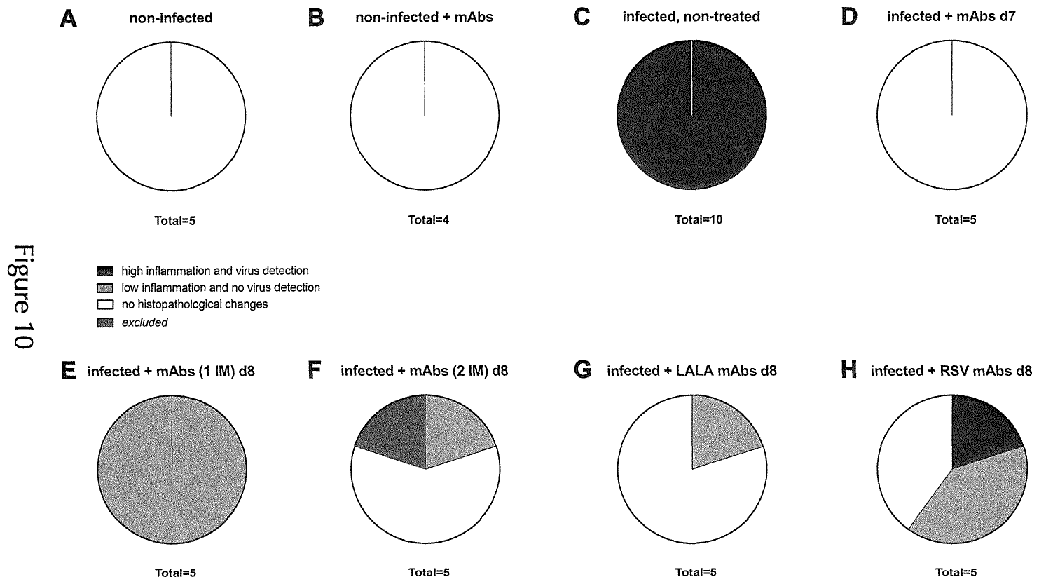

The histological analysis of brain samples for inflammation and for the detection of virus at the end of the study, showed that in mice treated with the RVC58+RVC20 human monoclonal antibodies a low level of inflammation in the absence of detected virus was observed. Conversely, most of the animals treated with the LALA version of the antibodies (RVC58-LALA+RVC20-LALA human monoclonal antibodies) showed no histopathological signs of inflammation (FIG. 10).

This experiment shows that the therapeutic activity of anti-lyssavirus antibodies against symptomatic lyssavirus infection, such as rabies, relied both on the unique modality of administration based on peripheral and CNS routes, as well as on their Fc-independent antiviral mechanism(s) of action.

TABLE OF SEQUENCES AND SEQ ID NUMBERS (SEQUENCE LISTING):

| | | |
|---|---|---|
| SEQ | | |
| ID NO | Description | Sequence |

RVA122 ANTIBODY

| | | |
|---|---|---|
| 1 | CDRH1 aa | gdsmnnfy |
| 2 | CDRH2 aa | iyysgtt |
| 3 | CDRH3 aa | ardsgdyvsyyyygmdv |
| 4 | CDRL1 aa | ssnigsny |
| 5 | CDRL2 aa | ksd |
| 6 | CDRL2 long aa | LIYksdKRP |
| 7 | CDRL3 aa | aawdnrlsgwl |
| 8 | CDRH1 nuc | GGTGACTCCATGAATAATTTCTAC |
| 9 | CDRH2 nuc | ATCTATTACAGTGGGACCACC |
| 10 | CDRH3 nuc | GCGAGAGACTCCGGTGACTACGTCAGCTACTACTATTATG |
| | | GTATGGACGTC |
| 11 | CDRL1 nuc | AGCTCCAACATCGGAAGTAATTAT |
| 12 | CDRL2 nuc | AAGAGTGAT |
| 13 | CDRL2 long nuc | cttatttacAAGAGTGATaagcggccc |
| 14 | CDRL3 nuc | GCAGCATGGGATAACAGGCTGAGTGGTTGGCTC |
| 15 | heavy chain aa | QVHLQESGPGLVKPSETLSLTCTVSgdsmnnfyWGWIRQP |
| | | AGKGLEWIGYiyysgttNYNPSLKSRVTISIDTSKNQFSL |
| | | KVNSVTAADTAVYYCardsgdyvsyyyygmdvWGPGTTVT |
| | | VSS |
| 16 | light chain aa | QSVLTQSPSASDTPGQRVTISCSGSssnigsnyVYWYQQF |
| | | PGTAPKLLIYksdKRPSGVPDRFSGSTSGTSASLAISGLR |
| | | SEDEADYYCaawdnrlsgwlFGGGTKETVL |
| 17 | heavy chain nuc | caggtgcacctgcaggagtcgggcccaggactggtgaagc |
| | | cttcggagaccctgtccctcacctgcactgtctctGGTGA |
| | | CTCCATGAATAATTTCTACtggggctggatccggcagccc |
| | | gcagggaagggactggagtggattggatatATCTATTACA |
| | | GTGGGACCACCaactacaacccctccctcaagagtcgagt |
| | | caccatatcaatagacacgtccaagaaccaattctccctg |
| | | aaggtgaactctgtgaccgctgcggacacggccgtgtatt |
| | | attgtGCGAGAGACTCCGGTGACTACGTCAGCTACTACTA |
| | | TTATGGTATGGACGTCtggggcccagggaccacggtcacc |
| | | gtctcctcag |
| 18 | light chain nuc | cagtctgtgctgactcagtcaccctcagcgtctgataccc |
| | | ccgggcagagggtcaccatctcttgttctggaagcAGCTC |
| | | CAACATCGGAAGTAATTATgtgtattggtaccagcagttc |
| | | ccaggaacggcccccaaactccttatttacAAGAGTGATa |
| | | agcggccctcaggggtccctgaccgattctctggctccac |
| | | gtctggcacctcagcctccctggccatcagtgggctccgg |
| | | tccgaagatgaggctgattattactgtGCAGCATGGGATA |
| | | ACAGGCTGAGTGGTTGGCTCttcggcggagggacgaagct |
| | | gaccgtcctag |

RVA144 ANTIBODY

| | | |
|---|---|---|
| 19 | CDRH1 aa | ggsisstify |
| 20 | CDRH2 aa | vyynght |
| 21 | CDRH3 aa | arpstydysigr |
| 22 | CDRL1aa | ssnigagyd |
| 23 | CDRL2 aa | gnt |
| 24 | CDRL2 long aa | LIYgntKRP |
| 25 | CDRL3 aa | qsfdsslsawv |
| 26 | CDRH1 nuc | GGTGGTTCCATCAGCAGTACTATTTTCTAC |
| 27 | CDRH2 nuc | GTCTATTATAATGGACACACC |
| 28 | CDRH3 nuc | GCGAGACCCTCAACATATGACTACAGTATTGGGCGC |
| 29 | CDRL1 nuc | AGCTCCAACATCGGGGCAGGTTATGAT |
| 30 | CDRL2 nuc | GGTAACACC |
| 31 | CDRL2 long nuc | ctcatctatGGTAACACCaagcggccc |
| 32 | CDRL3 nuc | CAGTCCTTTGACAGCAGCCTGAGTGCTTGGGTA |
| 33 | heavy chain aa | QLQLQESGPGLVKPSETLSLTCTVSggsisstifyWGWIR |
| | | QPPGKGLEWIGSvyynghtYYNPSLKSRVAISIDKSKNQF |
| | | SLRLNSVTAADTAVYYCarpstydysigrWGQGTLVTVSS |
| 34 | light chain aa | QSVLTQPPSVSRAPGQRVTISCTGSssnigagydVHWYQQ |
| | | LPGTAPKLLIYgntKRPSGVPDRFSGSKSGTSASLAITGL |
| | | LTEDEADYYCqsfdsslsawvFGGGTKLTVL |
| 35 | heavy chain nuc | cagctgcagctgcaggagtcgggcccaggactggtgaagc |
| | | cctcggagaccctgtccctcacttgcactgtctctGGTGG |
| | | TTCCATCAGCAGTACTATTTTCTACtggggctggatccgc |
| | | cagcccccagggaagggactggagtggattgggagtGTCT |
| | | ATTATAATGGACACACCtactacaatccgtccctcaagag |
| | | tcgagtcgccatatccattgacaagtccaagaaccagttc |
| | | tccctgaggcttaactctgtgaccgccgcggacacggctg |
| | | tatattactgtGCGAGACCCTCAACATATGACTACAGTAT |
| | | TGGGCGCtggggccagggaaccctggtcaccgtctcctca |
| | | g |
| 36 | light chain nuc | cagtccgtgctgacgcagccgccctcagtgtctcgggccc |
| | | cagggcagagggtcaccatctcctgcactgggagcAGCTC |
| | | CAACATCGGGGCAGGTTATGATgtccactggtaccagcaa |
| | | cttccaggaacagcccccaaactcctcatctatGGTAACA |
| | | CCaagcggccctcaggggtccctgaccgattctctggctc |
| | | caagtctggcacctcagcctccctggccatcactgggctc |
| | | ctgactgaggatgaggctgattattactgccAGTCCTTTG |
| | | ACAGCAGCCTGAGTGCTTGGGTAttcggcggagggaccaa |
| | | actgaccgtcctgg |

RVB185 ANTIBODY

| | | |
|---|---|---|
| 37 | CDRH1 aa | gapvsgvnsy |
| 38 | CDRH2 aa | ikysgst |
| 39 | CDRH3 aa | arqstmtgrdy |
| 40 | CDRL1 aa | rsnigshp |
| 41 | CDRL2 aa | gds |
| 42 | CDRL2 long aa | LIYgdsQRP |
| 43 | CDRL3 aa | aawddslsglwv |
| 44 | CDRH1 nuc | GGTGCCCCCGTCAGTGGTGTTAACTCCTAC |
| 45 | CDRH2 nuc | ATCAAGTACAGTGGGAGCACC |
| 46 | CDRH3 nuc | GCCAGACAAAGTACTATGACGGGCCGGGACTAC |
| 47 | CDRL1 nuc | AGATCCAACATCGGAAGCCATCCT |
| 48 | CDRL2 nuc | GGTGATAGT |
| 49 | CDRL2 long nuc | ctcatctatGGTGATAGTcagcgaccc |
| 50 | CDRL3 nuc | GCAGCATGGGATGACAGCCTGAGTGGCCTTTGGGTG |
| 51 | heavy chain aa | QVQLQESGPGLVKPSETLSLTCSVSgapvsgvnsyWVWIR |
| | | QPPGKGLEWIATikysgstHRSPSLRSRVTISVDTSKNQF |
| | | SLELSSVTAADTAVYYCarqstmtgrdyWGQGTLVTVSS |
| 52 | light chain aa | QSVLTQPPSASGTPGQRVTISCSGSrsnigshpVNWYQQL |
| | | PGAAPKLLIYgdsQRPSGVPDRFSGSKSGPSASLAISGLQ |
| | | SEDEADYYCaawddslsglwvFGGGTKLTVL |
| 53 | heavy chain nuc | caggtgcagctgcaggagtcgggcccaggactggtgaagc |
| | | cttcggagaccctgtccctcacctgcagtgtctccGGTGC |
| | | CCCCGTCAGTGGTGTTAACTCCTACtgggtgtggatccgc |
| | | cagccccccgggaaggggctggagtggattgcgactATCA |
| | | AGTACAGTGGGAGCACCcaccgtagcccgtcgctcaggag |
| | | tcgagtcaccatatccgtagacacgtccaagaatcagttc |
| | | tccctggagctgagctctgtgaccgccgctgacacggctg |
| | | tatattactgtGCCAGACAAAGTACTATGACGGGCCGGGA |
| | | CTACtggggccagggaaccctggtcaccgtctcctcag |
| 54 | light chain nuc | cagtctgtgctgactcagccaccctcagcgtctgggaccc |
| | | ccgggcagagggtcaccatctcttgttctggaagcAGATC |
| | | CAACATCGGAAGCCATCCTgtaaactggtaccagcagctc |
| | | ccgggagcggcccccaagctcctcatctatGGTGATAGTc |
| | | agcgaccctcaggggtccctgaccgattctctggctccaa |
| | | gtctggcccctcagcctccctggccatcagtggactccag |
| | | tctgaagatgaggctgattattactgtGCAGCATGGGATG |
| | | ACAGCCTGAGTGGCCTTTGGGTGttcggcggagggaccaa |
| | | gctgaccgtcctaa |

RVB492 ANTIBODY

| | | |
|---|---|---|
| 55 | CDRH1 aa | gfsfssya |
| 56 | CDRH2 aa | lnsidhrt |
| 57 | CDRH3 aa | argvglwfgelswnyfdy |
| 58 | CDRL1 aa | sndiggyny |
| 59 | CDRL2 aa | yvn |
| 60 | CDRL2 long aa | MIFyvnKRP |
| 61 | CDRL3 aa | csfagsysl |
| 62 | CDRH1 nuc | GGATTCAGCTTTAGCAGCTATGCC |
| 63 | CDRH2 nuc | CTTAATTCTATTGATCATAGAACA |
| 64 | CDRH3 nuc | GCTCGGGGGGTGGGACTATGGTTCGGTGAATTATCCTGGA |
| | | ATTACTTTGACTAC |
| 65 | CDRL1 nuc | AGCAATGATATTGGTGGTTATAACTAT |
| 66 | CDRL2 nuc | TATGTCAAT |
| 67 | CDRL2 long nuc | atgatttttTATGTCAATaagcggccc |
| 68 | CDRL3 nuc | TGCTCATTTGCAGGCAGTTACTCCTTA |
| 69 | heavy chain | EVQLMESGGGLVQPGGSMRLYCAASgfsfssyaMSWVRQA |
| | variant 1 aa | PGKGLEWVSSlnsidhrtDYADSVKGRFTISRDNSKNTLY |
| | | LQMDSLRVEDSAMYYCargvglwfgelswnyfdyWGQGTL |
| | | VTVSS |
| 70 | heavy chain | EVQLVQSGGGLVQPGGSMRLYCAASgfsfssyaMSWVRQA |
| | variant 2 aa | PGKGLEWVSSlnsidhrtDYADSVKGRFTISRDNSKNTLY |
| | | LQMDSLRVEDSAMYYCargvglwfgelswnyfdyWGQGTL |
| | | VTVSS |
| 71 | light chain aa | QSALTQPRSVSGSPGQSVTISCTGTsndiggynyVSWYQQ |
| | | HPGKAPKLMIFyvnKRPSGVPDRFSGSKSGNTASLTISGL |
| | | QAEDEADYYCcsfagsyslFGRGTKLTVL |
| 72 | heavy chain | gaggtgcagctgatggagtctgggggaggcctggtacagc |
| | variant 1 nuc | cgggggggtccatgagactctactgtgcagcctctGGATT |
| | | CAGCTTTAGCAGCTATGCCatgagctgggtccgccaggct |
| | | ccagggaaggggctcgagtgggtctcaagtCTTAATTCTA |
| | | TTGATCATAGAACAgactatgcagactccgtgaagggccg |
| | | gttcaccatctccagagacaattccaagaacaccctgtat |
| | | ttacaaatggacagcctgagagtcgaggactcggccatgt |
| | | attactgtGCTCGGGGGGTGGGACTATGGTTCGGTGAATT |
| | | ATCCTGGAATTACTTTGACTACtggggccagggaaccctg |
| | | gtcaccgtctcctcag |
| 73 | heavy chain | gaggtgcagctggtgcagtctgggggaggcctggtacagc |
| | variant 2 nuc | cgggggggtccatgagactctactgtgcagcctctGGATT |
| | | CAGCTTTAGCAGCTATGCCatgagctgggtccgccaggct |
| | | ccagggaaggggctcgagtgggtctcaagtCTTAATTCTA |
| | | TTGATCATAGAACAgactatgcagactccgtgaagggccg |
| | | gttcaccatctccagagacaattccaagaacaccctgtat |
| | | ttacaaatggacagcctgagagtcgaggactcggccatgt |
| | | attactgtGCTCGGGGGGTGGGACTATGGTTCGGTGAATT |
| | | ATCCTGGAATTACTTTGACTACtggggccagggaaccctg |
| | | gtcaccgtctcctcag |
| 74 | light chain nuc | cagtctgccctgactcagcctcgctcagtgtccgggtctc |
| | | ctggacagtcagtcaccatctcctgcactggaaccAGCAA |
| | | TGATATTGGTGGTTATAACTATgtctcctggtaccaacaa |
| | | cacccaggcaaagcccccaaactcatgatttttTATGTCA |
| | | ATaagcggccctcaggggtccctgatcgcttctctggctc |
| | | caagtctggcaacacggcctccctgaccatctctgggctc |
| | | caggctgaggatgaagctgattattactgcTGCTCATTTG |
| | | CAGGCAGTTACTCCTTAttcggcagagggaccaagctgac |
| | | cgtcctag |

RVC3 ANTIBODY

| | | |
|---|---|---|
| 75 | CDRH1 aa | tftfrnya |
| 76 | CDRH2 aa | isasgsst |
| 77 | CDRH3 aa | akfahdfwsgysyfds |
| 78 | CDRL1 aa | qsvnsn |
| 79 | CDRL2 aa | gas |
| 80 | CDRL2 long aa | LIYgasTRA |
| 81 | CDRL3 aa | qqynnwnsit |
| 82 | CDRH1 nuc | ACATTCACGTTTAGAAACTATGCC |
| 83 | CDRH2 nuc | ATTAGTGCTAGTGGTAGTAGCACG |
| 84 | CDRH3 nuc | GCGAAATTTGCTCACGATTTTTGGAGTGGTTATTCTTACT |
| | | TTGACTCC |
| 85 | CDRL1 nuc | CAGAGTGTTAACAGCAAC |
| 86 | CDRL2 nuc | GGTGCATCC |
| 87 | CDRL2 long nuc | ctcatctatGGTGCATCCaccagggcc |
| 88 | CDRL3 nuc | CAGCAGTATAATAATTGGGTTTCGATCACC |
| 89 | heavy chain aa | EVQLLESGGGLVQPGGSLRLSCAAStftfrnyaMSWVRQA |
| | | PGKGLDWVSGisasgsstNYAASLKGRFTISRDNSKNTLY |
| | | LQMNSLRAEDTAVYYCakfahdfwsgysyfdsWGQGTLVT |
| | | VSS |
| 90 | light chain aa | EIVMTQSPATLSVSPGERATLSCRAGqsvnsnLAWYQQKP |
| | | GQAPRLLIYgasTRATGIPARFSGSGSGTEFTLTISSLQS |
| | | EDFAVYYCqqynnwvsitFGQGTRLEIK |
| 91 | heavy chain nuc | gaggtgcagctgttggagtctgggggaggcctggtgcagc |
| | | cgggggggtccctgagactctcctgtgcagcctctACATT |
| | | CACGTTTAGAAACTATGCCatgtcctgggtccgccaggct |
| | | ccagggaaggggctggactgggtctcagggATTAGTGCTA |
| | | GTGGTAGTAGCACGaattatgcagcctccctgaagggccg |
| | | atttaccatctccagagacaattccaagaacacattgtat |
| | | ctgcaaatgaacagcctgagagccgaggacacggccgtct |
| | | attactgtGCGAAATTTGCTCACGATTTTTGGAGTGGTTA |
| | | TTCTTACTTTGACTCCtggggccagggaaccctggtcacc |
| | | gtctcctcag |
| 92 | light chain nuc | gaaatagtgatgacgcagtctccagccaccctgtctgtgt |
| | | ctccaggggaaagagccaccctctcctgcagggccggtCA |
| | | GAGTGTTAACAGCAACttagcctggtaccagcagaaacct |
| | | gggcaggctcccagactcctcatctatGGTGCATCCacca |
| | | gggccactggtatcccagccaggttcagtggcagtgggtc |
| | | tgggacagagttcactctcaccatcagcagcctgcagtct |
| | | gaagattttgcagtttattactgtCAGCAGTATAATAATT |
| | | GGGTTTCGATCACCttcggccaagggacacgactggagat |
| | | taaac |

RVC20 ANTIBODY

| | | |
|---|---|---|
| 93 | CDRH1 aa | ggsfssgsys |
| 94 | CDRH2 aa | iyysgst |
| 95 | CDRH3 aa | argtysdfwsgspldy |
| 96 | CDRL1 aa | qgisny |
| 97 | CDRL2 aa | aas |
| 98 | CDRL2 long aa | LIYaasSLQ |
| 99 | CDRL3 aa | qqydtyplt |
| 100 | CDRH1 nuc | GGTGGCTCCTTCAGCAGTGGAAGTTACTCC |
| 101 | CDRH2 nuc | ATCTATTACAGTGGGAGCACT |
| 102 | CDRH3 nuc | GCGAGAGGCACGTATTCCGATTTTTGGAGTGGTTCCCCTT |
| | | TAGACTAC |
| 103 | CDRL1 nuc | CAGGGCATTAGCAATTAT |
| 104 | CDRL2 nuc | GCTGCATCC |
| 105 | CDRL2 long nuc | ctgatctatGCTGCATCCagtttgcaa |
| 106 | CDRL3 nuc | CAACAGTATGATACTTACCCTCTCACT |
| 107 | heavy chain aa | QVQLQESGPGLVKPSQTLSLTCTVSggsfssgsysWNWIR |
| | | QHPGKGLEWIGYiyysgstYYNPSLKSRVTMSVHTSKNQF |
| | | SLKLNSITAADTAVYYCargtysdfwsgspldyWGQGTLV |
| | | TVSS |
| 108 | light chain aa | DIQMTQSPSSLSASVGDRVTITCRASqgisnyLAWFQQKP |
| | | GKAPKSLIYaasSLQSGVPSRFSGSGSGTDFTLTINSLQP |
| | | EDFVTYFCqqydtypltFGGGTKVEIK |
| 109 | heavy chain nuc | caggtgcagctgcaggagtcgggcccaggactggtgaagc |
| | | cttcacagaccctgtccctcacctgcactgtctccGGTGG |
| | | CTCCTTCAGCAGTGGAAGTTACTCCtggaactggatccgc |
| | | cagcacccagggaagggcctggagtggattgggtacATCT |
| | | ATTACAGTGGGAGCACTtattacaacccgtccctcaagag |
| | | tcgagttaccatgtcagtacacacgtctaagaaccagttc |
| | | tccctgaagctgaactctataactgccgcggacacggccg |
| | | tgtattactgtGCGAGAGGCACGTATTCCGATTTTTGGAG |
| | | TGGTTCCCCTTTAGACTACtggggccagggaaccctggtc |
| | | accgtctcctcag |
| 110 | light chain nuc | gacatccagatgacccagtctccatcctcactgtctgcat |
| | | ctgtaggagacagagtcaccatcacttgtcgggcgagtCA |
| | | GGGCATTAGCAATTATttagcctggtttcagcagaaacca |
| | | gggaaagcccctaagtccctgatctatGCTGCATCCagtt |
| | | tgcaaagtggggtcccatcaaggttcagcggcagtggatc |
| | | tgggacagatttcactctcaccatcaacagcctgcagcct |
| | | gaagattttgtaacttatttctgcCAACAGTATGATACTT |
| | | ACCCTCTCACTttcggcggagggaccaaggtggagatcaa |
| | | ac |

RVC21 ANTIBODY

| | | |
|---|---|---|
| 111 | CDRH1 aa | ggsisnpnyy |
| 112 | CDRH2 aa | iyyngyt |
| 113 | CDRH3 aa | atqstmttiaghy |
| 114 | CDRL1 aa | tsnignsy |
| 115 | CDRL2 aa | dnn |
| 116 | CDRL2 long aa | LIYdnnKRP |
| 117 | CDRL3 aa | gtwdsslnayv |
| 118 | CDRH1 nuc | GGTGGCTCCATCAGCAACCCTAACTACTAC |
| 119 | CDRH2 nuc | ATCTATTATAATGGGTACACC |
| 120 | CDRH3 nuc | GCGACGCAATCTACGATGACTACCATAGCGGGCCACTAC |
| 121 | CDRL1 nuc | ACATCCAACATTGGGAATTCTTAT |
| 122 | CDRL2 nuc | GACAATAAT |
| 123 | CDRL2 long nuc | ctcatttatGACAATAATaagcgaccc |
| 124 | CDRL3 nuc | GGAACATGGGACAGCAGCCTGAATGCTTATGTC |
| 125 | heavy chain aa | QLQLQESGPGLVKPSETLSLTCTVSggsisnpnyyWGWIR |
| | | QPPGKGLEWIGSiyyngytYYNPSLKSRVTISVDKSKDQF |
| | | FLKMTSLTAADTAVYYCatqstmttiaghyWGQGTLVTVS |
| | | S |
| 126 | light chain aa | QSVLTQAPSVSAAPGLKVTISCSGStsnignsyVSWYQQL |
| | | PGTAPKLLIYdnnKRPSGIPDRFSGSKSDTSATLGITGLQ |
| | | TGDEADYYCgtwdsslnayvFGTGTKVTVL |
| 127 | heavy chain nuc | cagctgcagctgcaggagtcgggcccaggactggtgaagc |
| | | cttcggagaccctgtccctcacgtgcactgtctctGGTGG |
| | | CTCCATCAGCAACCCTAACTACTACtggggctggatccgc |
| | | cagcccccagggaagggtctggaatggattgggagtATCT |
| | | ATTATAATGGGTACACCtactacaacccgtccctcaagag |
| | | tcgagttaccatatccgtggacaagtccaaggaccagttc |
| | | tttctgaagatgacctctctgaccgccgcagacacggctg |
| | | tgtattactgtGCGACGCAATCTACGATGACTACCATAGC |
| | | GGGCCACTACtggggccagggaaccctggtcaccgtctcc |
| | | tcag |
| 128 | light chain nuc | cagtctgtattgacgcaggcgccctcagtgtctgcggccc |
| | | caggactaaaggtcaccatctcctgctctggaagcACATC |
| | | CAACATTGGGAATTCTTATgtatcctggtaccagcagctc |
| | | ccaggaacagcccccaaactcctcatttatGACAATAATa |
| | | agcgaccctcagggattcctgaccgattctctggctccaa |
| | | gtctgacacgtcagccaccctgggcatcaccggactccag |
| | | actggggacgaggccgattattactgcGGAACATGGGACA |
| | | GCAGCCTGAATGCTTATGTCttcggaactgggaccaaggt |
| | | caccgtcctag |

RVC38 ANTIBODY

| | | |
|---|---|---|
| 129 | CDRH1 aa | ggtfssya |
| 130 | CDRH2 aa | impmfvaa |
| 131 | CDRH3 aa | argdgynykwyfdl |
| 132 | CDRL1 aa | qdisny |
| 133 | CDRL2 aa | aas |
| 134 | CDRL2 long aa | LIYaasTLQ |
| 135 | CDRL3 aa | qqldtyvalt |
| 136 | CDRH1 nuc | ggaggcaccttcagcagctatgcc |
| 137 | CDRH2 nuc | atcatgcctatgtttgtggcggca |
| 138 | CDRH3 nuc | gcgagaggggatggctacaattacaagtggtattttgacc |
| | | tt |
| 139 | CDRL1 nuc | caggacattagtaattat |
| 140 | CDRL2 nuc | gctgcatcc |
| 141 | CDRL2 long nuc | ctgatctatgctgcatccactttgcaa |
| 142 | CDRL3 nuc | caacagcttgatacttacgtcgcgctcact |
| 143 | heavy chain aa | EVQLVQSGAEVKKPGSSVRVSCKASggtfssyaISWVRQA |
| | | PGLGLEWMGGimpmfvaaNYAQNFQGRVTVSVDKSTNTAY |
| | | MEMHNLRSDDTAMTYCargdgynykwyfdlWGQGTLVTVS |
| | | S |
| 144 | light chain aa | DIQLTQSPSFLSASVGDRVTITCRASqdisnyLAWYQQKP |
| | | GKPPKLLIYaasTLQRGVPSRFSGSGSGSEFTLTISSLQP |
| | | EDFATYYCqqldtyvaltFGGGTKVEIK |
| 145 | heavy chain nuc | gaggtgcagctggtgcagtctggggctgaggtgaagaagc |
| | | ctgggtcctcggtgagggtctcctgcaaggcttctggagg |
| | | caccttcagcagctatgccatcagctgggtgcgacaggcc |
| | | cctgggctagggcttgagtggatgggagggatcatgccta |
| | | tgtttgtggcggcaaactacgcacagaacttccagggcag |
| | | agtcacggtttctgtggacaaatccacgaacaccgcctat |
| | | atggagatgcacaacctgagatctgacgacacggccatgt |
| | | attactgtgcgagaggggatggctacaattacaagtggta |
| | | ttttgacctttggggccagggaaccctagtcaccgtctcc |
| | | tcag |
| 146 | light chain nuc | gacatccagttgacccagtctccatccttcctgtctgcat |
| | | ctgtaggagacagagtcaccatcacttgccgggccagtca |
| | | ggacattagtaattatttagcctggtatcagcaaaaacca |
| | | gggaagccccctaaactcctgatctatgctgcatccactt |
| | | tgcaaaggggggtcccatcaaggttcagtggcagtggatc |
| | | tgggtcagaattcactctcacaatcagcagcctgcagcct |
| | | gaagattttgcaacttattactgtcaacagcttgatactt |
| | | acgtcgcgctcactttcggcggagggaccaaggtggagat |
| | | caaac |

RVC44 ANTIBODY

| | | |
|---|---|---|
| 147 | CDRH1 aa | gftfssys |
| 148 | CDRH2 aa | isttgtyi |
| 149 | CDRH3 aa | arrsaialagtqrafdi |
| 150 | CDRL1 aa | qninny |
| 151 | CDRL2 aa | aas |
| 152 | CDRL2 long aa | LIYaasSLH |
| 153 | CDRL3 aa | qqsysnpwt |
| 154 | CDRH1 nuc | GGCTTCACCTTTAGTAGTTATAGT |
| 155 | CDRH2 nuc | ATCAGTACTACTGGTACTTACATA |
| 156 | CDRH3 nuc | GCGAGACGGTCGGCCATAGCACTGGCTGGTACGCAGCGTG |
| | | CTTTTGATATC |
| 157 | CDRL1 nuc | CAGAACATTAACAACTAT |
| 158 | CDRL2 nuc | GCTGCATCC |
| 159 | CDRL2 long nuc | ctgatctatGCTGCATCCagtttacat |
| 160 | CDRL3 nuc | caacagagttacagtaacccttggacg |
| 161 | heavy chain aa | EVQLVQSGGGLVKPGGSLRLSCAASgftfssysMSWVRQA |
| | | PGKGLEWVSSisttgtyiYYADSVEGRFSISRDSARSSLF |
| | | LQMNSLRAEDTAVYYCarrsaialagtqrafdiWGPGTNV |
| | | IVSS |
| 162 | light chain aa | DIQMTQSPSSLSASVGDRVTITCRASqninnyLNWYQQKL |
| | | GKAPKLLIYaasSLHSGVPSRFSASGSGTDFILTISNLQP |
| | | EDCATYYCqqsysnpwtFGQGTKVEIK |
| 163 | heavy chain nuc | gaggtgcagctggtgcagtctgggggaggcctggtcaagc |
| | | ctggggggtccctgagactctcctgtgcagcctctGGCTT |
| | | CACCTTTAGTAGTTATAGTatgagttgggtccgccaggct |
| | | ccagggaagggcctggagtgggtctcatccATCAGTACTA |
| | | CTGGTACTTACATAtactacgcagactcagtggagggccg |
| | | attctccatttccagagacagcgccaggagctctctgttt |
| | | ctgcaaatgaacagcctgagagccgaggacacggctgtct |
| | | attactgtGCGAGACGGTCGGCCATAGCACTGGCTGGTAC |
| | | GCAGCGTGCTTTTGATATCtggggcccagggacaaacgtc |
| | | atcgtctcttcag |
| 164 | light chain nuc | gacatccagatgacccagtctccatcttccctgtctgcat |
| | | ctgtaggagacagagtcaccatcacttgccgggcaagtCA |
| | | GAACATTAACAACTATttaaattggtatcagcagaaacta |
| | | gggaaagcccctaagctcctgatctatGCTGCATCCagtt |
| | | tacatagtggggtcccatcaaggttcagtgccagtggatc |
| | | tgggacagatttcattctgaccatcagtaatctgcaacct |
| | | gaagattgtgcaacttactactgtcaacagagttacagta |
| | | acccttggacgttcggccaagggaccaaggtggaaatcaa |
| | | ac |

RVC58 ANTIBODY

| | | |
|---|---|---|
| 165 | CDRH1 aa | gftfstya |
| 166 | CDRH2 aa | isdrggsr |
| 167 | CDRH3 aa | ardiappynyyfygmdv |
| 168 | CDRL1 aa | ssdigafny |
| 169 | CDRL2 aa | evs |
| 170 | CDRL2 long aa | IIYevsNRP |
| 171 | CDRL3 aa | nsytssstql |
| 172 | CDRH1 nuc | GGATTCACCTTTAGCACCTATGCC |
| 173 | CDRH2 nuc | ATTAGTGATAGAGGTGGTAGTAGA |
| 174 | CDRH3 nuc | GCGAGAGATATTGCCCCCCCATATAACTACTACTTCTACGGTATGGAC |
| | | GTC |
| 175 | CDRL1 nuc | AGCAGTGACATTGGTGCTTTTAACTAT |
| 176 | CDRL2 nuc | GAGGTCAGT |
| 177 | CDRL2 long nuc | ataatttatGAGGTCAGTaatcggccc |
| 178 | CDRL3 nuc | AACTCATATACAAGCAGCAGCACTCAGTTA |
| 179 | heavy chain aa | EVQLVESGGGLVQPGGSLRLSCAASgftfstyaMNWVRQA |
| | | PGKGLEWVSGisdrggsrYYAGSVKGRFTISRDNSKNTLF |
| | | LQMNSLRAEDTAVYYCardiappynyyfygmdvWGRGTTV |
| | | TVSS |
| 180 | light chain aa | QSALTQPASVSGSPGQSITISCTGTssdigafnyVSWYQQ |
| | | HPGKAPKLIIYevsNRPSGVSNRFSGSKSGNTASLTISGL |
| | | QAEDEADYYCnsytssstqlFGGGTKLTVL |
| 181 | heavy chain nuc | gaggtgcagctggtggagtctgggggaggcttggtacagcctgggggg |
| | | tccctgagactctcctgtgcggcctctGGATTCACCTTTAGCACCTAT |
| | | GCCatgaattgggtccgccaggctccagggaaggggctggagtgggtc |
| | | tcaggtATTAGTGATAGAGGTGGTAGTAGAtactacgcaggctccgtg |
| | | aagggccggttcaccatctccagagacaattccaagaacacgctgttt |
| | | ctgcaaatgaacagcctgagagccgaggacacggccgtatattactgt |
| | | GCGAGAGATATTGCCCCCCCATATAACTACTACTTCTACGGTATGGAC |
| | | GTCtggggccgagggaccacggtcaccgtctcctcag |
| 182 | light chain nuc | cagtctgccctgactcagcctgcctccgtgtctgggtctc |
| | | ctggacagtcgatcaccatctcctgcactggtaccAGCAG |
| | | TGACATTGGTGCTTTTAACTATgtctcttggtaccaacag |
| | | cacccaggcaaagcccccaaactcataatttatGAGGTCA |
| | | GTaatcggccctcaggggtttctaatcgcttctctggctc |
| | | caagtctggcaacacggcctccctgaccatctctgggctc |
| | | caggctgaggacgaggctgattattactgcAACTCATATA |
| | | CAAGCAGCAGCACTCAGTTAttcggcggagggaccaagct |
| | | gaccgtcctag |

RVC68 ANTIBODY

| | | |
|---|---|---|
| 183 | CDRH1 aa | ggsisehh |
| 184 | CDRH2 aa | ifhsgst |
| 185 | CDRH3 aa | aravstyyyyyidv |
| 186 | CDRL1 aa | qdisnw |
| 187 | CDRL2 aa | aas |
| 188 | CDRL2 long aa | LIYaasSLQ |
| 189 | CDRL3 aa | qqaksfplt |
| 190 | CDRH1 nuc | GGTGGCTCCATTAGTGAGCACCAC |
| 191 | CDRH2 nuc | ATCTTTCACAGTGGGAGTACC |
| 192 | CDRH3 nuc | GCGAGAGCGGTGTCTACTTACTACTACTATTACATAGACG |
| | | TC |
| 193 | CDRL1 nuc | CAGGATATTAGCAACTGG |
| 194 | CDRL2 nuc | GCTGCGTCC |
| 195 | CDRL2 long nuc | ctgatctatGCTGCGTCCagtttgcaa |
| 196 | CDRL3 nuc | CAACAGGCTAAGAGTTTCCCTCTTACT |
| 197 | heavy chain aa | QVQLQESGPRLVKPSETLSLTCTFSggsisehhWSWLRQS |
| | | PGKGLEWIGYifhsgstNYNPSLKSRVNISLDKSKNQFSL |
| | | KLSSVTAADTAVYFCaravstyyyyyidvWGQGTTVTVSS |
| 198 | light chain aa | DIQMTQSPSSVSASVGDRVTITCRASqdisnwLAWYQQKP |
| | | GKAPKLLIYaasSLQSGISSRFSGGGSGTDTTLTISSLQP |
| | | EDFASYYCqqaksfpltFGQGTKLEIK |
| 199 | heavy chain nuc | caggtgcagctacaggagtcgggcccaagactggtgaagc |
| | | cctcggagaccctgtccctcacctgcactttctctGGTGG |
| | | CTCCATTAGTGAGCACCACtggagctggctccggcagtcc |
| | | ccagggaagggactggagtggattggatatATCTTTCACA |
| | | GTGGGAGTACCaactacaacccctccctcaagagtcgagt |
| | | caacatatcattagacaagtccaagaaccagttctccctg |
| | | aagctgagttctgtgaccgctgcggacacggccgtgtatt |
| | | tctgtGCGAGAGCGGTGTCTACTTACTACTACTATTACAT |
| | | AGACGTCtggggccaagggaccacggtcaccgtctcctca |
| | | g |
| 200 | light chain nuc | gacatccagatgacccagtctccatcttccgtgtctgcat |
| | | ctgtaggagacagagtcaccatcacttgtcgggcgagtCA |
| | | GGATATTAGCAACTGGttagcctggtatcagcagaaacca |
| | | gggaaagcccctaaactcctgatctatGCTGCGTCCagtt |
| | | tgcaaagtgggatctcatctaggttcagcggcggtggctc |
| | | tgggacagatttcactctcaccatcagcagcctgcagcct |
| | | gaagattttgcaagttactactgtCAACAGGCTAAGAGTT |
| | | TCCCTCTTACTtttggccaggggaccaagctggagatcaa |
| | | ac |

RVC111 ANTIBODY

| | | |
|---|---|---|
| 201 | CDRH1 aa | GFSFSSYV |
| 202 | CDRH2 aa | ISYDGSNK |
| 203 | CDRH3 aa | ARGSGTQTPLFDY |
| 204 | CDRL1 aa | QSITSW |
| 205 | CDRL2 aa | DDS |
| 206 | CDRL2 long aa | LIYDDSTLE |
| 207 | CDRL3 aa | QQYESYSGT |
| 208 | CDRH1 nuc | ggattctccttcagtagctatgtt |
| 209 | CDRH2 nuc | atatcatatgatggaagtaataaa |
| 210 | CDRH3 nuc | gcgagagggtccggaacccaaactcccctctttgactac |
| 211 | CDRL1 nuc | cagagtattactagctgg |
| 212 | CDRL2 nuc | gatgactcc |
| 213 | CDRL2 long nuc | ctgatctatgatgactccactttggaa |
| 214 | CDRL3 nuc | caacagtatgagagttattcagggacg |
| 215 | heavy chain aa | QVQLVESGGGVVQPGRSLRLSCAASGFSFSSYVMYWVRQA |
| | | PGKGLEWVTIISYDGSNKYYADSVKGRFTISRDNSKNTLY |
| | | LQMNSLRAEDTAVYYCARGSGTQTPLFDYWGQGTLVTVSS |
| 216 | light chain aa | DIQMTQSPSTLSASVGDRVTITCRANQSITSWVAWYQQMP |
| | | GRAPKLLIYDDSTLESGVPSRFSGSGSGTEFTLTISSLQP |
| | | DDFATYYCQQYESYSGTFGQGTKVEIK |
| 217 | heavy chain nuc | caggtgcaactggtggagtctgggggaggcgtggtccagc |
| | | ctgggaggtccctgagactctcctgtgcagcctctggatt |
| | | ctccttcagtagctatgttatgtactgggtccgccaggct |
| | | ccaggcaaggggctggagtgggtgacaattatatcatatg |
| | | atggaagtaataaatactacgcagactccgtgaagggccg |
| | | attcaccatctccagagacaattccaagaacacgctgtat |
| | | ctgcaaatgaacagcctgagagctgaggacacggctgtct |
| | | attactgtgcgagagggtccggaacccaaactcccctctt |
| | | tgactactggggccagggaaccctggtcaccgtctcctca |
| | | g |
| 218 | light chain nuc | gacatccagatgacccagtctccttccaccctgtctgcat |
| | | ctgtgggagacagagtcaccatcacttgccgggccaatca |
| | | gagtattactagctgggtggcctggtatcagcagatgcca |
| | | gggagagcccctaaactcctgatctatgatgactccactt |
| | | tggaaagtggggtcccatcaaggttcagcggcagtggatc |
| | | tgggacagaattcactctcaccatcagcagcctgcagcct |
| | | gatgattttgcaacttattactgccaacagtatgagagtt |
| | | attcagggacgttcggccaagggaccaaggtggaaatcaa |
| | | ac |

Constant regions

| | | |
|---|---|---|
| 219 | IgG1 CH1-CH2- | ASTKGPSVFPLAPSSKSTSGGTAALGCLVKDYFPEPVTVSWNS |
| | CH3 LALA aa | GALTSGVHTFPAVLQSSGLYSLSSVVTVPSSSLGTQTYICNVN |
| | | HKPSNTKVDKKVEPKSCDKTHTCPPCPAPEAAGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSRDELTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 220 | IgG CK aa | RTVAAPSVFIFPPSDEQLKSGTASVVCLLNNFYPREAKVQWKV |
| | | DNALQSGNSQESVTEQDSKDSTYSLSSTLTLSKADYEKHKVYA |
| | | CEVTHQGLSSPVTKSFNRGEC |
| 221 | IgG CL aa | GQPKAAPSVTLFPPSSEELQANKATLVCLISDFYPGAVTVAWK |
| | | ADSSPVKAGVETTTPSKQSNNKYAASSYLSLTPEQWKSHRSYS |
| | | CQVTHEGSTVEKTVAPTECS |
| 222 | IgG1 CH1-CH2- | gcgtcgaccaagggcccatcggtcttccccctggcaccctcctccaagagcacctct |
| | CH3 LALA nucl | gggggcacagcggccctgggctgcctggtcaaggactacttccccgaacctgtgacg |
| | | gtctcgtggaactcaggcgccctgaccagcggcgtgcacaccttcccggctgtccta |
| | | cagtcctcaggactctactccctcagcagcgtggtgaccgtgccctccagcagcttg |
| | | ggcacccagacctacatctgcaacgtgaatcacaagcccagcaacaccaaggtggac |
| | | aagagagttgagcccaaatcttgtgacaaaactcacacatgcccaccgtgcccagca |
| | | cctgaaGCCGCGgggggaccgtcagtcttcctcttccccccaaaacccaaggacacc |
| | | ctcatgatctcccggacccctgaggtcacatgcgtggtggtggacgtgagccacgaa |
| | | gaccctgaggtcaagttcaactggtacgtggacggcgtggaggtgcataatgccaag |
| | | acaaagccgcgggaggagcagtacaacagcacgtaccgtgtggtcagcgtcctcacc |
| | | gtcctgcaccaggactggctgaatggcaaggagtacaagtgcaaggtctccaacaaa |
| | | gccctcccagcccccatcgagaaaaccatctccaaagccaaagggcagccccgagaa |
| | | ccacaggtgtacaccctgcccccatcccgggaggagatgaccaagaaccaggtcagc |
| | | ctgacctgcctggtcaaaggcttctatcccagcgacatcgccgtggagtgggagagc |
| | | aatgggcagccggagaacaactacaagaccacgcctcccgtgctggactccgacggc |
| | | tccttcttcctctatagcaagctcaccgtggacaagagcaggtggcagcaggggaac |
| | | gtcttctcatgctccgtgatgcatgaggctctgcacaaccactacacgcagaagagc |
| | | ctctccctgtccccgggtaaa |
| 223 | IgG CK nucl | cgTacGgtggctgcaccatctgtcttcatcttcccgccatctgatgagcagttgaaa |
| | | tctggaactgcctctgttgtgtgcctgctgaataacttctatcccagagaggccaaa |
| | | gtacagtggaaggtggataacgccctccaatcgggtaactcccaggagagtgtcaca |
| | | gagcaggacagcaaggacagcacctacagcctcagcagcaccctgacgctgagcaaa |
| | | gcagactacgagaaacacaaagtctacgcctgcgaagtcacccatcagggcctgagc |
| | | tcgcccgtcacaaagagcttcaacaggggagagtgt |
| 224 | IgG CL nucl | ggtcagcccaaggctgccccctcggtcactctgttcccgccctcctctgaggagctt |
| | | caagccaacaaggccacactggtgtgtctcataagtgacttctacccgggagccgtg |
| | | acagtggcttggaaagcagatagcagccccgtcaaggcgggagtggagaccaccaca |
| | | ccctccaaacaaagcaacaacaagtacgcggccagcagctatctgagcctgacgcct |
| | | gagcagtggaagtcccacagaagctacagctgccaggtcacgcatgaagggagcacc |
| | | gtggagaagacagtggcccctacagaatgttca |

Primers

| | | |
|---|---|---|
| 225 | Forward primer | CTG ACG TAG CAC TGG CAG AC |
| 226 | Reverse primer | AGT CGA CCT CCG TTC ATC AT |

1. A method of treating a lyssavirus infection in a subject, the method comprising administering an anti-lyssavirus antibody, or an antigen-binding fragment thereof into a central nervous system (CNS) and peripherally.

2. The method of claim 1, comprising administering the antibody, or the antigen-binding fragment thereof, into the CNS, peripherally, or both for the first time at least five days, at least six days, at least seven days, or at least eight days after exposure of the subject to a lyssavirus.

3-5. (canceled)

6. The method of claim 1, comprising administering the antibody, or the antigen-binding fragment thereof, into the CNS, peripherally, or both for the first time after symptoms of lyssavirus infection occur in the subject.

7. The method of claim 1, comprising administering the antibody, or the antigen-binding fragment thereof, peripherally and into the CNS on the same day, at about the same time, or concurrently.

8-9. (canceled)

10. The method of claim 1, comprising a single peripheral administration of the antibody, or the antigen-binding fragment thereof.

11. The method of claim 1, comprising repeatedly administering the antibody, or the antigen-binding fragment thereof peripherally.

12. The method of claim 1, comprising administering the antibody, or the antigen-binding fragment thereof, into the CNS continuously for at least 15 minutes, at least 30 minutes, at least 1 hour, at least 6 hours, at least 12 hours, at least 24 hours, at least 2 days, at least 3 days, at least 4 days, or at least 5 days.

13-21. (canceled)

22. The method of claim 1, comprising administering the antibody, or the antigen-binding fragment thereof, into the CNS daily or every second day for at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days.

23. The method of claim 1, comprising administering the antibody, or the antigen-binding fragment thereof, into the CNS intrathecally, intracerebroventricularly, intracerebrally, epidurally, transnasally, intranasally, or perspinally.

24. The method of claim 1, comprising peripherally administering the antibody, or the antigen-binding fragment thereof systemically or locally.

25. (canceled)

26. The method of claim 1, comprising peripherally administering the antibody, or the antigen-binding fragment thereof intramuscularly, intravenously, intradermally, or subcutaneously.

27. The method of claim 1, wherein the lyssavirus infection is rabies and the anti-lyssavirus antibody, or the antigen-binding fragment thereof, is an anti-RABV antibody or an antigen-binding fragment thereof.

28. The method of claim 1, wherein the antibody, or the antigen-binding fragment thereof, binds to lyssavirus glycoprotein G.

29. (canceled)

30. The method of claim 27, wherein the antibody, or the antigen-binding fragment thereof, binds to antigenic site I or antigenic site III of glycoprotein G of RABV.

31. The method of claim 1, wherein the method comprise administering a first anti-lyssavirus antibody, or an antigen-binding fragment thereof, in combination with a second anti-lyssavirus antibody, or an antigen-binding fragment thereof.

32. The method of claim 31, comprising administering the first and second anti-lyssavirus antibodies, or the antigen-binding fragments thereof, in combination in the same pharmaceutical composition.

33. (canceled)

34. The method of claim 31, wherein the first anti-lyssavirus antibody, or the antigen-binding fragment thereof, binds to antigenic site I of glycoprotein G of RABV and the second anti-lyssavirus antibody, or the antigen-binding fragment thereof, binds to antigenic site III of glycoprotein G of RABV.

35. The method of claim 31, comprising administering the first anti-lyssavirus antibody, or the antigen-binding fragment thereof, and the second anti-lyssavirus antibody, or the antigen-binding fragment thereof, in combination at equimolar amounts.

36. The method of claim 1, wherein the antibody, or the antigen-binding fragment thereof, comprises a monoclonal antibody, a human antibody, a purified antibody, a single chain antibody, a Fab, a Fab′, a F(ab′)2, a Fv, and/or a scFv.

37. (canceled)

38. The method of claim 1, wherein the antibody, or the antigen-binding fragment thereof, neutralizes lyssavirus infection by (i) RABV and (ii) at least 50% of non-RABV lyssaviruses selected from the group consisting of DUVV, EBLV-1, EBLV-2, ABLV, IRKV, KHUV, ARAV, LBV, MOK, SHIV, BBLV, WCBV and IKOV with an IC₅₀ of less than 10000 ng/ml.

39. (canceled)

40. The method of claim 1, wherein the antibody, or the antigen-binding fragment thereof, comprises: (i) heavy chain CDRH1, CDRH2, and CDRH3 amino acid sequences and light chain CDRL1, CDRL2, and CDRL3 amino acid sequences of SEQ ID NOs: 93-97 and 99 or of SEQ ID NOs: 93-96 and 98-99, respectively; (ii) heavy chain CDRH1, CDRH2, and CDRH3 amino acid sequences and light chain CDRL1, CDRL2, and CDRL3 amino acid sequences of SEQ ID NOs: 165-169 and 171 or of SEQ ID NOs: 165-168 and 170-171, respectively; (iii) heavy chain CDRH1, CDRH2, and CDRH3 amino acid sequences and light chain CDRL1, CDRL2, and CDRL3 amino acid sequences of SEQ ID NOs: 1-5 and 7 or of SEQ ID NOs: 1-4 and 6-7, respectively; (iv) heavy chain CDRH1, CDRH2, and CDRH3 amino acid sequences and light chain CDRL1, CDRL2, and CDRL3 amino acid sequences of SEQ ID NOs: 19-23 and 25 or of SEQ ID NOs: 19-22 and 24-25, respectively; (v) heavy chain CDRH1, CDRH2, and CDRH3 amino acid sequences and light chain CDRL1, CDRL2, and CDRL3 amino acid sequences of SEQ ID NOs: 37-41 and 43 or of SEQ ID NOs: 37-40 and 42-43, respectively; (vi) heavy chain CDRH1, CDRH2, and CDRH3 amino acid sequences and light chain CDRL1, CDRL2, and CDRL3 amino acid sequences of SEQ ID NOs: 55-59 and 61 or of SEQ ID NOs: 55-58 and 60-61, respectively; (vii) heavy chain CDRH1, CDRH2, and CDRH3 amino acid sequences and light chain CDRL1, CDRL2, and CDRL3 amino acid sequences of SEQ ID NOs: 75-79 and 81 or of SEQ ID NOs: 75-78 and 80-81, respectively; (viii) heavy chain CDRH1, CDRH2, and CDRH3 amino acid sequences and light chain CDRL1, CDRL2, and CDRL3 amino acid sequences of SEQ ID NOs: 111-115 and 117 or of SEQ ID NOs: 111-114 and 116-117, respectively; (ix) heavy chain CDRH1, CDRH2, and CDRH3 amino acid sequences and light chain CDRL1, CDRL2, and CDRL3 amino acid sequences of SEQ ID NOs: 129-133 and 135 or of SEQ ID NOs: 129-132 and 134-135, respectively; (x) heavy chain CDRH1, CDRH2, and CDRH3 amino acid sequences and light chain CDRL1, CDRL2, and CDRL3 amino acid sequences of SEQ ID NOs: 147-151 and 153 or of SEQ ID NOs: 147-150 and 152-153, respectively; (xi) heavy chain CDRH1, CDRH2, and CDRH3 amino acid sequences and light chain CDRL1, CDRL2, and CDRL3 amino acid sequences of SEQ ID NOs: 183-187 and 189 or of SEQ ID NOs: 183-186 and 188-189, respectively; or (xii) heavy chain CDRH1, CDRH2, and CDRH3 amino acid sequences and light chain CDRL1, CDRL2, and CDRL3 amino acid sequences of SEQ ID NOs: 201-205 and 207 or of SEQ ID NOs: 201-204 and 206-207, respectively.

41. The method of claim 40, wherein the antibody, or the antigen-binding fragment thereof, comprises heavy chain CDRH1, CDRH2, and CDRH3 amino acid sequences and light chain CDRL1, CDRL2, and CDRL3 amino acid sequences of SEQ ID NOs: 93-97 and 99 or of SEQ ID NOs: 93-96 and 98-99, respectively.

42. (canceled)

43. The method of claim 40, wherein the antibody, or the antigen-binding fragment thereof, comprises: (i) a heavy chain variable region having the amino acid sequence of SEQ ID NO: 107 and a light chain variable region having the amino acid sequence of SEQ ID NO: 108; or (ii) a heavy chain variable region having the amino acid sequence of SEQ ID NO: 179 and a light chain variable region having the amino acid sequence of SEQ ID NO: 180; or (iii) a heavy chain variable region having the amino acid sequence of SEQ ID NO: 15 and a light chain variable region having to the amino acid sequence of SEQ ID NO: 16; or (iv) a heavy chain variable region having the amino acid sequence of SEQ ID NO: 33 and a light chain variable region having the amino acid sequence of SEQ ID NO: 34; or (v) a heavy chain variable region having the amino acid sequence of SEQ ID NO: 51 and a light chain variable region having amino acid sequence of SEQ ID NO: 52; or (vi) a heavy chain variable region having the amino acid sequence of SEQ ID NO: 69 and a light chain variable region having the amino acid sequence of SEQ ID NO: 71; or (vii) a heavy chain variable region having the amino acid sequence of SEQ ID NO: 70 and a light chain variable region having the amino acid sequence of SEQ ID NO: 71; or (viii) a heavy chain variable region having the amino acid sequence of SEQ ID NO: 89 and a light chain variable region having the amino acid sequence of SEQ ID NO: 90; or (ix) a heavy chain variable region having the amino acid sequence of SEQ ID NO: 125 and a light chain variable region having the amino acid sequence of SEQ ID NO: 126; or (x) a heavy chain variable region the amino acid sequence of SEQ ID NO: 143 and a light chain variable region having the amino acid sequence of SEQ ID NO: 144; or (xi) a heavy chain variable region having the amino acid sequence of SEQ ID NO: 161 and a light chain variable region having the amino acid sequence of SEQ ID NO: 162; or (xii) a heavy chain variable region having the amino acid sequence of SEQ ID NO: 197 and a light chain variable region having the amino acid sequence of SEQ ID NO: 198; or (xiii) a heavy chain variable region having the amino acid sequence of SEQ ID NO: 215 and a light chain variable region having the amino acid sequence of SEQ ID NO: 216.

44. The method of claim 43, wherein the antibody, or the antigen-binding fragment thereof, comprises a heavy chain variable region the amino acid sequence of SEQ ID NO: 107 and a light chain variable region having the amino acid sequence of SEQ ID NO: 108 or a heavy chain variable region having the amino acid sequence of SEQ ID NO: 179 and a light chain variable region having the amino acid sequence of SEQ ID NO: 180.

45-60. (canceled)

61. The method of claim 1, comprising administering the antibody, or the antigen-binding fragment thereof, in combination with an anti-lyssavirus vaccine, an antiviral agent, interferon-alpha and/or ketamine.

62. The method of claim 61, wherein the anti-lyssavirus vaccine is a rabies vaccine or the antiviral agent ribavirin.

63. (canceled)

64. The method of claim 1, comprising administering the antibody, or the antigen-binding fragment thereof, without concomitant and/or subsequent administration of an anti-lyssavirus vaccine.

65-71. (canceled)

72. An anti-lyssavirus antibody, or an antigen-binding fragment thereof, comprising an Fc moiety comprising a CH2 domain and a CH2 L4A mutation and/or a CH2 L5A mutation.

73-96. (canceled)

97. A nucleic acid molecule comprising a polynucleotide encoding the antibody, or the antigen binding fragment thereof, according claim 72.

98. (canceled)

99. A vector comprising the nucleic acid molecule according to claim 97.

100. A cell expressing the antibody, or the antigen binding fragment thereof, according to claim 97.

101. A pharmaceutical composition comprising the antibody, or the antigen binding fragment thereof, according claim 1, and a pharmaceutically acceptable excipient, diluent or carrier.

102-127. (canceled)

128. The method of claim 1, wherein the antibody, or an antigen-binding fragment thereof, comprises an Fc moiety comprising a CH2 domain and a CH2 L4A mutation and/or a CH2 L5A mutation.

129. A method of treating a lyssavirus infection in a subject, the method comprising administering an anti-lyssavirus antibody, or an antigen-binding fragment thereof, into a central nervous system of the subject, wherein the subject has been administered, is being administered, or will be administered the anti-lyssavirus antibody, or an antigen-binding fragment thereof peripherally.

130. A method of treating a lyssavirus infection in a subject, the method comprising administering an anti-lyssavirus antibody, or an antigen-binding fragment thereof, peripherally in the subject, where the anti-lyssavirus antibody, or antigen-binding fragment thereof has been administered, is being administered, or will be administered into a central nervous system (CNS) of the subject.


SEQUENCE LISTING (226 sequences)

SEQ ID NO: 1 - protein, 8 aa, Artificial Sequence
  RVA122 CDRH1 aa
GDSMNNFY

SEQ ID NO: 2 - protein, 7 aa, Artificial Sequence
  RVA122 CDRH2 aa
IYYSGTT

SEQ ID NO: 3 - protein, 17 aa, Artificial Sequence
  RVA122 CDRH3 aa
ARDSGDYVSYYYYGMDV

SEQ ID NO: 4 - protein, 8 aa, Artificial Sequence
  RVA122 CDRL1 aa
SSNIGSNY

SEQ ID NO: 5 - skipped

SEQ ID NO: 6 - protein, 9 aa, Artificial Sequence
  RVA122 CDRL2 long aa
LIYKSDKRP

SEQ ID NO: 7 - protein, 11 aa, Artificial Sequence
  RVA122 CDRL3 aa
AAWDNRLSGWL

SEQ ID NO: 8 - DNA, 24 bp, Artificial Sequence
  RVA122 CDRH1 nuc
ggtgactccatgaataatttctac

SEQ ID NO: 9 - DNA, 21 bp, Artificial Sequence
  RVA122 CDRH2 nuc
atctattacagtgggaccacc

SEQ ID NO: 10 - DNA, 51 bp, Artificial Sequence
  RVA122 CDRH3 nuc
gcgagagactccggtgactacgtcagctactactattatggtatggacgtc

SEQ ID NO: 11 - DNA, 24 bp, Artificial Sequence
  RVA122 CDRL1 nuc
agctccaacatcggaagtaattat

SEQ ID NO: 12 - skipped

SEQ ID NO: 13 - DNA, 27 bp, Artificial Sequence
  RVA122 CDRL2 long nuc
cttatttacaagagtgataagcggccc

SEQ ID NO: 14 - DNA, 33 bp, Artificial Sequence
  RVA122 CDRL3 nuc
gcagcatgggataacaggctgagtggttggctc

SEQ ID NO: 15 - protein, 123 aa, Artificial Sequence
  RVA122 heavy chain aa
QVHLQESGPGLVKPSETLSLTCTVSGDSMNNFYWGWIRQPAGKGLEWIGYIYYSGTTNYNPSLKSRVTISIDTSKNQFSLKVNSVTAADTAVYYCARDSGDYVSYYYYGMDVWGPGTTVTVSS

SEQ ID NO: 16 - protein, 110 aa, Artificial Sequence
  RVA122 light chain aa
QSVLTQSPSASDTPGQRVTISCSGSSSNIGSNYVYWYQQFPGTAPKLLIYKSDKRPSGVPDRFSGSTSGTSASLAISGLRSEDEADYYCAAWDNRLSGWLFGGGTKLTVL

SEQ ID NO: 17 - DNA, 370 bp, Artificial Sequence
  RVA122 heavy chain nuc
caggtgcacctgcaggagtcgggcccaggactggtgaagccttcggagaccctgtccctcacctgcactgtctctggtgactccatgaataatttctactggggctggatccggcagcccgcagggaagggactggagtggattggatatatctattacagtgggaccaccaactacaacccctccctcaagagtcgagtcaccatatcaatagacacgtccaagaaccaattctccctgaaggtgaactctgtgaccgctgcggacacggccgtgtattattgtgcgagagactccggtgactacgtcagctactactattatggtatggacgtctggggcccagggaccacggtcaccgtctcctcag

SEQ ID NO: 18 - DNA, 331 bp, Artificial Sequence
  RVA122 light chain nuc
cagtctgtgctgactcagtcaccctcagcgtctgatacccccgggcagagggtcaccatctcttgttctggaagcagctccaacatcggaagtaattatgtgtattggtaccagcagttcccaggaacggcccccaaactccttatttacaagagtgataagcggccctcaggggtccctgaccgattctctggctccacgtctggcacctcagcctccctggccatcagtgggctccggtccgaagatgaggctgattattactgtgcagcatgggataacaggctgagtggttggctcttcggcggagggacgaagctgaccgtcctag

SEQ ID NO: 19 - protein, 10 aa, Artificial Sequence
  RVA144 CDRH1 aa
GGSISSTIFY

SEQ ID NO: 20 - protein, 7 aa, Artificial Sequence
  RVA144 CDRH2 aa
VYYNGHT

SEQ ID NO: 21 - protein, 12 aa, Artificial Sequence
  RVA144 CDRH3 aa
ARPSTYDYSIGR

SEQ ID NO: 22 - protein, 9 aa, Artificial Sequence
  RVA144 CDRL1 aa
SSNIGAGYD

SEQ ID NO: 23 - skipped

SEQ ID NO: 24 - protein, 9 aa, Artificial Sequence
  RVA144 CDRL2 long aa
LIYGNTKRP

SEQ ID NO: 25 - protein, 11 aa, Artificial Sequence
  RVA144 CDRL3 aa
QSFDSSLSAWV

SEQ ID NO: 26 - DNA, 30 bp, Artificial Sequence
  RVA144 CDRH1 nuc
ggtggttccatcagcagtactattttctac

SEQ ID NO: 27 - DNA, 21 bp, Artificial Sequence
  RVA144 CDRH2 nuc
gtctattataatggacacacc

SEQ ID NO: 28 - DNA, 36 bp, Artificial Sequence
  RVA144 CDRH3 nuc
gcgagaccctcaacatatgactacagtattgggcgc

SEQ ID NO: 29 - DNA, 27 bp, Artificial Sequence
  RVA144 CDRL1 nuc
agctccaacatcggggcaggttatgat

SEQ ID NO: 30 - skipped

SEQ ID NO: 31 - DNA, 27 bp, Artificial Sequence
  RVA144 CDRL2 long nuc
ctcatctatggtaacaccaagcggccc

SEQ ID NO: 32 - DNA, 33 bp, Artificial Sequence
  RVA144 CDRL3 nuc
cagtcctttgacagcagcctgagtgcttgggta

SEQ ID NO: 33 - protein, 120 aa, Artificial Sequence
  RVA144 heavy chain aa
QLQLQESGPGLVKPSETLSLTCTVSGGSISSTIFYWGWIRQPPGKGLEWIGSVYYNGHTYYNPSLKSRVAISIDKSKNQFSLRLNSVTAADTAVYYCARPSTYDYSIGRWGQGTLVTVSS

SEQ ID NO: 34 - protein, 111 aa, Artificial Sequence
  RVA144 light chain aa
QSVLTQPPSVSRAPGQRVTISCTGSSSNIGAGYDVHWYQQLPGTAPKLLIYGNTKRPSGVPDRFSGSKSGTSASLAITGLLTEDEADYYCQSFDSSLSAWVFGGGTKLTVL

SEQ ID NO: 35 - DNA, 361 bp, Artificial Sequence
  RVA144 heavy chain nuc
cagctgcagctgcaggagtcgggcccaggactggtgaagccctcggagaccctgtccctcacttgcactgtctctggtggttccatcagcagtactattttctactggggctggatccgccagcccccagggaagggactggagtggattgggagtgtctattataatggacacacctactacaatccgtccctcaagagtcgagtcgccatatccattgacaagtccaagaaccagttctccctgaggcttaactctgtgaccgccgcggacacggctgtatattactgtgcgagaccctcaacatatgactacagtattgggcgctggggccagggaaccctggtcaccgtctcctcag

SEQ ID NO: 36 - DNA, 334 bp, Artificial Sequence
  RVA144 light chain nuc
cagtccgtgctgacgcagccgccctcagtgtctcgggccccagggcagagggtcaccatctcctgcactgggagcagctccaacatcggggcaggttatgatgtccactggtaccagcaacttccaggaacagcccccaaactcctcatctatggtaacaccaagcggccctcaggggtccctgaccgattctctggctccaagtctggcacctcagcctccctggccatcactgggctcctgactgaggatgaggctgattattactgccagtcctttgacagcagcctgagtgcttgggtattcggcggagggaccaaactgaccgtcctgg

SEQ ID NO: 37 - protein, 10 aa, Artificial Sequence
  RVB185 CDRH1 aa
GAPVSGVNSY

SEQ ID NO: 38 - protein, 7 aa, Artificial Sequence
  RVB185 CDRH2 aa
IKYSGST

SEQ ID NO: 39 - protein, 11 aa, Artificial Sequence
  RVB185 CDRH3 aa
ARQSTMTGRDY

SEQ ID NO: 40 - protein, 8 aa, Artificial Sequence
  RVB185 CDRL1 aa
RSNIGSHP

SEQ ID NO: 41 - skipped

SEQ ID NO: 42 - protein, 9 aa, Artificial Sequence
  RVB185 CDRL2 long aa
LIYGDSQRP

SEQ ID NO: 43 - protein, 12 aa, Artificial Sequence
  RVB185 CDRL3 aa
AAWDDSLSGLWV

SEQ ID NO: 44 - DNA, 30 bp, Artificial Sequence
  RVB185 CDRH1 nuc
ggtgcccccgtcagtggtgttaactcctac

SEQ ID NO: 45 - DNA, 21 bp, Artificial Sequence
  RVB185 CDRH2 nuc
atcaagtacagtgggagcacc

SEQ ID NO: 46 - DNA, 33 bp, Artificial Sequence
  RVB185 CDRH3 nuc
gccagacaaagtactatgacgggccgggactac

SEQ ID NO: 47 - DNA, 24 bp, Artificial Sequence
  RVB185 CDRL1 nuc
agatccaacatcggaagccatcct

SEQ ID NO: 48 - skipped

SEQ ID NO: 49 - DNA, 27 bp, Artificial Sequence
  RVB185 CDRL2 long nuc
ctcatctatggtgatagtcagcgaccc

SEQ ID NO: 50 - DNA, 36 bp, Artificial Sequence
  RVB185 CDRL3 nuc
gcagcatgggatgacagcctgagtggcctttgggtg

SEQ ID NO: 51 - protein, 119 aa, Artificial Sequence
  RVB185 heavy chain aa
QVQLQESGPGLVKPSETLSLTCSVSGAPVSGVNSYWVWIRQPPGKGLEWIATIKYSGSTHRSPSLRSRVTISVDTSKNQFSLELSSVTAADTAVYYCARQSTMTGRDYWGQGTLVTVSS

SEQ ID NO: 52 - protein, 111 aa, Artificial Sequence
  RVB185 light chain aa
QSVLTQPPSASGTPGQRVTISCSGSRSNIGSHPVNWYQQLPGAAPKLLIYGDSQRPSGVPDRFSGSKSGPSASLAISGLQSEDEADYYCAAWDDSLSGLWVFGGGTKLTVL

SEQ ID NO: 53 - DNA, 358 bp, Artificial Sequence
  RVB185 heavy chain nuc
caggtgcagctgcaggagtcgggcccaggactggtgaagccttcggagaccctgtccctcacctgcagtgtctccggtgcccccgtcagtggtgttaactcctactgggtgtggatccgccagccccccgggaaggggctggagtggattgcgactatcaagtacagtgggagcacccaccgtagcccgtcgctcaggagtcgagtcaccatatccgtagacacgtccaagaatcagttctccctggagctgagctctgtgaccgccgctgacacggctgtatattactgtgccagacaaagtactatgacgggccgggactactggggccagggaaccctggtcaccgtctcctcag

SEQ ID NO: 54 - DNA, 334 bp, Artificial Sequence
  RVB185 light chain nuc
cagtctgtgctgactcagccaccctcagcgtctgggacccccgggcagagggtcaccatctcttgttctggaagcagatccaacatcggaagccatcctgtaaactggtaccagcagctcccgggagcggcccccaagctcctcatctatggtgatagtcagcgaccctcaggggtccctgaccgattctctggctccaagtctggcccctcagcctccctggccatcagtggactccagtctgaagatgaggctgattattactgtgcagcatgggatgacagcctgagtggcctttgggtgttcggcggagggaccaagctgaccgtcctaa

SEQ ID NO: 55 - protein, 8 aa, Artificial Sequence
  RVB492 CDRH1 aa
GFSFSSYA

SEQ ID NO: 56 - protein, 8 aa, Artificial Sequence
  RVB492 CDRH2 aa
LNSIDHRT

SEQ ID NO: 57 - protein, 18 aa, Artificial Sequence
  RVB492 CDRH3 aa
ARGVGLWFGELSWNYFDY

SEQ ID NO: 58 - protein, 9 aa, Artificial Sequence
  RVB492 CDRL1 aa
SNDIGGYNY

SEQ ID NO: 59 - skipped

SEQ ID NO: 60 - protein, 9 aa, Artificial Sequence
  RVB492 CDRL2 long aa
MIFYVNKRP

SEQ ID NO: 61 - protein, 9 aa, Artificial Sequence
  RVB492 CDRL3 aa
CSFAGSYSL

SEQ ID NO: 62 - DNA, 24 bp, Artificial Sequence
  RVB492 CDRH1 nuc
ggattcagctttagcagctatgcc

SEQ ID NO: 63 - DNA, 24 bp, Artificial Sequence
  RVB492 CDRH2 nuc
cttaattctattgatcatagaaca

SEQ ID NO: 64 - DNA, 54 bp, Artificial Sequence
  RVB492 CDRH3 nuc
gctcggggggtgggactatggttcggtgaattatcctggaattactttgactac

SEQ ID NO: 65 - DNA, 27 bp, Artificial Sequence
  RVB492 CDRL1 nuc
agcaatgatattggtggttataactat

SEQ ID NO: 66 - skipped

SEQ ID NO: 67 - DNA, 27 bp, Artificial Sequence
  RVB492 CDRL2 long nuc
atgattttttatgtcaataagcggccc

SEQ ID NO: 68 - DNA, 27 bp, Artificial Sequence
  RVB492 CDRL3 nuc
tgctcatttgcaggcagttactcctta

SEQ ID NO: 69 - protein, 125 aa, Artificial Sequence
  RVB492 heavy chain variant 1 aa
EVQLMESGGGLVQPGGSMRLYCAASGFSFSSYAMSWVRQAPGKGLEWVSSLNSIDHRTDYADSVKGRFTISRDNSKNTLYLQMDSLRVEDSAMYYCARGVGLWFGELSWNYFDYWGQGTLVTVSS

SEQ ID NO: 70 - protein, 125 aa, Artificial Sequence
  RVB492 heavy chain variant 2 aa
EVQLVQSGGGLVQPGGSMRLYCAASGFSFSSYAMSWVRQAPGKGLEWVSSLNSIDHRTDYADSVKGRFTISRDNSKNTLYLQMDSLRVEDSAMYYCARGVGLWFGELSWNYFDYWGQGTLVTVSS

SEQ ID NO: 71 - protein, 109 aa, Artificial Sequence
  RVB492 light chain aa
QSALTQPRSVSGSPGQSVTISCTGTSNDIGGYNYVSWYQQHPGKAPKLMIFYVNKRPSGVPDRFSGSKSGNTASLTISGLQAEDEADYYCCSFAGSYSLFGRGTKLTVL

SEQ ID NO: 72 - DNA, 376 bp, Artificial Sequence
  RVB492 heavy chain variant 1 nuc
gaggtgcagctgatggagtctgggggaggcctggtacagccgggggggtccatgagactctactgtgcagcctctggattcagctttagcagctatgccatgagctgggtccgccaggctccagggaaggggctcgagtgggtctcaagtcttaattctattgatcatagaacagactatgcagactccgtgaagggccggttcaccatctccagagacaattccaagaacaccctgtatttacaaatggacagcctgagagtcgaggactcggccatgtattactgtgctcggggggtgggactatggttcggtgaattatcctggaattactttgactactggggccagggaaccctggtcaccgtctcctcag

SEQ ID NO: 73 - DNA, 376 bp, Artificial Sequence
  RVB492 heavy chain variant 2 nuc
gaggtgcagctggtgcagtctgggggaggcctggtacagccgggggggtccatgagactctactgtgcagcctctggattcagctttagcagctatgccatgagctgggtccgccaggctccagggaaggggctcgagtgggtctcaagtcttaattctattgatcatagaacagactatgcagactccgtgaagggccggttcaccatctccagagacaattccaagaacaccctgtatttacaaatggacagcctgagagtcgaggactcggccatgtattactgtgctcggggggtgggactatggttcggtgaattatcctggaattactttgactactggggccagggaaccctggtcaccgtctcctcag

SEQ ID NO: 74 - DNA, 328 bp, Artificial Sequence
  RVB492 light chain nuc
cagtctgccctgactcagcctcgctcagtgtccgggtctcctggacagtcagtcaccatctcctgcactggaaccagcaatgatattggtggttataactatgtctcctggtaccaacaacacccaggcaaagcccccaaactcatgattttttatgtcaataagcggccctcaggggtccctgatcgcttctctggctccaagtctggcaacacggcctccctgaccatctctgggctccaggctgaggatgaagctgattattactgctgctcatttgcaggcagttactccttattcggcagagggaccaagctgaccgtcctag

SEQ ID NO: 75 - protein, 8 aa, Artificial Sequence
  RVC3 CDRH1 aa
TFTFRNYA

SEQ ID NO: 76 - protein, 8 aa, Artificial Sequence
  RVC3 CDRH2 aa
ISASGSST

SEQ ID NO: 77 - protein, 16 aa, Artificial Sequence
  RVC3 CDRH3 aa
AKFAHDFWSGYSYFDS

SEQ ID NO: 78 - protein, 6 aa, Artificial Sequence
  RVC3 CDRL1 aa
QSVNSN

SEQ ID NO: 79 - skipped

SEQ ID NO: 80 - protein, 9 aa, Artificial Sequence
  RVC3 CDRL2 long aa
LIYGASTRA

SEQ ID NO: 81 - protein, 10 aa, Artificial Sequence
  RVC3 CDRL3 aa
QQYNNWVSIT

SEQ ID NO: 82 - DNA, 24 bp, Artificial Sequence
  RVC3 CDRH1 nuc
acattcacgtttagaaactatgcc

SEQ ID NO: 83 - DNA, 24 bp, Artificial Sequence
  RVC3 CDRH2 nuc
attagtgctagtggtagtagcacg

SEQ ID NO: 84 - DNA, 48 bp, Artificial Sequence
  RVC3 CDRH3 nuc
gcgaaatttgctcacgatttttggagtggttattcttactttgactcc

SEQ ID NO: 85 - DNA, 18 bp, Artificial Sequence
  RVC3 CDRL1 nuc
cagagtgttaacagcaac

SEQ ID NO: 86 - skipped

SEQ ID NO: 87 - DNA, 27 bp, Artificial Sequence
  RVC3 CDRL2 long nuc
ctcatctatggtgcatccaccagggcc

SEQ ID NO: 88 - DNA, 30 bp, Artificial Sequence
  RVC3 CDRL3 nuc
cagcagtataataattgggtttcgatcacc

SEQ ID NO: 89 - protein, 123 aa, Artificial Sequence
  RVC3 heavy chain aa
EVQLLESGGGLVQPGGSLRLSCAASTFTFRNYAMSWVRQAPGKGLDWVSGISASGSSTNYAASLKGRFTISRDNSKNTLYLQMNSLRAEDTAVYYCAKFAHDFWSGYSYFDSWGQGTLVTVSS

SEQ ID NO: 90 - protein, 108 aa, Artificial Sequence
  RVC3 light chain aa
EIVMTQSPATLSVSPGERATLSCRAGQSVNSNLAWYQQKPGQAPRLLIYGASTRATGIPARFSGSGSGTEFTLTISSLQSEDFAVYYCQQYNNWVSITFGQGTRLEIK

SEQ ID NO: 91 - DNA, 370 bp, Artificial Sequence
  RVC3 heavy chain nuc
gaggtgcagctgttggagtctgggggaggcctggtgcagccgggggggtccctgagactctcctgtgcagcctctacattcacgtttagaaactatgccatgtcctgggtccgccaggctccagggaaggggctggactgggtctcagggattagtgctagtggtagtagcacgaattatgcagcctccctgaagggccgatttaccatctccagagacaattccaagaacacattgtatctgcaaatgaacagcctgagagccgaggacacggccgtctattactgtgcgaaatttgctcacgatttttggagtggttattcttactttgactcctggggccagggaaccctggtcaccgtctcctcag

SEQ ID NO: 92 - DNA, 325 bp, Artificial Sequence
  RVC3 light chain nuc
gaaatagtgatgacgcagtctccagccaccctgtctgtgtctccaggggaaagagccaccctctcctgcagggccggtcagagtgttaacagcaacttagcctggtaccagcagaaacctgggcaggctcccagactcctcatctatggtgcatccaccagggccactggtatcccagccaggttcagtggcagtgggtctgggacagagttcactctcaccatcagcagcctgcagtctgaagattttgcagtttattactgtcagcagtataataattgggtttcgatcaccttcggccaagggacacgactggagattaaac

SEQ ID NO: 93 - protein, 10 aa, Artificial Sequence
  RVC20 CDRH1 aa
GGSFSSGSYS

SEQ ID NO: 94 - protein, 7 aa, Artificial Sequence
  RVC20 CDRH2 aa
IYYSGST

SEQ ID NO: 95 - protein, 16 aa, Artificial Sequence
  RVC20 CDRH3 aa
ARGTYSDFWSGSPLDY

SEQ ID NO: 96 - protein, 6 aa, Artificial Sequence
  RVC20 CDRL1 aa
QGISNY

SEQ ID NO: 97 - skipped

SEQ ID NO: 98 - protein, 9 aa, Artificial Sequence
  RVC20 CDRL2 long aa
LIYAASSLQ

SEQ ID NO: 99 - protein, 9 aa, Artificial Sequence
  RVC20 CDRL3 aa
QQYDTYPLT

SEQ ID NO: 100 - DNA, 30 bp, Artificial Sequence
  RVC20 CDRH1 nuc
ggtggctccttcagcagtggaagttactcc

SEQ ID NO: 101 - DNA, 21 bp, Artificial Sequence
  RVC20 CDRH2 nuc
atctattacagtgggagcact

SEQ ID NO: 102 - DNA, 48 bp, Artificial Sequence
  RVC20 CDRH3 nuc
gcgagaggcacgtattccgatttttggagtggttcccctttagactac

SEQ ID NO: 103 - DNA, 18 bp, Artificial Sequence
  RVC20 CDRL1 nuc
cagggcattagcaattat

SEQ ID NO: 104 - skipped

SEQ ID NO: 105 - DNA, 27 bp, Artificial Sequence
  RVC20 CDRL2 long nuc
ctgatctatgctgcatccagtttgcaa

SEQ ID NO: 106 - DNA, 27 bp, Artificial Sequence
  RVC20 CDRL3 nuc
caacagtatgatacttaccctctcact

SEQ ID NO: 107 - protein, 124 aa, Artificial Sequence
  RVC20 heavy chain aa
QVQLQESGPGLVKPSQTLSLTCTVSGGSFSSGSYSWNWIRQHPGKGLEWIGYIYYSGSTYYNPSLKSRVTMSVHTSKNQFSLKLNSITAADTAVYYCARGTYSDFWSGSPLDYWGQGTLVTVSS

SEQ ID NO: 108 - protein, 107 aa, Artificial Sequence
  RVC20 light chain aa
DIQMTQSPSSLSASVGDRVTITCRASQGISNYLAWFQQKPGKAPKSLIYAASSLQSGVPSRFSGSGSGTDFTLTINSLQPEDFVTYFCQQYDTYPLTFGGGTKVEIK

SEQ ID NO: 109 - DNA, 373 bp, Artificial Sequence
  RVC20 heavy chain nuc
caggtgcagctgcaggagtcgggcccaggactggtgaagccttcacagaccctgtccctcacctgcactgtctccggtggctccttcagcagtggaagttactcctggaactggatccgccagcacccagggaagggcctggagtggattgggtacatctattacagtgggagcacttattacaacccgtccctcaagagtcgagttaccatgtcagtacacacgtctaagaaccagttctccctgaagctgaactctataactgccgcggacacggccgtgtattactgtgcgagaggcacgtattccgatttttggagtggttcccctttagactactggggccagggaaccctggtcaccgtctcctcag

SEQ ID NO: 110 - DNA, 322 bp, Artificial Sequence
  RVC20 light chain nuc
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaggttcagcggcagtggatctgggacagatttcactctcaccatcaacagcctgcagcctgaagattttgtaacttatttctgccaacagtatgatacttaccctctcactttcggcggagggaccaaggtggagatcaaac

SEQ ID NO: 111 - protein, 10 aa, Artificial Sequence
  RVC21 CDRH1 aa
GGSISNPNYY

SEQ ID NO: 112 - protein, 7 aa, Artificial Sequence
  RVC21 CDRH2 aa
IYYNGYT

SEQ ID NO: 113 - protein, 13 aa, Artificial Sequence
  RVC21 CDRH3 aa
ATQSTMTTIAGHY

SEQ ID NO: 114 - protein, 8 aa, Artificial Sequence
  RVC21 CDRL1 aa
TSNIGNSY

SEQ ID NO: 115 - skipped

SEQ ID NO: 116 - protein, 9 aa, Artificial Sequence
  RVC21 CDRL2 long aa
LIYDNNKRP

SEQ ID NO: 117 - protein, 11 aa, Artificial Sequence
  RVC21 CDRL3 aa
GTWDSSLNAYV

SEQ ID NO: 118 - DNA, 30 bp, Artificial Sequence
  RVC21 CDRH1 nuc
ggtggctccatcagcaaccctaactactac

SEQ ID NO: 119 - DNA, 21 bp, Artificial Sequence
  RVC21 CDRH2 nuc
atctattataatgggtacacc

SEQ ID NO: 120 - DNA, 39 bp, Artificial Sequence
  RVC21 CDRH3 nuc
gcgacgcaatctacgatgactaccatagcgggccactac

SEQ ID NO: 121 - DNA, 24 bp, Artificial Sequence
  RVC21 CDRL1 nuc
acatccaacattgggaattcttat

SEQ ID NO: 122 - skipped

SEQ ID NO: 123 - DNA, 27 bp, Artificial Sequence
  RVC21 CDRL2 long nuc
ctcatttatgacaataataagcgaccc

SEQ ID NO: 124 - DNA, 33 bp, Artificial Sequence
  RVC21 CDRL3 nuc
ggaacatgggacagcagcctgaatgcttatgtc

SEQ ID NO: 125 - protein, 121 aa, Artificial Sequence
  RVC21 heavy chain aa
QLQLQESGPGLVKPSETLSLTCTVSGGSISNPNYYWGWIRQPPGKGLEWIGSIYYNGYTYYNPSLKSRVTISVDKSKDQFFLKMTSLTAADTAVYYCATQSTMTTIAGHYWGQGTLVTVSS

SEQ ID NO: 126 - protein, 110 aa, Artificial Sequence
  RVC21 light chain aa
QSVLTQAPSVSAAPGLKVTISCSGSTSNIGNSYVSWYQQLPGTAPKLLIYDNNKRPSGIPDRFSGSKSDTSATLGITGLQTGDEADYYCGTWDSSLNAYVFGTGTKVTVL

SEQ ID NO: 127 - DNA, 364 bp, Artificial Sequence
  RVC21 heavy chain nuc
cagctgcagctgcaggagtcgggcccaggactggtgaagccttcggagaccctgtccctcacgtgcactgtctctggtggctccatcagcaaccctaactactactggggctggatccgccagcccccagggaagggtctggaatggattgggagtatctattataatgggtacacctactacaacccgtccctcaagagtcgagttaccatatccgtggacaagtccaaggaccagttctttctgaagatgacctctctgaccgccgcagacacggctgtgtattactgtgcgacgcaatctacgatgactaccatagcgggccactactggggccagggaaccctggtcaccgtctcctcag

SEQ ID NO: 128 - DNA, 331 bp, Artificial Sequence
  RVC21 light chain nuc
cagtctgtattgacgcaggcgccctcagtgtctgcggccccaggactaaaggtcaccatctcctgctctggaagcacatccaacattgggaattcttatgtatcctggtaccagcagctcccaggaacagcccccaaactcctcatttatgacaataataagcgaccctcagggattcctgaccgattctctggctccaagtctgacacgtcagccaccctgggcatcaccggactccagactggggacgaggccgattattactgcggaacatgggacagcagcctgaatgcttatgtcttcggaactgggaccaaggtcaccgtcctag

SEQ ID NO: 129 - protein, 8 aa, Artificial Sequence
  RVC38 CDRH1 aa
GGTFSSYA

SEQ ID NO: 130 - protein, 8 aa, Artificial Sequence
  RVC38 CDRH2 aa
IMPMFVAA

SEQ ID NO: 131 - protein, 14 aa, Artificial Sequence
  RVC38 CDRH3 aa
ARGDGYNYKWYFDL

SEQ ID NO: 132 - protein, 6 aa, Artificial Sequence
  RVC38 CDRL1 aa
QDISNY

SEQ ID NO: 133 - skipped

SEQ ID NO: 134 - protein, 9 aa, Artificial Sequence
  RVC38 CDRL2 long aa
LIYAASTLQ

SEQ ID NO: 135 - protein, 10 aa, Artificial Sequence
  RVC38 CDRL3 aa
QQLDTYVALT

SEQ ID NO: 136 - DNA, 24 bp, Artificial Sequence
  RVC38 CDRH1 nuc
ggaggcaccttcagcagctatgcc

SEQ ID NO: 137 - DNA, 24 bp, Artificial Sequence
  RVC38 CDRH2 nuc
atcatgcctatgtttgtggcggca

SEQ ID NO: 138 - DNA, 42 bp, Artificial Sequence
  RVC38 CDRH3 nuc
gcgagaggggatggctacaattacaagtggtattttgacctt

SEQ ID NO: 139 - DNA, 18 bp, Artificial Sequence
  RVC38 CDRL1 nuc
caggacattagtaattat

SEQ ID NO: 140 - skipped

SEQ ID NO: 141 - DNA, 27 bp, Artificial Sequence
  RVC38 CDRL2 long nuc
ctgatctatgctgcatccactttgcaa

SEQ ID NO: 142 - DNA, 30 bp, Artificial Sequence
  RVC38 CDRL3 nuc
caacagcttgatacttacgtcgcgctcact

SEQ ID NO: 143 - protein, 121 aa, Artificial Sequence
  RVC38 heavy chain aa
EVQLVQSGAEVKKPGSSVRVSCKASGGTFSSYAISWVRQAPGLGLEWMGGIMPMFVAANYAQNFQGRVTVSVDKSTNTAYMEMHNLRSDDTAMYYCARGDGYNYKWYFDLWGQGTLVTVSS

SEQ ID NO: 144 - protein, 108 aa, Artificial Sequence
  RVC38 light chain aa
DIQLTQSPSFLSASVGDRVTITCRASQDISNYLAWYQQKPGKPPKLLIYAASTLQRGVPSRFSGSGSGSEFTLTISSLQPEDFATYYCQQLDTYVALTFGGGTKVEIK

SEQ ID NO: 145 - DNA, 364 bp, Artificial Sequence
  RVC38 heavy chain nuc
gaggtgcagctggtgcagtctggggctgaggtgaagaagcctgggtcctcggtgagggtctcctgcaaggcttctggaggcaccttcagcagctatgccatcagctgggtgcgacaggcccctgggctagggcttgagtggatgggagggatcatgcctatgtttgtggcggcaaactacgcacagaacttccagggcagagtcacggtttctgtggacaaatccacgaacaccgcctatatggagatgcacaacctgagatctgacgacacggccatgtattactgtgcgagaggggatggctacaattacaagtggtattttgacctttggggccagggaaccctagtcaccgtctcctcag

SEQ ID NO: 146 - DNA, 325 bp, Artificial Sequence
  RVC38 light chain nuc
gacatccagttgacccagtctccatccttcctgtctgcatctgtaggagacagagtcaccatcacttgccgggccagtcaggacattagtaattatttagcctggtatcagcaaaaaccagggaagccccctaaactcctgatctatgctgcatccactttgcaaaggggggtcccatcaaggttcagtggcagtggatctgggtcagaattcactctcacaatcagcagcctgcagcctgaagattttgcaacttattactgtcaacagcttgatacttacgtcgcgctcactttcggcggagggaccaaggtggagatcaaac

SEQ ID NO: 147 - protein, 8 aa, Artificial Sequence
  RVC44 CDRH1 aa
GFTFSSYS

SEQ ID NO: 148 - protein, 8 aa, Artificial Sequence
  RVC44 CDRH2 aa
ISTTGTYI

SEQ ID NO: 149 - protein, 17 aa, Artificial Sequence
  RVC44 CDRH3 aa
ARRSAIALAGTQRAFDI

SEQ ID NO: 150 - protein, 6 aa, Artificial Sequence
  RVC44 CDRL1 aa
QNINNY

SEQ ID NO: 151 - skipped

SEQ ID NO: 152 - protein, 9 aa, Artificial Sequence
  RVC44 CDRL2 long aa
LIYAASSLH

SEQ ID NO: 153 - protein, 9 aa, Artificial Sequence
  RVC44 CDRL3 aa
QQSYSNPWT

SEQ ID NO: 154 - DNA, 24 bp, Artificial Sequence
  RVC44 CDRH1 nuc
ggcttcacctttagtagttatagt

SEQ ID NO: 155 - DNA, 24 bp, Artificial Sequence
  RVC44 CDRH2 nuc
atcagtactactggtacttacata

SEQ ID NO: 156 - DNA, 51 bp, Artificial Sequence
  RVC44 CDRH3 nuc
gcgagacggtcggccatagcactggctggtacgcagcgtgcttttgatatc

SEQ ID NO: 157 - DNA, 18 bp, Artificial Sequence
  RVC44 CDRL1 nuc
cagaacattaacaactat

SEQ ID NO: 158 - skipped

SEQ ID NO: 159 - DNA, 27 bp, Artificial Sequence
  RVC44 CDRL2 long nuc
ctgatctatgctgcatccagtttacat

SEQ ID NO: 160 - DNA, 27 bp, Artificial Sequence
  RVC44 CDRL3 nuc
caacagagttacagtaacccttggacg

SEQ ID NO: 161 - protein, 124 aa, Artificial Sequence
  RVC44 heavy chain aa
EVQLVQSGGGLVKPGGSLRLSCAASGFTFSSYSMSWVRQAPGKGLEWVSSISTTGTYIYYADSVEGRFSISRDSARSSLFLQMNSLRAEDTAVYYCARRSAIALAGTQRAFDIWGPGTNVIVSS

SEQ ID NO: 162 - protein, 107 aa, Artificial Sequence
  RVC44 light chain aa
DIQMTQSPSSLSASVGDRVTITCRASQNINNYLNWYQQKLGKAPKLLIYAASSLHSGVPSRFSASGSGTDFILTISNLQPEDCATYYCQQSYSNPWTFGQGTKVEIK

SEQ ID NO: 163 - DNA, 373 bp, Artificial Sequence
  RVC44 heavy chain nuc
gaggtgcagctggtgcagtctgggggaggcctggtcaagcctggggggtccctgagactctcctgtgcagcctctggcttcacctttagtagttatagtatgagttgggtccgccaggctccagggaagggcctggagtgggtctcatccatcagtactactggtacttacatatactacgcagactcagtggagggccgattctccatttccagagacagcgccaggagctctctgtttctgcaaatgaacagcctgagagccgaggacacggctgtctattactgtgcgagacggtcggccatagcactggctggtacgcagcgtgcttttgatatctggggcccagggacaaacgtcatcgtctcttcag

SEQ ID NO: 164 - DNA, 322 bp, Artificial Sequence
  RVC44 light chain nuc
gacatccagatgacccagtctccatcttccctgtctgcatctgtaggagacagagtcaccatcacttgccgggcaagtcagaacattaacaactatttaaattggtatcagcagaaactagggaaagcccctaagctcctgatctatgctgcatccagtttacatagtggggtcccatcaaggttcagtgccagtggatctgggacagatttcattctgaccatcagtaatctgcaacctgaagattgtgcaacttactactgtcaacagagttacagtaacccttggacgttcggccaagggaccaaggtggaaatcaaac

SEQ ID NO: 165 - protein, 8 aa, Artificial Sequence
  RVC58 CDRH1 aa
GFTFSTYA

SEQ ID NO: 166 - protein, 8 aa, Artificial Sequence
  RVC58 CDRH2 aa
ISDRGGSR

SEQ ID NO: 167 - protein, 17 aa, Artificial Sequence
  RVC58 CDRH3 aa
ARDIAPPYNYYFYGMDV

SEQ ID NO: 168 - protein, 9 aa, Artificial Sequence
  RVC58 CDRL1 aa
SSDIGAFNY

SEQ ID NO: 169 - skipped

SEQ ID NO: 170 - protein, 9 aa, Artificial Sequence
  RVC58 CDRL2 long aa
IIYEVSNRP

SEQ ID NO: 171 - protein, 10 aa, Artificial Sequence
  RVC58 CDRL3 aa
NSYTSSSTQL

SEQ ID NO: 172 - DNA, 24 bp, Artificial Sequence
  RVC58 CDRH1 nuc
ggattcacctttagcacctatgcc

SEQ ID NO: 173 - DNA, 24 bp, Artificial Sequence
  RVC58 CDRH2 nuc
attagtgatagaggtggtagtaga

SEQ ID NO: 174 - DNA, 51 bp, Artificial Sequence
  RVC58 CDRH3 nuc
gcgagagatattgcccccccatataactactacttctacggtatggacgtc

SEQ ID NO: 175 - DNA, 27 bp, Artificial Sequence
  RVC58 CDRL1 nuc
agcagtgacattggtgcttttaactat

SEQ ID NO: 176 - skipped

SEQ ID NO: 177 - DNA, 27 bp, Artificial Sequence
  RVC58 CDRL2 long nuc
ataatttatgaggtcagtaatcggccc

SEQ ID NO: 178 - DNA, 30 bp, Artificial Sequence
  RVC58 CDRL3 nuc
aactcatatacaagcagcagcactcagtta

SEQ ID NO: 179 - protein, 124 aa, Artificial Sequence
  RVC58 heavy chain aa
EVQLVESGGGLVQPGGSLRLSCAASGFTFSTYAMNWVRQAPGKGLEWVSGISDRGGSRYYAGSVKGRFTISRDNSKNTLFLQMNSLRAEDTAVYYCARDIAPPYNYYFYGMDVWGRGTTVTVSS

SEQ ID NO: 180 - protein, 110 aa, Artificial Sequence
  RVC58 light chain aa
QSALTQPASVSGSPGQSITISCTGTSSDIGAFNYVSWYQQHPGKAPKLIIYEVSNRPSGVSNRFSGSKSGNTASLTISGLQAEDEADYYCNSYTSSSTQLFGGGTKLTVL

SEQ ID NO: 181 - DNA, 373 bp, Artificial Sequence
  RVC58 heavy chain nuc
gaggtgcagctggtggagtctgggggaggcttggtacagcctggggggtccctgagactctcctgtgcggcctctggattcacctttagcacctatgccatgaattgggtccgccaggctccagggaaggggctggagtgggtctcaggtattagtgatagaggtggtagtagatactacgcaggctccgtgaagggccggttcaccatctccagagacaattccaagaacacgctgtttctgcaaatgaacagcctgagagccgaggacacggccgtatattactgtgcgagagatattgcccccccatataactactacttctacggtatggacgtctggggccgagggaccacggtcaccgtctcctcag

SEQ ID NO: 182 - DNA, 331 bp, Artificial Sequence
  RVC58 light chain nuc
cagtctgccctgactcagcctgcctccgtgtctgggtctcctggacagtcgatcaccatctcctgcactggtaccagcagtgacattggtgcttttaactatgtctcttggtaccaacagcacccaggcaaagcccccaaactcataatttatgaggtcagtaatcggccctcaggggtttctaatcgcttctctggctccaagtctggcaacacggcctccctgaccatctctgggctccaggctgaggacgaggctgattattactgcaactcatatacaagcagcagcactcagttattcggcggagggaccaagctgaccgtcctag

SEQ ID NO: 183 - protein, 8 aa, Artificial Sequence
  RVC68 CDRH1 aa
GGSISEHH

SEQ ID NO: 184 - protein, 7 aa, Artificial Sequence
  RVC68 CDRH2 aa
IFHSGST

SEQ ID NO: 185 - protein, 14 aa, Artificial Sequence
  RVC68 CDRH3 aa
ARAVSTYYYYYIDV

SEQ ID NO: 186 - protein, 6 aa, Artificial Sequence
  RVC68 CDRL1 aa
QDISNW

SEQ ID NO: 187 - skipped

SEQ ID NO: 188 - protein, 9 aa, Artificial Sequence
  RVC68 CDRL2 long aa
LIYAASSLQ

SEQ ID NO: 189 - protein, 9 aa, Artificial Sequence
  RVC68 CDRL3 aa
QQAKSFPLT

SEQ ID NO: 190 - DNA, 24 bp, Artificial Sequence
  RVC68 CDRH1 nuc
ggtggctccattagtgagcaccac

SEQ ID NO: 191 - DNA, 21 bp, Artificial Sequence
  RVC68 CDRH2 nuc
atctttcacagtgggagtacc

SEQ ID NO: 192 - DNA, 42 bp, Artificial Sequence
  RVC68 CDRH3 nuc
gcgagagcggtgtctacttactactactattacatagacgtc

SEQ ID NO: 193 - DNA, 18 bp, Artificial Sequence
  RVC68 CDRL1 nuc
caggatattagcaactgg

SEQ ID NO: 194 - skipped

SEQ ID NO: 195 - DNA, 27 bp, Artificial Sequence
  RVC68 CDRL2 long nuc
ctgatctatgctgcgtccagtttgcaa

SEQ ID NO: 196 - DNA, 27 bp, Artificial Sequence
  RVC68 CDRL3 nuc
caacaggctaagagtttccctcttact

SEQ ID NO: 197 - protein, 120 aa, Artificial Sequence
  RVC68 heavy chain aa
QVQLQESGPRLVKPSETLSLTCTFSGGSISEHHWSWLRQSPGKGLEWIGYIFHSGSTNYNPSLKSRVNISLDKSKNQFSLKLSSVTAADTAVYFCARAVSTYYYYYIDVWGQGTTVTVSS

SEQ ID NO: 198 - protein, 107 aa, Artificial Sequence
  RVC68 light chain aa
DIQMTQSPSSVSASVGDRVTITCRASQDISNWLAWYQQKPGKAPKLLIYAASSLQSGISSRFSGGGSGTDFTLTISSLQPEDFASYYCQQAKSFPLTFGQGTKLEIK

SEQ ID NO: 199 - DNA, 361 bp, Artificial Sequence
  RVC68 heavy chain nuc
caggtgcagctacaggagtcgggcccaagactggtgaagccctcggagaccctgtccctcacctgcactttctctggtggctccattagtgagcaccactggagctggctccggcagtccccagggaagggactggagtggattggatatatctttcacagtgggagtaccaactacaacccctccctcaagagtcgagtcaacatatcattagacaagtccaagaaccagttctccctgaagctgagttctgtgaccgctgcggacacggccgtgtatttctgtgcgagagcggtgtctacttactactactattacatagacgtctggggccaagggaccacggtcaccgtctcctcag

SEQ ID NO: 200 - DNA, 322 bp, Artificial Sequence
  RVC68 light chain nuc
gacatccagatgacccagtctccatcttccgtgtctgcatctgtaggagacagagtcaccatcacttgtcgggcgagtcaggatattagcaactggttagcctggtatcagcagaaaccagggaaagcccctaaactcctgatctatgctgcgtccagtttgcaaagtgggatctcatctaggttcagcggcggtggctctgggacagatttcactctcaccatcagcagcctgcagcctgaagattttgcaagttactactgtcaacaggctaagagtttccctcttacttttggccaggggaccaagctggagatcaaac

SEQ ID NO: 201 - protein, 8 aa, Artificial Sequence
  RVC111 CDRH1 aa
GFSFSSYV

SEQ ID NO: 202 - protein, 8 aa, Artificial Sequence
  RVC111 CDRH2 aa
ISYDGSNK

SEQ ID NO: 203 - protein, 13 aa, Artificial Sequence
  RVC111 CDRH3 aa
ARGSGTQTPLFDY

SEQ ID NO: 204 - protein, 6 aa, Artificial Sequence
  RVC111 CDRL1 aa
QSITSW

SEQ ID NO: 205 - skipped

SEQ ID NO: 206 - protein, 9 aa, Artificial Sequence
  RVC111 CDRL2 long aa
LIYDDSTLE

SEQ ID NO: 207 - protein, 9 aa, Artificial Sequence
  RVC111 CDRL3 aa
QQYESYSGT

SEQ ID NO: 208 - DNA, 24 bp, Artificial Sequence
  RVC111 CDRH1 nuc
ggattctccttcagtagctatgtt

SEQ ID NO: 209 - DNA, 24 bp, Artificial Sequence
  RVC111 CDRH2 nuc
atatcatatgatggaagtaataaa

SEQ ID NO: 210 - DNA, 39 bp, Artificial Sequence
  RVC111 CDRH3 nuc
gcgagagggtccggaacccaaactcccctctttgactac

SEQ ID NO: 211 - DNA, 18 bp, Artificial Sequence
  RVC111 CDRL1 nuc
cagagtattactagctgg

SEQ ID NO: 212 - skipped

SEQ ID NO: 213 - DNA, 27 bp, Artificial Sequence
  RVC111 CDRL2 long nuc
ctgatctatgatgactccactttggaa

SEQ ID NO: 214 - DNA, 27 bp, Artificial Sequence
  RVC111 CDRL3 nuc
caacagtatgagagttattcagggacg

SEQ ID NO: 215 - protein, 120 aa, Artificial Sequence
  RVC111 heavy chain aa
QVQLVESGGGVVQPGRSLRLSCAASGFSFSSYVMYWVRQAPGKGLEWVTIISYDGSNKYYADSVKGRFTISRDNSKNTLYLQMNSLRAEDTAVYYCARGSGTQTPLFDYWGQGTLVTVSS

SEQ ID NO: 216 - protein, 107 aa, Artificial Sequence
  RVC111 light chain aa
DIQMTQSPSTLSASVGDRVTITCRANQSITSWVAWYQQMPGRAPKLLIYDDSTLESGVPSRFSGSGSGTEFTLTISSLQPDDFATYYCQQYESYSGTFGQGTKVEIK

SEQ ID NO: 217 - DNA, 361 bp, Artificial Sequence
  RVC111 heavy chain nuc
caggtgcaactggtggagtctgggggaggcgtggtccagcctgggaggtccctgagactctcctgtgcagcctctggattctccttcagtagctatgttatgtactgggtccgccaggctccaggcaaggggctggagtgggtgacaattatatcatatgatggaagtaataaatactacgcagactccgtgaagggccgattcaccatctccagagacaattccaagaacacgctgtatctgcaaatgaacagcctgagagctgaggacacggctgtctattactgtgcgagagggtccggaacccaaactcccctctttgactactggggccagggaaccctggtcaccgtctcctcag

SEQ ID NO: 218 - DNA, 322 bp, Artificial Sequence
  RVC111 light chain nuc
gacatccagatgacccagtctccttccaccctgtctgcatctgtgggagacagagtcaccatcacttgccgggccaatcagagtattactagctgggtggcctggtatcagcagatgccagggagagcccctaaactcctgatctatgatgactccactttggaaagtggggtcccatcaaggttcagcggcagtggatctgggacagaattcactctcaccatcagcagcctgcagcctgatgattttgcaacttattactgccaacagtatgagagttattcagggacgttcggccaagggaccaaggtggaaatcaaac

SEQ ID NO: 219 - protein, 330 aa, Artificial Sequence
  IgG1 CH1-CH2-CH3 LALA aa
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 107 aa, Artificial Sequence [sequence omitted]

SEQ ID NO: 221 - protein, 106 aa, Artificial Sequence
  IgG CL aa
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 222 - DNA, 990 bp, Artificial Sequence
  IgG1 CH1-CH2-CH3 LALA nucl
gcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 223 - DNA, 321 bp, Artificial Sequence
  IgG CK nucl
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 224 - DNA, 318 bp, Artificial Sequence
  IgG CL nucl
ggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 225 - DNA, 20 bp, Artificial Sequence
  Primer
ctgacgtagcactggcagac

SEQ ID NO: 226 - DNA, 20 bp, Artificial Sequence
  Primer
agtcgacctccgttcatcat